SEQUENCE LISTING (214 sequences)

SEQ ID NO: 1 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 5 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 6 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 7 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 8 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 227 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 227 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 14 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 15 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 16 - protein, 208 aa, Homo sapiens [sequence omitted]

SEQ ID NO: 17 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 18 - protein, 123 aa, Homo sapiens
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 19 - protein, 100 aa, Homo sapiens
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYR

SEQ ID NO: 20 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 21 - protein, 110 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVL

SEQ ID NO: 22 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 23 - protein, 174 aa, Homo sapiens
PAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 24 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 25 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 26 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 27 - protein, 133 aa, Homo sapiens [sequence omitted]

SEQ ID NO: 28 - protein, 251 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 29 - protein, 524 aa, Homo sapiens
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 30 - protein, 347 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 31 - protein, 9 aa, synthetic construct
  REGION 1..9: note = Epitope tag
YPYDVPDYA

SEQ ID NO: 32 - protein, 8 aa, synthetic construct
  REGION 1..8: note = Epitopetag
DYKDDDDK

SEQ ID NO: 33 - protein, 10 aa, synthetic construct
  REGION 1..10: note = Epitope tag
EQKLISEEDL

SEQ ID NO: 34 - protein, 5 aa, synthetic construct
  REGION 1..5: note = 5X HIS TAG
HHHHH

SEQ ID NO: 35 - protein, 6 aa, synthetic construct
  REGION 1..6: note = 6X HIS TAG
HHHHHH

SEQ ID NO: 36 - protein, 8 aa, synthetic construct
  REGION 1..8: note = STREP TAG
WSHPQFEK

SEQ ID NO: 37 - protein, 5 aa, synthetic construct
  REGION 1..5: note = cellulose binding domain
RYIRS

SEQ ID NO: 38 - protein, 4 aa, synthetic construct
  REGION 1..4: note = Phe-His-His-Thr
FHHT

SEQ ID NO: 39 - protein, 17 aa, synthetic construct
  REGION 1..17: note = WEAAAREACCRECCARAPEPTIDE
WEAAAREACCRECCARA

SEQ ID NO: 40 - protein, 8 aa, synthetic construct
  REGION 1..8: note = LEVLFQGP CLEAVAGE SITE
LEVLFQGP

SEQ ID NO: 41 - protein, 7 aa, synthetic construct
  REGION 1..7: note = ENLYTQS CLEAVAGE SITE
ENLYTQS

SEQ ID NO: 42 - protein, 5 aa, synthetic construct
  REGION 1..5: note = DDDDKCLEAVAGE SITE
DDDDK

SEQ ID NO: 43 - protein, 4 aa, synthetic construct
  REGION 1..4: note = LVPR CLEAVAGE SITE
LVPR

SEQ ID NO: 44 - protein, 22 aa, synthetic construct
  REGION 1..22: note = GSGATNFSLLKQAGDVEENPGPCLEAVAGE SITE
GSGATNFSLLKQAGDVEENPGP

SEQ ID NO: 45-48 - skipped (4 entries)

SEQ ID NO: 49 - protein, 7 aa, synthetic construct
  REGION 1..7: note = SULFATASE AUXILIARY MOTIF
AALLTGR

SEQ ID NO: 50 - protein, 7 aa, synthetic construct
  REGION 1..7: note = SULFATASE AUXILIARY MOTIF
SQLLTGR

SEQ ID NO: 51 - protein, 7 aa, synthetic construct
  REGION 1..7: note = SULFATASE AUXILIARY MOTIF
AAFMTGR

SEQ ID NO: 52 - protein, 7 aa, synthetic construct
  REGION 1..7: note = SULFATASE AUXILIARY MOTIF
AAFLTGR

SEQ ID NO: 53 - protein, 7 aa, synthetic construct
  REGION 1..7: note = SULFATASE AUXILIARY MOTIF
GSLFTGR

SEQ ID NO: 54 - skipped

SEQ ID NO: 55 - protein, 5 aa, synthetic construct
  REGION 1..5: note = 5X Glycine - GGGGG
GGGGG

SEQ ID NO: 56 - protein, 4 aa, synthetic construct
  REGION 1..4: note = 4X Glycine
GGGG

SEQ ID NO: 57 - protein, 5 aa, synthetic construct
  REGION 1..5: note = 5X Alanine
AAAAA

SEQ ID NO: 58 - protein, 4 aa, synthetic construct
  REGION 1..4: note = 4X Alanine
AAAA

SEQ ID NO: 59 - protein, 6 aa, synthetic construct
  REGION 1..6: note = LPETGGSORTASE SITE
LPETGG

SEQ ID NO: 60 - protein, 6 aa, synthetic construct
  REGION 1..6: note = LPETAA SORTASE SITE
LPETAA

SEQ ID NO: 61 - protein, 5 aa, synthetic construct
  REGION 1..5: note = THROMBIN CLEAVAGE SITE
LVPRG

SEQ ID NO: 62 - protein, 5 aa, synthetic construct
  REGION 1..5: note = TRANSGLUTAMINASE SITE
LLQGG

SEQ ID NO: 63 - protein, 4 aa, synthetic construct
  REGION 1..4: note = TRANSGLUTAMINASE SITE
LLQG

SEQ ID NO: 64 - protein, 6 aa, synthetic construct
  REGION 1..6: note = TRANSGLUTAMINASE SITE
LSLSQG

SEQ ID NO: 65 - protein, 6 aa, synthetic construct
  REGION 1..6: note = TRANSGLUTAMINASE SITE
LLQLQG

SEQ ID NO: 66 - protein, 5 aa, synthetic construct
  REGION 1..5: note = GSGGS PEPTIDE
GSGGS

SEQ ID NO: 67 - protein, 4 aa, synthetic construct
  REGION 1..4: note = GGGS PEPTIDE
GGGS

SEQ ID NO: 68 - protein, 4 aa, synthetic construct
  REGION 1..4: note = GGSG PEPTIDE
GGSG

SEQ ID NO: 69 - protein, 5 aa, synthetic construct
  REGION 1..5: note = GGSGG PEPTIDE
GGSGG

SEQ ID NO: 70 - protein, 5 aa, synthetic construct
  REGION 1..5: note = GSGSG PEPTIDE
GSGSG

SEQ ID NO: 71 - protein, 5 aa, synthetic construct
  REGION 1..5: note = GSGGGPEPTIDE
GSGGG

SEQ ID NO: 72 - protein, 5 aa, synthetic construct
  REGION 1..5: note = GGGSG PEPTIDE
GGGSG

SEQ ID NO: 73 - protein, 5 aa, synthetic construct
  REGION 1..5: note = GSSSG PEPTIDE
GSSSG

SEQ ID NO: 74 - protein, 5 aa, synthetic construct
  REGION 1..5: note = GSSSS PEPTIDE
GSSSS

SEQ ID NO: 75 - protein, 5 aa, synthetic construct
  REGION 1..5: note = AAAGG PEPTIDE
AAAGG

SEQ ID NO: 76 - protein, 5 aa, synthetic construct
  REGION 1..5: note = GGGGS PEPTIDE
GGGGS

SEQ ID NO: 77 - protein, 15 aa, synthetic construct
  REGION 1..15: note = GCGASGGGGSGGGGS PEPTIDE
GCGASGGGGSGGGGS

SEQ ID NO: 78 - protein, 20 aa, synthetic construct
  REGION 1..20: note = GCGGSGGGGSGGGGSGGGGS PEPTIDE
GCGGSGGGGSGGGGSGGGGS

SEQ ID NO: 79 - protein, 15 aa, synthetic construct
  REGION 1..15: note = GCGGSGGGGSGGGGSPEPTIDE
GCGGSGGGGSGGGGS

SEQ ID NO: 80 - protein, 9 aa, Human papillomavirus type 16
LLMGTLGIV

SEQ ID NO: 81 - protein, 8 aa, Human papillomavirus type 16
TLGIVCPI

SEQ ID NO: 82 - protein, 10 aa, Human papillomavirus type 16
YMLDLQPETT

SEQ ID NO: 83 - protein, 9 aa, Human papillomavirus type 16
YMLDLQPET

SEQ ID NO: 84 - protein, 17 aa, Homo sapiens
GAGSLQPLALEGSLQKR

SEQ ID NO: 85 - protein, 13 aa, Influenza A virus
PKYVKQNTLKLAT

SEQ ID NO: 86 - protein, 33 aa, synthetic construct
  REGION 1..33: note = LEUCINE-ZIPPER POLYPEPTIDE
RMKQIEDKIEEILSKIYHIENEIARIKKLIGER

SEQ ID NO: 87 - protein, 31 aa, synthetic construct
  REGION 1..31: note = LEUCINE-ZIPPER POLYPEPTIDE
LSSIEKKLEEITSQLIQISNELTLIRNELAQ

SEQ ID NO: 88 - protein, 31 aa, synthetic construct
  REGION 1..31: note = LEUCINE-ZIPPER POLYPEPTIDE
LSSIEKKLEEITSQLIQIRNELTLIRNELAQ

SEQ ID NO: 89 - protein, 31 aa, synthetic construct; identical to SEQ ID NO: 88
  REGION 1..31: note = LEUCINE-ZIPPER POLYPEPTIDE

SEQ ID NO: 90 - protein, 31 aa, synthetic construct
  REGION 1..31: note = LEUCINE-ZIPPER POLYPEPTIDE
LSSIEKKLEEITSQLQQIRNELTLIRNELAQ

SEQ ID NO: 91 - protein, 31 aa, synthetic construct
  REGION 1..31: note = LEUCINE-ZIPPER POLYPEPTIDE
LSSLEKKLEELTSQLIQLRNELTLLRNELAQ

SEQ ID NO: 92 - protein, 31 aa, synthetic construct
  REGION 1..31: note = LEUCINE-ZIPPER POLYPEPTIDE
ISSLEKKIEELTSQIQQLRNEITLLRNEIAQ

SEQ ID NO: 93 - protein, 47 aa, synthetic construct
  REGION 1..47: note = LEUCINE-ZIPPER POLYPEPTIDE
LEIEAAFLERENTALETRVAELRQRVQRLRNRVSQYRTRYGPLGGGK

SEQ ID NO: 94 - protein, 64 aa, synthetic construct
  REGION 1..64: note = COLLAGEN OLIGOMERIZATION POLYPEPTIDE
VTAFSNMDDMLQKAHLVIEGTFIYLRDSTEFFIRVRDGWKKLQLGELIPIPADSPPPPALSSNP

SEQ ID NO: 95 - protein, 31 aa, synthetic construct
  REGION 1..31: note = COILED-COIL DIMERIZATION POLYPEPTIDE
LKSVENRLAVVENQLKTVIEELKTVKDLLSN

SEQ ID NO: 96 - protein, 31 aa, synthetic construct
  REGION 1..31: note = COILED-COIL DIMERIZATION POLYPEPTIDE
LARIEEKLKTIKAQLSEIASTLNMIREQLAQ

SEQ ID NO: 97 - protein, 31 aa, synthetic construct
  REGION 1..31: note = COILED-COIL DIMERIZATION POLYPEPTIDE
VSRLEEKVKTLKSQVTELASTVSLLREQVAQ

SEQ ID NO: 98 - protein, 30 aa, synthetic construct
  REGION 1..30: note = COILED-COIL DIMERIZATION POLYPEPTIDE
IQSEKKIEDISSLIGQIQSEITLIRNEIAQ

SEQ ID NO: 99 - protein, 31 aa, synthetic construct
  REGION 1..31: note = COILED-COIL DIMERIZATION POLYPEPTIDE
LMSLEKKLEELTQTLMQLQNELSMLKNELAQ

SEQ ID NO: 100 - protein, 18 aa, synthetic construct
  REGION 1..18: note = DIMERIZATION POLYPEPTIDE
VDLEGSTSNGRQCAGIRL

SEQ ID NO: 101 - protein, 27 aa, synthetic construct
  REGION 1..27: note = DIMERIZATION POLYPEPTIDE
EDDVTTTEELAPALVPPPKGTCAGWMA

SEQ ID NO: 102 - protein, 27 aa, synthetic construct
  REGION 1..27: note = DIMERIZATION POLYPEPTIDE
GHDQETTTQGPGVLLPLPKGACTGQMA

SEQ ID NO: 103 - protein, 5 aa, synthetic construct
  REGION 1..5: note = SCAFFOLD PEPTIDE Val-Pro-Gly-X-Gly PENTAMERIC REPEAT
  UNSURE 4: note = Xaa is any amino acid other than proline.
VPGXG

SEQ ID NO: 104 - protein, 254 aa, Homo sapiens
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTENVVCALGLTVGLVGIIIGTIFIIKGLRKSNAAERRGPL

SEQ ID NO: 105 - protein, 178 aa, Homo sapiens
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 106 - protein, 79 aa, Homo sapiens
VIIQAEFYLNPDQSGEFMFDFDGDEIFHVDMAKKETVWRLEEFGRFASFEAQGALANIAVDKANLEIMTKRSNYTPITN

SEQ ID NO: 107 - protein, 266 aa, Homo sapiens [sequence omitted]

SEQ ID NO: 108 - protein, 266 aa, Homo sapiens [sequence omitted]

SEQ ID NO: 109 - protein, 266 aa, Homo sapiens
MVCLKLPGGSCMTALTVTLMVLSSPLALAGDTRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 110 - protein, 266 aa, Homo sapiens
MVCLKLPGGSCMTALTVTLMVLSSPLALSGDTRPRFLWQPKRECHFFNGTERVRFLDRYFYNQEESVRFDSDVGEFRAVTELGRPDAEYWNSQKDILEQARAAVDTYCRHNYGVVESFTVQRRVQPKVTVYPSKTQPLQHHNLLVCSVSGFYPGSIEVRWFLNGQEEKAGMVSTGLIQNGDWTFQTLVMLETVPRSGEVYTCQVEHPSVTSPLTVEWRARSESAQSKMLSGVGGFVLGLLFLGAGLFIYFRNQKGHSGLQPTGFLS

SEQ ID NO: 111 - protein, 266 aa, Homo sapiens [sequence omitted]

SEQ ID NO: 112 - protein, 266 aa, Homo sapiens [sequence omitted]

SEQ ID NO: 113 - protein, 266 aa, Homo sapiens
MVCLRLPGGSCMAVLTVTLMVLSSPLALAGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 114 - protein, 266 aa, Homo sapiens [sequence omitted]

SEQ ID NO: 115 - protein, 266 aa, Homo sapiens
MVCLKLPGGSCMAALTVTLMVLSSPLALAGDTQPRFLWQGKYKCHFFNGTERVQFLERLFYNQEEFVRFDSDVGEYRAVTELGRPVAESWNSQKDILEDRRGQVDTVCRHNYGVGESFTVQRRVHPEVTVYPAKTQPLQHHNLLVCSVSGFYPGSIEVRWFRNGQEEKAGVVSTGLIQNGDWTFQTLVMLETVPRSGEVYTCQVEHPSVMSPLTVEWRARSESAQSKMLSGVGGFVLGLLFLGAGLFIYFRNQKGHSGLQPTGFLS

SEQ ID NO: 116 - protein, 266 aa, Homo sapiens [sequence omitted]

SEQ ID NO: 117 - protein, 266 aa, Homo sapiens
MVCLKLPGGSSLAALTVTLMVLSSRLAFAGDTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSKMLSGVGGFVLGLLFLGAGLFIYFRNQKGHSGLQPTGFLS

SEQ ID NO: 118 - protein, 266 aa, Homo sapiens [sequence omitted]

SEQ ID NO: 119 - protein, 266 aa, Homo sapiens [sequence omitted]

SEQ ID NO: 120 - protein, 266 aa, Homo sapiens
MVCLKLPGGSCMAALTVTLTVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSKMLSGVGGFVLGLLFLGTGLFIYFRNQKGHSGLQPTGLLS

SEQ ID NO: 121 - protein, 266 aa, Homo sapiens
MVCLKLPGGSYMAKLTVTLMVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS

SEQ ID NO: 122 - protein, 261 aa, Homo sapiens
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 123 - protein, 263 aa, Homo sapiens
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHTGAPEPILRDWTPGLSPMQTLKVSVSAVTLGLGLIIFSLGVISWRRAGHSSYTPLPGSNYSEGWHIS

SEQ ID NO: 124 - protein, 250 aa, Homo sapiens
MALRAGLVLGFHTLMTLLSPQEAGATKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHWELQVPIPPPDAMETLVCALGLAIGLVGFLVGTVLIIMGTYVSSVPR

SEQ ID NO: 125 - protein, 273 aa, Homo sapiens
MGSGWVPWVVALLVNLTRLDSSMTQGTDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEWRAQSEYSWRKMLSGIAAFLLGLIFLLVGIVIQLRAQKGYVRTQMSGNEVSRAVLLPQSC

SEQ ID NO: 126 - protein, 260 aa, Homo sapiens
MRPEDRMFHIRAVILRALSLAFLLSLRGAGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHWEAQEPIQMPETTETVLCALGLVLGLVGIIVGTVLIIKSLRSGHDPRAQGTL

SEQ ID NO: 127 - protein, 258 aa, Homo sapiens
MMVLQVSAAPRTVALTALLMVLLTSVVQGRATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEWKAQSDSARSKTLTGAGGFVLGLIICGVGIFMHRRSKKVQRGSA

SEQ ID NO: 128 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 129 - protein, 255 aa, Homo sapiens
MILNKALLLGALALTAVMSPCGGEDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETLVCALGLSVGLMGIVVGTVFIIQGLRSVGASRHQGLL

SEQ ID NO: 130 - protein, 269 aa, Homo sapiens
MSWKKALRIPGDLRVATVTLMLAMLSSLLAEGRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGPQGPPPAGLLH

SEQ ID NO: 131 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 132 - protein, 227 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRPRGPPPAGLLH

SEQ ID NO: 133 - protein, 264 aa, Homo sapiens
MALQIPGGFWAAAVTVMLVMLSTPVAEARDFPKDFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEWRAQSESAQSKMLSGIGGFVLGLIFLGLGLIIRHRGQKGPRGPPPAGLLH

SEQ ID NO: 134 - protein, 191 aa, Homo sapiens
VPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYW

SEQ ID NO: 135 - protein, 98 aa, Homo sapiens
VPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSR

SEQ ID NO: 136 - protein, 93 aa, Homo sapiens
GFPIAEVFTLKPLEFGKPNTLVCFVSNLFPPMLTVNWQHHSVPVEGFGPTFVSAVDGLSFQAFSYLNFTPEPSDIFSCIVTHEIDRYTAIAYW

SEQ ID NO: 137 - protein, 179 aa, Homo sapiens
TKADHMGSYGPAFYQSYGASGQFTHEFDEEQLFSVDLKKSEAVWRLPEFGDFARFDPQGGLAGIAAIKAHLDILVERSNRSRAINVPPRVTVLPKSRVELGQPNILICIVDNIFPPVINITWLRNGQTVTEGVAQTSFYSQPDHLFRKFHYLPFVPSAEDVYDCQVEHWGLDAPLLRHW

SEQ ID NO: 138 - protein, 85 aa, Homo sapiens
TKADHMGSYGPAFYQSYGASGQFTHEFDEEQLFSVDLKKSEAVWRLPEFGDFARFDPQGGLAGIAAIKAHLDILVERSNRSRAIN

SEQ ID NO: 139 - protein, 94 aa, Homo sapiens
VPPRVTVLPKSRVELGQPNILICIVDNIFPPVINITWLRNGQTVTEGVAQTSFYSQPDHLFRKFHYLPFVPSAEDVYDCQVEHWGLDAPLLRHW

SEQ ID NO: 140 - protein, 181 aa, Homo sapiens
AGAIKADHVSTYAAFVQTHRPTGEFMFEFDEDEMFYVDLDKKETVWHLEEFGQAFSFEAQGGLANIAILNNNLNTLIQRSNHTQATNDPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 141 - protein, 85 aa, Homo sapiens
AIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 142 - protein, 94 aa, Homo sapiens
DPPEVTVFPKEPVELGQPNTLICHIDKFFPPVLNVTWLCNGELVTEGVAESLFLPRTDYSFHKFHYLTFVPSAEDFYDCRVEHWGLDQPLLKHW

SEQ ID NO: 143 - protein, 181 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 144 - protein, 87 aa, Homo sapiens
EDIVADHVASCGVNLYQFYGPSGQYTHEFDGDEQFYVDLERKETAWRWPEFSKFGGFDPQGALRNMAVAKHNLNIMIKRYNSTAATN

SEQ ID NO: 145 - protein, 94 aa, Homo sapiens
EVPEVTVFSKSPVTLGQPNTLICLVDNIFPPVVNITWLSNGQSVTEGVSETSFLSKSDHSFFKISYLTFLPSADEIYDCKVEHWGLDQPLLKHW

SEQ ID NO: 146 - protein, 181 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATNEVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 147 - protein, 87 aa, Homo sapiens
EDIVADHVASYGVNFYQSHGPSGQYTHEFDGDEEFYVDLETKETVWQLPMFSKFISFDPQSALRNMAVGKHTLEFMMRQSNSTAATN

SEQ ID NO: 148 - protein, 94 aa, Homo sapiens
EVPEVTVFSKFPVTLGQPNTLICLVDNIFPPVVNITWLSNGHSVTEGVSETSFLSKSDHSFFKISYLTFLPSADEIYDCKVEHWGLDEPLLKHW

SEQ ID NO: 149 - protein, 197 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSK

SEQ ID NO: 150 - protein, 94 aa, Homo sapiens
DTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 151 - protein, 100 aa, Homo sapiens
YPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESA

SEQ ID NO: 152 - protein, 197 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRVHPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 153 - protein, 94 aa, Homo sapiens
DTRPRFLELRKSECHFFNGTERVRYLDRYFHNQEEFLRFDSDVGEYRAVTELGRPVAESWNSQKDLLEQKRGRVDNYCRHNYGVGESFTVQRRV

SEQ ID NO: 154 - protein, 103 aa, Homo sapiens
HPQVTVYPAKTQPLQHHNLLVCSVSGFYPGSIEVRWFRNGQEEKAGVVSTGLIQNGDWTFQTLVMLETVPRSGEVYTCQVEHPSVTSALTVEWRARSESAQSK

SEQ ID NO: 155 - protein, 208 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRVQPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 156 - protein, 105 aa, Homo sapiens
TVLSSPLALAGDTQPRFLEQAKCECHFLNGTERVWNLIRYIYNQEEYARYNSDLGEYQAVTELGRPDAEYWNSQKDLLERRRAEVDTYCRYNYGVVESFTVQRRV

SEQ ID NO: 157 - protein, 103 aa, Homo sapiens
QPKVTVYPSKTQPLQHHNLLVCSVNGFYPGSIEVRWFRNGQEEKAGVVSTGLIQNGDWTFQTLVMLETVPRSGEVYTCQVEHPSMMSPLTVQWSARSESAQSK

SEQ ID NO: 158 - protein, 207 aa, Homo sapiens
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 159 - protein, 105 aa, Homo sapiens
MVLSSPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRV

SEQ ID NO: 160 - protein, 102 aa, Homo sapiens
EPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQS

SEQ ID NO: 161 - protein, 189 aa, Homo sapiens
GGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHTGAPEPILRDW

SEQ ID NO: 162 - protein, 94 aa, Homo sapiens
GGFVAHVESTCLLDDAGTPKDFTYCISFNKDLLTCWDPEENKMAPCEFGVLNSLANVLSQHLNQKDTLMQRLRNGLQNCATHTQPFWGSLTNRT

SEQ ID NO: 163 - protein, 95 aa, Homo sapiens
RPPSVQVAKTTPFNTREPVMLACYVWGFYPAEVTITWRKNGKLVMPHSSAHKTAQPNGDWTYQTLSHLALTPSYGDTYTCVVEHTGAPEPILRDW

SEQ ID NO: 164 - protein, 188 aa, Homo sapiens
TDSPEDFVIQAKADCYFTNGTEKVQFVVRFIFNLEEYVRFDSDVGMFVALTKLGQPDAEQWNSRLDLLERSRQAVDGVCRHNYRLGAPFTVGRKVQPEVTVYPERTPLLHQHNLLHCSVTGFYPGDIKIKWFLNGQEERAGVMSTGPIRNGDWTFQTVVMLEMTPELGHVYTCLVDHSSLLSPVSVEW

SEQ ID NO: 165 - protein, 94 aa, Homo sapiens
TDSPEDFVIQAKADCYFTNGTEKVQFVVRFIFNLEEYVRFDSDVGMFVALTKLGQPDAEQWNSRLDLLERSRQAVDGVCRHNYRLGAPFTVGRK

SEQ ID NO: 166 - protein, 94 aa, Homo sapiens
VQPEVTVYPERTPLLHQHNLLHCSVTGFYPGDIKIKWFLNGQEERAGVMSTGPIRNGDWTFQTVVMLEMTPELGHVYTCLVDHSSLLSPVSVEW

SEQ ID NO: 167 - protein, 186 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRRVQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 168 - protein, 92 aa, Homo sapiens
RATPENYLFQGRQECYAFNGTQRFLERYIYNREEFARFDSDVGEFRAVTELGRPAAEYWNSQKDILEEKRAVPDRMCRHNYELGGPMTLQRR

SEQ ID NO: 169 - protein, 94 aa, Homo sapiens
VQPRVNVSPSKKGPLQHHNLLVCHVTDFYPGSIQVRWFLNGQEETAGVVSTNLIRNGDWTFQILVMLEMTPQQGDVYTCQVEHTSLDSPVTVEW

SEQ ID NO: 170 - protein, 188 aa, Homo sapiens
RDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 171 - protein, 94 aa, Homo sapiens
RDSPEDFVFQFKGMCYFTNGTERVRLVTRYIYNREEYARFDSDVGVYRAVTPQGRPDAEYWNSQKEVLEGTRAELDTVCRHNYEVAFRGILQRR

SEQ ID NO: 172 - protein, 94 aa, Homo sapiens
VEPTVTISPSRTEALNHHNLLVCSVTDFYPGQIKVRWFRNDQEETAGVVSTPLIRNGDWTFQILVMLEMTPQRGDVYTCHVEHPSLQSPITVEW

SEQ ID NO: 173 - protein, 182 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTVTISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 174 - protein, 93 aa, Homo sapiens
DFLVQFKGMCYFTNGTERVRGVARYIYNREEYGRFDSDVGEFQAVTELGRSIEDWNNYKDFLEQERAAVDKVCRHNYEAELRTTLQRQVEPTV

SEQ ID NO: 175 - protein, 89 aa, Homo sapiens
TISPSRTEALNHHNLLVCSVTDFYPAQIKVRWFRNDQEETAGVVSTSLIRNGDWTFQILVMLEITPQRGDIYTCQVEHPSLQSPITVEW

SEQ ID NO: 176 - protein, 94 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHW

SEQ ID NO: 177 - protein, 92 aa, Homo sapiens
DTRPRFLWQHKFECHFFNGTERVRLLERCIYNQEESVRFDSDVGEYRAVTELGRPDAEYWNSQKDLLEQRRAAVDTYCRHNYGVGESFTVQR

SEQ ID NO: 178 - protein, 84 aa, Homo sapiens
IKEEHVIIQAEFYLNPDQSGEFMFDFDGDEIFHVDMAKKETVWRLEEFGRFASFEAQGALANIAVDKANLEIMTKRSNYTPITN

SEQ ID NO: 179 - protein, 98 aa, Homo sapiens
VPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 180 - protein, 47 aa, synthetic construct
  REGION 1..47: note = Leucine zipper
LEIRAAFLRQRNTALRTEVAELEQEVQRLENEVSQYETRYGPLGGGK

SEQ ID NO: 181 - protein, 227 aa, Homo sapiens; identical to SEQ ID NO: 11

SEQ ID NO: 182 - protein, 103 aa, Homo sapiens
PKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKM

SEQ ID NO: 183 - protein, 105 aa, Homo sapiens
VEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKM

SEQ ID NO: 184 - protein, 14 aa, Cytomegalovirus sp.
LPLKMLNIPSINVH

SEQ ID NO: 185 - protein, 92 aa, Homo sapiens
DTRPRFLWQHKFECHFFNGTERVRLLERCIYNQEESVRFDSDVGEYRAVTELGRPAAEYWNSQKDLLEQRRAAVDTYCRHNYGVGESFTVQR

SEQ ID NO: 186 - protein, 105 aa, Homo sapiens
VYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKM

SEQ ID NO: 187 - protein, 227 aa, Homo sapiens; identical to SEQ ID NO: 12

SEQ ID NO: 188 - protein, 96 aa, Homo sapiens
DTRPRFLWQHKFECHFFNGTERVRLLERCIYNQEESVRFDSDVGEYRAVTELGRPDAEYWNSQKDLLEQRRAAVDTYCRHNYGVGESFTVQRRVEP

SEQ ID NO: 189 - DNA, 1485 bp, synthetic construct
  misc_feature 1..1485: note = CONSTRUCT 1408 ENCODING A PROTEIN COMPRISING A MHC CLASS IIPROTEIN SEQUENCE
  source 1..1485: mol_type = other DNA
  CDS 1..1479
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgggtggaggtggctcaggaggcggcggcagcggtggaggagggagcctggagatcgaggccgccttcctggagcgggagaacaccgccctggagacccgggtggccgagctgcggcagcgggtgcagcggctgcggaaccgggtgtcccagtaccggacccggtacggccccctgggcggcggcaagtagtga

SEQ ID NO: 190 - protein, 493 aa, synthetic construct
  REGION 1..493: note = Synthetic Construct
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMGGGGSGGGGSGGGGSLEIEAAFLERENTALETRVAELRQRVQRLRNRVSQYRTRYGPLGGGK

SEQ ID NO: 191 - DNA, 1923 bp, synthetic construct
  misc_feature 1..1923: note = CONSTRUCT 1452 ENCODING A PROTEIN COMPRISING A MHC CLASS IIPROTEIN SEQUENCE
  source 1..1923: mol_type = other DNA
  CDS 1..1923: note = CONSTRUCT ENDODING A PROTEIN COMPRISING A MHC CLASS II PROTEINSEQUENCE
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactggtggaggtggctcaggaggcggcggcagcctggagatccgggccgccttcctgcggcagcggaacaccgccctgcggaccgaggtggccgagctggagcaggaggtgcagcggctggagaacgaggtgtcccagtacgagacccggtacggccccctgggcggcggcaagggcggatcagcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 192 - protein, 639 aa, synthetic construct
  REGION 1..639: note = Synthetic Construct
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGGGSGGGGSLEIRAAFLRQRNTALRTEVAELEQEVQRLENEVSQYETRYGPLGGGKGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 193 - DNA, 2139 bp, synthetic construct
  misc_feature 1..2139: note = CONSTRUCT 1559 ENCODING A PROTEIN COMPRISING A MHC CLASS IIPROTEIN SEQUENCE
  source 1..2139: mol_type = other DNA
  CDS 1..2139
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggaggcggaggatctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctggaggcggaggatctggaggcggaggatctggtggtggaggttctggtggtgggggatctggaggcggaggatctggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 194 - protein, 711 aa, synthetic construct
  REGION 1..711: note = Synthetic Construct
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRRVEPGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSGGGGSGGGGSGGGGSGGGGSGGGGSGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 195 - DNA, 2982 bp, synthetic construct
  misc_feature 1..2982: note = CONSTRUCT 1599 ENCODING A PROTEIN COMPRISING A MHC CLASS IIPROTEIN SEQUENCE
  source 1..2982: mol_type = other DNA
  CDS 1..2982
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggaggcggaggatctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 196 - protein, 992 aa, synthetic construct
  REGION 1..992: note = Synthetic Construct
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRRVEPGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 197 - DNA, 2667 bp, synthetic construct
  misc_feature 1..2667: note = CONSTRUCT 1601 ENCODING A PROTEIN COMPRISING A MHC CLASS IIPROTEIN SEQUENCE
  source 1..2667: mol_type = other DNA
  CDS 1..2667
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 198 - protein, 887 aa, synthetic construct
  REGION 1..887: note = Synthetic Construct
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRRVEPGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 199 - DNA, 1926 bp, synthetic construct
  misc_feature 1..1926: note = CONSTRUCT 1637ENCODING A PROTEIN COMPRISING A MHC CLASS IIPROTEIN SEQUENCE
  source 1..1926: mol_type = other DNA
  CDS 1..1926
atgtacaggatgcaactcctgtcttgcattgcactaagtcttgcacttgtcacaaacagtcttccgctcaaaatgcttaacataccttccattaatgtccacggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgcagccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactggtggaggtggctcaggaggcggcggcagcctggagatccgggccgccttcctgcggcagcggaacaccgccctgcggaccgaggtggccgagctggagcaggaggtgcagcggctggagaacgaggtgtcccagtacgagacccggtacggccccctgggcggcggcaagggcggatcagcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 200 - protein, 640 aa, synthetic construct
  REGION 1..640: note = Synthetic Construct
MYRMQLLSCIALSLALVTNSLPLKMLNIPSINVHGGGGSGSGGGGSGGGGGDTRPRFLWQHKFECHFFNGTERVRLLERCIYNQEESVRFDSDVGEYRAVTELGRPAAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGGGSGGGGSLEIRAAFLRQRNTALRTEVAELEQEVQRLENEVSQYETRYGPLGGGKGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 201 - DNA, 1938 bp, synthetic construct
  misc_feature 1..1938: note = CONSTRUCT 1639 ENCODING A PROTEIN COMPRISING A MHC CLASS IIPROTEIN SEQUENCE
  source 1..1938: mol_type = other DNA
  CDS 1..1938
atgtacaggatgcaactcctgtcttgcattgcactaagtcttgcacttgtcacaaacagtggggcagggtcactccaaccgttggcgctggaaggctctcttcagaagcgaggtggaggtggcggatccggctccggtggaggtggctcaggaggcggcggcggggacacccgaccacgtttcttggagcaggttaaacatgagtgtcatttcttcaacgggacggagcgggtgcggttcctggacagatacttctatcaccaagaggagtacgtgcgcttcgacagcgacgtgggggagtaccgggcggtgacggagctggggcggcctgatgccgagtactggaacagccagaaggacctcctggagcagaagcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactggtggaggtggctcaggaggcggcggcagcctggagatccgggccgccttcctgcggcagcggaacaccgccctgcggaccgaggtggccgagctggagcaggaggtgcagcggctggagaacgaggtgtcccagtacgagacccggtacggccccctgggcggcggcaagggcggatcagcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 202 - protein, 644 aa, synthetic construct
  REGION 1..644: note = Synthetic Construct
MYRMQLLSCIALSLALVTNSGAGSLQPLALEGSLQKRGGGGGSGSGGGGSGGGGGDTRPRFLEQVKHECHFFNGTERVRFLDRYFYHQEEYVRFDSDVGEYRAVTELGRPDAEYWNSQKDLLEQKRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETGGGGSGGGGSLEIRAAFLRQRNTALRTEVAELEQEVQRLENEVSQYETRYGPLGGGKGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 203 - DNA, 1485 bp, synthetic construct
  misc_feature 1..1485: note = CONSTRUCT 1640ENCODING A PROTEIN COMPRISING A MHC CLASS IIPROTEIN SEQUENCE
  source 1..1485: mol_type = other DNA
  CDS 1..1485
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgtctatcctgaggtgactgtgtatcctgcaaagacccagcccctgcagcaccacaacctcctggtctgctctgtgaatggtttctatccagccagcattgaagtcaggtggttccggaacggccaggaagagaagactggggtggtgtccacaggcctgatccagaatggagactggaccttccagaccctggtgatgctggaaacagttcctcggagtggagaggtttacacctgccaagtggagcacccaagcctgacgagccctctcacagtggaatggagagcacggtctgaatctgcacagagcaagatgggtggaggtggctcaggaggcggcggcagcggtggaggagggagcctggagatcgaggccgccttcctggagcgggagaacaccgccctggagacccgggtggccgagctgcggcagcgggtgcagcggctgcggaaccgggtgtcccagtaccggacccggtacggccccctgggcggcggcaagtagtga

SEQ ID NO: 204 - protein, 493 aa, synthetic construct
  REGION 1..493: note = Synthetic Construct
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSVYPEVTVYPAKTQPLQHHNLLVCSVNGFYPASIEVRWFRNGQEEKTGVVSTGLIQNGDWTFQTLVMLETVPRSGEVYTCQVEHPSLTSPLTVEWRARSESAQSKMGGGGSGGGGSGGGGSLEIEAAFLERENTALETRVAELRQRVQRLRNRVSQYRTRYGPLGGGK

SEQ ID NO: 205 - DNA, 1812 bp, synthetic construct
  misc_feature 1..1812: note = CONSTRUCT 1659 ENCODING A PROTEIN COMPRISING A MHC CLASS IIPROTEIN SEQUENCE
  source 1..1812: mol_type = other DNA
  CDS 1..1812
atgtacaggatgcaactcctgtcttgcattgcactaagtcttgcacttgtcacaaacagtccgaaatatgtaaaacagaataccctgaaattggcaacaggaggtggcggatccggctccggtggaggtggctcaggaggcggcggcggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctggaggcggaggatctggaggcggaggatctggtggtggaggttctggtggtgggggatctggaggcggaggatctggaggcggaggatctgcagctgcgggtggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgcaagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaaggcggatcacatcaccatcaccatcaccatcactagtga

SEQ ID NO: 206 - protein, 602 aa, synthetic construct
  REGION 1..602: note = Synthetic Construct
MYRMQLLSCIALSLALVTNSPKYVKQNTLKLATGGGGSGSGGGGSGGGGGDTRPRFLWQHKFECHFFNGTERVRLLERCIYNQEESVRFDSDVGEYRAVTELGRPDAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAGGGGSGGGGSGGGGSGGGGSGGGGSGGGGSAAAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHHHH

SEQ ID NO: 207 - DNA, 1479 bp, synthetic construct
  misc_feature 1..1479: note = CONSTRUCT 1661 ENCODING A PROTEIN COMPRISING A MHC CLASS IIPROTEIN SEQUENCE
  source 1..1479: mol_type = other DNA
  CDS 1..1479
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgggtggaggtggctcaggaggcggcggcagcggtggaggagggagcctggagatcgaggccgccttcctggagcgggagaacaccgccctggagacccgggtggccgagctgcggcagcgggtgcagcggctgcggaaccgggtgtcccagtaccggacccggtacggccccctgggcggcggcaagtagtga

SEQ ID NO: 208 - protein, 491 aa, synthetic construct
  REGION 1..491: note = Synthetic Construct
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMGGGGSGGGGSGGGGSLEIEAAFLERENTALETRVAELRQRVQRLRNRVSQYRTRYGPLGGGK

SEQ ID NO: 209 - DNA, 1293 bp, synthetic construct
  misc_feature 1..1293: note = CONSTRUCT 1664ENCODING A PROTEIN COMPRISING A MHC CLASS IIPROTEIN SEQUENCE
  source 1..1293: mol_type = other DNA
  CDS 1..1293
atgtacaggatgcaactcctgtcttgcattgcactaagtcttgcacttgtcacaaacagtgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctgcacctacttcaagttctacaaagaaaacacagctacaactggaggcattactgctggatttacagatgattttgaatggaattaataattacaagaatcccaaactcaccaggatgctcacagcaaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggaggcggaggatctggtggtggaggttctggtggtgggggatctggaggcggaggatctcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgtagtga

SEQ ID NO: 210 - protein, 429 aa, synthetic construct
  REGION 1..429: note = Synthetic Construct
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKM

SEQ ID NO: 211 - DNA, 984 bp, synthetic construct
  misc_feature 1..984: note = CONSTRUCT 1705 ENCODING A PROTEIN COMPRISING A MHC CLASS IIPROTEIN SEQUENCE
  source 1..984: mol_type = other DNA
  CDS 1..984
atgtacaggatgcaactcctgtcttgcattgcactaagtcttgcacttgtcacaaacagtccgaaatatgtaaaacagaataccctgaaattggcaacaggaggtggcggatccggtggaggtggctcaggaggcggcggctctggggacacccgaccacgtttcttgtggcagcataagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggggtggtggaggttcaatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacaaacagccctgtggaactgagagagcccaacgtcctcatctgtttcatagacaagtctaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgct

SEQ ID NO: 212 - protein, 328 aa, synthetic construct
  REGION 1..328: note = Synthetic Construct
MYRMQLLSCIALSLALVTNSPKYVKQNTLKLATGGGGSGGGGSGGGGSGDTRPRFLWQHKFECHFFNGTERVRLLERCIYNQEESVRFDSDVGEYRAVTELGRPDAEYWNSQKDLLEQRRAAVDTYCRHNYGVGESFTVQRGGGGSIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKSTPPVVNVTWLRNGKPVTTGVSETVFLPREDHLFRKFHYLPFLPSTEDVYDCRVEHWGLDEPLLKHWEFDA

SEQ ID NO: 213 - DNA, 2064 bp, synthetic construct
  misc_feature 1..2064: note = CONSTRUCT 1711 ENCODING A PROTEIN COMPRISING A MHC CLASS IIPROTEIN SEQUENCE
  source 1..2064: mol_type = other DNA
  CDS 1..2064
atgtacaggatgcaactcctgtcttgcattgcactaagtcttgcacttgtcacaaacagtgcccctacttccagctccaccaagaagacgcagcttcagctggaagcactgctgctcgatctgcagatgatactgaatggcattaacaactacaaaaaccccaagctcactcgcatgctgaccgctaaattctacatgcccaagaaggctacggaactgaagcacctgcagtgccttgaggaggaactcaagccactcgaggaggtgctgaacctggcacagtcaaagaactttcacctgcggccaagagacctgatttcgaacatcaacgtgattgtgctggaattgaagggctcagaaactacgttcatgtgcgagtacgccgacgaaactgctactatcgtggagttcttgaaccgctggatcacgttctgccagagcattatttcaactcttaccggtggaggtggttctggaggtggtggatcaggaggaggtggctccgggggtggaggtagcgctcccacgtcatcctccactaaaaagacccagctgcaactcgaggcactgttgctggacctccagatgattctgaacggaatcaacaactataagaacccgaagctgactagaatgttgactgccaaattttatatgccaaagaaggcaactgagttgaagcatctgcaatgcctggaagaggagctgaagccactggaagaggtgcttaacctcgctcagtccaagaacttccatctgcgcccacgggaccttatctccaacattaacgtgatcgtgctggaactgaagggatccgaaaccacttttatgtgcgaatacgctgacgaaaccgccactatcgtcgagttcctgaacaggtggatcaccttctgccagtccattatctccaccctcaccggtggaggtggttctggaggtggtggatcaggaggaggtggctccgggggtggaggtagcgttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgggaggcggaggatctggaggcggaggatctggtggtggaggttctggtggtgggggatctggaggcggaggatctggaggcggaggatctgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcacgaggctctgcacaaccactacacgcagaagtccctctccctgtctccgggtaaa

SEQ ID NO: 214 - protein, 688 aa, synthetic construct
  REGION 1..688: note = Synthetic Construct
MYRMQLLSCIALSLALVTNSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKMGGGGSGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0064668 A1
(43) Pub. Date: Mar. 2, 2023

(54) Antigen-Presenting Polypeptides with Chemical Conjugation Sites and Methods of Use Thereof

(71) Applicant: Cue Biopharma, Inc., Boston, MA (US)

(72) Inventors: Ronald D. SEIDEL, III, Natick, MA (US); Rodolfo J. CHAPARRO, Cambridge, MA (US); John F. ROSS, Cambridge, MA (US)

(21) Appl. No.: 17/867,651

(22) Filed: Jul. 18, 2022

Related U.S. Application Data
(63) Continuation of application No. 16/812,166, filed on Mar. 6, 2020, now abandoned.
Application No. 16/812,166 is a continuation of application No. PCT/US2018/049802, filed on Sep. 6, 2018.
(60) Provisional application No. 62/555,575, filed on Sep. 7, 2017.

(51) Int. Cl. C07K 14/74; A61K 38/00; C07K 14/55

(52) U.S. Cl. CPC C07K 14/70539; A61K 38/00; C07K 14/55; C07K 2319/30; C07K 2319/73; C07K 2319/74

(57) ABSTRACT

The present disclosure provides antigen-presenting polypeptides, including single-chain antigen-presenting polypeptides and multimeric antigen-presenting polypeptides comprising one or more chemical conjugation sites for incorporation of, for example, epitope containing polypeptides. The present disclosure provides nucleic acids comprising nucleotide sequences encoding antigen-presenting polypeptides comprising one or more chemical conjugation sites, as well as cells genetically modified with the nucleic acids. The single-chain and multimeric antigen-presenting polypeptides and their epitope conjugates are useful for modulating the activity of a T-cell, and accordingly, the present disclosure provides methods of modulating activity of a T-cell in vitro and in vivo as a method of treatment.

### CROSS-REFERENCE TO RELATED APPLICATIONS

This application claims the benefit of U.S. Provisional Application No. 62/555,575, which was filed Sep. 7, 2017.

### INCORPORATION OF SEQUENCE LISTING

This application contains a sequence listing submitted electronically via EFS-web, which serves as both the paper copy and the computer readable form (CRF) and consists of a file entitled “123640-8002WO00_seqlist.txt”, which was created on Sep. 2, 2018, which is 403,085 bytes in size, and which is herein incorporated by reference in its entirety.

### INTRODUCTION

Central to the proper functioning of the mammalian immune system are the coordinated activities and communications between two specialized cell types, antigen-presenting cells (“APCs”) and T-cells. APCs serve to capture and break the proteins from foreign organisms, or abnormal proteins (e.g., from genetic mutation(s) in cancer cells), into smaller fragments suitable as signals for scrutiny by the larger immune system, including T-cells. In particular, APCs break down proteins into small peptide fragments, which are then paired with proteins of the major histocompatibility complex (“MHC”) and displayed on the cell surface. Cell surface display of a MHC together with a peptide fragment, also known as a T-cell epitope, provides the underlying scaffold surveilled by T-cells, allowing for specific recognition. The peptide fragments can be pathogen-derived, tumor-derived, or derived from natural host proteins (self-proteins). Moreover, APCs can recognize other foreign components, such as bacterial toxins, viral proteins, viral DNA, viral RNA, etc., whose presence denotes an escalated threat level. The APCs relay this information to T-cells through costimulatory signals in order to generate a more effective response.

T cells recognize peptide-major histocompatibility complexes (“pMHC”) through a specialized cell surface receptor, the T-cell receptor (“TCR”). The TCR is unique to each T-cell; as a consequence, each T-cell is highly specific for a particular pMHC target. In order to adequately address the universe of potential threats, a very large number (˜10,000,000) of distinct T-cells with distinct TCRs exist in the human body. Further, any given T-cell, specific for a particular T-cell peptide, is initially a very small fraction of the total T-cell population. Although normally dormant and in limited numbers, T-cells bearing specific TCRs can be readily activated and amplified by APCs to generate highly potent T-cell responses that involve many millions of T-cells. Such activated T-cell responses are capable of attacking and clearing viral infections, bacterial infections, and other cellular threats including tumors, as illustrated below. Conversely, the broad, non-specific activation of overly active T-cell responses against self or shared antigens can give rise to T-cells inappropriately attacking and destroying healthy tissues or cells.

MHC proteins are referred to as human leukocyte antigens (HLA) in humans. HLA class II gene loci include HLA-DM (HLA-DMA and HLA-DMB that encode HLA-DM α chain and HLA-DM β chain, respectively), HLA-DO (HLA-DOA and HLA-DOB that encode HLA-DO α chain and HLA-DO β chain, respectively), HLA-DP (HLA-DPA and HLA-DPB that encode HLA-DP α chain and HLA-DP β chain, respectively), HLA-DQ (HLA-DQA and HLA-DQB that encode HLA-DQ α chain and HLA-DQ β chain, respectively), and HLA-DR (HLA-DRA and HLA-DRB that encode HLA-DR α chain and HLA-DR β chain, respectively).

### SUMMARY

The present disclosure provides T-cell modulatory antigen-presenting polypeptide(s) referred to as a “TMAPP” (singular) or “TMAPPs” (plural). TMAPPs include single-chain T-cell modulatory antigen-presenting polypeptide(s) denoted as a “sc-TMAPP” (singular) or “sc-TMAPPs” (plural), and multimeric T-cell modulatory antigen-presenting polypeptide(s) denoted as a “m-TMAPP” (singular) or “m-TMAPPs” (plural). The disclosure includes and provides for TMAPPs having at least one chemical conjugation site where an epitope presenting molecule (also referred to herein as a “peptide epitope,” “peptide antigen,” or “epitope-presenting peptide” or simply as an “epitope”) and/or a payload (e.g., a therapeutic) may be covalently attached. The disclosure further provides for TMAPPs (including both sc-TMAPPs and m-TMAPPs) having an epitope covalently bound that are denoted as an epitope conjugate(s) (e.g., a TMAPP-epitope conjugate, or more specifically, a sc-TMAPP-epitope conjugate, or a m-TMAPP-epitope conjugate). In other embodiments, the TMAPPs have one or more immunomodulatory polypeptide sequences referred to as “MOD(s)” (e.g., an IL-2 and/or CD80 polypeptide sequences) covalently associated (e.g., cotranslated) with a peptide of a TMAPP. The one or more MODs may be wildtype and/or variant MODs covalently associated (e.g., cotranslated) with a peptide of a TMAPP. In some embodiments, the TMAPPs do not have any MODs covalently associated (e.g., cotranslated) with the TMAPPs. Depending on the number of chemical conjugation sites and the order of the conjugation reactions, TMAPPs and their epitope conjugates may have chemical conjugation sites for payload(s) and/or epitope(s). In an embodiment, at least one chemical conjugation site is placed for covalently associating an epitope and/or a a second chemical conjugation site for covalently associating a payload. In an embodiment, a TMAPP has a chemical conjugation site for the covalent attachment of an epitope (e.g., a polypeptide antigen for binding and recognition by a T-cell receptor) and lacks a chemical conjugation site for payload. In another embodiment, the TMAPP is an epitope conjugate (e.g., a sc-TMAPP or a m-TMAPP-epitope conjugate) having a covalently attached epitope at a chemical conjugation site, such that the epitope can be bound and recognized by a T-cell receptor, but lacks a payload and/or chemical conjugation sites for payload. The present disclosure includes and provides for nucleic acids comprising nucleotide sequences encoding unconjugated TMAPPs of the present disclosure that have not been subjected to conjugation reaction with an epitope or payload, as well as cells genetically modified with those nucleic acids.

By providing a chemical conjugation site for the incorporation of an epitope in TMAPPs, such TMAPPs that are unconjugated to an epitope may be used as a T-cell receptor (“TCR”) presentation platform into which various epitopes (e.g., peptide antigens) may be covalently bound, and the resulting epitope conjugate used for modulating the activity of a T-cell bearing a TCR specific to the epitope. The effect of TMAPP-epitope conjugates on T-cells with TCRs specific to the epitope conjugate depends on which, if any, MODs are present in the TMAPP. In the absence of any stimulatory MOD in the TMAPP-epitope conjugate, prolonged exposure to the TMAPP-epitope conjugate may result in T-cell anergy or suppression of T-cell stimulation. The action of TMAPP-epitope conjugates having MODs (e.g., IL-2, CD80, 4-1BBL . . . polypeptides) depends on the stimulatory or inhibitory effect of the MODs. MOD-containing TMAPP-epitope conjugates function as a means of selectively delivering the MODs to T-cells specific for the conjugated (covalently bound) epitope, resulting in MOD-driven T-cell responses (e.g., proliferation of epitope specific T-cells). The combination of the reduced affinity of the MOD(s) for their Co-MOD(s), and the affinity of the epitope for a TCR, provides for enhanced selectivity of a TMAPP-epitope conjugate while retaining the activity of the MODs. Accordingly, the present disclosure provides methods of modulating the activity of T-cells in vitro and in vivo, and the use of TMAPPs as therapeutics in methods of treatment.

### BRIEF DESCRIPTION OF THE DRAWINGS

FIG. 1 provides a schematic depiction of MHC Class II alpha- and beta-chains with a peptide (peptide comprising an epitope).

FIGS. 2A-2C provide schematic depictions of examples of TMAPP-epitope conjugates, with the epitope covalently bound to the MHC Class II β1 polypeptide via a linker (shown as a line) between those elements. The epitope conjugates in FIGS. 2A and 2B are m-TMAPPs, and the epitope conjugate in FIG. 2C is a sc-TMAPP.

FIGS. 3A-3B provide a schematic depiction of a MOD-containing m-TMAPP-epitope conjugate (FIG. 3A); and a crystal structure of the human Class II MHC protein HLA-DR1 complexed with an influenza virus peptide (FIG. 3B). In FIG. 3A, the epitopes are covalently bound to the MHC Class II β1 polypeptide via a linker (shown as a line) between the epitope and the β1 polypeptide.

FIGS. 4A-4C depict gel analysis (FIG. 4A), expression levels (FIG. 4B), and descriptions (FIG. 4C) of exemplary molecules with structures and organization similar to TMAPP-epitope conjugates, however, the molecules in this figure were prepared by expressing a nucleic acid sequence that included the epitope, rather than by attaching the epitope by chemical conjugation.

FIGS. 5A-5B provide schematic depictions of a m-TMAPP (FIG. 5A, left) and a sc-TMAPP (FIG. 5A, right) without MOD polypeptides, and a sc-TMAPP with one or two variant MOD polypeptides (FIG. 5B). The unmarked rectangle in FIG. 5A represents a dimerization domain (e.g., a bZIP polypeptide). In FIG. 5B, the arrows pointing to the dashed lines indicate possible positions of a MOD polypeptide(s).

FIG. 6 provides an amino acid sequence of a HLA Class II DRA α chain.

FIGS. 7A-7J provide amino acid sequences of HLA Class II DRB1 β chains.

FIGS. 8A-8C provide amino acid sequences of HLA Class II DRB3 β chains.

FIG. 9 provides an amino acid sequence of a HLA Class II DRB4 β chain.

FIG. 10 provides an amino acid sequence of a HLA Class II DRB5 β chain.

FIG. 11 provides an amino acid sequence of a HLA Class II DMA α chain.

FIG. 12 provides an amino acid sequence of a HLA Class II DMB β chain.

FIG. 13 provides an amino acid sequence of a HLA Class II DOA α chain.

FIG. 14 provides an amino acid sequence of a HLA Class II DOB β chain.

FIG. 15 provides an amino acid sequence of a HLA Class II DPA1 α chain.

FIG. 16 provides an amino acid sequence of a HLA Class II DPB1 β chain.

FIG. 17 provides an amino acid sequence of a HLA Class II DQA1 α chain.

FIG. 18 provides an amino acid sequence of a HLA Class II DQA2 α chain.

FIGS. 19A-19B provide amino acid sequences of HLA Class II DQB1 β chains.

FIGS. 20A-20B provide amino acid sequences of HLA Class II DQB2 β chains.

FIGS. 21A-21G provide amino acid sequences of immunoglobulin Fc polypeptides (SEQ ID NOs:1-12).

FIGS. 22A-22L provide schematic depictions of exemplary m-TMAPP-epitope conjugates of the present disclosure, with epitopes bound through a chemical conjugation site denoted by “cc” at the N-terminus of a MOD or a MHC Class II polypeptide, or within or at the N-terminus of an optional linker (shown as a line) attached thereto as indicated by the arrows.

FIGS. 23A-23I provide schematic depictions of exemplary sc-TMAPPs of the present disclosure with epitopes bound through a chemical conjugation site denoted by “cc” at the N-terminus of the β1 polypeptide, a MOD polypeptide, or within or at the N-terminus of a linker attached thereto as indicated by the arrows. FIGS. 23C and 23F both show a MOD-epitope polypeptide joined to the MHC Class II β1 polypeptide.

FIG. 24 depicts production of molecules 1-3 with structures and organization related to a sc-TMAPP-epitope conjugate, and molecules 4-7 related to two-peptide chain m-TMAPP-epitope conjugates. The molecules shown in this figure were prepared using nucleic acid expression constructs containing the corresponding nucleotide sequences and expressing the proteins (including the epitope) in vitro, instead of conjugating the epitopes (hemagglutinin (HA), CMV, and proinsulin peptides) with the corresponding unconjugated TMAPP. The gel analysis on the left shows the intact proteins were produced in detectable amounts.

FIGS. 25A-25B provide the amino acid sequence (FIG. 25A) of one polypeptide chain of a molecule with the structure and organization similar to a polypeptide of a m-TMAPP-epitope conjugate; however, the molecule shown in this figure is prepared by translating the nucleotide sequence of FIG. 25B.

FIGS. 26A-26B provide the amino acid sequence (FIG. 26A) of an exemplary polypeptide chain of a molecule with the structure and organization similar to an epitope-containing polypeptide of a m-TMAPP-epitope conjugate; however, the molecule shown in this figure is prepared by translating the nucleotide sequence (FIG. 26B) including a leader sequence and the epitope, rather than by attaching the epitope by chemical conjugation.

FIGS. 27A-27B provide the amino acid sequence (FIG. 27A) of an exemplary polypeptide chain of a molecule with the structure and organization similar to a MOD-less sc-TMAPP-epitope conjugate; however, the molecule shown in this figure is prepared by translating the nucleotide sequence (FIG. 27B) including a leader sequence and the epitope, rather than by attaching the epitope by chemical conjugation.

FIGS. 28A-28B provide the amino acid sequence (FIG. 28A) of an exemplary polypeptide chain of a molecule with the structure and organization similar to an epitope-containing polypeptide of a sc-TMAPP-epitope conjugate; however, the molecule shown in this figure is prepared by translating the nucleotide sequence (FIG. 28B) including a leader sequence and the epitope, rather than by attaching the epitope by chemical conjugation.

FIGS. 29A-29B provide the amino acid sequence (FIG. 29A) of an exemplary polypeptide chain of a molecule with the structure and organization similar to an epitope-containing polypeptide of a sc-TMAPP-epitope conjugate; however, the molecule shown in this figure is prepared by translating the nucleotide sequence (FIG. 29B) including a leader sequence and the epitope, rather than by attaching the epitope by chemical conjugation.

FIGS. 30A-30B provide the amino acid sequence (FIG. 30A) of an exemplary polypeptide chain of a molecule with the structure and organization similar to an epitope-containing polypeptide of a m-TMAPP-epitope conjugate; however, the molecule shown in this figure is prepared by translating the nucleotide sequence (FIG. 30B) including a leader sequence and the epitope, rather than by attaching the epitope by chemical conjugation.

FIGS. 31A-31B provide the amino acid sequence (FIG. 31A) of an exemplary polypeptide chain of a molecule with the structure and organization similar to an epitope-containing polypeptide of a m-TMAPP-epitope conjugate; however, the molecule shown in this figure is prepared by translating the nucleotide sequence (FIG. 31B) including a leader sequence and the epitope, rather than by attaching the epitope by chemical conjugation.

FIGS. 32A-32B provide the amino acid sequence (FIG. 32A) of an exemplary polypeptide chain of a molecule with the structure and organization similar to a polypeptide of a m-TMAPP-epitope conjugate; however, the molecule shown in this figure is prepared by translating the nucleotide sequence of FIG. 32B.

FIGS. 33A-33B provide the amino acid sequence (FIG. 33A) of an exemplary polypeptide chain of a molecule with the structure and organization similar to an epitope-containing polypeptide of a m-TMAPP-epitope conjugate; however, the molecule shown in this figure is prepared by translating the nucleotide sequence (FIG. 33B) including a leader sequence and the epitope, rather than by attaching the epitope by chemical conjugation.

FIGS. 34A-34B provide the amino acid sequence (FIG. 34A) of an exemplary polypeptide chain of a molecule with the structure and organization similar to a polypeptide of a m-TMAPP-epitope conjugate; however, the molecule shown in this figure is prepared by translating the nucleotide sequence of FIG. 34B.

FIGS. 35A-35B provide the amino acid sequence (FIG. 35A) of an exemplary polypeptide chain of a molecule with the structure and organization similar to a polypeptide of a m-TMAPP-epitope conjugate; however, the molecule shown in this figure is prepared by translating the nucleotide sequence of FIG. 35B.

FIG. 36 depicts gel analysis, expression levels, and descriptions of exemplary molecules with structures and organization similar to a MOD-containing TMAPP-epitope conjugate with tandem IL-2 MOD sequences and its MOD-less counterpart. The molecules in this figure were prepared by expressing a nucleic acid sequence that included the epitope, rather than by attaching the epitope by chemical conjugation. The gel analysis shows intact peptides were made in detectable amounts. The epitope (HA) was from hemagglutinin.

FIGS. 37A-37B provide the amino acid sequence (FIG. 37A) of an exemplary polypeptide chain of a molecule with the structure and organization similar to an epitope-containing polypeptide of a m-TMAPP-epitope conjugate; however, the molecule shown in this figure is prepared by translating the nucleotide sequence (FIG. 37B) including a leader sequence and the epitope, rather than by attaching the epitope by chemical conjugation.

FIGS. 38A-38B provide the amino acid sequence (FIG. 38A) of an exemplary polypeptide chain of a molecule with the structure and organization similar to a polypeptide of a m-TMAPP-epitope conjugate; however, the molecule shown in this figure is prepared by translating the nucleotide sequence of FIG. 34B.

FIG. 39 depicts gel analysis and a schematic description of an exemplary molecule with a structure and organization similar to a MOD-containing TMAPP-epitope conjugate. The molecule includes tandem IL-2 MOD sequences and bZIP dimerization domains. The molecule in this figure was prepared by expressing a nucleic acid sequence that included the proinsulin epitope peptide coding sequence, rather than by attaching the epitope by chemical conjugation. The gel analysis shows intact peptides were made in detectable amounts, with the higher molecular weight bands in the non-reducing lane indicating formation of higher order structures.

FIG. 40 shows a schematic of hydrazinyl indoles reacting with an aldehyde containing polypeptide adapted from U.S. Pat. No. 9,310,374.

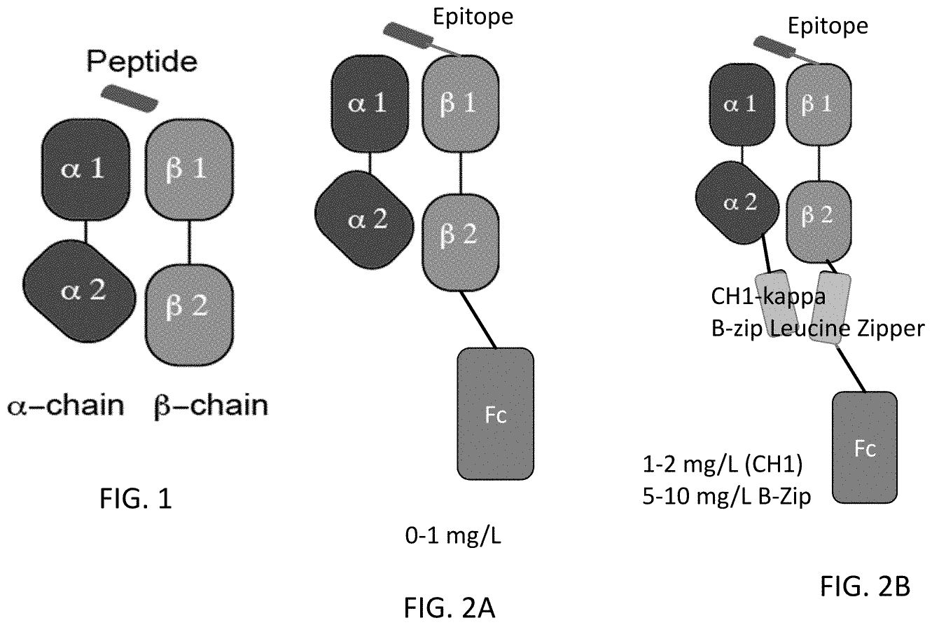

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two polynucleotides or polypeptides. Sequence identity can be determined in a number of different ways, for example, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10.

“Naturally occurring amino acid,” unless stated otherwise, means: L (Leu, leucine), A (Ala, alanine), G (Gly, glycine), S (Ser, serine), V (Val, valine), F (Phe, phenylalanine), Y (Tyr, tyrosine), H (His, histidine), R (Arg, arginine), N (Asn, asparagine), E (Glu, glutamic acid), D (Asp, asparagine), C (Cys, cysteine), Q (Gln, glutamine), I (Ile, isoleucine), M (Met, methionine), P (Pro, proline), T (Thr, threonine), K (Lys, lysine), and W (Trp, tryptophan). Although both selenocysteine and hydroxyproline are naturally occurring amino acids, they are specifically referred to in any instance where they are intended to be encompassed and are not otherwise included in naturally occurring amino acids as used herein. The term “amino acid” may be abbreviated as “aa” and used in both the singular and plural case as will be clear from the context; where “aas” is used it refers to the plural case.

“Non-natural amino acids” are any amino acid other than the naturally occurring amino acids recited above, selenocysteine, and hydroxyproline.

“Chemical conjugation” as used herein means formation of a covalent bond. “Chemical conjugation site” as used herein means a location in a polypeptide at which a covalent bond can be formed, including any contextual elements (e.g., surrounding amino acid sequences) that are required or assist in the formation of a covalent bond to the polypeptide. Accordingly, a site comprising a group of amino acids that directs enzymatic modification, and ultimately covalent bond formation at an amino acid within the group, may also be referred to as a chemical conjugation site. In some instances, as will be clear from the context, the term chemical conjugation site may be used to refer to a location where covalent bond formation or chemical modification has already occurred.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consists of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“Binding” as used herein (e.g., with reference to binding of a molecule such as a TMAPP comprising one or more MODs to one or more polypeptide (e.g., a T-cell receptor and a cognate co-immunomodulatory polypeptide (Co-MOD) on a T-cell) refers to a non-covalent interaction(s) between the molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, or less than 10⁻¹² M. “Affinity” refers to the strength of non-covalent binding, increased binding affinity being correlated with a lower K_{D}. “Specific binding” generally refers to, e.g., binding between a ligand molecule and its binding site or “receptor” with an affinity of at least about 10⁻⁷ M or greater, (e.g., less than 5×10⁻⁷ M, less than 10⁻⁸M, less than 5×10⁻⁸ M, less than 10⁻⁹ M, less than 10⁻¹⁰ M, less than 10⁻¹¹ M, or less than 10⁻¹² M and greater affinity, or in a range from 10⁻⁷ to 10⁻⁹ or from 10⁻⁹ to 10⁻¹²). “Non-specific binding” generally refers to the binding of a ligand to something other than its designated binding site or “receptor,” typically with an affinity of less than about 10⁻⁷ M (e.g., binding with an affinity of less than about 10⁻⁶ M, less than about 10⁻⁵ M, less than about 10⁻⁴ M). However, in some contexts, e.g., binding between a TCR and a peptide/MHC complex, “specific binding” can be in the range of from 1 μM to 100 μM, or from 100 μM to 1 mM. “Covalent binding” as used herein means the formation of one or more covalent chemical bonds between two different molecules.

The terms “immunological synapse” or “immune synapse” as used herein generally refer to the natural interface between two interacting immune cells of an adaptive immune response including, e.g., the interface between an antigen-presenting cell (APC) or target cell and an effector cell, e.g., a lymphocyte, an effector T-cell, a natural killer cell, and the like. An immunological synapse between an APC and a T-cell is generally initiated by the interaction of a T-cell antigen receptor and MHC molecules, e.g., as described in Bromley et al., Annu. Rev Immunol. 2001, 19:375-96, the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to as a “MOD” or “co-stimulatory polypeptide”), as used herein, includes a polypeptide on an APC (e.g., a dendritic cell, a B cell, and the like), or a portion of a polypeptide on an APC, that specifically binds a Co-MOD on a T-cell, thereby providing a signal which, in addition to the primary signal provided by, for instance, binding of a TCR/CD3 complex with a MHC polypeptide loaded with peptide, mediates a T-cell response, including, but not limited to, proliferation, activation, differentiation, and the like. MODs include, but are not limited to, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, lymphotoxin beta receptor, 3/TR6, ILT3, ILT4, HVEM, an agonist or antibody that binds to Toll ligand receptor and a ligand that specifically binds to B7-H3. A co-stimulatory polypeptide also encompasses, inter alia, an antibody that specifically binds with a cognate co-stimulatory molecule present on a T-cell, such as, but not limited to, IL-2, CD27, CD28, 4-1BB, OX40, CD30, CD40, PD-1, ICOS, lymphocyte function-associated antigen-1 (LFA-1), CD2, LIGHT, NKG2C, B7-H3, and a ligand that specifically binds to CD83.

The term TMAPP is generic to, and includes, both TMAPPs with a single polypeptide chain (sc-TMAPPs) or with more than one (e.g., two) polypeptide chains (m-TMAPPs) unless stated otherwise. The terms sc-TMAPPs and m-TMAPPs include both molecules with chemical conjugation sites and molecules in the form of an epitope conjugate; whether or not they contain a MOD. In those instances where a reference to only TMAPPs that contain a MOD is intended, terms such as a “MOD-containing TMAPP,” “TMAPP comprising a MOD,” and the like are employed. In those instances where this disclosure specifically refers to a TMAPP that does not contain a MOD, terms such as “MOD-less TMAPP” or a “TMAPP without a MOD” and the like are employed. Accordingly, TMAPP as used herein, or a reference to “any TMAPP” or “all TMAPPs,” is generic to sc-TMAPPs with one or more chemical conjugation sites, sc-TMAPP-epitope conjugates, m-TMAPPs with one or more chemical conjugation sites, and m-TMAPP-epitope conjugates; including those that are MOD-less, MOD-containing, or unconjugated. The term “unconjugated TMAPP(s)” refers to TMAPPs that have not been conjugated (covalently linked) to an epitope and/or payload (e.g., non-epitope molecule such as a label), and therefore comprise at least one chemical conjugation site.

An “immunomodulatory domain” of a MOD-containing TMAPP is that portion of a TMAPP (a MOD peptide sequence) that binds a Co-MOD, which may be present on a target T-cell.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector” and “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, at least 1,000-fold greater or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

The terms “treatment,” “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, e.g., arresting its development; or (c) relieving the disease, e.g., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

Before the present invention is further described, it is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that the range includes each intervening value, to the tenth of the lower limit of the range, unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where a range includes upper and/or lower limits, ranges excluding either or both of those limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that, as used herein and in the appended claims, the singular forms “a,” “an,” and “the” include plural referents unless the context clearly dictates otherwise. Thus, for example, reference to “a Treg” includes a plurality of such Tregs and reference to “the MHC Class II alpha chain” includes reference to one or more MHC Class II alpha chains and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publications by virtue of prior invention. Further, the dates of publication provided may be different from the actual publication dates, which may need to be independently confirmed.

### DETAILED DESCRIPTION

The present disclosure includes and provides TMAPPs, including sc-TMAPPs and m-TMAPPs comprising at least one chemical conjugation site. The disclosure further provides for TMAPPs having an epitope containing molecule conjugated (covalently bound), directly or indirectly through a bond with a chemical conjugation site as an epitope conjugate (e.g., a sc-TMAPP-epitope conjugate or a m-TMAPP-epitope conjugate). Where an epitope (e.g., a peptide capable of being bound and recognized by a T-cell receptor) is covalently attached to form a TMAPP-epitope conjugate, the conjugate may be specifically bound by a T-cell receptor in an antigen specific manner. The present disclosure additionally provides nucleic acids comprising nucleotide sequences encoding unconjugated TMAPPs of the present disclosure with chemical conjugation sites, as well as cells genetically modified with the nucleic acids. A TMAPP of the present disclosure comprising at least one chemical conjugation site may be used as a molecular scaffold into which various epitopes (e.g., peptides comprising a sequence that serves as an epitope) may be covalently bound, and the resulting epitope conjugate used for modulating activity of a T-cell. Accordingly, the present disclosure provides methods of modulating activity of a T-cell (or population of T-cells) in vitro and in vivo (in human and/or non-human hosts), and methods of treatment in which the activity of T-cells is modulated.

MOD-Less TMAPPs

The present disclosure provides TMAPPs comprising at least one chemical conjugation site, including sc-TMAPPs comprising at least one chemical conjugation site, and m-TMAPPs comprising at least one chemical conjugation site that do not comprise MOD polypeptide sequences (MOD-less TMAPPs). MOD-less TMAPPs are discussed here and in the following sections directed to MOD-less m-TMAPPs (including MOD-less m-TMAPP embodiments A-L) and MOD-less sc-TMAPPs (including MOD-less sc-TMAPP embodiments A′-H′). TMAPPs that contain MODs as part of a polypeptide sequence including one or more MHC Class II polypeptide sequences are described following the description of MODs and variant MODs (e.g., a variant MOD with reduced affinity for its Co-MOD).

Naturally occurring Class II MHC polypeptides comprise an α chain and a β chain. “Class II MHC polypeptides” include human leukocyte antigen (HLA) α- and β-chains. MHC Class II polypeptides include MHC Class II DP α and β polypeptides, DM α and β polypeptides, DOA α and β polypeptides, DOB α and β polypeptides, DQ α and β polypeptides, and DR α and β polypeptides. As used herein, a Class II MHC polypeptide can comprise a class II MHC α chain polypeptide, a class II MHC β chain polypeptide, or only a portion of a class II MHC α or β chain polypeptide. For example, a Class II MHC polypeptide can be a polypeptide that includes: i) only the α1 domain of a class II MHC α chain polypeptide; ii) only the α2 domain of a class II MHC α chain; iii) only the α1 domain and an α2 domain of a class II MHC α chain; iv) only the β1 domain of a class II MHC β chain; v) only the β2 domain of a class II MHC β chain; vi) only the β1 domain and the β2 domain of a class II MHC β chain; vii) the α1 domain of a class II MHC α chain, the β1 domain of a class II MHC β chain, and the β2 domain of a class II MHC; and the like.

Class II MHC polypeptides include allelic forms. The HLA locus is highly polymorphic in nature. As disclosed in the Nomenclature for Factors of the HLA System 2000 (Hum. Immunol., 62(4):419-68, 2001), there are 221 HLA-DRB 1 alleles, 19 DRB3 alleles, 89 DRB4 alleles, 14 DRB5 alleles, 19 DQA1 alleles and 39 DQB1 alleles, with new alleles being discovered continuously. A 2007 update by the WHO nomenclature Committee for Factors of the HLA System (www.anthonynolan.com/HIG/) showed there are 3 DRA alleles, 494 DRB 1 alleles, 1 DRB2 allele, 44 DRB3 alleles, 13 DRB4 alleles, 18 DRB5 alleles, 3 DRB6 alleles, 2 DRB7 alleles, 10 DRB8 alleles, 1 DRB9 allele, 34 DQA1 alleles, 83 DQB1 alleles, 23 DPA1 alleles, 126 DPB1 alleles, 4 DMA alleles, 7 DMB alleles, 12 DOA alleles and 9 DOB alleles. As used herein, the term Class II MHC polypeptide includes allelic forms of any known Class II MHC polypeptide.

MOD-Less Multimeric-TMAPPs (MOD-Less m-TMAPPs)

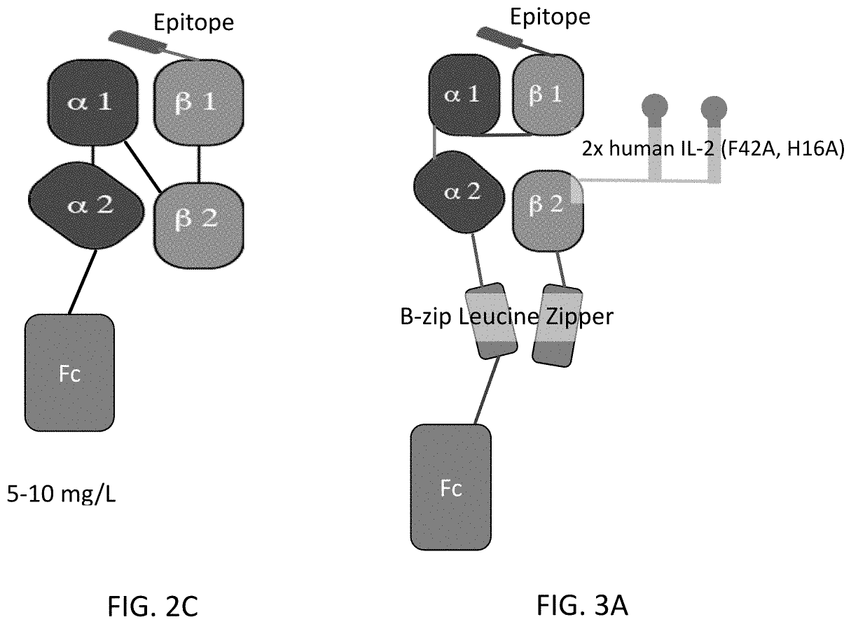

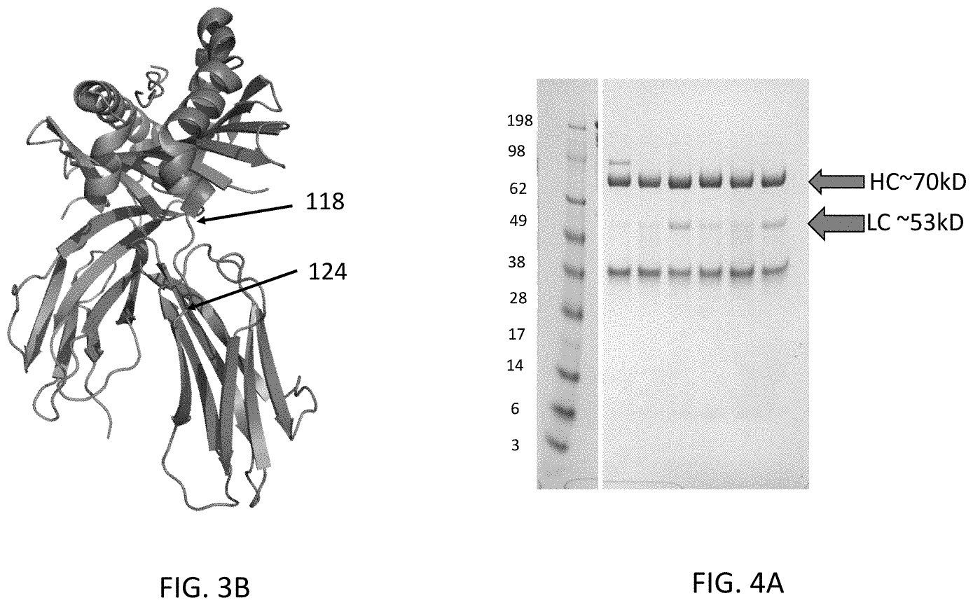

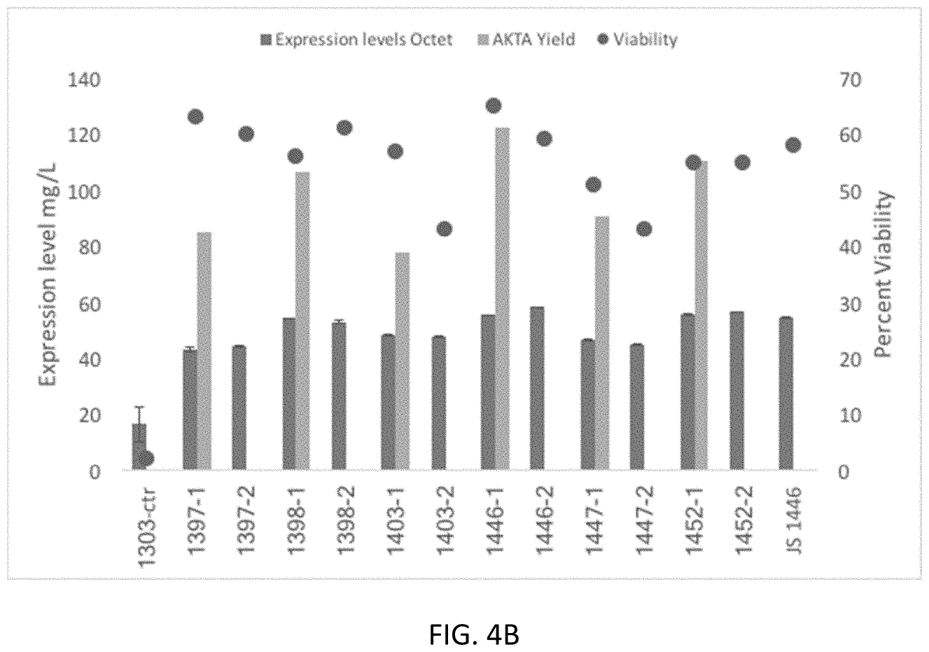

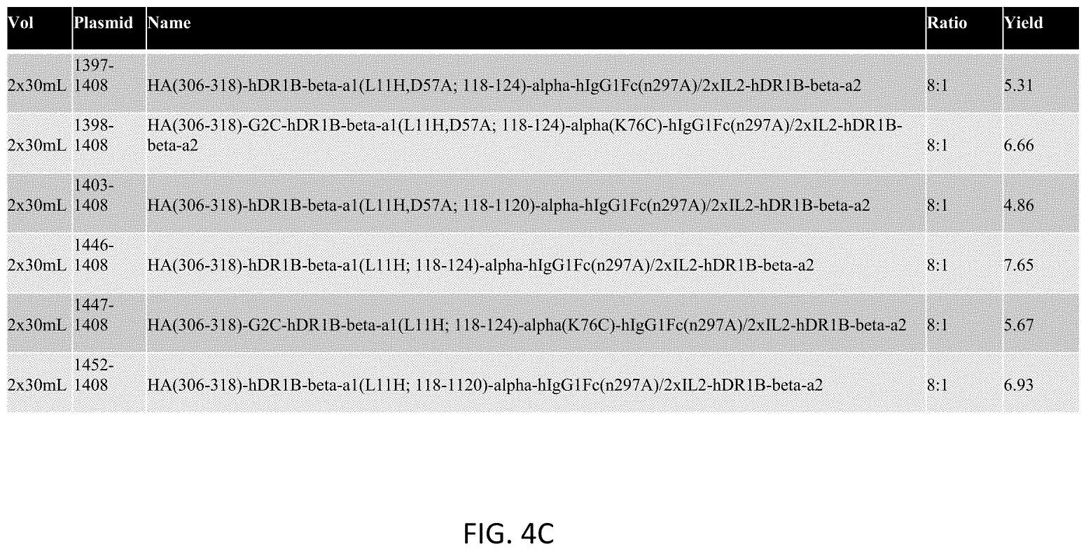

A TMAPP (including those having a chemical conjugation site, or its epitope conjugate) that comprises two (or more) polypeptide chains is denoted as a m-TMAPP. In an embodiment, the m-TMAPPs comprise MHC Class II α1, α2, and β1 polypeptide sequences, but do not comprise any MODs. In another embodiment, the m-TMAPPs comprise MHC Class II α1, α2, β1, and β2 polypeptide sequences, but do not comprise any MODs. mTMAPPs that do not comprise a MOD are denoted as, for example, MOD-less m-TMAPPs. In an embodiment, where a m-TMAPP (e.g., MOD-less m-TMAPPs) comprises two polypeptides (a first and second polypeptide), each of those polypeptides comprises at least one MHC Class II polypeptide. In some cases, the two polypeptide chains are covalently linked to one another, e.g., via a disulfide bond. In other instances, the two polypeptide chains are not covalently linked to one another; and in some of these cases, each of the two polypeptide chains comprises a member of a dimerization pair. Examples of MOD-less mTMAPP epitope conjugates with an epitope peptide covalently attached to the MHC Class II β1 polypeptide by a linker are depicted schematically in FIG. 2A and FIG. 2B.

The MOD-less m-TMAPPs of embodiments A to L comprise at least one chemical conjugation site. In any of embodiments A through L, the chemical conjugation sites may be placed within or at the termini (N- and/or C-terminus) a recited polypeptide of the MOD-less m-TMAPP (e.g., Class II MHC α1, α2, β1 and/or β2 polypeptide sequences, scaffolds, and dimerization domains etc.). Chemical conjugation sites may also be included within or at the ends of linkers (e.g., optional linkers) attached to or inserted between any of the recited polypeptides of a m-TMAPP (e.g., the MHC Class II polypeptide sequences, scaffolds, and dimerization domains etc.), including at N- or C-terminal end(s) of a linker located at the N- or C-terminus of a m-TMAPP polypeptide. In an embodiment, at least one chemical conjugation site is located within or at a N- or C-terminal end of a MOD-less m-TMAPP polypeptide or a linker located at the N- or C-terminus of a first or second polypeptide of a m-TMAPP. In an embodiment, at least one chemical conjugation site is located within or at the N-terminal end of a MOD-less m-TMAPP polypeptide, or a linker (e.g., an optional linker) located at the N-terminus of a first or second polypeptide of the MOD-less m-TMAPP.

In some embodiments, at least one chemical conjugation site (e.g., for an epitope containing peptide) of a MOD-less mTMAPP is located: (a) at the N-terminus of a first or second polypeptide of a MOD-less m-TMAPP molecule, where a MHC Class II α1, α2, β1, or β2 polypeptide sequence is located; or (b) within or at the N-terminus of a linker (e.g., an optional linker) located at the N-terminus of the first or second polypeptide (e.g., the “optional linker” recited in embodiments A-L). In one such embodiment, the MHC Class II β1 polypeptide or an optional linker is located at the N-terminus of the first or second polypeptide, and either the MHC Class II β1 polypeptide or the linker comprises the chemical conjunction site.

When a m-TMAPP (e.g., a MOD-less m-TMAPP of any of embodiments A to L) is converted to an epitope conjugate, the MOD-less m-TMAPP-epitope conjugate comprises an epitope covalently attached at one or more of the chemical conjugation sites. After conjugation the m-TMAPP may contain additional conjugation sites (e.g., for conjugation of a payload).

Some MOD-less m-TMAPPs comprising at least one chemical conjugation site are provided in embodiments A-L:

(A) In an embodiment, a MOD-less m-TMAPP having a chemical conjugation site comprises: a) a first polypeptide comprising, in order from N-terminus to C-terminus: i) a MHC Class II α1 polypeptide; and ii) a MHC Class II α2 polypeptide; and b) a second polypeptide comprising, in order from N-terminus to C-terminus: i) an optional linker; ii) a MHC Class II β1 polypeptide; and iii) a MHC Class II β2 polypeptide; wherein when the antigen-presenting polypeptide has not been conjugated to an epitope the optional linker and/or the MHC Class II β1 polypeptide may comprise one or more chemical conjugation sites (e.g., for epitope attachment). In some cases, the first and/or second polypeptides may comprise a linker (e.g., a peptide linker) between any one or more of the recited elements.

(B) In an embodiment, a MOD-less m-TMAPP having a chemical conjugation site comprises: a) a first polypeptide comprising, in order from N-terminus to C-terminus: i) a MHC Class II α1 polypeptide; and ii) a MHC Class II α2 polypeptide; and b) a second polypeptide comprising, in order from N-terminus to C-terminus: i) an optional linker; ii) a MHC Class II β1 polypeptide; iii) a MHC Class II β2 polypeptide; and iv) an immunoglobulin or non-immunoglobulin scaffold polypeptide; wherein when the antigen-presenting polypeptide has not been conjugated to an epitope the optional linker and/or the MHC Class II β1 polypeptide may comprise one or more chemical conjugation sites. In some cases, the first and/or second polypeptides may comprise a linker (e.g., a peptide linker) between any one or more of the recited elements.

(C) In an embodiment, a MOD-less m-TMAPP having a chemical conjugation site comprises: a) a first polypeptide comprising, in order from N-terminus to C-terminus: i) a MHC Class II α1 polypeptide; and ii) a MHC Class II α2 polypeptide; and b) a second polypeptide comprising, in order from N-terminus to C-terminus: i) an optional linker; ii) a MHC Class II β1 polypeptide; iii) a MHC Class II β2 polypeptide; and iv) an immunoglobulin (e.g., (Ig) Fc) polypeptide wherein when the antigen-presenting polypeptide has not been conjugated to an epitope the optional linker and/or the MHC Class II β1 polypeptide may comprise one or more chemical conjugation sites (e.g., for epitope attachment). In some cases, the first and/or second polypeptides may comprise a linker (e.g., a peptide linker) between any one or more of the recited elements. In some cases, the second polypeptide optionally comprises a linker between the MHC Class II β1 polypeptide and the Class II β2 polypeptide, and/or the Class II β2 polypeptide and the immunoglobulin polypeptide.

(D) In an embodiment, a MOD-less m-TMAPP having a chemical conjugation site comprises: a) a first polypeptide comprising, in order from N-terminus to C-terminus: i) a MHC Class II α1 polypeptide; ii) a MHC Class II α2 polypeptide; and iii) a first member of a dimerizer pair; and b) a second polypeptide comprising, in order from N-terminus to C-terminus: i) an optional linker; ii) a MHC Class II β1 polypeptide; iii) a MHC Class II β2 polypeptide; and iv) a second member of the dimerizer pair; wherein when the antigen-presenting polypeptide has not been conjugated to an epitope the optional linker and/or the MHC Class II β1 polypeptide may comprise one or more chemical conjugation sites (e.g., for epitope attachment); wherein the first and second members of the dimerizer pair bind to one another non-covalently. In some cases, the first and second members of the dimerizer pair bind to one another non-covalently without the need for a dimerization agent. In some cases, the first and second members of the dimerizer pair bind to one another non-covalently in the presence of a dimerizer agent. In some cases, the first and/or second polypeptides may comprise a linker (e.g., a peptide linker) between any one or more of the recited elements.

(E) In an embodiment, a MOD-less m-TMAPP having a chemical conjugation site comprises: a) a first polypeptide comprising, in order from N-terminus to C-terminus: i) a MHC Class II α1 polypeptide; ii) a MHC Class II α2 polypeptide; and iii) a first member of a dimerizer pair; and b) a second polypeptide comprising, in order from N-terminus to C-terminus: i) an optional linker; ii) a MHC Class II β1 polypeptide; iii) a MHC Class II β2 polypeptide; iv) a second member of the dimerizer pair; and v) an immunoglobulin or non-immunoglobulin scaffold polypeptide; wherein when the antigen-presenting polypeptide has not been conjugated to an epitope the optional linker and/or the MHC Class II β1 polypeptide may comprise one or more chemical conjugation sites (e.g., for epitope attachment); and wherein the first and second members of the dimerizer pair bind to one another non-covalently). In some cases, the first and/or second polypeptides may comprise a linker (e.g., a peptide linker) between any one or more of the recited elements. In some cases, the second polypeptide optionally comprises a linker between the MHC Class II β1 polypeptide and the Class II β2 polypeptide, the Class II β2 polypeptide and the second member of the dimerizer pair, and/or the second member of the dimerizer pair and the immunoglobulin or non-immunoglobulin scaffold polypeptide.

(F) In an embodiment, a MOD-less m-TMAPP having a chemical conjugation site comprises: a) a first polypeptide comprising, in order from N-terminus to C-terminus: i) a MHC Class II α1 polypeptide; ii) a MHC Class II α2 polypeptide; and iii) a first member of a dimerizer pair; and b) a second polypeptide comprising, in order from N-terminus to C-terminus: i) an optional linker; ii) a MHC Class II β1 polypeptide; iii) a MHC Class II β2 polypeptide; iv) a second member of the dimerizer pair; and v) an Ig Fc polypeptide; wherein when the antigen-presenting polypeptide has not been conjugated to an epitope the optional linker and/or the MHC Class II β1 polypeptide may comprise one or more chemical conjugation sites (e.g., for epitope attachment); and wherein the first and second members of the dimerizer pair bind to one another non-covalently In some cases, the first and/or second polypeptides may comprise a linker (e.g., a peptide linker) between any one or more of the recited elements. In some cases, the second polypeptide optionally comprises a linker between the MHC Class II β1 polypeptide and the Class II β2 polypeptide, the Class II β2 polypeptide and the second member of the dimerizer pair, and/or the second member of the dimerizer pair and the Ig Fc polypeptide.

(G) In an embodiment, a MOD-less m-TMAPP having a chemical conjugation site comprises: a) a first polypeptide comprising, in order from N-terminus to C-terminus: i) a MHC Class II α1 polypeptide; ii) a MHC Class II α2 polypeptide; and iii) a first leucine zipper polypeptide; and b) a second polypeptide comprising, in order from N-terminus to C-terminus: i) an optional linker; ii) a MHC Class II β1 polypeptide; iii) a MHC Class II β2 polypeptide; iv) a second leucine zipper polypeptide; and v) an Ig Fc polypeptide; wherein when the antigen-presenting polypeptide has not been conjugated to an epitope the optional linker and/or the MHC Class II β1 polypeptide may comprise one or more chemical conjugation sites (e.g., for epitope attachment); and wherein the first and second members of the leucine zipper pair bind to one another non-covalently. In some cases, the first and/or second polypeptides may comprise a linker (e.g., a peptide linker) between any one or more of the recited elements. In some cases, the second polypeptide optionally comprises a linker between the peptide antigen and the MHC Class II β1 polypeptide. In some cases, the second polypeptide comprises a linker between the MHC Class II β1 polypeptide and the Class II β2 polypeptide, the Class II β2 polypeptide and the second leucine zipper polypeptide, and/or the second leucine zipper polypeptide and the Ig Fc polypeptide; and/or the first polypeptide optionally comprises a linker between the MHC Class II α2 polypeptide and the first leucine zipper polypeptide (first member of the dimerizing pair).

(H) In an embodiment, a MOD-less m-TMAPP having a chemical conjugation site comprises: a) a first polypeptide comprising, in order from N-terminus to C-terminus: i) an optional linker; ii) a MHC Class II β1 polypeptide; iii) a MHC Class II α1 polypeptide; iv) a MHC Class II α2 polypeptide; and v) a first member of a dimerizing pair; and b) a second polypeptide comprising, in order from N-terminus to C-terminus: i) a MHC Class II β2 polypeptide; and ii) a second member of the dimerizing pair; wherein when the antigen-presenting polypeptide has not been conjugated to an epitope the optional linker and/or the MHC Class II β1 polypeptide may comprise one or more chemical conjugation sites (e.g., for epitope attachment); and wherein the first and second members of the leucine zipper pair bind to one another non-covalently. In some cases, the first and/or second polypeptides may comprise a linker (e.g., a peptide linker) between any one or more of the recited elements. In some cases, the first polypeptide optionally comprises a linker between the MHC Class II β1 polypeptide and the MHC Class II α1 polypeptide, the MHC Class II α1 polypeptide and the MHC Class II α2 polypeptide, and/or the MHC Class II α2 polypeptide and the first member of the dimerizing pair; and/or the second polypeptide optionally comprises a linker between the MHC Class II β2 polypeptide and the second member of the dimerizer pair.

(I) In an embodiment, a MOD-less m-TMAPP having a chemical conjugation site comprises: a) a first polypeptide comprising, in order from N-terminus to C-terminus: i) an optional linker; ii) a MHC Class II β1 polypeptide; iii) a MHC Class II α1 polypeptide; iv) a MHC Class II α2 polypeptide; v) a first member of a dimerizing pair; and vi) an immunoglobulin or non-immunoglobulin scaffold polypeptide; and b) a second polypeptide comprising, in order from N-terminus to C-terminus: i) a MHC Class II β2 polypeptide; and ii) a second member of the dimerizing pair; wherein when the antigen-presenting polypeptide has not been conjugated to an epitope the optional linker and/or the MHC Class II β1 polypeptide may comprise one or more chemical conjugation sites (e.g., for epitope attachment); and wherein the first and second members of the dimerizing pair bind to one another non-covalently. In some cases, the first and/or second polypeptides may comprise a linker (e.g., a peptide linker) between any one or more of the recited elements. In some cases, the first polypeptide optionally comprises a linker between the MHC Class II β1 polypeptide and the MHC Class II α1 polypeptide, the MHC Class II α1 polypeptide and the MHC Class II α2 polypeptide, the MHC Class II α2 polypeptide and the first member of the dimerizing pair, and/or the first member of the dimerizing pair and the immunoglobulin or non-immunoglobulin scaffold polypeptide; and/or the second polypeptide optionally comprises a linker between the MHC Class II β2 polypeptide and the second member of the dimerizer pair.

(J) In some cases, a MOD-less m-TMAPP having a chemical conjugation site comprises: a) a first polypeptide comprising, in order from N-terminus to C-terminus: i) an optional linker; ii) a MHC Class II β1 polypeptide; iii) a MHC Class II α1 polypeptide; iv) a MHC Class II α2 polypeptide; v) a first member of a dimerizing pair; and vi) an Ig Fc polypeptide; and b) a second polypeptide comprising, in order from N-terminus to C-terminus: i) a MHC Class II β2 polypeptide; and ii) a second member of the dimerizing pair; wherein when the antigen-presenting polypeptide has not been conjugated to an epitope the optional linker and/or the MHC Class II β1 polypeptide may comprise one or more chemical conjugation sites (e.g., for epitope attachment); and wherein the first and second members of the dimerizing pair bind to one another non-covalently. In some cases, the first and/or second polypeptides may comprise a linker (e.g., a peptide linker) between any one or more of the recited elements.

(K) In some cases, a MOD-less m-TMAPP having a chemical conjugation site comprises: a) a first polypeptide comprising, in order from N-terminus to C-terminus: i) an optional linker; ii) a MHC Class II β1 polypeptide; iii) a MHC Class II α1 polypeptide; iv) a MHC Class II α2 polypeptide; v) a first leucine zipper polypeptide; and vi) an Ig Fc polypeptide; and b) a second polypeptide comprising, in order from N-terminus to C-terminus: i) a MHC Class II β2 polypeptide; and ii) a second leucine zipper polypeptide; wherein when the antigen-presenting polypeptide has not been conjugated to an epitope the optional linker and/or the MHC Class II β1 polypeptide may comprise one or more chemical conjugation sites (e.g., for epitope attachment). In some cases, the first and/or second polypeptides may comprise a linker (e.g., a peptide linker) between any one or more of the recited elements. In some cases, the first polypeptide comprises the optional linker between the peptide antigen and the MHC Class II β1 polypeptide. In some cases, the first polypeptide comprises a linker between the MHC Class II β1 polypeptide and the MHC Class II α1 polypeptide. In some cases, the first polypeptide comprises a linker between the MHC Class II α2 polypeptide and the first member of the dimerizing pair. In some cases, the second polypeptide comprises a linker between the MHC Class II β2 polypeptide and the second member of the dimerizing pair.

(L) In some cases, a MOD-less m-TMAPP having a chemical conjugation site comprises at least two linear polypeptides that together are comprised of four polypeptide components: i) a MHC Class II β1 polypeptide, which may have an optional linker on its N-terminus; ii) a MHC Class II β2 polypeptide; iii) a MHC Class II α1 polypeptide; and iv) a MHC Class II α2 polypeptide; wherein one of the two polypeptides has at its N-terminus the MHC Class II β1 polypeptide with, or without, an optional linker on its N-terminus; wherein when the antigen-presenting polypeptide has not been conjugated to an epitope, the optional linker and/or the MHC Class II β1 polypeptide may comprise one or more chemical conjugation sites (e.g., for attachment of an epitope), such as at the N-terminus of the MHC Class II β1 polypeptide or a linker attached thereto.

Any of the MOD-less TMAPP constructs described in embodiments A-L may further comprise as components of either or both of its first and second polypeptide chains one or more: independently selected immunoglobulin (e.g., an immunoglobulin (Ig) Fc polypeptide), or non-immunoglobulin, scaffold polypeptides; and/or a dimerizer (e.g., leucine zipper) polypeptide; all of which are discussed in the disclosure that follows. The first and/or second polypeptides may also comprise a linker (e.g., a peptide linker, suitable examples of which are described below) between any of the recited elements.

A MOD-less m-TMAPP (e.g., any of the above-mentioned MOD-less m-TMAPPs) having at least one chemical conjugation site (e.g., at a first or second polypeptide N-terminus, or within the optional linker) may be reacted with an epitope to produce a MOD-less m-TMAPP-epitope conjugate having the epitope covalently bound at one or more chemical conjugation sites (e.g., one chemical conjugation site that permits the epitope to be bound and recognized by a TCR). After conjugation, the MOD-less m-TMAPP-epitope conjugates may contain additional chemical conjugation sites (e.g., for conjugation of a payload). Accordingly, the specification also provides for and includes such MOD-less m-TMAPP epitope conjugates.

Single-Chain TMAPPs or “sc-TMAPPs” without MODs (MOD-Less sc-TMAPPs)

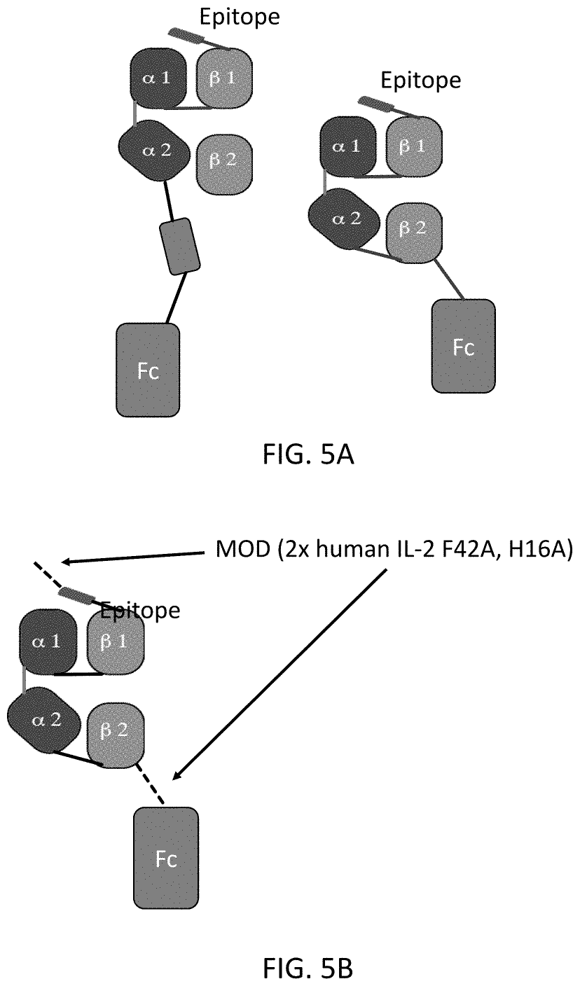

A TMAPP (including those having a chemical conjugation site, or its epitope conjugate) that comprises a single polypeptide chain is denoted as a sc-TMAPP. In an embodiment, the sc-TMAPPs comprise MHC Class II α1, α2, and β1 polypeptide sequences, but does not comprise any MODs. In another embodiment, the sc-TMAPPs comprise MHC Class II α1, α2, β1, and β2 polypeptide sequences, but does not comprise any MODs. sc-TMAPPs that do not comprise a MOD are denoted as MOD-less sc-TMAPPs. Examples of MOD-less sc-TMAPP epitope conjugates with an epitope peptide covalently attached to the MHC Class II β1 polypeptide are depicted schematically in FIG. 2C and in the construct shown on the right side of FIG. 5A.

The MOD-less sc-TMAPPs of embodiments A′ to H′ comprise at least one chemical conjugation site. In any of embodiments A through H that follow, the chemical conjugation sites may be placed within or at the termini (N- and/or C-terminus) of a recited polypeptide of the MOD-less sc-TMAPP (e.g., Class II MHC α1, α2, and β1 polypeptide sequences, scaffolds, and dimerization domains etc.). Chemical conjugation sites may also be included within or at the ends of linkers (e.g., optional linkers) attached to or inserted between any of the recited polypeptides of a MOD-less sc-TMAPP (e.g., the MHC Class II polypeptide sequences, scaffolds, and dimerization domains etc.), including at N- or C-terminal end(s) of a linker located at the N- or C-terminus of a MOD-less sc-TMAPP polypeptide. In an embodiment, at least one chemical conjugation site is located within or at a N- or C-terminal end of a MOD-less sc-TMAPP polypeptide or a linker located at the N- or C-terminus of the MOD-less sc-TMAPP polypeptide. In an embodiment, at least one chemical conjugation site is located within or at the N-terminus of a linker (e.g., an optional linker) located at the N-terminus of the MOD-less sc-TMAPP polypeptide.

In some embodiments, at least one chemical conjugation site (e.g., for an epitope containing peptide) of a MOD-less sc-TMAPP is located: (a) at the N-terminus of the MOD-less sc-TMAPP polypeptide, where a MHC Class II α1, α2, β1, or β2 polypeptide sequence is located; or (b) within or at the N-terminus of a linker (e.g., an optional linker) located at the N-terminus of the MOD-less sc-TMAPP polypeptide (e.g., the “optional linker” recited in embodiments A′-H′). In one such embodiment, the MHC Class II β1 polypeptide, or an optional linker, is located at the N-terminus of the MOD-less sc-TMAPP polypeptide, and either the MHC Class II β1 polypeptide or the linker comprises the chemical conjugation site.

When a sc-TMAPP (e.g., a MOD-less sc-TMAPP of any of embodiments A′ to H′) is converted to an epitope conjugate, the MOD-less sc-TMAPP-epitope conjugate comprises an epitope covalently attached at one or more of the chemical conjugation sites. After conjugation the MOD-less sc-TMAPP may contain additional conjugation sites (e.g., for conjugation of a payload).

In any of the MOD-less sc-TMAPP embodiments A′ through H′, the chemical conjugation sites may be placed within a polypeptide of the MOD-less sc-TMAPP, or at the termini (N- and/or C-terminus) of the MOD-less sc-TMAPP, including in linkers attached or inserted between any of the recited elements of the MOD-less sc-TMAPP (e.g., the MHC Class II polypeptide sequences, scaffolds, and dimerization domains) (including at N- or C-terminal end(s) of a linker located at the N- or C-terminus of a sc-TMAPP polypeptide).

Some MOD-less sc-TMAPPs comprising at least one chemical conjugation site are provided in embodiments A′-H′:

(A′) In an embodiment, a sc-TMAPP having a chemical conjugation site, or its epitope conjugate, comprises, in order from N-terminus to C-terminus: i) an optional linker; ii) a MHC Class II β1 polypeptide; iii) a MHC Class II β2 polypeptide; iv) a MHC Class II α1 polypeptide; and v) a MHC Class II α2 polypeptide; wherein when the antigen-presenting polypeptide has not been conjugated to an epitope the optional linker and/or the MHC Class II β1 polypeptide may comprise one or more chemical conjugation sites (e.g., for epitope attachment).

(B′) In an embodiment, a sc-TMAPP having a chemical conjugation site, or its epitope conjugate, comprises, in order from N-terminus to C-terminus: i) an optional linker; ii) a MHC Class II β1 polypeptide; iii) a MHC Class II β2 polypeptide; iv) a MHC Class II α1 polypeptide; v) a MHC Class II α2 polypeptide; and vi) an immunoglobulin or non-immunoglobulin scaffold polypeptide; wherein when the antigen-presenting polypeptide has not been conjugated to an epitope the optional linker and/or the MHC Class II β1 polypeptide may comprise one or more chemical conjugation sites (e.g., for epitope attachment).

(C′) In an embodiment, a sc-TMAPP having a chemical conjugation site, or its epitope conjugate, comprises, in order from N-terminus to C-terminus: i) an optional linker; ii) a MHC Class II β1 polypeptide; iii) a MHC Class II β2 polypeptide; iv) a MHC Class II α1 polypeptide; v) a MHC Class II α2 polypeptide; and vi) an Ig Fc polypeptide; wherein when the antigen-presenting polypeptide has not been conjugated to an epitope the optional linker and/or the MHC Class II β1 polypeptide may comprise one or more chemical conjugation sites (e.g., for epitope attachment). In some cases, the antigen-presenting polypeptide comprises a linker between the peptide antigen and the MHC Class II β1 polypeptide. In some cases, the antigen-presenting polypeptide comprises a linker between the MHC Class II β2 polypeptide and the MHC Class II α1 polypeptide. In some cases, the antigen-presenting polypeptide comprises a linker between the MHC Class II α2 polypeptide and the immunoglobulin or non-immunoglobulin scaffold.

(D′) In an embodiment, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; and v) a MHC Class II β2 polypeptide; wherein when the antigen-presenting polypeptide has not been conjugated to an epitope the optional linker and/or the MHC Class II β1 polypeptide may comprise one or more chemical conjugation sites (e.g., for epitope attachment).

(E′) In an embodiment, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; and vi) an immunoglobulin or non-immunoglobulin scaffold polypeptide; wherein when the antigen-presenting polypeptide has not been conjugated to an epitope the optional linker and/or the MHC Class II β1 polypeptide may comprise one or more chemical conjugation sites (e.g., for epitope attachment).

(F′) In an embodiment, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; and vi) an Ig Fc polypeptide; wherein when the antigen-presenting polypeptide has not been conjugated to an epitope the optional linker and/or the MHC Class II β1 polypeptide comprise one or more chemical conjugation sites. In some cases, the antigen-presenting polypeptide comprises a linker between the peptide antigen and the MHC Class II β1 polypeptide. In some cases, the antigen-presenting polypeptide comprises a linker between the MHC Class II β1 polypeptide and the MHC Class II α1 polypeptide. In some cases, the antigen-presenting polypeptide comprises a linker between the MHC Class II α2 polypeptide and the MHC Class II β2 polypeptide. In some cases, the antigen-presenting polypeptide comprises a linker between the MHC Class II β2 polypeptide and the Ig or non-Ig scaffold.

(G′) In an embodiment, a sc-TMAPP having a chemical conjugation site, or its epitope conjugate, comprises, in order from N-terminus to C-terminus: i) an optional linker; ii) a HLA β1 polypeptide; iii) a HLA α1 polypeptide; iv) a HLA α2 polypeptide; v) a HLA β2 polypeptide; and vi) an Ig Fc polypeptide; wherein when the antigen-presenting polypeptide has not been conjugated to an epitope the optional linker and/or the MHC Class II β1 polypeptide may comprise one or more chemical conjugation sites (e.g., for epitope attachment).

As one non-limiting example, a sc-TMAPP having a chemical conjugation site, or its epitope conjugate, can comprise, in order from N-terminus to C-terminus: i) an optional linker; ii) a HLA DRB 1 β1 polypeptide; iii) a HLA DRA α1 polypeptide; iv) a HLA DRA α2 polypeptide; v) a HLA DRB β2 polypeptide; and vi) an IgG1 Fc polypeptide; wherein when the antigen-presenting polypeptide has not been conjugated to an epitope the optional linker and/or the MHC Class II β1 polypeptide comprise one or more chemical conjugation sites. When the sc-TMAPP is converted to an epitope conjugate, it comprises an epitope covalently attached at one or more of the chemical conjugation sites. In some cases, the epitope to be conjugated to the sc-TMAPP polypeptide is a hemagglutinin epitope (PKYVKQNTLKLAT; SEQ ID NO:85). In other instances, the epitope is, not PKYVKQNTLKLAT (SEQ ID NO:85); instead, the sc-TMAPP is substituted with a different epitope.

(H′) In some cases, the sc-TMAPPs are linear polypeptides comprised of four polypeptide components: i) a MHC Class II β1 polypeptide, which may have an optional linker on its N-terminus; ii) a MHC Class II β2 polypeptide; iii) a MHC Class II α1 polypeptide; and iv) a MHC Class II α2 polypeptide; wherein the MHC Class II β1 polypeptide or the optional linker on its N-terminus, is located at the N-terminus of the sc-TMAPP polypeptide.

The MOD-less sc-TMAPP polypeptides described in embodiments A′-H′ may further comprise one or more: independently selected immunoglobulin (e.g., an immunoglobulin (Ig) Fc polypeptide), or non-immunoglobulin, scaffold polypeptides; and/or a dimerizer (e.g., leucine zipper) polypeptide; all of which are discussed below. The MOD-less sc-TMAPP polypeptide may also comprise a linker (e.g., a peptide linker, suitable examples of which are described below) between any of the recited elements.

Any of the above-mentioned MOD-less sc-TMAPPs having at least one chemical conjugation site (e.g., at the N-terminus, or within the optional linker) may be reacted with a suitable epitope peptide to produce a MOD-less sc-TMAPP-epitope conjugate having the epitope covalently bound at one or more chemical conjugation sites (e.g, one chemical conjugation site) that permit the epitope to be bound and recognized by a TCR). Accordingly, the specification provides for and includes such MOD-less sc-TMAPPs epitope conjugates.

MHC Class II Alpha Chains

MHC Class II alpha chains comprise an α1 domain and an α2 domain. In some cases, the α1 domain and the α2 domain present in an APC are from the same MHC Class II α chain polypeptide. In some cases, the α1 domain and the α2 domain present in an APC are from two different MHC Class II α chain polypeptides.

MHC Class II alpha chains suitable for inclusion in any TMAPP of the present disclosure lack a signal peptide. A MHC Class II alpha chain suitable for inclusion in any TMAPP of the present disclosure (e.g., a MOD-containing or MOD-less sc- or m-TMAPP having a chemical conjugation site or its epitope conjugate) can have a length of from about 60 amino acids (aa) to about 200 aa; for example, a MHC Class II alpha chain suitable for inclusion in any TMAPP of the present disclosure can have a length of from about 60 aa to about 80 aa, from about 80 aa to about 100 aa, from about 100 aa to about 120 aa, from about 120 aa to about 140 aa, from about 140 aa to about 160 aa, from about 160 aa to about 180 aa, or from about 180 aa to about 200 aa. A MHC Class II α1 domain suitable for inclusion in any TMAPP of the present disclosure can have a length of from about 30 aa to about 95 aa; for example, a MHC Class II α1 domain suitable for inclusion in any TMAPP of the present disclosure can have a length of from about 30 aa to about 40 aa, from about 40 aa to about 50 aa, from about 50 aa to about 60 aa, from about 60 aa to about 70 aa, from about 70 aa to about 80 aa, from about 80 aa to about 90 aa, or from about 90 aa to about 95 aa. A MHC Class II α2 domain suitable for inclusion in any TMAPP of the present disclosure can have a length of from about 30 aa to about 95 aa; for example, a MHC Class II α2 domain suitable for inclusion in any TMAPP of the present disclosure can have a length of from about 30 aa to about 40 aa s, from about 40 aa to about 50 aa, from about 50 aa to about 60 aa, from about 60 aa to about 70 aa, from about 70 aa to about 80 aa, from about 80 aa to about 90 aa, or from about 90 aa to about 95 aa.

DRA

In some cases, a suitable MHC Class II α chain polypeptide for inclusion in any TMAPP of the present disclosure is a DRA polypeptide. A DRA polypeptide can have at least 85%, at least 90%, at least 95%, at least 98%, at least 99%, or 100% amino acid sequence identity with amino acids 26-203 of the DRA amino acid sequence depicted in FIG. 6. In some cases, the DRA polypeptide has a length of about 178 amino acids (e.g., 175, 176, 177, 178, 179, or 180 amino acids).

A DRA polypeptide includes allelic variants, e.g., naturally occurring allelic variants. Thus, in some cases, a suitable DRA polypeptide comprises the following amino acid sequence: IKEEH VIIQAEFYLN PDQSGEFMFD FDGDEIFHVD MAKKETVWRL EEFGRFASFE AQGALANIAV DKANLEIMTK RSNYTPITNV PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:105, which is amino acids 26-203 of (SEQ ID NO:104), or an allelic variant thereof.

A suitable DRA α1 domain comprises an amino acid sequence having at least 85%, at least 90%, at least 95%, at least 98%, at least 99%, or 100% amino acid sequence identity to the following amino acid sequence: VIIQAEFYLN PDQSGEFMFD FDGDEIFHVD MAKKETVWRL EEFGRFASFE AQGALANIAV DKANLEIMTK RSNYTPITN (SEQ ID NO:106); and can have a length of about 84 amino acids (e.g., 80, 81, 82, 83, 84, 85, or 86 amino acids). A suitable DRA α1 domain can comprise the following amino acid sequence: VIIQAEFYLN PDQSGEFMFD FDGDEIFHVD MAKKETVWRL EEFGRFASFE AQGALANIAV DKANLEIMTK RSNYTPITN (SEQ ID NO:106), or a naturally-occurring allelic variant.

A suitable DRA α2 domain comprises an amino acid sequence having at least 85%, at least 90%, at least 95%, at least 98%, at least 99%, or 100% amino acid sequence identity to the following amino acid sequence: V PPEVTVLTNSPVELREPNVL ICFIDKFTPP VVNVTWLRNG KPVTTGVSET VFLPREDHLF RKFHYLPFLPSTEDVYDCRV EHWGLDEPLL KHW (SEQ ID NO:176/); and can have a length of about 94 amino acids (e.g., 90, 91, 92, 93, 94, 95, 96, 97, or 98 amino acids).

DMA

In some cases, a suitable MHC Class II α chain polypeptide for inclusion in any TMAPP of the present disclosure is a DMA polypeptide. A DMA polypeptide can have at least 85%, at least 90%, at least 95%, at least 98%, at least 99%, or 100% amino acid sequence identity with amino acids 27-217 of the DMA amino acid sequence depicted in FIG. 11. In some cases, the DMA polypeptide has a length of about 191 amino acids (e.g., 188, 189, 190, 191, 192, or 193 amino acids).

A “DMA polypeptide” includes allelic variants, e.g., naturally occurring allelic variants. Thus, in some cases, a suitable DMA polypeptide comprises the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSRGFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:134), or an allelic variant thereof.

A suitable DMA α1 domain comprises an amino acid sequence having at least 85%, at least 90%, at least 95%, at least 98%, at least 99%, or 100% amino acid sequence identity to the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO:135); and can have a length of about 98 amino acids (e.g., 94, 95, 96, 97, 98, 99, 100, or 101 amino acids). A suitable DMA α1 domain can comprise the following amino acid sequence: VPEA PTPMWPDDLQ NHTFLHTVYC QDGSPSVGLS EAYDEDQLFF FDFSQNTRVP RLPEFADWAQ EQGDAPAILF DKEFCEWMIQ QIGPKLDGKI PVSR (SEQ ID NO:135), or a naturally-occurring allelic variant thereof.

A suitable DMA α2 domain comprises an amino acid sequence having at least 85%, at least 90%, at least 95%, at least 98%, at least 99%, or 100% amino acid sequence identity to the following amino acid sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:136); and can have a length of about 93 amino acids (e.g., 90, 91, 92, 93, 94, 95, 96, or 97 amino acids). A suitable DMA α2 domain can comprise the following amino acid sequence: GFPIAE VFTLKPLEFG KPNTLVCFVS NLFPPMLTVN WQHHSVPVEG FGPTFVSAVD GLSFQAFSYL NFTPEPSDIF SCIVTHEIDR YTAIAYW (SEQ ID NO:136), or a naturally-occurring allelic variant thereof.

DOA

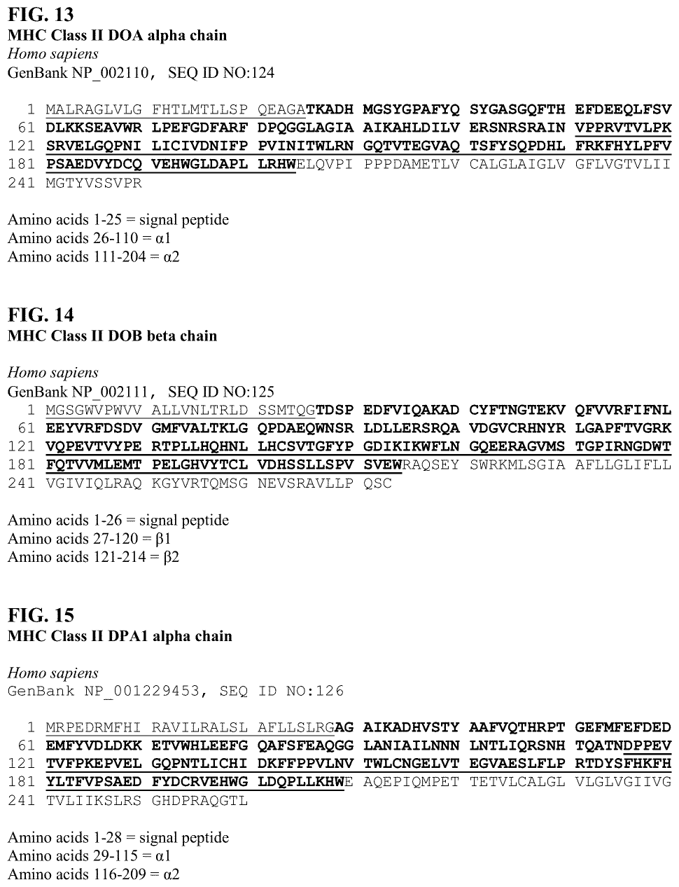

In some cases, a suitable MHC Class II α chain polypeptide for inclusion in any TMAPP of the present disclosure is a DOA polypeptide. A DOA polypeptide can have at least 85%, at least 90%, at least 95%, at least 98%, at least 99%, or 100% amino acid sequence identity with amino acids 26-204 of the DOA amino acid sequence depicted in FIG. 13. In some cases, the DOA polypeptide has a length of about 179 amino acids (e.g., 175, 176, 177, 178, 179, 180, 181, or 182 amino acids).

A “DOA polypeptide” includes allelic variants, e.g., naturally occurring allelic variants. Thus, in some cases, a suitable DOA polypeptide comprises the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:137), or an allelic variant thereof.

A suitable DOA α1 domain comprises an amino acid sequence having at least 85%, at least 90%, at least 95%, at least 98%, at least 99%, or 100% amino acid sequence identity to the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO:138); and can have a length of about 85 amino acids (e.g., 83, 84, 85, 86, 87, or 88 amino acids). A suitable DOA α1 domain can comprise the following amino acid sequence: TKADH MGSYGPAFYQ SYGASGQFTH EFDEEQLFSV DLKKSEAVWR LPEFGDFARF DPQGGLAGIA AIKAHLDILV ERSNRSRAIN (SEQ ID NO:138), or a naturally-occurring allelic variant.

A suitable DOA α2 domain comprises an amino acid sequence having at least 85%, at least 90%, at least 95%, at least 98%, at least 99%, or 100% amino acid sequence identity to the following amino acid sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:139); and can have a length of about 94 amino acids (e.g., 91, 92, 93, 94, 95, 96, or 97 amino acids). A suitable DOA α2 domain can comprise the following amino acid sequence: VPPRVTVLPK SRVELGQPNI LICIVDNIFP PVINITWLRN GQTVTEGVAQ TSFYSQPDHL FRKFHYLPFV PSAEDVYDCQ VEHWGLDAPL LRHW (SEQ ID NO:139), or a naturally-occurring allelic variant thereof.

DPA1

In some cases, a suitable MHC Class II α chain polypeptide for inclusion in any TMAPP of the present disclosure is a DPA1 polypeptide. A DPA1 polypeptide can have at least 85%, at least 90%, at least 95%, at least 98%, at least 99%, or 100% amino acid sequence identity with amino acids 29-209 of the DPA1 amino acid sequence depicted in FIG. 15. In some cases, the DPA1 polypeptide has a length of about 181 amino acids (e.g., 178, 179, 180, 181, 182, 183, or 184 amino acids).

A “DPA1 polypeptide” includes allelic variants, e.g., naturally occurring allelic variants. Thus, in some cases, a suitable DPA1 polypeptide comprises the following amino acid sequence: AG AIKADHVSTY AAFVQTHRPT GEFMFEFDED EMFYVDLDKK ETVWHLEEFG QAFSFEAQGG LANIAILNNN LNTLIQRSNH TQATNDPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:140), or an allelic variant thereof.

A suitable DPA1 α1 domain comprises an amino acid sequence having at least 85%, at least 90%, at least 95%, at least 98%, at least 99%, or 100% amino acid sequence identity to the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO:141); and can have a length of about 87 amino acids (e.g., 84, 85, 86, 87, 88, or 89 amino acids). A suitable DPA1 α1 domain can comprise the following amino acid sequence: AIKADHVSTY AAFVQTHRPT GEFMFEFDED EMFYVDLDKK ETVWHLEEFG QAFSFEAQGG LANIAILNNN LNTLIQRSNH TQATN (SEQ ID NO:141), or a naturally-occurring allelic variant.

A suitable DPA1 α2 domain comprises an amino acid sequence having at least 85%, at least 90%, at least 95%, at least 98%, at least 99%, or 100% amino acid sequence identity to the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:142); and can have a length of about 91-97 amino acids (e.g., 91, 92, 93, 94, 95, 96, or 97 amino acids). A suitable DPA1 α2 domain can comprise the following amino acid sequence: DPPEV TVFPKEPVEL GQPNTLICHI DKFFPPVLNV TWLCNGELVT EGVAESLFLP RTDYSFHKFH YLTFVPSAED FYDCRVEHWG LDQPLLKHW (SEQ ID NO:142), or a naturally-occurring allelic variant thereof.

DQA1

In some cases, a suitable MHC Class II α chain polypeptide for inclusion in any TMAPP of the present disclosure is a DQA1 polypeptide. A DQA1 polypeptide can have at least 85%, at least 90%, at least 95%, at least 98%, at least 99%, or 100% amino acid sequence identity with amino acids 24-204 of the DQA1 amino acid sequence depicted in FIG. 17. In some cases, the DQA1 polypeptide has a length of about 181 amino acids (e.g., 177, 178, 179, 180, 181, 182, or 183 amino acids).

A “DQA1 polypeptide” includes allelic variants, e.g., naturally occurring allelic variants. Thus, in some cases, a suitable DQA1 polypeptide comprises the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:143), or an allelic variant thereof.

A suitable DQA1 α1 domain comprises an amino acid sequence having at least 85%, at least 90%, at least 95%, at least 98%, at least 99%, or 100% amino acid sequence identity to the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:144); and can have a length of about 87 amino acids (e.g., 84, 85, 86, 87, 88, or 89 amino acids). A suitable DQA1 α1 domain can comprise the following amino acid sequence: EDIVADH VASCGVNLYQ FYGPSGQYTH EFDGDEQFYV DLERKETAWR WPEFSKFGGF DPQGALRNMA VAKHNLNIMI KRYNSTAATN (SEQ ID NO:144), or a naturally-occurring allelic variant.

A suitable DQA1 α2 domain comprises an amino acid sequence having at least 85%, at least 90%, at least 95%, at least 98%, at least 99%, or 100% amino acid sequence identity to the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:145); and can have a length of about 94 amino acids (e.g., 91, 92, 93, 94, 95, 96, or 97 amino acids). A suitable DQA1 α2 domain can comprise the following amino acid sequence: EVPEVTVFSK SPVTLGQPNT LICLVDNIFP PVVNITWLSN GQSVTEGVSE TSFLSKSDHS FFKISYLTFL PSADEIYDCK VEHWGLDQPL LKHW (SEQ ID NO:145), or a naturally-occurring allelic variant thereof.

DQA2

In some cases, a suitable MHC Class II α chain polypeptide for inclusion in any TMAPP of the present disclosure is a DQA2 polypeptide. A DQA2 polypeptide can have at least 85%, at least 90%, at least 95%, at least 98%, at least 99%, or 100% amino acid sequence identity with amino acids 24-204 of the DQA2 amino acid sequence depicted in FIG. 18. In some cases, the DQA2 polypeptide has a length of about 181 amino acids (e.g., 177, 178, 179, 180, 181, 182, or 183 amino acids).

A “DQA2 polypeptide” includes allelic variants, e.g., naturally occurring allelic variants. Thus, in some cases, a suitable DQA2 polypeptide comprises the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:146), or an allelic variant thereof.

A suitable DQA2 α1 domain comprises an amino acid sequence having at least 85%, at least 90%, at least 95%, at least 98%, at least 99%, or 100% amino acid sequence identity to the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN (SEQ ID NO:147); and can have a length of about 87 amino acids (e.g., 84, 85, 86, 87, 88, or 89 amino acids). A suitable DQA2 α1 domain can comprise the following amino acid sequence: EDIVADH VASYGVNFYQ SHGPSGQYTH EFDGDEEFYV DLETKETVWQ LPMFSKFISF DPQSALRNMA VGKHTLEFMM RQSNSTAATN (SEQ ID NO:147), or a naturally-occurring allelic variant.

A suitable DQA2 α2 domain comprises an amino acid sequence having at least 85%, at least 90%, at least 95%, at least 98%, at least 99%, or 100% amino acid sequence identity to the following amino acid sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:148); and can have a length of about 94 amino acids (e.g., 91, 92, 93, 94, 95, 96, or 97 amino acids). A suitable DQA2 α2 domain can comprise the following amino acid sequence: EVPEVTVFSK FPVTLGQPNT LICLVDNIFP PVVNITWLSN GHSVTEGVSE TSFLSKSDHS FFKISYLTFL PSADEIYDCK VEHWGLDEPL LKHW (SEQ ID NO:148), or a naturally-occurring allelic variant thereof.

MHC Class II Beta Chains

MHC Class II beta chains comprise a β1 domain and a β2 domain. In some cases, the β1 domain and the β2 domain present in an APC are from the same MHC Class II β chain polypeptide. In some cases, the β1 domain and the β2 domain present in an APC are from two different MHC Class II chain polypeptides.

MHC Class II beta chains suitable for inclusion in any TMAPP of the present disclosure lack a signal peptide. A MHC Class II beta chain suitable for inclusion in any TMAPP of the present disclosure (e.g., a MOD-containing or MOD-less sc- or m-TMAPP having a chemical conjugation site or its epitope conjugate) can have a length of from about 60 aa to about 210 aa; for example, a MHC Class II beta chain suitable for inclusion in any TMAPP of the present disclosure can have a length of from about 60 aa to about 80 aa, from about 80 aa to about 100 aa, from about 100 aa to about 120 aa, from about 120 aa to about 140 aa, from about 140 aa to about 160 as, from about 160 aa to about 180 aa, from about 180 aa to about 200 aa, or from about 200 aa to about 210 aa. A MHC Class II β1 domain suitable for inclusion in any TMAPP of the present disclosure can have a length of from about 30 aa to about 105 aa; for example, a MHC Class II β1 domain suitable for inclusion in any TMAPP of the present disclosure can have a length of from about 30 aa to about 40 aa, from about 40 aa to about 50 aa, from about 50 aa to about 60 aa, from about 60 aa to about 70 aa, from about 70 aa to about 80 aa, from about 80 aa to about 90 aa, from about 90 aa to about 95 aa, from about 95 aa to about 100 aa, or from about 100 aa to about 105 aa. A MHC Class II β2 domain suitable for inclusion in any TMAPP of the present disclosure can have a length of from about 30 aa to about 105 aa; for example, a MHC Class II β2 domain suitable for inclusion in any TMAPP of the present disclosure can have a length of from about 30 aa to about 40 aa, from about 40 aa to about 50 aa, from about 50 aa to about 60 aa, from about 60 aa to about 70 aa, from about 70 aa to about 80 aa, from about 80 aa to about 90 aa, from about 90 aa to about 95 aa, from about 95 aa to about 100 aa, or from about 100 aa to about 105 aa.

DRB1

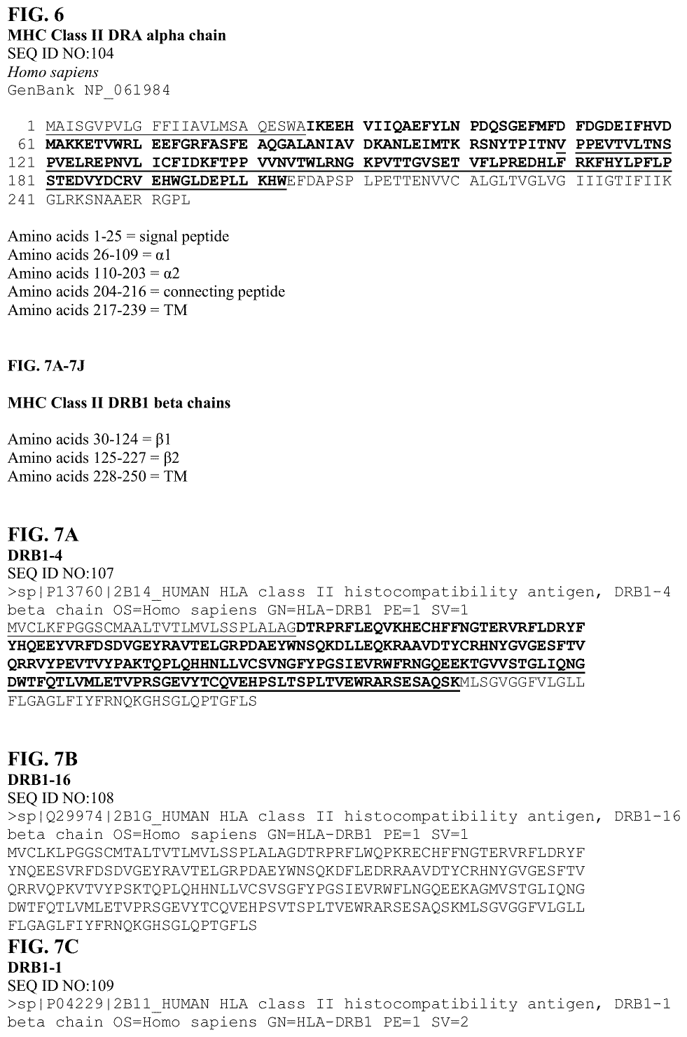

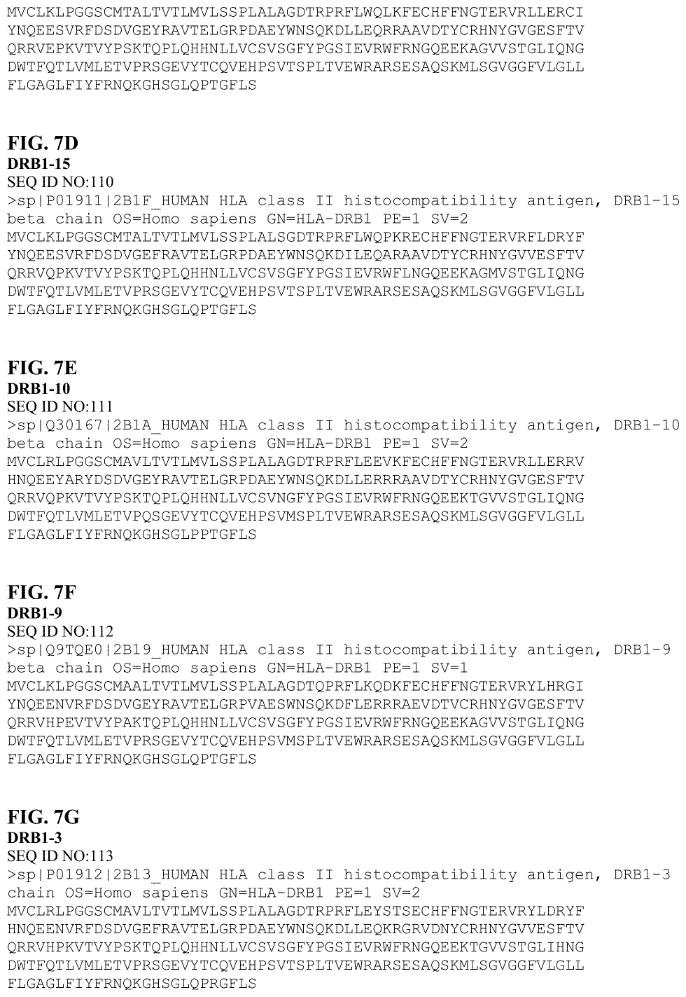

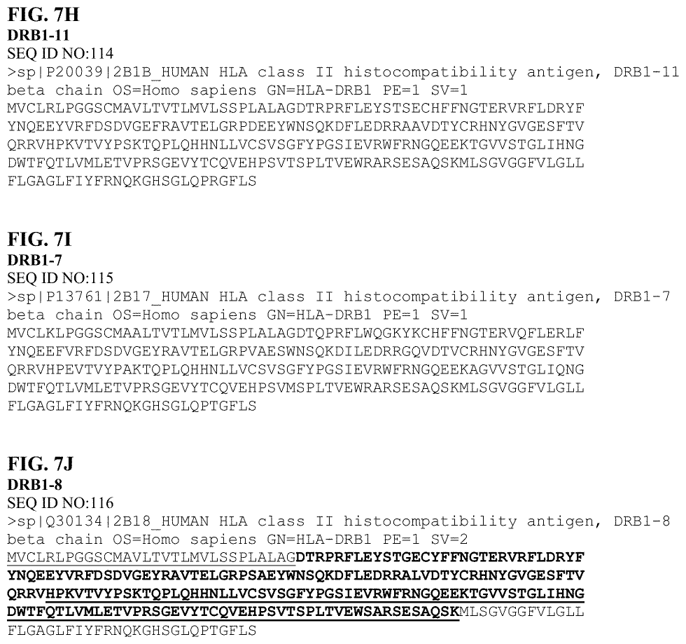

In some cases, a suitable MHC Class II β chain polypeptide for inclusion in any TMAPP of the present disclosure is a DRB1 polypeptide. A DRB1 polypeptide can have at least 85%, at least 90%, at least 95%, at least 98%, at least 99%, or 100% amino acid sequence identity with amino acids 30-227 of the DRB1 amino acid sequence depicted in any one of FIGS. 7A-7J. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7A. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7B. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7C. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7D. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7E. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7F. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7G. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7H. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7I. In some cases, a DRB1 polypeptide can have at least 85%, at least 90%, at least 95%, at least 98%, at least 99%, or 100% amino acid sequence identity with amino acids 30-227 of the DRB1 amino acid sequence depicted in FIG. 7J. In some cases, the DRB1 polypeptide has a length of about 198 amino acids (e.g., 195, 196, 197, 198, 199, 200, 201, or 202 amino acids).

A “DRB1 polypeptide” includes allelic variants, e.g., naturally occurring allelic variants. Thus, in some cases, a suitable DRB1 polypeptide comprises the following amino acid sequence: DTRPRFLEQV KHECHFFNGT ERVRFLDRYF YHQEEYVRFD SDVGEYRAVT ELGRPDAEYW NSQKDLLEQK RAAVDTYCRH NYGVGESFTV QRRVYPEVTV YPAKTQPLQH HNLLVCSVNG FYPGSIEVRW FRNGQEEKTG VVSTGLIQNG DWTFQTLVML ETVPRSGEVY TCQVEHPSLT SPLTVEWRAR SESAQSK (SEQ ID NO:149), or an allelic variant thereof.

A suitable DRB1 β1 domain comprises an amino acid sequence having at least 85%, at least 90%, at least 95%, at least 98%, at least 99%, or 100% amino acid sequence identity to the following amino acid sequence: DTRPRFLEQV KHECHFFNGT ERVRFLDRYF YHQEEYVRFD SDVGEYRAVT ELGRPDAEYW NSQKDLLEQK RAAVDTYCRH NYGVGESFTV QRRV (SEQ ID NO:150); and can have a length of about 95 amino acids (e.g., 92, 93, 94, 95, 96, 97, or 98 amino acids). A suitable DRB1 β1 domain can comprise the following amino acid sequence: DTRPRFLEQV KHECHFFNGT ERVRFLDRYF YHQEEYVRFD SDVGEYRAVT ELGRPDAEYW NSQKDLLEQK RAAVDTYCRH NYGVGESFTV QRRV (SEQ ID NO:150), or a naturally-occurring allelic variant.

A suitable DRB1 β2 domain comprises an amino acid sequence having at least 85%, at least 90%, at least 95%, at least 98%, at least 99%, or 100%, amino acid sequence identity to the following amino acid sequence: YPEVTVYPAK TQPLQHHNLL VCSVNGFYPG SIEVRWFRNG QEEKTGVVST GLIQNGDWTF QTLVMLETVP RSGEVYTCQV EHPSLTSPLT VEWRARSESA QSK (SEQ ID NO:151); and can have a length of about 103 amino acids (e.g., 100, 101, 102, 103, 104, 105, or 106 amino acids). A suitable DRB1 β2 domain can comprise the following amino acid sequence: YPEVTVYPAK TQPLQHHNLL VCSVNGFYPG SIEVRWFRNG QEEKTGVVST GLIQNGDWTF QTLVMLETVP RSGEVYTCQV EHPSLTSPLT VEWRARSESA QSK (SEQ ID NO:151), or a naturally-occurring allelic variant thereof.

DRB3

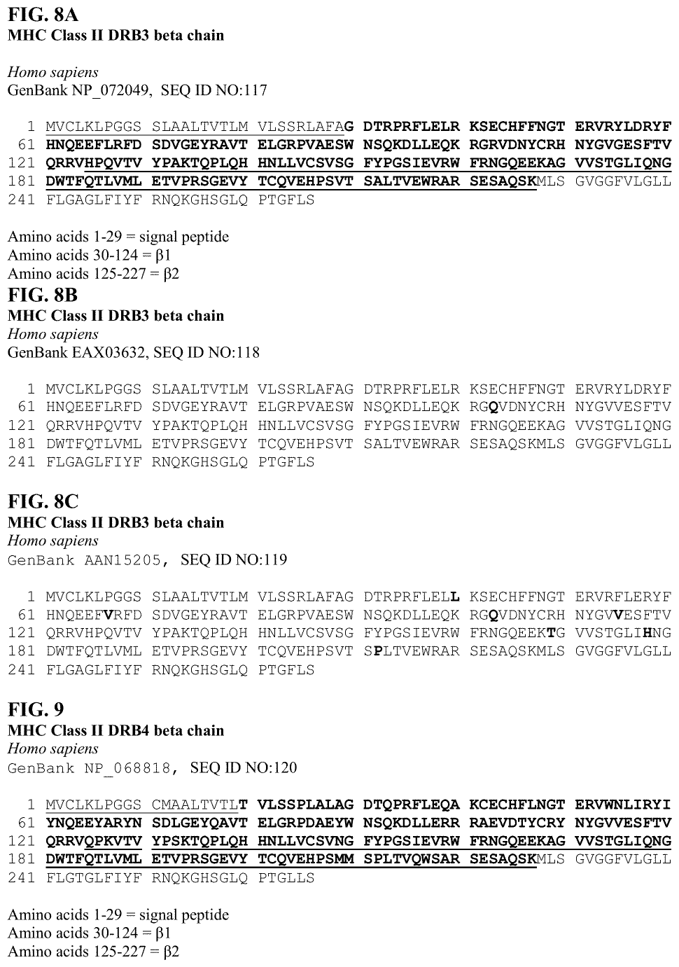

In some cases, a suitable MHC Class II β chain polypeptide for inclusion in any TMAPP of the present disclosure is a DRB3 polypeptide. A DRB3 polypeptide can have at least 85%, at least 90%, at least 95%, at least 98%, at least 99%, or 100% amino acid sequence identity with amino acids 30-227 of the DRB3 amino acid sequence depicted in any one of FIGS. 8A-8C. In some cases, a DRB3 polypeptide can have at least 85%, at least 90%, at least 95%, at least 98%, at least 99%, or 100% amino acid sequence identity with amino acids 30-227 of the DRB3 amino acid sequence depicted in FIG. 8A. In some cases, a DRB3 polypeptide can have at least 85%, at least 90%, at least 95%, at least 98%, at least 99%, or 100% amino acid sequence identity with amino acids 30-227 of the DRB3 amino acid sequence depicted in FIG. 8B. In some cases, a DRB3 polypeptide can have at least 85%, at least 90%, at least 95%, at least 98%, at least 99%, or 100% amino acid sequence identity with amino acids 30-227 of the DRB3 amino acid sequence depicted in FIG. 8C. In some cases, the DRB3 polypeptide has a length of about 198 amino acids (e.g., 195, 196, 197, 198, 199, 200, 201, or 202 amino acids).

A “DRB3 polypeptide” includes allelic variants, e.g., naturally occurring allelic variants. Thus, in some cases, a suitable DRB3 polypeptide comprises the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRVHPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:152), or an allelic variant thereof.

A suitable DRB3 β1 domain comprises an amino acid sequence having at least 85%, at least 90%, at least 95%, at least 98%, at least 99%, or 100% amino acid sequence identity to the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:153); and can have a length of about 95 amino acids (e.g., 93, 94, 95, 96, 97, or 98 amino acids). A suitable DRB3 β1 domain can comprise the following amino acid sequence: DTRPRFLELR KSECHFFNGT ERVRYLDRYF HNQEEFLRFD SDVGEYRAVT ELGRPVAESW NSQKDLLEQK RGRVDNYCRH NYGVGESFTV QRRV (SEQ ID NO:153), or a naturally-occurring allelic variant.

A suitable DRB3 β2 domain comprises an amino acid sequence having at least 85%, at least 90%, at least 95%, at least 98%, at least 99%, or 100% amino acid sequence identity to the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:154); and can have a length of about 103 amino acids (e.g., 100, 101, 102, 103, 104, or 105 amino acids). A suitable DRB3 β2 domain can comprise the following amino acid sequence: HPQVTV YPAKTQPLQH HNLLVCSVSG FYPGSIEVRW FRNGQEEKAG VVSTGLIQNG DWTFQTLVML ETVPRSGEVY TCQVEHPSVT SALTVEWRAR SESAQSK (SEQ ID NO:154), or a naturally-occurring allelic variant thereof.

DRB4

In some cases, a suitable MHC Class II β chain polypeptide for inclusion in any TMAPP is a DRB4 polypeptide. A DRB4 polypeptide can have at least 85%, at least 90%, at least 95%, at least 98%, at least 99%, or 100% amino acid sequence identity with amino acids 30-227 of the DRB4 amino acid sequence depicted in FIG. 9. In some cases, the DRB4 polypeptide has a length of about 198 amino acids (e.g., 195, 196, 197, 198, 199, 200, 201, or 202 amino acids).

A “DRB4 polypeptide” includes allelic variants, e.g., naturally occurring allelic variants. Thus, in some cases, a suitable DRB4 polypeptide comprises the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRVQPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:155), or an allelic variant thereof.

A suitable DRB4 β1 domain comprises an amino acid sequence having at least 85%, at least 90%, at least 95%, at least 98%, at least 99%, or 100% amino acid sequence identity to the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:156); and can have a length of about 95 amino acids (e.g., 93, 94, 95, 96, 97, or 98 amino acids). A suitable DRB4 β1 domain can comprise the following amino acid sequence: T VLSSPLALAG DTQPRFLEQA KCECHFLNGT ERVWNLIRYI YNQEEYARYN SDLGEYQAVT ELGRPDAEYW NSQKDLLERR RAEVDTYCRY NYGVVESFTV QRRV (SEQ ID NO:156), or a naturally-occurring allelic variant.

A suitable DRB4 β2 domain comprises an amino acid sequence having at least 85%, at least 90%, at least 95%, at least 98%, at least 99%, or 100% amino acid sequence identity to the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:157); and can have a length of about 103 amino acids (e.g., 100, 101, 102, 103, 104, or 105 amino acids). A suitable DRB4 β2 domain can comprise the following amino acid sequence: QPKVTV YPSKTQPLQH HNLLVCSVNG FYPGSIEVRW FRNGQEEKAG VVSTGLIQNG DWTFQTLVML ETVPRSGEVY TCQVEHPSMM SPLTVQWSAR SESAQSK (SEQ ID NO:157), or a naturally-occurring allelic variant thereof.

DRB5

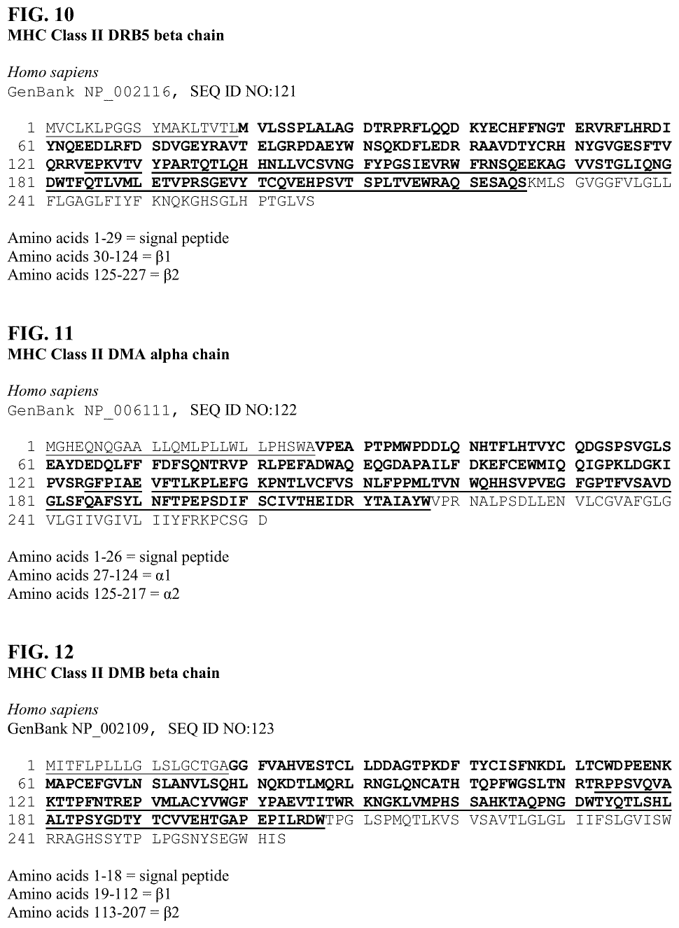

In some cases, a suitable MHC Class II β chain polypeptide for inclusion in any TMAPP of the present disclosure is a DRB5 polypeptide. A DRB5 polypeptide can have at least 85%, at least 90%, at least 95%, at least 98%, at least 99%, or 100% amino acid sequence identity with amino acids 30-227 of the DRB5 amino acid sequence depicted in FIG. 10. In some cases, the DRB5 polypeptide has a length of about 198 amino acids (e.g., 195, 196, 197, 198, 199, 200, 201, or 202 amino acids).

A “DRB5 polypeptide” includes allelic variants, e.g., naturally occurring allelic variants. Thus, in some cases, a suitable DRB5 polypeptide comprises the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRVEPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:158), or an allelic variant thereof.

A suitable DRB5 β1 domain comprises an amino acid sequence having at least 85%, at least 90%, at least 95%, at least 98%, at least 99%, or 100% amino acid sequence identity to the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO:159); and can have a length of about 95 amino acids (e.g., 93, 94, 95, 96, 97, or 98 amino acids). A suitable DRB5 β1 domain can comprise the following amino acid sequence: M VLSSPLALAG DTRPRFLQQD KYECHFFNGT ERVRFLHRDI YNQEEDLRFD SDVGEYRAVT ELGRPDAEYW NSQKDFLEDR RAAVDTYCRH NYGVGESFTV QRRV (SEQ ID NO:159), or a naturally-occurring allelic variant.

A suitable DRB5 β2 domain comprises an amino acid sequence having at least 85%, at least 90%, at least 95%, at least 98%, at least 99%, or 100% amino acid sequence identity to the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:160); and can have a length of about 103 amino acids (e.g., 100, 101, 102, 103, 104, or 105 amino acids). A suitable DRB5 β32 domain can comprise the following amino acid sequence: EPKVTV YPARTQTLQH HNLLVCSVNG FYPGSIEVRW FRNSQEEKAG VVSTGLIQNG DWTFQTLVML ETVPRSGEVY TCQVEHPSVT SPLTVEWRAQ SESAQS (SEQ ID NO:160), or a naturally-occurring allelic variant thereof.

DMB

In some cases, a suitable MHC Class II β chain polypeptide for inclusion in any TMAPP of the present disclosure is a DMB polypeptide. A DMB polypeptide can have at least 85%, at least 90%, at least 95%, at least 98%, at least 99%, or 100% amino acid sequence identity with amino acids 19-207 of the DMB amino acid sequence depicted in FIG. 12. In some cases, the DMB polypeptide has a length of about 189 amino acids (e.g., 187, 188, 189, 190, or 191 amino acids).

A “DMB polypeptide” includes allelic variants, e.g., naturally occurring allelic variants. Thus, in some cases, a suitable DMB polypeptide comprises the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RTRPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:161), or an allelic variant thereof.

A suitable DMB β1 domain comprises an amino acid sequence having at least 85%, at least 90%, at least 95%, at least 98%, at least 99%, or 100% amino acid sequence identity to the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO:162); and can have a length of about 94 amino acids (e.g., 92, 93, 94, 95, 96, or 97 amino acids). A suitable DMB β1 domain can comprise the following amino acid sequence: GG FVAHVESTCL LDDAGTPKDF TYCISFNKDL LTCWDPEENK MAPCEFGVLN SLANVLSQHL NQKDTLMQRL RNGLQNCATH TQPFWGSLTN RT (SEQ ID NO:162), or a naturally-occurring allelic variant.

A suitable DMB β2 domain comprises an amino acid sequence having at least 85%, at least 90%, at least 95%, at least 98%, at least 99%, or 100% amino acid sequence identity to the following amino acid sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:163); and can have a length of about 95 amino acids (e.g., 93, 94, 95, 96, 97, or 98 amino acids). A suitable DMB β2 domain can comprise the following amino acid sequence: RPPSVQVA KTTPFNTREP VMLACYVWGF YPAEVTITWR KNGKLVMPHS SAHKTAQPNG DWTYQTLSHL ALTPSYGDTY TCVVEHTGAP EPILRDW (SEQ ID NO:163), or a naturally-occurring allelic variant thereof.

DOB

In some cases, a suitable MHC Class II β chain polypeptide for inclusion in any TMAPP of the present disclosure is a DOB polypeptide. A DOB polypeptide can have at least 85%, at least 90%, at least 95%, at least 98%, at least 99%, or 100% amino acid sequence identity with amino acids 27-214 of the DOB amino acid sequence depicted in FIG. 14. In some cases, the DOB polypeptide has a length of about 188 amino acids (e.g., 186, 187, 188, 189, or 190 amino acids).

A “DOB polypeptide” includes allelic variants, e.g., naturally occurring allelic variants. Thus, in some cases, a suitable DOB polypeptide comprises the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:164), or an allelic variant thereof.

A suitable DOB β1 domain comprises an amino acid sequence having at least 85%, at least 90%, at least 95%, at least 98%, at least 99%, or 100% amino acid sequence identity to the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK (SEQ ID NO:165); and can have a length of about 94 amino acids (e.g., 92, 93, 94, 95, 96, or 97 amino acids). A suitable DOB β1 domain can comprise the following amino acid sequence: TDSP EDFVIQAKAD CYFTNGTEKV QFVVRFIFNL EEYVRFDSDV GMFVALTKLG QPDAEQWNSR LDLLERSRQA VDGVCRHNYR LGAPFTVGRK (SEQ ID NO:165), or a naturally-occurring allelic variant.

A suitable DOB β2 domain comprises an amino acid sequence having at least 85%, at least 90%, at least 95%, at least 98%, at least 99%, or 100% amino acid sequence identity to the following amino acid sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:166); and can have a length of about 94 amino acids (e.g., 92, 93, 94, 95, 96, or 97 amino acids). A suitable DOB β2 domain can comprise the following amino acid sequence: VQPEVTVYPE RTPLLHQHNL LHCSVTGFYP GDIKIKWFLN GQEERAGVMS TGPIRNGDWT FQTVVMLEMT PELGHVYTCL VDHSSLLSPV SVEW (SEQ ID NO:166), or a naturally-occurring allelic variant thereof.

DPB1

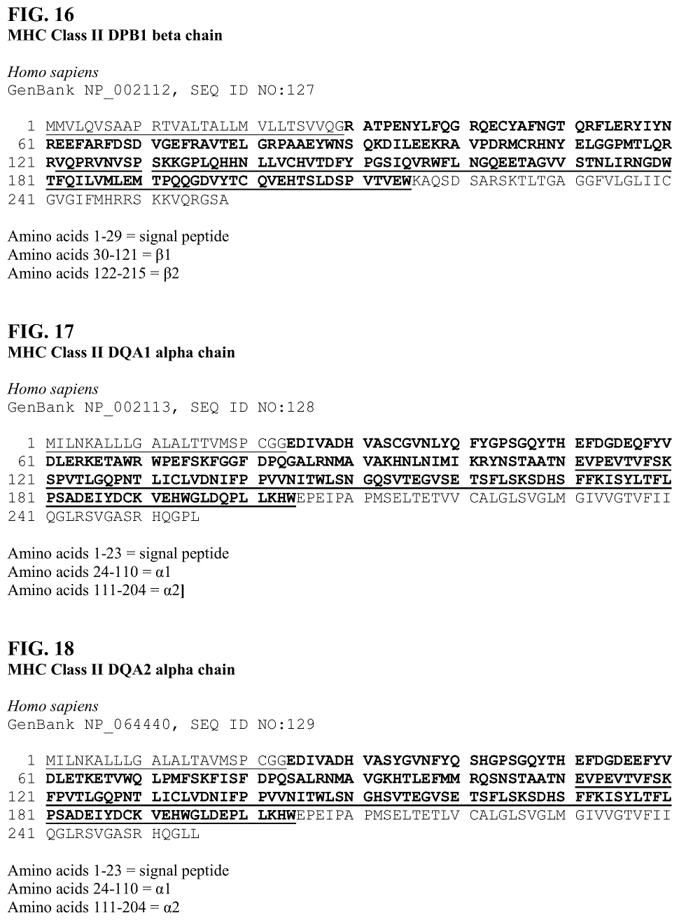

In some cases, a suitable MHC Class II β chain polypeptide for inclusion in any TMAPP of the present disclosure is a DPB1 polypeptide. A DPB1 polypeptide can have at least 85%, at least 90%, at least 95%, at least 98%, at least 99%, or 100% amino acid sequence identity with amino acids 30-215 of the DPB1 amino acid sequence depicted in FIG. 16. In some cases, the DPB1 polypeptide has a length of about 186 amino acids (e.g., 184, 185, 186, 187, or 188 amino acids).

A “DPB1 polypeptide” includes allelic variants, e.g., naturally occurring allelic variants. Thus, in some cases, a suitable DPB1 polypeptide comprises the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR RVQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:167), or an allelic variant thereof.

A suitable DPB1 β1 domain comprises an amino acid sequence having at least 85%, at least 90%, at least 95%, at least 98%, at least 99%, or 100% amino acid sequence identity to the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:168); and can have a length of about 92 amino acids (e.g., 90, 91, 92, 93, or 94 amino acids). A suitable DPB1 β1 domain can comprise the following amino acid sequence: R ATPENYLFQG RQECYAFNGT QRFLERYIYN REEFARFDSD VGEFRAVTEL GRPAAEYWNS QKDILEEKRA VPDRMCRHNY ELGGPMTLQR R (SEQ ID NO:168), or a naturally-occurring allelic variant.

A suitable DPB1 β2 domain comprises an amino acid sequence having at least 85%, at least 90%, at least 95%, at least 98%, at least 99%, or 100% amino acid sequence identity to the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:169); and can have a length of about 94 amino acids (e.g., 92, 93, 94, 95, 96, or 97 amino acids). A suitable DPB1 β2 domain can comprise the following amino acid sequence: VQPRVNVSP SKKGPLQHHN LLVCHVTDFY PGSIQVRWFL NGQEETAGVV STNLIRNGDW TFQILVMLEM TPQQGDVYTC QVEHTSLDSP VTVEW (SEQ ID NO:169), or a naturally-occurring allelic variant thereof.

DQB1

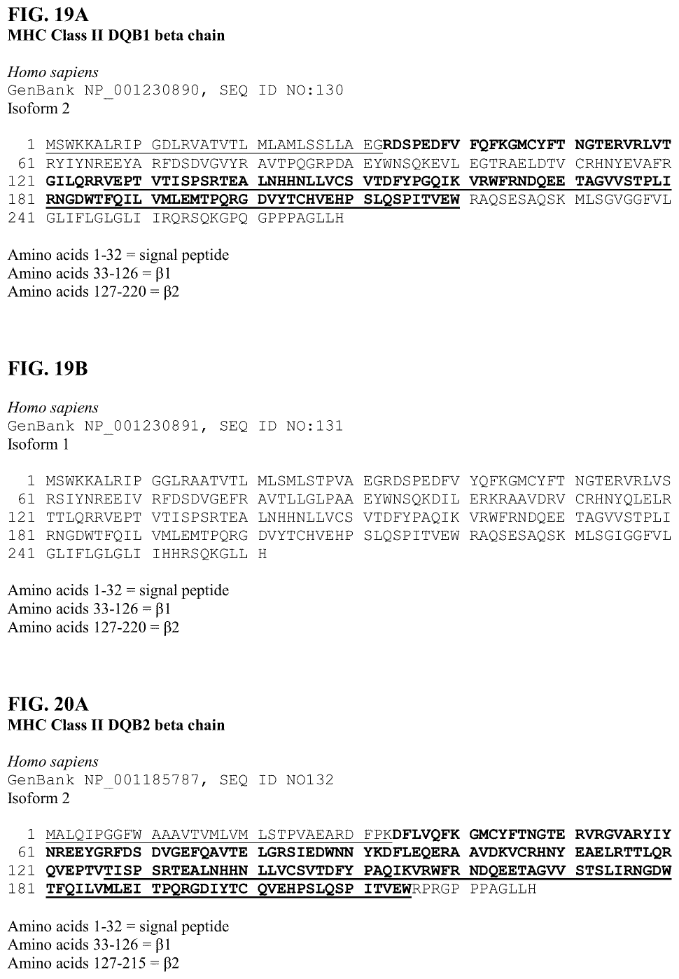

In some cases, a suitable MHC Class II β chain polypeptide for inclusion in any TMAPP of the present disclosure is a DQB1 polypeptide. A DQB1 polypeptide can have at least 85%, at least 90%, at least 95%, at least 98%, at least 99%, or 100% amino acid sequence identity with amino acids 33-220 of the DQB1 amino acid sequence depicted in FIG. 19A or FIG. 19B. In some cases, the DQB1 polypeptide has a length of about 188 amino acids (e.g., 186, 187, 188, 189, 190, 191, or 192 amino acids).

A “DQB1 polypeptide” includes allelic variants, e.g., naturally occurring allelic variants. Thus, in some cases, a suitable DQB1 polypeptide comprises the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRRVEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:170), or an allelic variant thereof.

A suitable DQB1 β1 domain comprises an amino acid sequence having at least 85%, at least 90%, at least 95%, at least 98%, at least 99%, or 100% amino acid sequence identity to the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:171); and can have a length of about 94 amino acids (e.g., 92, 93, 94, 95, or 96 amino acids). A suitable DQB1 β1 domain can comprise the following amino acid sequence: RDSPEDFV FQFKGMCYFT NGTERVRLVT RYIYNREEYA RFDSDVGVYR AVTPQGRPDA EYWNSQKEVL EGTRAELDTV CRHNYEVAFR GILQRR (SEQ ID NO:171), or a naturally-occurring allelic variant.

A suitable DQB1 β2 domain comprises an amino acid sequence having at least 85%, at least 90%, at least 95%, at least 98%, at least 99%, or 100% amino acid sequence identity to the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:172); and can have a length of about 94 amino acids (e.g., 92, 93, 94, 95, or 96 amino acids). A suitable DQB1 β2 domain can comprise the following amino acid sequence: VEPT VTISPSRTEA LNHHNLLVCS VTDFYPGQIK VRWFRNDQEE TAGVVSTPLI RNGDWTFQIL VMLEMTPQRG DVYTCHVEHP SLQSPITVEW (SEQ ID NO:172), or a naturally-occurring allelic variant thereof.

DQB2

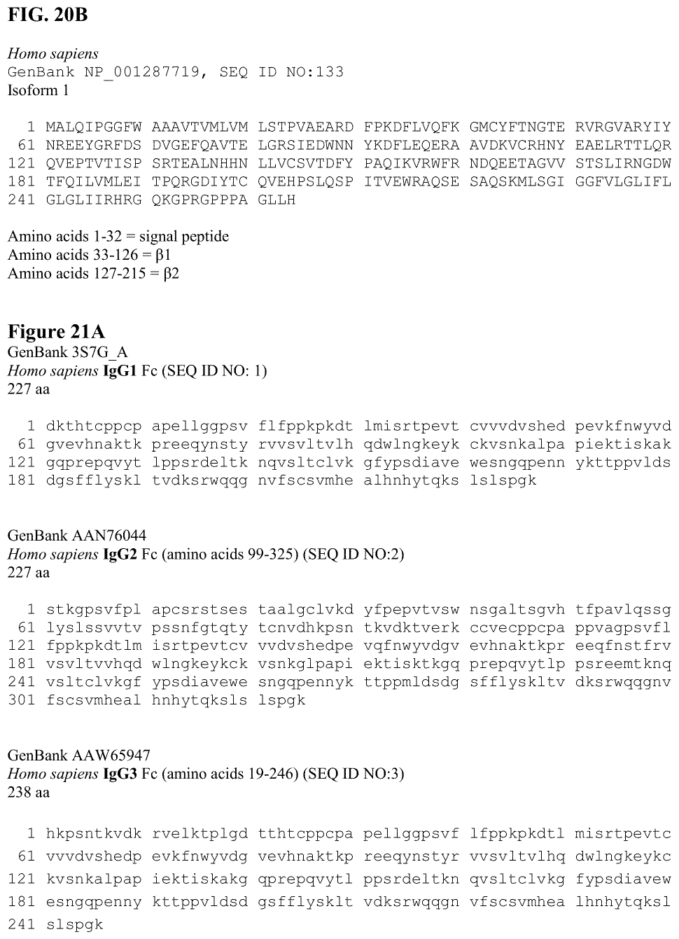

In some cases, a suitable MHC Class II β chain polypeptide for inclusion in any TMAPP of the present disclosure is a DQB2 polypeptide. A DQB2 polypeptide can have at least 85%, at least 90%, at least 95%, at least 98%, at least 99%, or 100% amino acid sequence identity with amino acids 33-215 of the DQB2 amino acid sequence depicted in FIG. 20A or FIG. 20B. In some cases, the DQB2 polypeptide has a length of about 178 amino acids (e.g., 175, 176, 177, 178, 179, 180, 181, or 182 amino acids).

A “DQB2 polypeptide” includes allelic variants, e.g., naturally occurring allelic variants. Thus, in some cases, a suitable DQB2 polypeptide comprises the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTVTISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:173), or an allelic variant thereof.

A suitable DQB2 β1 domain comprises an amino acid sequence having at least 85%, at least 90%, at least 95%, at least 98%, at least 99%, or 100% amino acid sequence identity to the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTV (SEQ ID NO:174); and can have a length of about 94 amino acids (e.g., 92 93, 94, 95, 96, or 97 amino acids). A suitable DQB2 β1 domain can comprise the following amino acid sequence: DFLVQFK GMCYFTNGTE RVRGVARYIY NREEYGRFDS DVGEFQAVTE LGRSIEDWNN YKDFLEQERA AVDKVCRHNY EAELRTTLQR QVEPTV (SEQ ID NO:174), or a naturally-occurring allelic variant.

A suitable DQB2 β2 domain comprises an amino acid sequence having at least 85%, at least 90%, at least 95%, at least 98%, at least 99%, or 100% amino acid sequence identity to the following amino acid sequence: TISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:175); and can have a length of about 94 amino acids (e.g., 92 93, 94, 95, 96, or 97 amino acids). A suitable DQB2 β2 domain can comprise the following amino acid sequence: TISP SRTEALNHHN LLVCSVTDFY PAQIKVRWFR NDQEETAGVV STSLIRNGDW TFQILVMLEI TPQRGDIYTC QVEHPSLQSP ITVEW (SEQ ID NO:175//), or a naturally-occurring allelic variant thereof.

Scaffold Polypeptides

An immunoglobulin or non-immunoglobulin scaffold (e.g., a Fc polypeptide, or another suitable scaffold polypeptide) can be incorporated into a polypeptide of any TMAPP of the present disclosure (e.g., a MOD-containing or MOD-less sc- or m-TMAPP having a chemical conjugation site or its epitope conjugate).

Suitable scaffold polypeptides include antibody-based scaffold polypeptides and non-antibody-based scaffolds. Non-antibody-based scaffolds include, e.g., albumin, an XTEN (extended recombinant) polypeptide, transferrin polypeptide, a Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:103), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see, also, Schellenberger et al. (2009) Nat Biotechnol. 27:1186. Suitable albumin polypeptides include, e.g., human serum albumin.

Suitable scaffold polypeptides will, in some cases, be half-life extending polypeptides. Thus, in some cases, a suitable scaffold polypeptide increases the in vivo half-life (e.g., the serum half-life) of the TMAPP, compared to a control TMAPP lacking the scaffold polypeptide. For example, in some cases, a scaffold polypeptide increases the in vivo half-life (e.g., the serum half-life) of the TMAPP, compared to a control TMAPP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, incorporating a Fc polypeptide into a TMAPP increases the in vivo half-life (e.g., the serum half-life) of the TMAPP, compared to a control TMAPP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

Fc Polypeptides

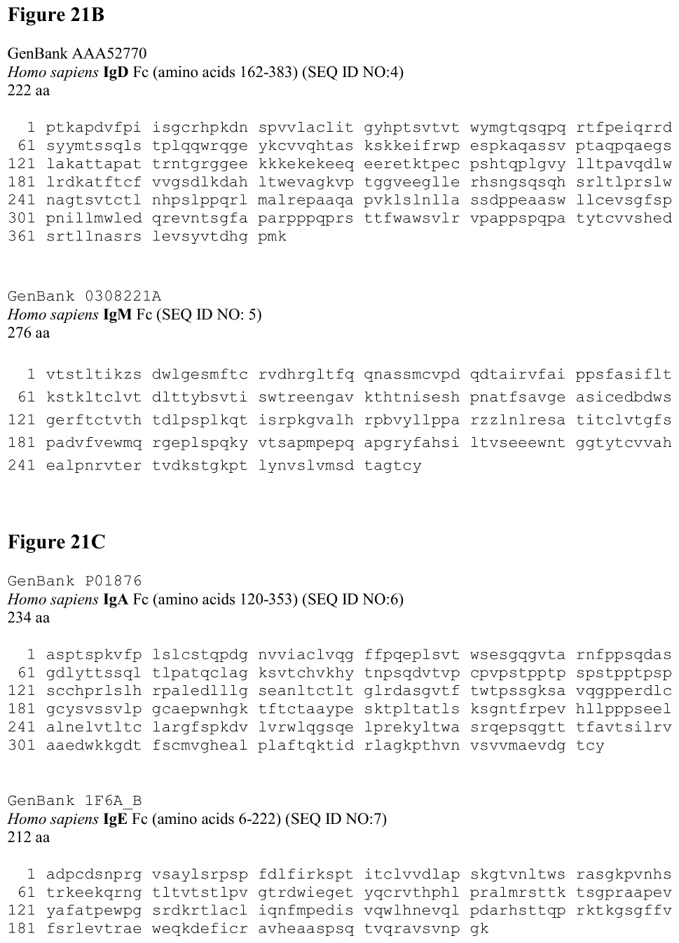

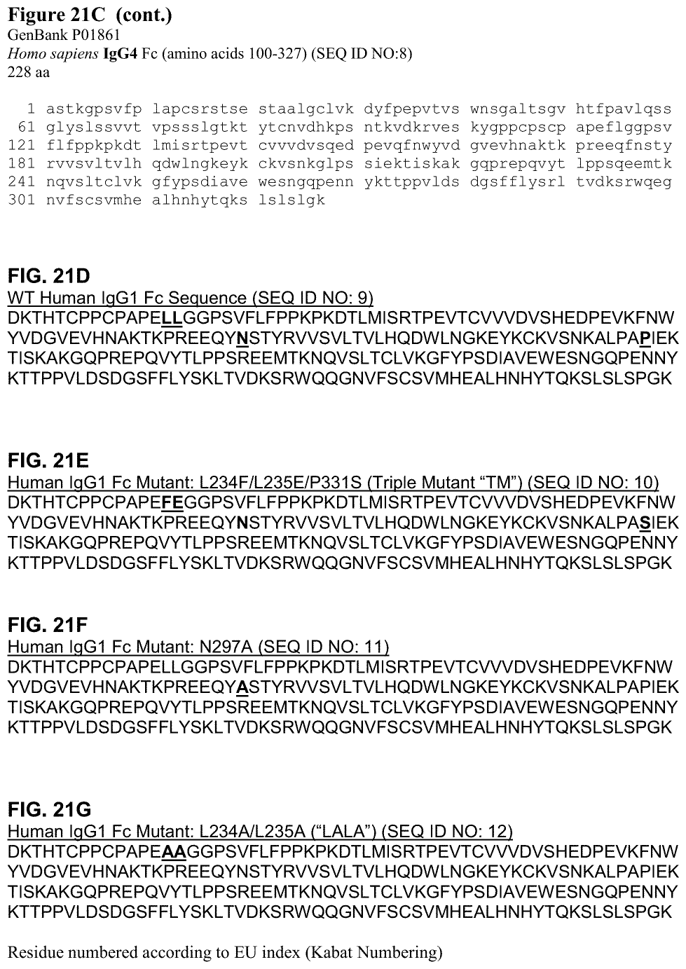

An Ig Fc polypeptide can be incorporated into a polypeptide of any TMAPP of the present disclosure (e.g., a MOD-containing or MOD-less sc- or m-TMAPP having a chemical conjugation site or its epitope conjugate). For example, where the TMAPP is a m-TMAPP, the first and/or the second polypeptide chain of the m-TMAPP may comprise a Fc polypeptide sequence; and where it is a sc-TMAPP, its polypeptide may comprise an Ig Fc polypeptide sequence. The Fc polypeptide sequence can be a human IgG1 Fc, a human IgG2 Fc, a human IgG3 Fc, a human IgG4 Fc, etc. In some cases, the Fc polypeptide comprises an amino acid sequence having at least about 70%, at least about 75%, at least about 80%, at least about 85%, at least about 90%, at least about 95%, at least about 98%, at least about 99%, or 100% amino acid sequence identity to an amino acid sequence of a Fc region depicted in FIGS. 21A-21G.

In some cases, a Fc polypeptide present in any TMAPP comprises an amino acid sequence having at least about 70%, at least about 75%, at least about 80%, at least about 85%, at least about 90%, at least about 95%, at least about 98%, at least about 99%, or 100% amino acid sequence identity to: (i) the human IgG1 Fc polypeptide depicted in FIG. 21A, or the IgG1 Fc polypeptide depicted in FIG. 21A comprising an N77A substitution; (ii) the human IgG2 Fc polypeptide depicted in FIG. 21A, or amino acids 99-325 of the human IgG2 Fc polypeptide depicted in FIG. 21A; (iii) the human IgG3 Fc polypeptide depicted in FIG. 21A, or amino acids 19-246 of the human IgG3 Fc polypeptide depicted in FIG. 21A; (iv) the human IgM Fc polypeptide depicted in FIG. 21B, or amino acids 1-276 of the human IgM Fc polypeptide depicted in FIG. 21B; (v) the human IgA Fc polypeptide depicted in FIG. 21C, or amino acids 1-234 of the human IgA Fc polypeptide depicted in FIG. 21C; or (vi) the human IgG4 Fc polypeptide depicted in FIG. 21C, or amino acids 100-327 of the human IgG4 Fc polypeptide depicted in FIG. 21C.

In some cases, the Fc polypeptide present in any TMAPP comprises: (i) the amino acid sequence depicted in FIG. 21A (human IgG1 Fc); (ii) the amino acid sequence depicted in FIG. 21A (human IgG1 Fc), except for a substitution of N297 with an amino acid other than asparagine; (iii) the amino acid sequence depicted in FIG. 21C (human IgG1 Fc comprising an N297A substitution); (iv) the amino acid sequence depicted in FIG. 21A (human IgG1 Fc), except for a substitution of either or both of L234 and L235 with an amino acid other than leucine; (v) the amino acid sequence depicted in FIG. 21E; (vi) the amino acid sequence depicted in FIG. 21F; (vii) the amino acid sequence depicted in FIG. 21G (human IgG1 Fc comprising an L234A substitution and an L235A substitution); (viii) the amino acid sequence depicted in FIG. 21A (human IgG1 Fc), except for a substitution of P331 with an amino acid other than proline; in some cases, the substitution is a P331S substitution; (ix) the amino acid sequence depicted in FIG. 21A (human IgG1 Fc), except for substitutions at L234 and L235 with amino acids other than leucine, and a substitution of P331 with an amino acid other than proline; (x) the amino acid sequence depicted in FIG. 21B (human IgG1 Fc comprising L234F, L235E, and P331S substitutions); or (xi) an IgG1 Fc polypeptide that comprises L234A and L235A substitutions.

Linkers

As noted above, any TMAPP (e.g., a MOD-containing or MOD-less sc- or m-TMAPP having a chemical conjugation site or its epitope conjugate) can include a linker peptide interposed between any two or more of the recited components of a TMAPP's polypeptide chain(s), e.g., between an epitope and a MHC polypeptide; between a MHC polypeptide and an Ig Fc polypeptide; between a first MHC polypeptide and a second MHC polypeptide; etc.

Suitable peptide linkers (also referred to as “spacers”) can be readily selected, and can be of any of a number of suitable lengths, such as from 1 amino acid (aa) to 35 aa, from 1 aa to 25 aa, from 2 aa to 15 aa, from 3 aa to 12 aa, from 3 aa to 20 aa, from 4 aa to 10 aa, from 5 aa to 9 aa, from 6 aa to 8 aa, from 7 aa to 8 aa, from from 8 aa to 15 aa, from 15 aa to 20 aa, from 25 aa to 35 aa, from 35 aa to 45 aa, or from 45 aa to 50 aa. A suitable linker can be a 1 aa, or a peptide 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 aa in length.

Exemplary linkers include glycine polymers (G)ₙ, glycine-serine polymers (including, for example, (GS)ₙ, (GSGGS)ₙ (SEQ ID NO:66) and (GGGS)ₙ (SEQ ID NO:67), where n is an integer of at least one (e.g., 1, 2, 3, 4, 5, 6, 7, 8. 9. or 10), glycine-alanine polymers, alanine-serine polymers, and other flexible linkers known in the art. As both Gly and Ser are relatively unstructured, their polymers can serve as a neutral tether between components; glycine accesses significantly more phi-psi space than even alanine, and is much less restricted than residues with longer side chains (see Scheraga, Rev. Computational Chem. 11173-142 (1992)). Exemplary linkers can also comprise amino acid sequences including, but not limited to, GGSG (SEQ ID NO:68), GGSGG (SEQ ID NO:69), GSGSG (SEQ ID NO:70), GSGGG (SEQ ID NO:71), GGGSG (SEQ ID NO:72), GSSSG (SEQ ID NO:73), and the like. Exemplary linkers can include, e.g., Gly(Ser₄)ₙ, where n is 1, 2, 3, 4, 5, 6, 7, 8, 9, or 10. In some cases, a linker comprises the amino acid sequence (GSSSS)ₙ (SEQ ID NO:74), where n is 3 or 4, or where n is 5 or 6. Exemplary linkers can include, e.g., (G₄Ser)ₙ, where n is 1, 2, 3, 4, 5, 6, 7, 8, 9, or 10. In some cases, a linker comprises the amino acid sequence AAAGG (SEQ ID NO:75).

In some cases, a linker comprises the amino acid sequence (GGGGS)ₙ (SEQ ID NO:76) where n is 1, 2, 3, 4, 5, 6, 7, 8, 9, or 10. In some cases, a linker polypeptide present in a m-TMAPP includes a cysteine residue that can form a disulfide bond with an epitope or a cysteine residue present in a second polypeptide of the m-TMAPP. In some cases, for example, the linker comprises the amino acid sequence GCGASGGGGSGGGGS (SEQ ID NO:77), the sequence GCGGSGGGGSGGGGSGGGGS (SEQ ID NO:78) or the sequence GCGGSGGGGSGGGGS (SEQ ID NO:79).

In some cases, a pair of cysteines and/or selenocysteines are present in a linker. Where a pair of cysteines and/or selenocysteines (including a cysteine selenocysteine pair) are present in a linker, they may be used as a chemical conjugation site for a bis-thiol reagent as discussed below, permitting the formation of an epitope conjugate or another form of drug conjugate.

Epitope-Presenting Peptides

A peptide epitope (also referred to herein as a “peptide antigen” or “epitope-presenting peptide” or simply an “epitope”) present in a TMAPP-epitope conjugate presents an epitope to a TCR on the surface of a T-cell. An epitope-presenting peptide can have a length of from about 4 amino acids (aa) to about 25 aa, e.g., the epitope can have a length of from 4 aa to about 10 aa, from 10 aa to about 15 aa, from 15 aa to about 20 aa, or from 20 aa to about 25 aa. For example, an epitope present in any TMAPP-epitope conjugate can have a length of 4, 5, 6, 7, 8, 9, 10, 11, 12, 13, 14, 15, 16, 17, 18, 19, 20, 21, 22, 23, 24, or 25 aa. In some cases, an epitope-presenting peptide present in a multimeric polypeptide has a length of from 5 aa to 10 aa, (e.g., 5 aa, 6 aa, 7 aa, 8 aa, 9 aa, or 10 aa).

An epitope-presenting peptide present in a TMAPP-epitope conjugate (e.g., a MOD-containing or MOD-less sc- or m-TMAPP-epitope conjugate) is specifically bound by a T-cell, i.e., the epitope is specifically bound by an epitope-specific T-cell. An epitope-specific T-cell binds an epitope-presenting peptide having a reference amino acid sequence, but does not substantially bind an epitope that differs from the reference amino acid sequence. For example, an epitope-specific T-cell binds an epitope-presenting peptide having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T-cell can bind an epitope-presenting peptide for which it is specific with an affinity of at least 10⁻⁷ M, at least 10⁻⁸ M, at least 10⁻⁹ M, or at least 10⁻¹⁰ M.

Suitable epitope-presenting peptides include, but are not limited to, epitope-presenting peptides present in a cancer-associated antigen. Cancer-associated antigens include, but are not limited to, α-folate receptor; carbonic anhydrase IX (CAIX); CD19; CD20; CD22; CD30; CD33; CD44v7/8; carcinoembryonic antigen (CEA); epithelial glycoprotein-2 (EGP-2); epithelial glycoprotein-40 (EGP-40); folate binding protein (FBP); fetal acetylcholine receptor; ganglioside antigen GD2; Her2/neu; IL-13R-a2; kappa light chain; LeY; L1 cell adhesion molecule; melanoma-associated antigen (MAGE); MAGE-A1; mesothelin; MUC1; NKG2D ligands; oncofetal antigen (h5T4); prostate stem cell antigen (PSCA); prostate-specific membrane antigen (PSMA); tumor-associate glycoprotein-72 (TAG-72); and vascular endothelial growth factor receptor-2 (VEGF-R2). See, e.g., Vigneron et al. (2013) Cancer Immunity 13:15; and Vigneron (2015) BioMed Res. Int'l Article ID 948501. In some cases, the epitope is a human papilloma virus E7 antigen epitope; see, e.g., Ramos et al. (2013) J. Immunother. 36:66.

In some cases, a suitable peptide epitope is a peptide fragment of from about 4 aa to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length of a MUC1 polypeptide, a human papillomavirus (HPV) E6 polypeptide, an LMP2 polypeptide, a HPV E7 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, a NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, a hTERT polypeptide, a sarcoma translocation breakpoint polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an alpha-fetoprotein (AFP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosin kinase (LCK) polypeptide, a high molecular weight melanoma associated antigen (HMW-MAA) polypeptide, an A-kinase anchoring protein-4 (AKAP-4) polypeptide, a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFI3) polypeptide, a MAD-CT-2 polypeptide, a Fos-related antigen-1 (FOSL) polypeptide, and a Wilms tumor-1 (WT1) polypeptide.

Amino acid sequences of cancer-associated antigens are known in the art; see, e.g., MUC1 (GenBank CAA56734); LMP2 (GenBank CAA47024); HPV E6 (GenBank AAD33252); HPV E7 (GenBank AHG99480); EGFRvIII (GenBank NP_001333870); HER-2/neu (GenBank AAI67147); MAGE-A3 (GenBank AAH11744); p53 (GenBank BAC16799); NY-ESO-1 (GenBank CAA05908); PSMA (GenBank AAH25672); CEA (GenBank AAA51967); melan/MART1 (GenBank NP_005502); Ras (GenBank NP_001123914); gp100 (GenBank AAC60634); bcr-abl (GenBank AAB60388); tyrosinase (GenBank AAB60319); survivin (GenBank AAC51660); PSA (GenBank CAD54617); hTERT (GenBank BAC11010); SSX (GenBank NP_001265620); Eph2A (GenBank NP_004422); PAP (GenBank AAH16344); ML-IAP (GenBank AAH14475); AFP (GenBank NP_001125); EpCAM (GenBank NP_002345); ERG (TMPRSS2 ETS fusion) (GenBank ACA81385); PAX3 (GenBank AAI01301); ALK (GenBank NP_004295); androgen receptor (GenBank NP_000035); cyclin B1 (GenBank CA099273); MYCN (GenBank NP_001280157); RhoC (GenBank AAH52808); TRP-2 (GenBank AAC60627); mesothelin (GenBank AAH09272); PSCA (GenBank AAH65183); MAGE A1 (GenBank NP_004979); CYP1B1 (GenBank AAM50512); PLAC1 (GenBank AAG22596); BORIS (GenBank NP_001255969); ETV6 (GenBank NP_001978); NY-BR1 (GenBank NP_443723); SART3 (GenBank NP_055521); carbonic anhydrase IX (GenBank EAW58359); PAX5 (GenBank NP_057953); OY-TES1 (GenBank NP_115878); sperm protein 17 (GenBank AAK20878); LCK (GenBank NP_001036236); HMW-MAA (GenBank NP_001888); AKAP-4 (GenBank NP_003877); SSX2 (GenBank CAA60111); XAGE1 (GenBank NP_001091073; XP_001125834; XP_001125856; and XP_001125872); B7H3 (GenBank NP_001019907; XP_947368; XP_950958; XP_950960; XP_950962; XP_950963; XP_950965; and XP_950967); LGMN1 (GenBank NP_001008530); TIE-2 (GenBank NP_000450); PAGE4 (GenBank NP_001305806); VEGFR2 (GenBank NP_002244); MAD-CT-1 (GenBank NP_005893 NP_056215); FAP (GenBank NP_004451); PDGFI3 (GenBank NP_002600); MAD-CT-2 (GenBank NP_001138574); FOSL (GenBank NP_005429); and WT-1 (GenBank NP_000369). These polypeptides are also discussed in, e.g., Cheever et al. (2009) Clin. Cancer Res. 15:5323, and references cited therein; Wagner et al. (2003) J. Cell. Sci. 116:1653; Matsui et al. (1990) Oncogene 5:249; Zhang et al. (1996) Nature 383:168.

In some cases, the epitope is HPV16E7/82-90 (LLMGTLGIV; SEQ ID NO:80). In some cases, the epitope is HPV16E7/86-93 (TLGIVCPI; SEQ ID NO:81). In some cases, the epitope is HPV16E7/11-20 (YMLDLQPETT; SEQ ID NO:82). In some cases, the epitope is HPV16E7/11-19 (YMLDLQPET; SEQ ID NO:83). See, e.g., Ressing et al. ((1995) J. Immunol. 154:5934) for additional suitable HPV epitopes.

In some cases, the peptide epitope is an epitope associated with or present in a “self” antigen (an autoantigen). Autoantigens include, e.g., aggrecan, alanyl-tRNA syntetase (PL-12), alpha beta crystallin, alpha fodrin (Sptan 1), alpha-actinin, α1 antichymotrypsin, α1 antitripsin, α1 microglobulin, alsolase, aminoacyl-tRNA synthetase, an amyloid, an annexin, an apolipoprotein, aquaporin, bactericidal/permeability-increasing protein (BPI), β-globin precursor BP1, β-actin, β-lactoglobulin A, β-2-glycoprotein I, β2-microglobulin, a blood group antigen, C reactive protein (CRP), calmodulin, calreticulin, cardiolipin, catalase, cathepsin B, a centromere protein, chondroitin sulfate, chromatin, collagen, a complement component, cytochrome C, cytochrome P450 2D6, cytokeratins, decorin, dermatan sulfate, DNA, DNA topoisomerase I, elastin, Epstein-Barr nuclear antigen 1 (EBNA1), elastin, entaktin, an extractable nuclear antigen, Factor I, Factor P, Factor B, Factor D, Factor H, Factor X, fibrinogen, fibronectin, formiminotransferase cyclodeaminase (LC-1), gliadin and amidated gliadin peptides (DGPs), gp210 nuclear envelope protein, GP2 (major zymogen granule membrane glycoprotein), glycoprotein gpIIb/IIIa, glial fibrillary acidic protein (GFAP), glycated albumin, glyceraldehyde 3-phosphate dehydrogenase (GAPDH), haptoglobin A2, heat shock proteins, hemocyanin, heparin, a histone, histidyl-tRNA synthetase (Jo-1), hordeins (e.g., C-hordeins, γ-hordeins, B-hordeins and/or D-hordeins), hyaluronidase, immunoglobulins, insulin, insulin receptor, an integrin, interstitial retinol-binding protein 3, intrinsic factor, Ku (p70/p80), lactate dehydrogenase, laminin, liver cytosol antigen type 1 (LC1), liver/kidney microsomal antigen 1 (LKM1), lysozyme, melanoma differentiation-associated protein 5 (MDAS), Mi-2 (chromodomain helicase DNA binding protein 4), a mitochondrial protein, muscarinic receptors, myelin-associated glycoprotein, myosin, myelin basic protein, myelin oligodendrocyte glycoprotein, myeloperoxidase (MPO), rheumatoid factor (IgM anti-IgG), neuron-specific enolase, nicotinic acetylcholine receptor A chain, nucleolin, a nucleoporin, nucleosome antigen, PM/Scl100, PM/Scl 75, pancreatic β-cell antigen, pepsinogen, peroxiredoxin 1, phosphoglucose isomerase, phospholipids, phosphotidyl inositol, platelet derived growth factors, polymerase beta (POLB), potassium channel KIR4.1, proliferating cell nuclear antigen (PCNA), proteinase-3, proteolipid protein, proteoglycan, prothrombin, recoverin, rhodopsin, ribonuclease, a ribonucleoprotein, ribosomes, a ribosomal phosphoprotein, RNA, an Sm protein, Sp100 nuclear protein, SRP54 (signal recognition particle 54 kDa), selectin, smooth muscle proteins, sphingomyelin, streptococcal antigens, superoxide dismutase, synovial joint proteins, T1F1 gamma collagen, threonyl-tRNA synthetase (PL-7), tissue transglutaminase, thyroid peroxidase, thyroglobulin, thyroid stimulating hormone receptor, transferrin, triosephosphate isomerase, tubulin, tumor necrosis alpha, topoisomerase, U1-dnRNP 68/70 kDa, U1-snRNP A, U1-snRNP C, U-snRNP B/B′, ubiquitin, vascular endothelial growth factor, vimentin, and vitronectin.

Antigens associated with type 1 diabetes (T1D) include, e.g., preproinsulin, proinsulin, insulin, insulin B chain, insulin A chain, 65 kDa isoform of glutamic acid decarboxylase (GAD65), 67 kDa isoform of glutamic acid decarboxylase (GAD67), tyrosine phosphatase (IA-2), heat-shock protein HSP65, islet-specific glucose-phosphatase catalytic subunit related protein (IGRP), islet antigen 2 (IA2), and zinc transporter (ZnT8). See, e.g., Mallone et al. (2011) Clin. Dev. Immunol. 2011:513210; and U.S. Patent Publication No. 2017/0045529. An antigen “associated with” a particular autoimmune disorder is an antigen that is a target of autoantibodies and/or autoreactive T-cells present in individuals with that autoimmune disorder, where such autoantibodies and/or autoreactive T-cells mediate a pathological state associated with the autoimmune disorder. A suitable epitope-presenting peptide for inclusion in a TMAPP-epitope conjugate (e.g., a MOD-containing or MOD-less sc- or m-TMAPP-epitope conjugate) can be an epitope-presenting peptide of from 4 amino acids to about 25 amino acids in length of any one of the aforementioned T1D-associated antigens. As one non-limiting example, an epitope-presenting peptide is proinsulin 73-90 (GAGSLQPLALEGSLQKR; SEQ ID NO:84).

Antigens associated with Grave's disease include, for example, thyroglobulin, thyroid peroxidase, and thyrotropin receptor (TSH-R). A suitable epitope-presenting peptide for inclusion in a TMAPP-epitope conjugate (e.g., a MOD-containing or MOD-less sc- or m-TMAPP-epitope conjugate) can be an epitope-presenting peptide of from 4 amino acids to about 25 amino acids in length of any one of the aforementioned Grave's disease-associated antigens.

Antigens associated with autoimmune polyendocrine syndrome include 17-alpha hydroxylase, histidine decarboxylase, tryptophan hydroxylase, and tyrosine hydroxylase. A suitable epitope-presenting peptide for inclusion in a TMAPP-epitope conjugate (e.g., a MOD-containing or MOD-less sc- or m-TMAPP-epitope conjugate) can be an epitope-presenting peptide of from 4 amino acids to about 25 amino acids in length of any one of the aforementioned autoimmune polyendocrine syndrome-associated antigens.

Antigens associated with rheumatoid arthritis include, e.g., collagen, vimentin, aggregan, and fibrinogen. A suitable epitope-presenting peptide for inclusion in a TMAPP-epitope conjugate (e.g., a MOD-containing or MOD-less sc- or m-TMAPP-epitope conjugate) can be an epitope-presenting peptide of from 4 amino acids to about 25 amino acids in length of any one of the aforementioned rheumatoid arthritis-associated antigens.

Antigens associated with Parkinson's disease include, e.g., α-synuclein. A suitable epitope-presenting peptide for inclusion in a TMAPP-epitope conjugate (e.g., a MOD-containing or MOD-less sc- or m-TMAPP-epitope conjugate) can be an epitope-presenting peptide of from 4 amino acids to about 25 amino acids in length of any aforementioned Parkinson's disease-associated antigens.

Antigens associated with multiple sclerosis include, e.g., myelin basic protein, myelin oligodendrocyte glycoprotein, and proteolipid protein. A suitable epitope-presenting peptide for inclusion in a TMAPP-epitope conjugate (e.g., a MOD-containing or MOD-less sc- or m-TMAPP-epitope conjugate) can be an epitope-presenting peptide of from 4 amino acids to about 25 amino acids in length of any one of the aforementioned multiple sclerosis-associated antigens.

Antigens associated with celiac disease include, e.g., tissue transglutaminase and gliadin. A suitable epitope-presenting peptide for inclusion in a TMAPP-epitope conjugate (e.g., a MOD-containing or MOD-less sc- or m-TMAPP-epitope conjugate) can be an epitope-presenting peptide of from 4 amino acids to about 25 amino acids in length of any one of the aforementioned celiac-associated antigens. Other antigens associated with celiac disease include, e.g., secalins, hordeins, avenins, and glutenins. Examples of secalins include rye secalins. Examples of hordeins include barley hordeins. Examples of glutenins include wheat glutenins. See, e.g., U.S. 2016/0279233.

TMAPPs Comprising an Immunomodulatory Domain (MOD)

Some single chain and multimeric TMAPPs (sc-TMAPPs and m-TMAPPs) of the present disclosure contain, in addition to MHC Class II polypeptides, one or more wild type and/or variant MODs, namely MOD-containing sc-TMAPPs and MOD-containing m-TMAPPs, either of which may comprise a chemical conjugation site for an epitope or be in the form of an epitope conjugate. Thus, the present disclosure provides T-cell modulatory antigen-presenting polypeptides. In some cases, the MOD-containing sc-TMAPPs and MOD-containing m-TMAPPs comprise two or more polypeptide chains that each have at least one of the MHC Class II α1, α2, β1, or β2 polypeptide sequences. In some cases, the MOD-containing sc-TMAPPs and MOD-containing m-TMAPPs comprise a single polypeptide chain that contains the MHC Class II α1, α2, and β1, or the MHC Class II α1, α2, β1, and β2 polypeptide sequences.

MOD-containing sc-TMAPPs and MOD-containing m-TMAPPs can modulate activity of a T-cell through their interaction with the corresponding Co-MODs on T-cells. Where the m-TMAPP or sc-TMAPP comprising a chemical conjugation site is converted to its epitope conjugate, it may modulate the activity of T-cells through both the TCR and the Co-MODs, provided the TCR recognizes and binds the TMAPP-presented epitope. Where variant MODs with reduced affinity for their Co-MODs are present in the MOD-containing TMAPP-epitope conjugate, the reduced affinity of the MOD for its Co-MOD, and the affinity of the epitope for a TCR, provides for enhanced selectivity of the MOD-containing TMAPP-epitope conjugate (e.g., the ratio of the epitope-specific T cell response to the epitope-non-specific T cell response may be increased with MODs have a reduced affinity for their Co-MOD, such as by least 2:1, at least 5:1, at least 10:1, at least 15:1, at least 20:1, at least 25:1, at least 50:1, or at least 100:1). In some cases, a TMAPP-epitope conjugate activates a CD8⁺ T-cell response, e.g., a CD8⁺ T-cell response to a cancer cell. In some cases, a TMAPP-epitope conjugate reduces activity of an autoreactive T-cell and/or an autoreactive B cell. In some cases, a TMAPP-epitope conjugate increases the number and/or activity of a regulator T-cell (Treg), resulting in reduced activity of an autoreactive T-cell and/or an autoreactive B cell.

MODs that are suitable for inclusion in a TMAPP (e.g., a sc- or m-TMAPPP) having a chemical conjugation site, or its epitope conjugate, include, but are not limited to, IL-2, transforming growth factor-beta (TGFβ), JAG1, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, lymphotoxin beta receptor, 3/TR6, ILT3, ILT4, and HVEM. In some cases, a MOD suitable for inclusion in a TMAPP having a chemical conjugation site, or its epitope conjugate, is a variant that comprises from 1 to 10 amino acid substitutions relative to its wild-type or naturally-occurring MOD, and that exhibits reduced affinity to its Co-MOD, compared to the affinity of the wild-type or naturally-occurring MOD for the Co-MOD.

m-TMAPPs Comprising One or More MODs—MOD-Containing m-TMAPPs

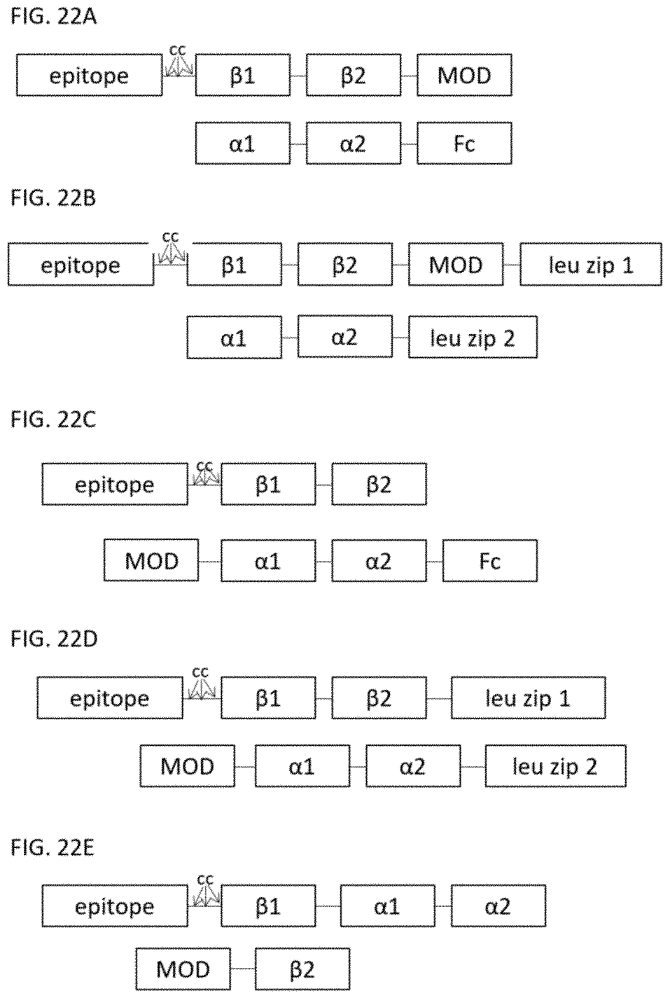

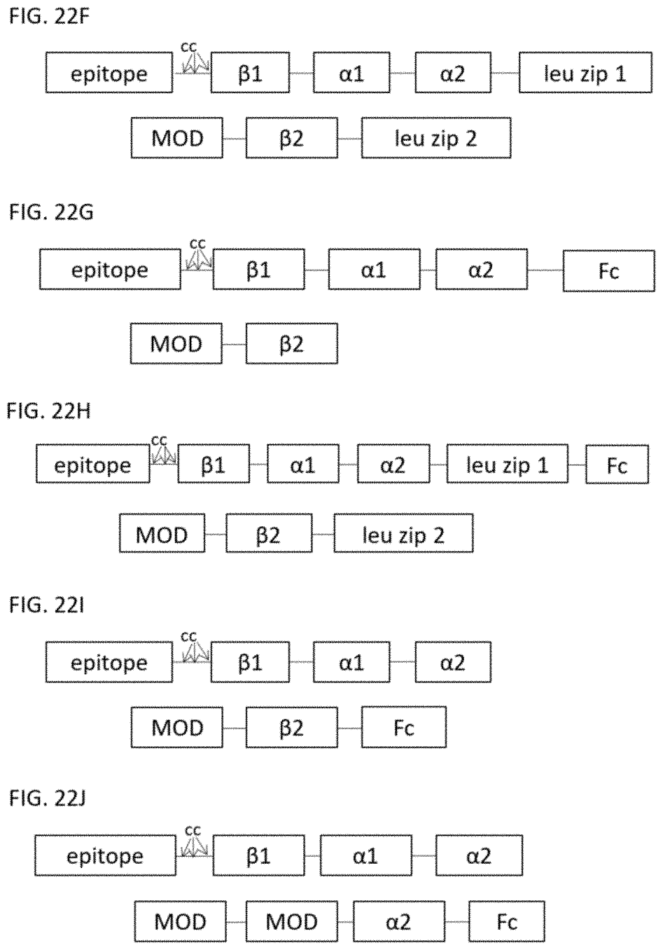

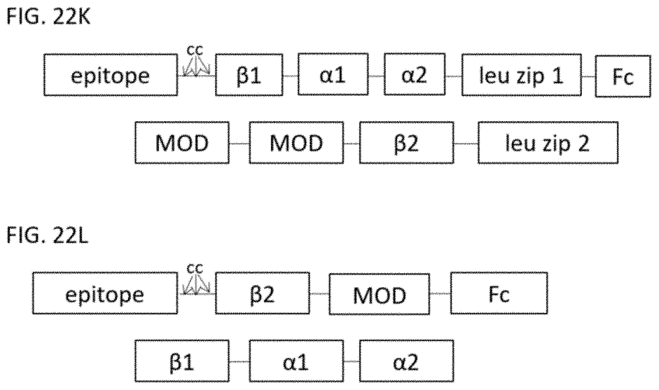

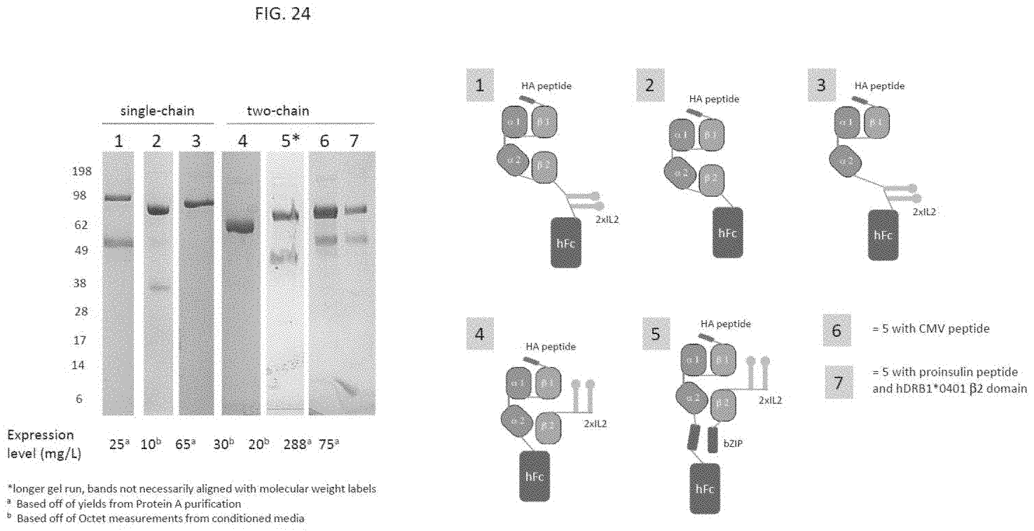

In an embodiment, MOD-containing m-TMAPP of the present disclosure having a chemical conjugation comprises: i) at least one chemical conjugation site at which the MOD-containing m-TMAPP can be conjugated to an epitope (e.g., a peptide recognized and bound by a TCR); ii) a MHC Class II α chain polypeptide (e.g., α1 and/or α2); iii) a MHC Class II β chain polypeptide (e.g., β1 and/or β2); and iv) a MOD (also referred to herein as a “MOD polypeptide” or a “MOD domain”). A MOD-containing m-TMAPP having a chemical conjugation site can further include one or both of: a dimerizer polypeptide; and an immunoglobulin scaffold (e.g., an Ig Fc polypeptide) or a non-immunoglobulin scaffold. Non-limiting examples of MOD-containing m-TMAPPs having an epitope covalently attached at a chemical conjugation site located on a linker placed at the N-terminus of a m-TMAPP polypeptide are depicted schematically in FIGS. 22A-22L and in FIG. 24 (see constructs 4 and 5 showing m-TMAPP-like constructs having a hemagglutinin (HA) epitope attached to a linker placed at the N-terminus of the MHC Class II β1 polypeptide). The MOD-containing m-TMAPP-epitope conjugate resulting from attaching an epitope has the epitope covalently attached (directly or indirectly) at a chemical conjugation site.

In some cases, a MOD-containing m-TMAPP having a chemical conjugation site (or its epitope conjugate) comprises a single wild-type or variant MOD. In some cases, a MOD-containing m-TMAPP having a chemical conjugation site comprises one or more wild-type or variant MODs (e.g., 2 or 3 independently selected wild-type or variant MODs). In some cases, MOD-containing m-TMAPPs having a chemical conjugation site comprise two independently selected wild-type and/or variant MODs. In some cases, MOD-containing m-TMAPPs having a chemical conjugation site comprise three independently selected wild-type and/or variant MODs. Where a MOD-containing m-TMAPP comprises 2, 3, or more MODs (which may be the same or selected independently), in some cases they are placed in tandem without being separated by a linker, in other cases at least two of the MODs (or each of the MODs) are separated from one another by a linker.

A MOD-containing m-TMAPP having a chemical conjugation site (or its epitope conjugate) can include one or more independently selected linkers between any two adjacent polypeptides, e.g., between an epitope and a MOD, between a MOD and a MHC Class II polypeptide, between two MHC Class II polypeptides, between a MOD and an Ig Fc polypeptide, etc. In some embodiments, the one or more linkers are located between one or more of: i) a MHC Class II polypeptide and an Ig Fc polypeptide, where such a linker is referred to herein as “L1”; ii) a MOD and a MHC Class II polypeptide, where such a linker is referred to herein as “L2”; iii) a first MOD and a second independently selected MOD, where such a linker is referred to herein as “L3”; iv) a conjugated epitope and a MHC Class II polypeptide in a MOD-containing m-TMAPP-epitope conjugate (in some cases appearing as an “optional linker” placed at the N-terminus or C-terminus of an unconjugated m-TMAPP having a chemical conjugation as part of the “optional linker”; v) a MHC Class II polypeptide and a dimerization polypeptide (e.g., a first or a second member of a dimerizing pair); and/or vi) a dimerization polypeptide (e.g., a first or a second member of a dimerizing pair) and an IgFc polypeptide. In some cases, an L1 linker comprises (GGGGS)n, where n is 1, 2, 3, 4, 5, 6, 7, or 8. In some cases, an L2 linker comprises (GGGGS)n, where n is 1, 2, 3, 4, 5, 6, 7, or 8. In some cases, an L3 linker comprises (GGGGS)n, where n is 1, 2, 3, 4, 5, 6, 7, or 8. Non-limiting examples of other linker polypeptides that may be employed are described in the section addressing linkers.

Five groups of MOD-containing m-TMAPPs listed as MOD-Containing m-TMAPPs—Embodiment Set 1 through Set 5 follow. As discussed above, the MOD-containing m-TMAPPs in those embodiments can include one or more independently selected linkers between any two adjacent polypeptides. In addition, the MOD-containing m-TMAPPs of those embodiments, as discussed above, may further include dimerizer polypeptide(s) and/or scaffold polypeptide(s) where they are not specifically recited.

MOD-Containing m-TMAPPs—Embodiment Set 1: In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; and iv) a MHC Class II α2 polypeptide; and b) a second polypeptide comprising (e.g., from N-terminus to C-terminus): i) a MOD; and ii) a MHC Class II β2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) an immunoglobulin or non-immunoglobulin scaffold polypeptide; and b) a second polypeptide comprising (e.g., from N-terminus to C-terminus): i) a MOD; and ii) a MHC Class II β2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) an Ig Fc polypeptide; and b) a second polypeptide comprising (e.g., from N-terminus to C-terminus): i) a MOD; and ii) a MHC Class II β2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) a first member of a dimerizer pair; and b) a second polypeptide comprising (e.g., from N-terminus to C-terminus): i) a MOD; ii) a MHC Class II β2 polypeptide; and iii) a second member of the dimerizer pair. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) a first leucine zipper polypeptide; and b) a second polypeptide comprising (e.g., from N-terminus to C-terminus): i) a MOD; ii) a MHC Class II β2 polypeptide; and iii) a second leucine zipper polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; v) a first leucine zipper polypeptide; and vi) an Ig Fc polypeptide; and b) a second polypeptide comprising (e.g., from N-terminus to C-terminus): i) a MOD; ii) a MHC Class II β2 polypeptide; and iii) a second leucine zipper polypeptide. In any one of the above embodiments, the TMAPP can include a single MOD. In any one of the above embodiments, the TMAPP can include 2 independently selected wild-type or variant MODs (which may be the same or different) that may be placed in tandem, separated by a linker, or in separate parts of the molecule. In any one of the above embodiments, the TMAPP can include 3 independently selected wild-type or variant MODs (which may be the same or different) that may be placed in tandem, separated by linkers, and/or in separate parts of the molecule.

For example, in some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; v) a first leucine zipper polypeptide; and vi) an Ig Fc polypeptide; and b) a second polypeptide comprising (e.g., from N-terminus to C-terminus): i) a first MOD; ii) a second independently selected MOD (e.g., wild-type or variant MOD); iii) a MHC Class II β2 polypeptide; and iv) a second leucine zipper polypeptide. In some cases, the first and the second MODs comprise the same amino acid sequences. As another example, in some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) an Ig Fc polypeptide; and b) a second polypeptide comprising (e.g., from N-terminus to C-terminus): i) a first MOD; ii) a second independently selected MOD (e.g., wild-type or variant MOD); and iii) a MHC Class II β2 polypeptide. In some cases, the first and the second MODs comprise the same amino acid sequences. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; and iv) a MHC Class II α2 polypeptide; and b) a second polypeptide comprising (e.g., from N-terminus to C-terminus): i) a MOD; ii) a MHC Class II β2 polypeptide; and iii) an Ig Fc polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; and iv) a MHC Class II α2 polypeptide; and b) a second polypeptide comprising (e.g., from N-terminus to C-terminus): i) a first MOD; ii) a second independently selected MOD (e.g., wild-type or variant MOD); iii) a MHC Class II β2 polypeptide; and iv) an Ig Fc polypeptide. In some cases, the first and the second MODs comprise the same amino acid sequence. In some cases, a MOD-containing m-TMAPP having a chemical conjugation site (or its epitope conjugate) comprises: a) a first polypeptide comprising, in order from N-terminus to C-terminus: i) a MOD; ii) a MHC Class II β1 polypeptide; iii) a MHC Class II α1 polypeptide; and iv) a MHC Class II α2 polypeptide; and b) a second polypeptide comprising (e.g., from N-terminus to C-terminus): i) an optional linker that when present is bound to ii) a MHC Class II β2 polypeptide; and iii) an Ig Fc polypeptide. In some cases, a MOD-containing m-TMAPP having a chemical conjugation site (or its epitope conjugate) comprises: a) a first polypeptide comprising, in order from N-terminus to C-terminus: i) a first MOD; ii) a second independently selected MOD (e.g., wild-type or variant MOD); iii) a MHC Class II β1 polypeptide; iv) a MHC Class II α1 polypeptide; and v) a MHC Class II α2 polypeptide; and b) a second polypeptide comprising, in order from N-terminus to C-terminus: i) an optional linker that when present is bound to ii) a MHC Class II β2 polypeptide; and iii) an Ig Fc polypeptide. In some cases, the first and the second MODs comprise the same amino acid sequence. Where a TMAPP of the present disclosure comprises two MODs, in some cases, the first MOD is linked to the second independently selected MOD (e.g., wild-type or variant MOD) by a linker (an L3 linker); e.g., a linker of from about 2 amino acids to 50 amino acids in length. Suitable L3 linkers include (GGGGS)n, where n is 1, 2, 3, 4, 5, 6, 7, or 8. In some cases, the TMAPP comprises a linker (an L1) between the MHC polypeptide and the Ig Fc polypeptide; where exemplary suitable linkers include (GGGGS)n, where n is 1, 2, 3, 4, 5, 6, 7, or 8. In some cases, the TMAPP comprises a linker (an L2) between the MOD and the MHC polypeptide, where exemplary suitable linkers include (GGGGS)n, where n is 1, 2, 3, 4, 5, 6, 7, or 8. Where the TMAPP comprises two MODs, in some cases, the two MODs are separated by a linker (an L3); where exemplary suitable linkers include (GGGGS)n, where n is 1, 2, 3, 4, 5, 6, 7, or 8.

MOD-Containing m-TMAPPs—Embodiment Set 2: In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II α1 polypeptide; iii) a MHC Class II α2 polypeptide; and iv) an immunoglobulin or non-immunoglobulin scaffold polypeptide; and b) a second polypeptide comprising, in order from N-terminus to C-terminus: i) a MOD; ii) a MHC Class II β1 polypeptide; and iii) a MHC Class II β2 polypeptide. In some cases, a MOD-containing m-TMAPP having a chemical conjugation site (or its epitope conjugate) comprises: a) a first polypeptide comprising, in order from N-terminus to C-terminus: i) a MOD; ii) a MHC Class II α1 polypeptide; and iii) a MHC Class II α2 polypeptide; and iv) an immunoglobulin or non-immunoglobulin scaffold polypeptide; and b) a second polypeptide comprising, in order from N-terminus to C-terminus: i) an optional linker that when present is bound to ii) a MHC Class II β1 polypeptide; and iii) a MHC Class II β2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II α1 polypeptide; and iii) a MHC Class II α2 polypeptide; and b) a second polypeptide comprising, in order from N-terminus to C-terminus: i) a MOD; ii) a MHC Class II β1 polypeptide; iii) a MHC Class II β2 polypeptide; and iv) an immunoglobulin or non-immunoglobulin scaffold polypeptide. In some cases, a MOD-containing m-TMAPP having a chemical conjugation site (or its epitope conjugate) comprises: a) a first polypeptide comprising, in order from N-terminus to C-terminus: i) a MOD; ii) a MHC Class II α1 polypeptide; and iii) a MHC Class II α2 polypeptide; and b) a second polypeptide comprising, in order from N-terminus to C-terminus: i) an optional linker that when present is bound to ii) a MHC Class II β1 polypeptide; iii) a MHC Class II β2 polypeptide; and iv) an immunoglobulin or non-immunoglobulin scaffold polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II α1 polypeptide; iii) a MHC Class II α2 polypeptide; iv) an immunoglobulin or non-immunoglobulin scaffold polypeptide; and v) a first member of a dimerizer pair (e.g., a first leucine zipper polypeptide); and b) a second polypeptide comprising, in order from N-terminus to C-terminus: i) a MOD; ii) a MHC Class II β1 polypeptide; iii) a MHC Class II β2 polypeptide; and iv) a second member of a dimerizer pair (e.g., a second leucine zipper polypeptide). In some cases, a MOD-containing m-TMAPP having a chemical conjugation site (or its epitope conjugate) comprises: a) a first polypeptide comprising, in order from N-terminus to C-terminus: i) a MOD; ii) a MHC Class II α1 polypeptide; iii) a MHC Class II α2 polypeptide; iv) an immunoglobulin or non-immunoglobulin scaffold polypeptide; and v) a first member of a dimerizer pair (e.g., a first leucine zipper polypeptide); and b) a second polypeptide comprising, in order from N-terminus to C-terminus: i) an optional linker that when present is bound to ii) a MHC Class II β1 polypeptide; iii) a MHC Class II β2 polypeptide; and iv) a second member of a dimerizer pair (e.g., a second leucine zipper polypeptide). In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II α1 polypeptide; iii) a MHC Class II α2 polypeptide; and iv) a first member of a dimerizer pair (e.g., a first leucine zipper polypeptide); and b) a second polypeptide comprising, in order from N-terminus to C-terminus: i) a MOD; ii) a MHC Class II β1 polypeptide; iii) a MHC Class II β2 polypeptide; iv) an immunoglobulin or non-immunoglobulin scaffold polypeptide; and v) a second member of a dimerizer pair (e.g., a second leucine zipper polypeptide). In some cases, a MOD-containing m-TMAPP having a chemical conjugation site (or its epitope conjugate) comprises: a) a first polypeptide comprising, in order from N-terminus to C-terminus: i) a MOD; ii) a MHC Class II α1 polypeptide; iii) a MHC Class II α2 polypeptide; and iv) a first member of a dimerizer pair (e.g., a first leucine zipper polypeptide); and b) a second polypeptide comprising, in order from N-terminus to C-terminus: i) an optional linker that when present is bound to ii) a MHC Class II β1 polypeptide; iii) a MHC Class II β2 polypeptide; iv) an immunoglobulin or non-immunoglobulin scaffold polypeptide; and v) a second member of a dimerizer pair (e.g., a second leucine zipper polypeptide). In any one of the above embodiments, the TMAPP can include 2 independently selected wild-type or variant MODs (which may be the same or different) that may be placed in tandem, separated by a linker, or in separate parts of the molecule. In any one of the above embodiments, the TMAPP can include 3 independently selected wild-type or variant MODs (which may be the same or different) that may be placed in tandem, separated by linkers, and/or in separate parts of the molecule. In some cases, the TMAPP comprises a linker (an L1) between the MHC polypeptide and the Ig Fc polypeptide where exemplary suitable linkers include (GGGGS)n, where n is 1, 2, 3, 4, 5, 6, 7, or 8. In some cases, the TMAPP comprises a linker (an L2) between the MOD and the MHC polypeptide, where exemplary suitable linkers include (GGGGS)n, where n is 1, 2, 3, 4, 5, 6, 7, or 8. Where the TMAPP comprises two MODs, in some cases, the two MODs are separated by a linker (an L3), where exemplary suitable linkers include (GGGGS)n, where n is 1, 2, 3, 4, 5, 6, 7, or 8.

MOD-Containing m-TMAPPs—Embodiment Set 3: In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II β2 polypeptide; and iv) a MOD; and b) a second polypeptide comprising, in order from N-terminus to C-terminus: i) a MHC Class II α1 polypeptide; and ii) a MHC Class II α2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II β2 polypeptide; and iv) a MOD; and b) a second polypeptide comprising, in order from N-terminus to C-terminus: i) a MHC Class II α1 polypeptide; ii) a MHC Class II α2 polypeptide; and iii) an immunoglobulin or non-immunoglobulin scaffold polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II β2 polypeptide; and iv) a MOD; and b) a second polypeptide comprising, in order from N-terminus to C-terminus: i) a MHC Class II α1 polypeptide; ii) a MHC Class II α2 polypeptide; and iii) an Ig Fc polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II β2 polypeptide; iv) a MOD; and v) a first member of a dimerizer pair; and b) a second polypeptide comprising, in order from N-terminus to C-terminus: i) a MHC Class II α1 polypeptide; ii) a MHC Class II α2 polypeptide; and iii) a second member of the dimerizer pair. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II β2 polypeptide; iv) a MOD; and v) a first leucine zipper polypeptide; and b) a second polypeptide comprising, in order from N-terminus to C-terminus: i) a MHC Class II α1 polypeptide; ii) a MHC Class II α2 polypeptide; and iii) a second leucine zipper polypeptide. In any one of the above embodiments, the TMAPP can include a single MOD. In any one of the above embodiments, the TMAPP can include 2 independently selected wild-type or variant MODs (which may be the same or different) that may be placed in tandem, separated by a linker, or in separate parts of the molecule. In any one of the above embodiments, the TMAPP can include 3 independently selected wild-type or variant MODs (which may be the same or different) that may be placed in tandem, separated by linkers, and/or in separate parts of the molecule. In some cases, the TMAPP comprises a linker (an L1) between the MHC polypeptide and the Ig Fc polypeptide; where exemplary suitable linkers include (GGGGS)n, where n is 1, 2, 3, 4, 5, 6, 7, or 8. In some cases, the TMAPP comprises a linker (an L2) between the MOD and the MHC polypeptide, where exemplary suitable linkers include (GGGGS)n, where n is 1, 2, 3, 4, 5, 6, 7, or 8. Where the TMAPP comprises two MODs, in some cases, the two MODs are separated by a linker (an L3), where exemplary suitable linkers include (GGGGS)n, where n is 1, 2, 3, 4, 5, 6, 7, or 8.

MOD-Containing m-TMAPPs—Embodiment Set 4: In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; and iii) a MHC Class II β2 polypeptide; and b) a second polypeptide comprising, in order from N-terminus to C-terminus: i) a MOD; ii) a MHC Class II α1 polypeptide; and iii) a MHC Class II α2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; and iii) a MHC Class II β2 polypeptide; and b) a second polypeptide comprising, in order from N-terminus to C-terminus: i) a MOD; ii) a MHC Class II α1 polypeptide; iii) a MHC Class II α2 polypeptide; and iv) an immunoglobulin or non-immunoglobulin scaffold polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; and iii) a MHC Class II β2 polypeptide; and b) a second polypeptide comprising, in order from N-terminus to C-terminus: i) a MOD; ii) a MHC Class II α1 polypeptide; iii) a MHC Class II α2 polypeptide; and iv) an Ig Fc polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II β2 polypeptide; and iv) a first member of a dimerizer pair; and b) a second polypeptide comprising, in order from N-terminus to C-terminus: i) a MOD; ii) a MHC Class II α1 polypeptide; iii) a MHC Class II α2 polypeptide; and iv) a second member of the dimerizer pair. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II β2 polypeptide; and iv) a first leucine zipper polypeptide; and b) a second polypeptide comprising, in order from N-terminus to C-terminus: i) a MOD; ii) a MHC Class II α1 polypeptide; iii) a MHC Class II α2 polypeptide; and iv) a second leucine zipper polypeptide. In any one of the above embodiments, the TMAPP can include a single MOD. In any one of the above embodiments, the TMAPP can include 2 independently selected wild-type or variant MODs (which may be the same or different) that may be placed in tandem, separated by a linker, or in separate parts of the molecule. In any one of the above embodiments, the TMAPP can include 3 independently selected wild-type or variant MODs (which may be the same or different) that may be placed in tandem, separated by linkers, and/or in separate parts of the molecule. In some cases, the TMAPP comprises a linker (an L1) between the MHC polypeptide and the Ig Fc polypeptide, where exemplary suitable linkers include (GGGGS)n, where n is 1, 2, 3, 4, 5, 6, 7, or 8. In some cases, the TMAPP comprises a linker (an L2) between the MOD and the MHC polypeptide, where exemplary suitable linkers include (GGGGS)n, where n is 1, 2, 3, 4, 5, 6, 7, or 8. Where the TMAPP comprises two MODs, in some cases, the two MODs are separated by a linker (an L3), where exemplary suitable linkers include (GGGGS)n, where n is 1, 2, 3, 4, 5, 6, 7, or 8.

MOD-Containing m-TMAPPs—Embodiment Set 5: In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; and iv) a MHC Class II α2 polypeptide; and b) a second polypeptide comprising, in order from N-terminus to C-terminus: i) a MOD; and ii) a MHC Class II β2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) an immunoglobulin or non-immunoglobulin scaffold polypeptide; and b) a second polypeptide comprising, in order from N-terminus to C-terminus: i) a MOD; and ii) a MHC Class II β2 polypeptide. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) an Ig Fc polypeptide; and b) a second polypeptide comprising, in order from N-terminus to C-terminus: i) a MOD; and ii) a MHC Class II β2 polypeptide. In some cases, a MOD containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) a first member of a dimerizer pair; and b) a second polypeptide comprising, in order from N-terminus to C-terminus: i) a MOD; ii) a MHC Class II β2 polypeptide; and iii) a second member of the dimerizer pair. In some cases, a MOD-containing m-TMAPP having a chemical conjugation site (or its epitope conjugate) comprises: a) a first polypeptide comprising, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) a first leucine zipper polypeptide; and b) a second polypeptide comprising, in order from N-terminus to C-terminus: i) a MOD; ii) a MHC Class II β2 polypeptide; and iii) a second leucine zipper polypeptide. In any one of the above embodiments, the TMAPP can include a single MOD. In any one of the above embodiments, the TMAPPe can include 2 independently selected wild-type or variant MODs (which may be the same or different) that may be placed in tandem, separated by a linker, or in separate parts of the molecule. In any one of the above embodiments, the TMAPP can include 3 independently selected wild-type or variant MODs (which may be the same or different) that may be placed in tandem, separated by linkers, and/or in separate parts of the molecule. In some cases, the TMAPP comprises a linker (an L1) between the MHC polypeptide and the Ig Fc polypeptide, where exemplary suitable linkers include (GGGGS)n, where n is 1, 2, 3, 4, 5, 6, 7, or 8. In some cases, the TMAPP comprises a linker (an L2) between the MOD and the MHC polypeptide, where exemplary suitable linkers include (GGGGS)n, where n is 1, 2, 3, 4, 5, 6, 7, or 8. Where the TMAPP comprises two MODs, in some cases, the two MODs are separated by a linker (an L3), where exemplary suitable linkers include (GGGGS)n, where n is 1, 2, 3, 4, 5, 6, 7, or 8.

A MOD-containing m-TMAPP (e.g., any of the above-mentioned MOD-containing m-TMAPPs) having at least one chemical conjugation site (e.g., at a first or second polypeptide N-terminus, or within the optional linker) may be reacted with an epitope to produce a MOD-containing m-TMAPP-epitope conjugate having the epitope covalently bound at one or more chemical conjugation sites (e.g., one chemical conjugation site that permits the epitope to be bound and recognized by a TCR). After conjugation the MOD-containing m-TMAPP-epitope conjugates may contain additional chemical conjugation sites (e.g., for conjugation of a payload). Accordingly, the specification also provides for and includes such MOD-containing m-TMAPP epitope conjugates.

Exemplary m-TMAPPs Comprising One or More MODs

The following are non-limiting embodiments of m-TMAPPs of the present disclosure having an epitope linked to a first and/or second polypeptide. It should be noted that any TMAPP to be administered to an individual in need thereof will generally not include a leader sequence or a histidine tag.

Example 1) In some cases, a m-TMAPP-epitope conjugate comprises: a) a first polypeptide comprising, in order from N-terminus to C-terminus: i) an epitope covalently bound (directly or indirectly via a linker) to ii) a linker; iii) a HLA polypeptide; iv) a HLA α1 polypeptide; v) a HLA α2 polypeptide; vi) a dimerizer polypeptide; and vii) an Ig Fc polypeptide; and b) a second polypeptide comprising, in order from N-terminus to C-terminus: i) a first MOD (e.g., a wild-type or a variant MOD); ii) a second independently selected MOD (e.g., a wild-type or a variant MOD); iii) a HLA β2 polypeptide; and iv) a dimerizer polypeptide. As one non-limiting example, a m-TMAPP-epitope conjugate can comprise: a) a first polypeptide comprising, in order from N-terminus to C-terminus: i) an epitope covalently bound (directly or indirectly via a linker) to ii) a linker; iii) a HLA DRB1 β1 polypeptide; iv) a HLA DRA α1 polypeptide; v) a HLA DRA α2 polypeptide; vi) a leucine zipper dimerizer polypeptide; and vii) an IgG1 Fc polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); iii) a HLA DRB β2 polypeptide; and iv) a leucine zipper dimerizer polypeptide. In some cases, the epitope is a hemagglutinin epitope, e.g., PKYVKQNTLKLAT (SEQ ID NO:85). In some cases, the epitope of the first polypeptide is not PKYVKQNTLKLAT (SEQ ID NO:85), but instead is substituted with a different epitope.

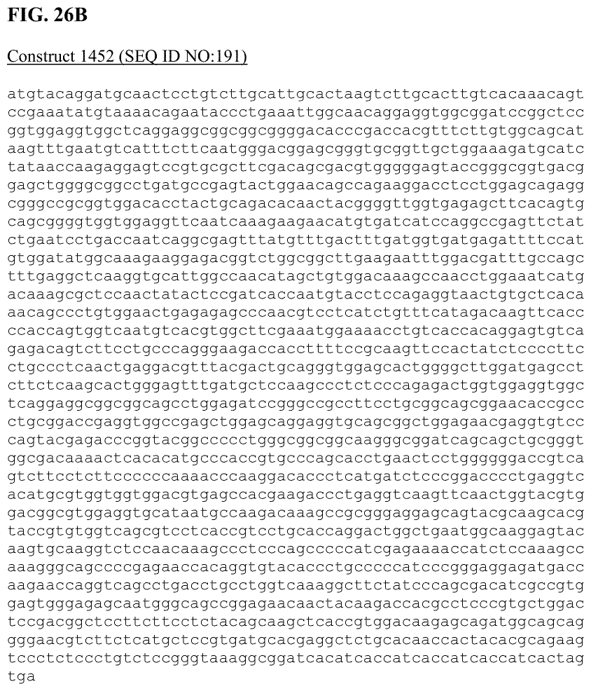

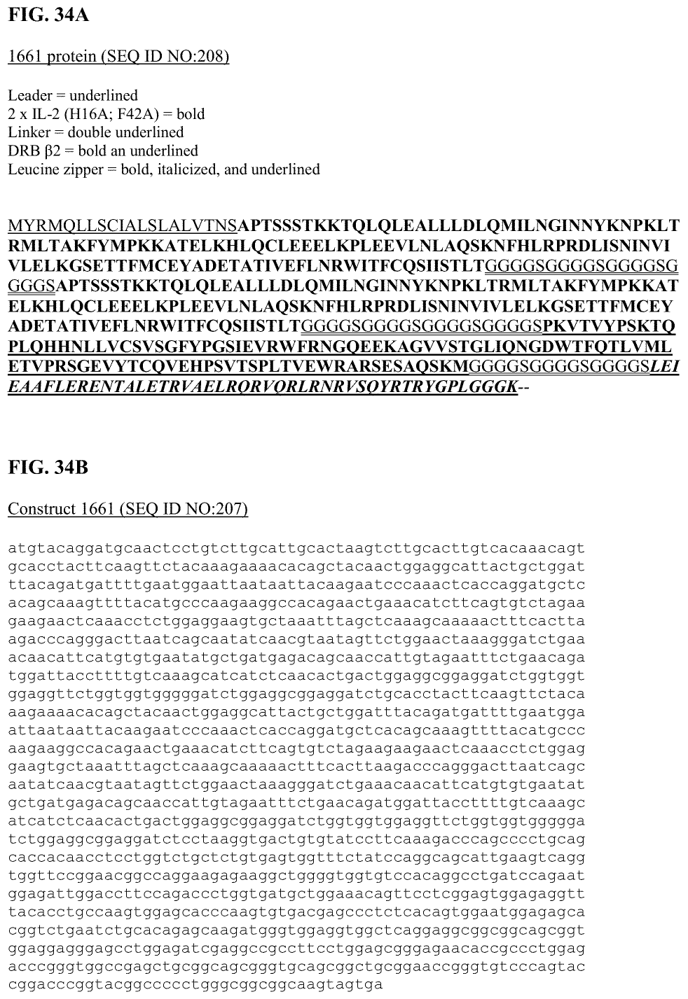

In some cases, the variant IL-2 polypeptide comprises the following amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:27 with H16A and F42A substitutions), where the H16A and F42A substitutions are underlined. In some cases, the HLA-DRB1 β1 polypeptide comprises the following amino acid sequence: DTRPRFLWQHKFECHFFNGTERVRLLERCIYNQEESVRFDSDVGEYRAVTELGRPDAEYWNSQ KDLLEQRRAAVDTYCRHNYGVGESFTVQR (SEQ ID NO:177). In some cases, the HLA DRA α1 polypeptide comprises the following amino acid sequence IKEEHVIIQAEFYLNPDQSGEFMFDFDGDEIFHVDMAKKETVWRLEEFGRFASFEAQGALANIA VDKANLEIMTKRSNYTPITN (SEQ ID NO:178). In some cases, the HLA DRA α2 polypeptide comprises the following amino acid sequence VPPEVTVLTNSPVELREPNVLICFIDKFTPPVVNVTWLRNGKPVTTGVSETVFLPREDHLFRKFH YLPFLPSTEDVYDCRVEHWGLDEPLLKHWEFDAPSPLPET (SEQ ID NO:179). In some cases, the leucine zipper dimerizer polypeptide comprises the following amino acid sequence: LEIRAAFLRQRNTALRTEVAELEQEVQRLENEVSQYETRYGPLGGGK (SEQ ID NO:180). In some cases, the IgG1 Fc polypeptide comprises the following amino acid sequence: DKTHTCPPCPAPELLGGPSVFLFPPKPKDTLMISRTPEVTCVVVDVSHEDPEVKFNWYVDGVEV HNAKTKPREEQYASTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO:181). The amino acid sequences of the first polypeptide may be organized in a fashion similar to amino acids 21 to 628 of protein/polypeptide construct 1452 depicted in FIG. 26A (note that in a TMAPP that has not been conjugated with an epitope there is a chemical conjugation site at the location where the epitope will be located, a mature TMAPP is without the leader sequence and may lack the C-terminal linker and histidine tag). The amino acid sequences of the second polypeptide may be organized in a fashion similar to amino acids 21 to 491 of protein/polypeptide construct 1661 depicted in FIG. 34A (without the leader sequence).

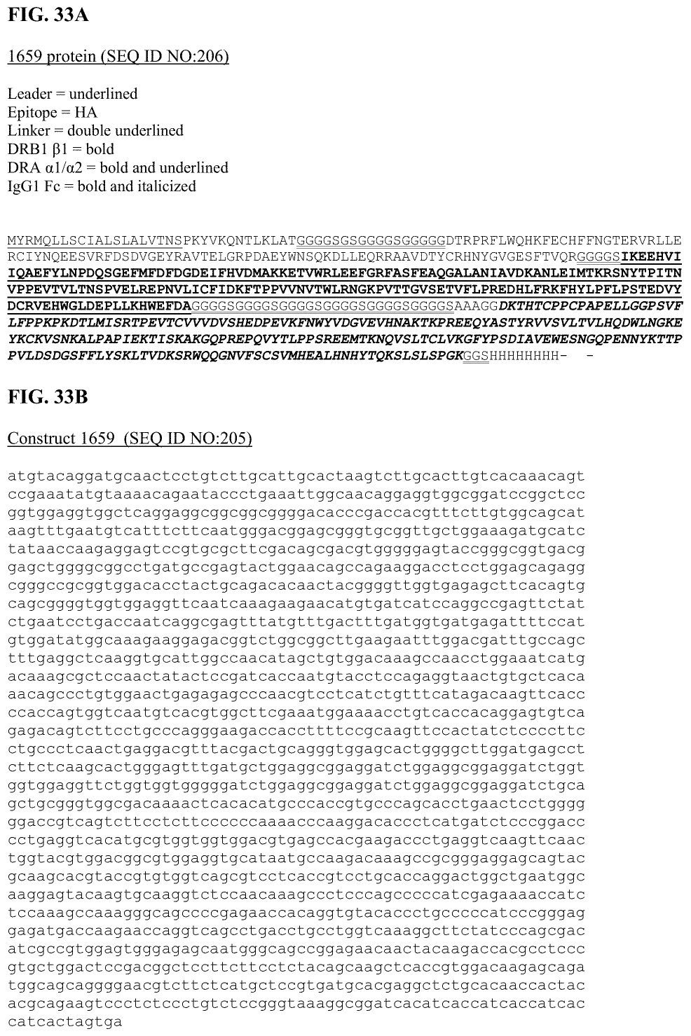

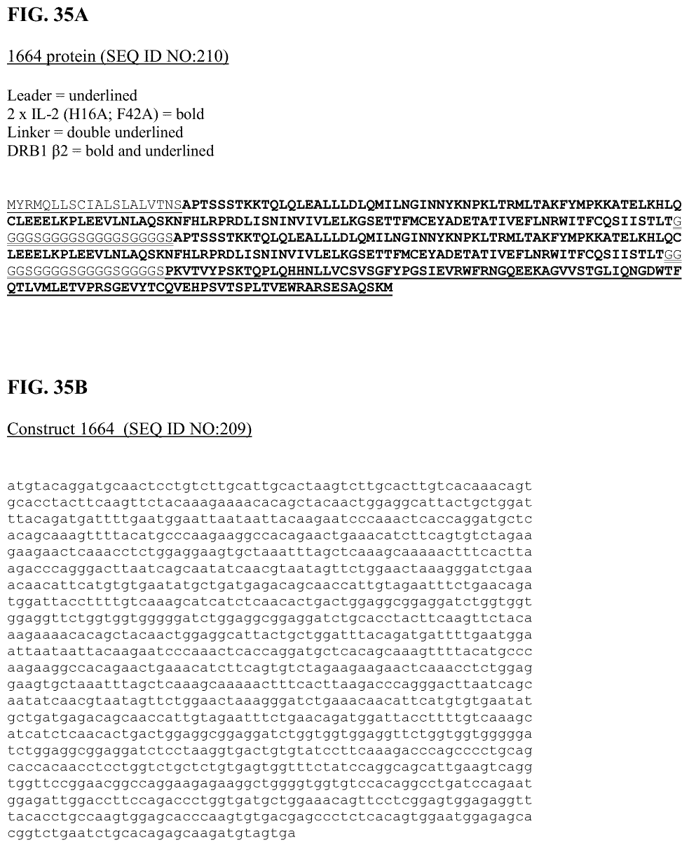

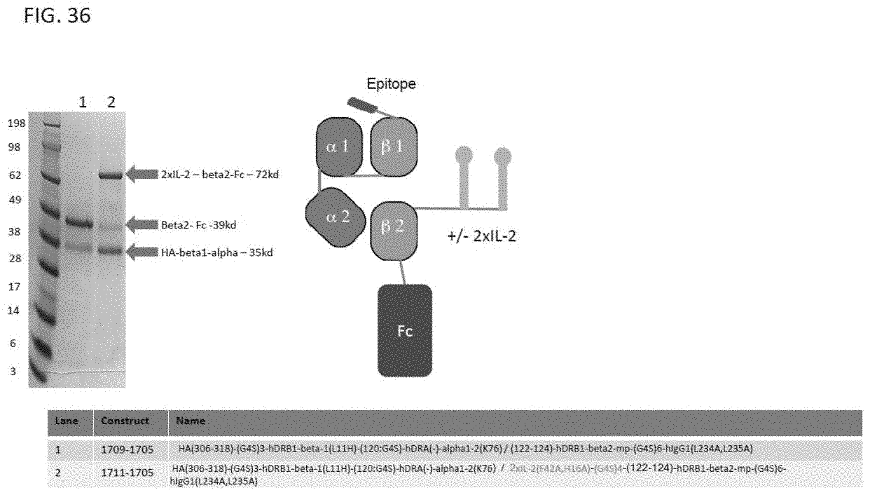

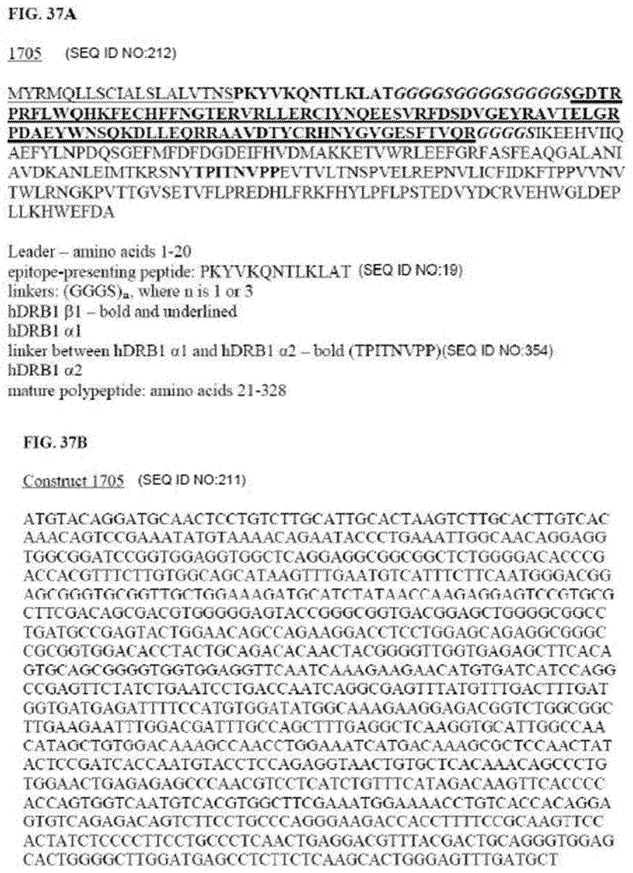

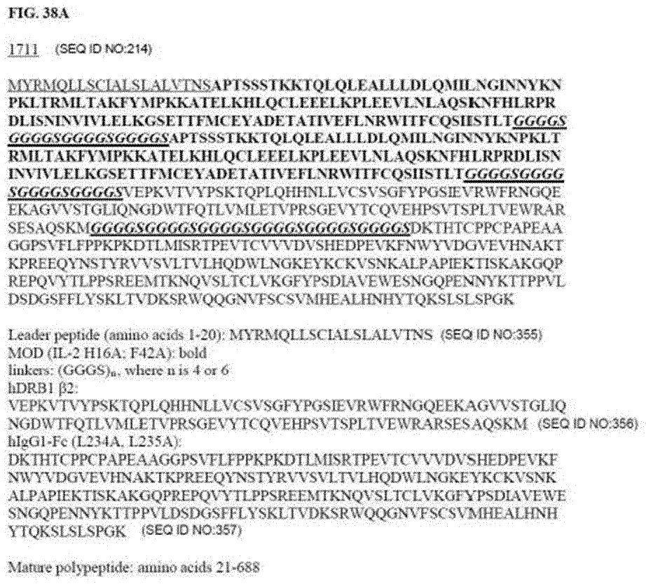

Example 2) In some cases, a m-TMAPP-epitope conjugate comprises: a) a first polypeptide comprising, in order from N-terminus to C-terminus: i) an epitope covalently bound (directly or indirectly via a linker) to ii) a HLA β1 polypeptide; iii) a HLA α1 polypeptide; iv) a HLA α2 polypeptide; and v) an Ig Fc polypeptide; and b) a second polypeptide comprising, in order from N-terminus to C-terminus: i) a first MOD (e.g., a variant MOD with reduced affinity for its Co-MOD); ii) a second independently selected MOD (e.g., a variant MOD with reduced affinity for its Co-MOD); and iii) a HLA β2 polypeptide. As one non-limiting example, a m-TMAPP-epitope conjugate can comprise: a) a first polypeptide comprising, in order from N-terminus to C-terminus: i) an epitope covalently bound (directly or indirectly via a linker) to ii) a HLA DRB1β1 polypeptide; iii) a HLA DRA α1 polypeptide; iv) a HLA DRA α2 polypeptide; and v) an IgG1 Fc polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); and iii) a HLA DRB1 β2 polypeptide. In some cases, the epitope is a hemagglutinin epitope, e.g., PKYVKQNTLKLAT (SEQ ID NO:85). In some cases, the epitope is not PKYVKQNTLKLAT (SEQ ID NO:85), but instead is substituted with a different epitope. In some cases, the HLA DRB1 polypeptide comprises the following amino acid sequence: DTRPRFLWQHKFECHFFNGTERVRLLERCIYNQEESVRFDSDVGEYRAVTELGRPDAEYWNSQ KDLLEQRRAAVDTYCRHNYGVGESFTVQR (SEQ ID NO:177). In some cases, the DRA α1 polypeptide comprises the following amino acid sequence: IKEEHVIIQAEFYLNPDQSGEFMFDFDGDEIFHVDMAKKETVWRLEEFGRFASFEAQGALANIA VDKANLEIMTKRSNYTPITN (SEQ ID NO:178). In some cases, the DRA α2 polypeptide comprises the following amino acid sequence: VPPEVTVLTNSPVELREPNVLICFIDKFTPPVVNVTWLRNGKPVTTGVSETVFLPREDHLFRKFH YLPFLPSTEDVYDCRVEHWGLDEPLLKHWEFDA (aa 1-98 of SEQ ID NO:179). In some cases, the IgG1 Fc polypeptide comprises the following amino acid sequence: DKTHTCPPCPAPELLGGPSVFLFPPKPKDTLMISRTPEVTCVVVDVSHEDPEVKFNWYVDGVEV HNAKTKPREEQYASTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO:181). In some cases, the variant IL-2 polypeptide comprises the following amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:27 with H16A and F42A substitutions), where the H16A and F42A substitutions are underlined. In some cases, the HLA DRB1 β2 polypeptide comprises the following amino acid sequence: PKVTVYPSKTQPLQHHNLLVCSVSGFYPGSIEVRWFRNGQEEKAGVVSTGLIQNGDWTFQTLV MLETVPRSGEVYTCQVEHPSVTSPLTVEWRARSESAQSKM (SEQ ID NO:182). The amino acid sequences of the first polypeptide may be organized in a fashion similar to amino acids 21 to 591 of protein/polypeptide construct 1659 depicted in FIG. 33A (note that in TMAPP that has not been conjugated with an epitope there is a chemical conjugation site in the epitope's place, and that a mature TMAPP is without the leader sequence and may lack the C-terminal linker and histidine tag). The amino acid sequences of the second polypeptide may be organized in a fashion similar to amino acids 21 to 429 of protein/polypeptide construct 1664 depicted in FIG. 35A (without the leader sequence).

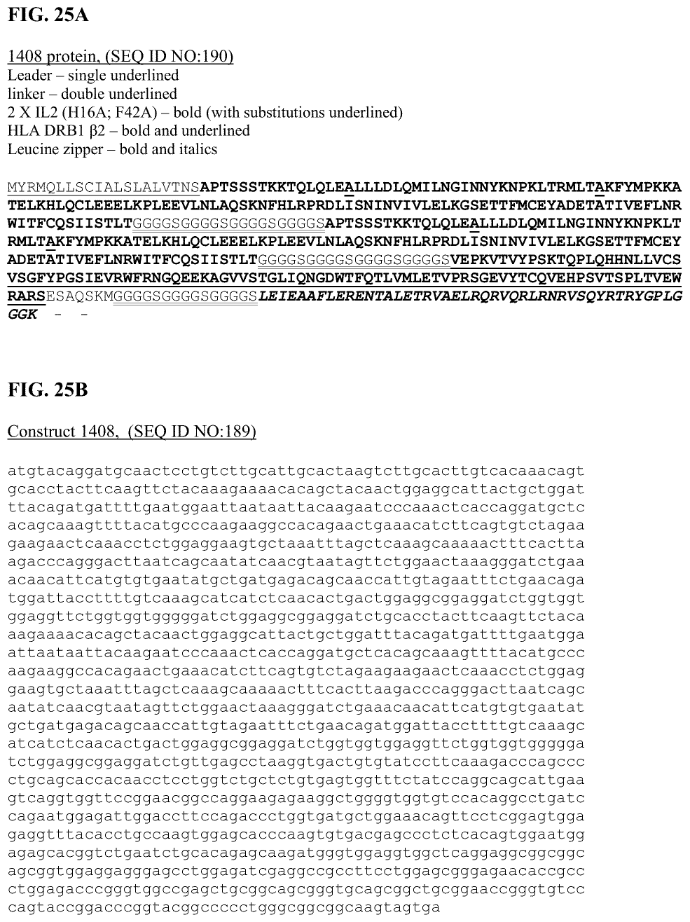

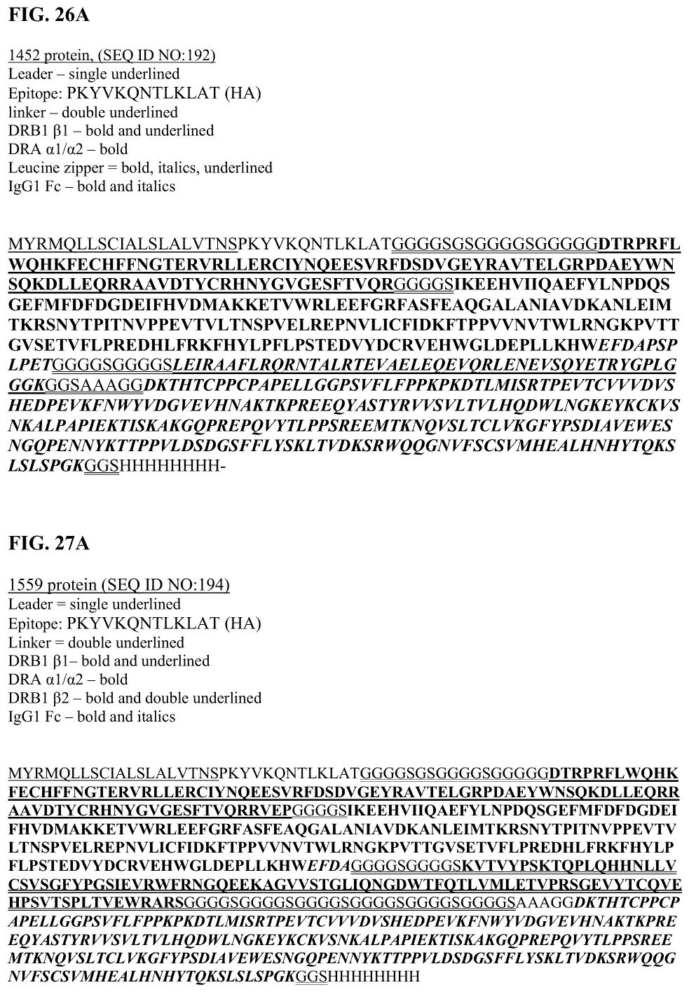

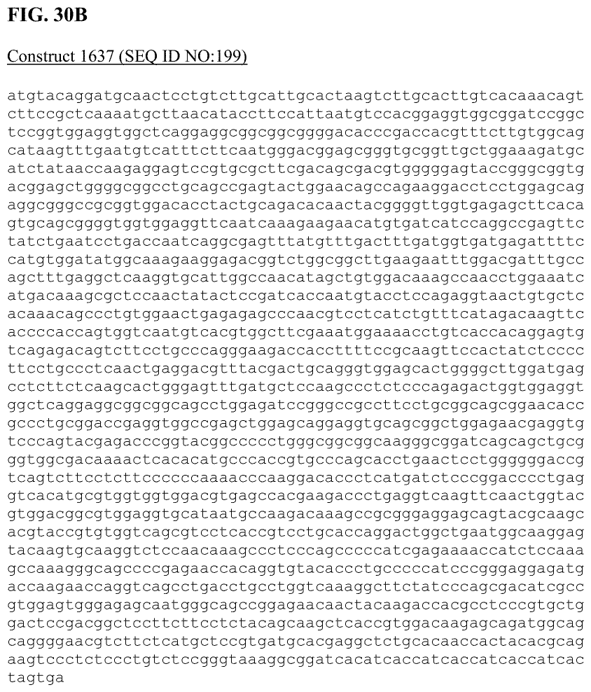

Example 3) In some cases, a m-TMAPP-epitope conjugate comprises: a) a first polypeptide comprising, in order from N-terminus to C-terminus: i) an epitope covalently bound (directly or indirectly via a linker) to ii) a HLA polypeptide; iii) a HLA α1 polypeptide; iv) a HLA α2 polypeptide; v) a dimerizer polypeptide; and vi) an Ig Fc polypeptide; and b) a second polypeptide comprising, in order from N-terminus to C-terminus: i) a first MOD (e.g., a variant MOD with reduced affinity for its Co-MOD); ii) a second independently selected MOD (e.g., a variant MOD with reduced affinity for its Co-MOD); iii) a HLA β2 polypeptide; and iv) a dimerizer polypeptide. As one non-limiting example, a m-TMAPP-epitope conjugate can comprise: a) a first polypeptide comprising, in order from N-terminus to C-terminus: i) an epitope covalently bound (directly or indirectly via a linker) to ii) a HLA DRB1 polypeptide; iii) a HLA DRA α1 polypeptide; iv) a HLA DRA α2 polypeptide; v) a leucine zipper dimerizer polypeptide; and vi) an IgG1 Fc polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); iii) a HLA DRB1β2 polypeptide; and iv) a leucine zipper dimerizer polypeptide. In some cases, the epitope is a cytomegalovirus (CMV) pp65 epitope (LPLKMLNIPSINVH; SEQ ID NO:184). In some cases, the first polypeptide does not include the epitope LPLKMLNIPSINVH (SEQ ID NO:184); instead, the epitope is substituted with a different epitope. In some cases, the HLA DRB polypeptide comprises the following amino acid sequence: DTRPRFLWQHKFECHFFNGTERVRLLERCIYNQEESVRFDSDVGEYRAVTELGRPAAEYWNSQ KDLLEQRRAAVDTYCRHNYGVGESFTVQR (SEQ ID NO:185). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGALANIA VDKANLEIMTKRSNYTPITN (SEQ ID NO:178). In some cases, the HLA DRA α2 polypeptide comprises the following amino acid sequence: VPPEVTVLTNSPVELREPNVLICFIDKFTPPVVNVTWLRNGKPVTTGVSETVFLPREDHLFRKFH YLPFLPSTEDVYDCRVEHWGLDEPLLKHWEFDAPSPLPETSEQ ID NO:179). In some cases, the leucine zipper polypeptide comprises the following amino acid sequence: LEIRAAFLRQRNTALRTEVAELEQEVQRLENEVSQYETRYGPLGGGK (SEQ ID NO:180). In some cases, the IgG1 Fc polypeptide comprises the following amino acid sequence: DKTHTCPPCPAPELLGGPSVFLFPPKPKDTLMISRTPEVTCVVVDVSHEDPEVKFNWYVDGVEV HNAKTKPREEQYASTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO:181). In some cases, the variant IL-2 polypeptide comprises the following amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:27 with H16A and F42A substitutions), where the H16A and F42A substitutions are underlined. In some cases, the HLA DRB1 β2 polypeptide comprises the following amino acid sequence: VEPKVTVYPSKTQPLQHHNLLVCSVSGFYPGSIEVRWFRNGQEEKAGVVSTGLIQNGDWTFQT LVMLETVPRSGEVYTCQVEHPSVTSPLTVEWRARSESAQSKM (SEQ ID NO:183). In some cases, the leucine zipper polypeptide comprises the following amino acid sequence: LEIEAAFLERENTALETRVAELRQRVQRLRNRVSQYRTRYGPLGGGK (SEQ ID NO:93). The amino acid sequences of the first polypeptide may be organized in a fashion similar to amino acids 21-629 of protein/polypeptide construct 1637 depicted in FIG. 30A (note that in a TMAPP that has not been conjugated with an epitope there is a chemical conjugation site at the location where the epitope will be located, a mature TMAPP is without the leader sequence and may lack the C-terminal linker and histidine tag). The amino acid sequences of the second polypeptide may be organized in a fashion similar to amino acids 21-493 of protein/polypeptide construct 1408 depicted in FIG. 25A, without the leader sequence.

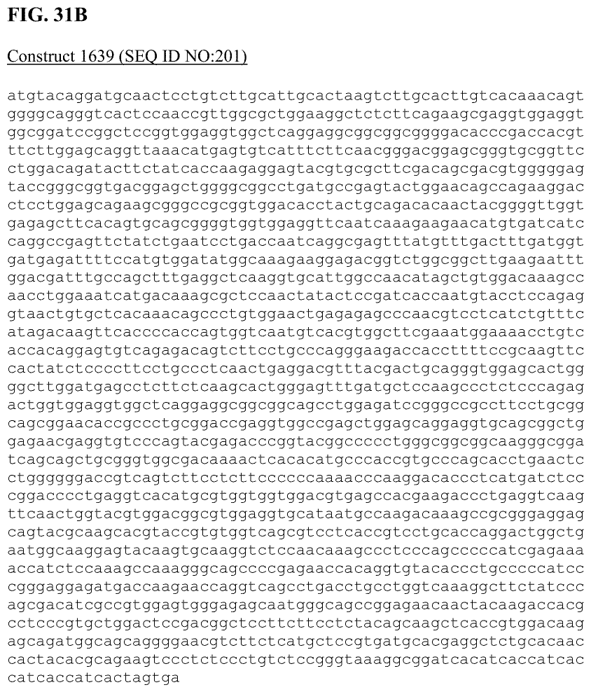

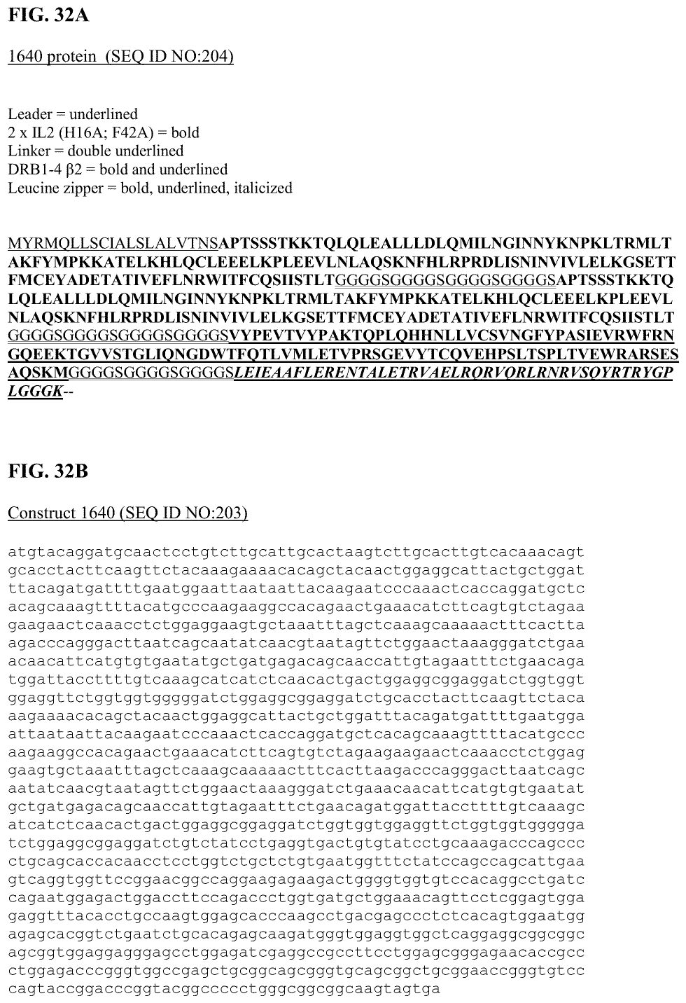

Example 4) In some cases, a m-TMAPP-epitope conjugate comprises: a) a first polypeptide comprising, in order from N-terminus to C-terminus: i) an epitope covalently bound (directly or indirectly via a linker) to ii) a HLA β1 polypeptide; iii) a HLA α1 polypeptide; iv) a HLA α2 polypeptide; v) a dimerizer polypeptide; and vi) an Ig Fc polypeptide; and b) a second polypeptide comprising, in order from N-terminus to C-terminus: i) a first MOD (e.g., a variant MOD with reduced affinity for its Co-MOD); ii) a second independently selected MOD (e.g., a variant MOD with reduced affinity for its Co-MOD); iii) a HLA β2 polypeptide; and iv) a dimerizer polypeptide. As one non-limiting example, a m-TMAPP-epitope conjugate can comprise: a) a first polypeptide comprising, in order from N-terminus to C-terminus: i) an epitope covalently bound (directly or indirectly via a linker) to ii) a HLA DRB1-4 polypeptide; iii) a HLA DRA α1 polypeptide; iv) a HLA DRA α2 polypeptide; v) a leucine zipper dimerizer polypeptide; and vi) an IgG1 Fc polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); iii) a HLA DRB1-4 β2 polypeptide; and iv) a leucine zipper dimerizer polypeptide. In some cases, the epitope is proinsulin 73-90 (GAGSLQPLALEGSLQKR; SEQ ID NO:84). In some cases, the epitope is not proinsulin 73-90 (GAGSLQPLALEGSLQKR; SEQ ID NO:84); instead, the epitope is substituted with a different epitope. In some cases, the HLA DRB1-4 β1 polypeptide comprises the following amino acid sequence: DTRPRFLEQVKHECHFFNGTERVRFLDRYFYHQEEYVRFDSDVGEYRAVTELGRPDAEYWNSQ KDLLEQKRAAVDTYCRHNYGVGESFTVQR (amino acids 1-92 of SEQ ID NO:150). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGALANIA VDKANLEIMTKRSNYTPITN (SEQ ID NO:178). In some cases, the HLA DRA α2 polypeptide comprises the following amino acid sequence: VPPEVTVLTNSPVELREPNVLICFIDKFTPPVVNVTWLRNGKPVTTGVSETVFLPREDHLFRKFH YLPFLPSTEDVYDCRVEHWGLDEPLLKHWEFDAPSPLPET (SEQ ID NO:179). In some cases, the leucine zipper polypeptide comprises the following amino acid sequence: LEIRAAFLRQRNTALRTEVAELEQEVQRLENEVSQYETRYGPLGGGK (SEQ ID NO:180). In some cases, the IgG1 Fc polypeptide comprises the following amino acid sequence: DKTHTCPPCPAPELLGGPSVFLFPPKPKDTLMISRTPEVTCVVVDVSHEDPEVKFNWYVDGVEV HNAKTKPREEQYASTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO:181). In some cases, the variant IL-2 polypeptide comprises the following amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:27 with H16A and F42A), where the H16A and F42A substitutions are underlined. In some cases, the HLA DRB1-4 β2 polypeptide comprises the following amino acid sequence: VYPEVTVYPAKTQPLQHHNLLVCSVNGFYPASIEVRWFRNGQEEKTGVVSTGLIQNGDWTFQT LVMLETVPRSGEVYTCQVEHPSLTSPLTVEWRARSESAQSKM (SEQ ID NO:186, which is related to SEQ ID NO:151 by the addition of a N-terminal Val and C-terminal Met). In some cases, the leucine zipper polypeptide comprises the following amino acid sequence: LEIEAAFLERENTALETRVAELRQRVQRLRNRVSQYRTRYGPLGGGK (SEQ ID NO:83). The amino acid sequences of the first polypeptide may be organized in a fashion similar to amino acids 21-633 of protein/polypeptide construct 1639 depicted in FIG. 31A (note that in a TMAPP that has not been conjugated with an epitope there is a chemical conjugation site at the location where the epitope will be located, a mature TMAPP is without the leader sequence and may lack the C-terminal linker and histidine tag). The amino acid sequences of the second polypeptide may be organized in a fashion similar to amino acids 21-493 of protein/polypeptide construct 1639 depicted in FIG. 32A (without the leader sequence).

Example 5) In some cases, a m-TMAPP-epitope conjugate of the present disclosure comprises: a) a first polypeptide comprising, in order from N-terminus to C-terminus: i) an epitope covalently bound (directly or indirectly via a linker) to ii) a HLA β1 polypeptide; iii) a HLA α1 polypeptide; and iv) a HLA α2 polypeptide; and b) a second polypeptide comprising, in order from N-terminus to C-terminus: i) a first MOD (e.g., a variant MOD with reduced affinity for its cognate Co-MOD); ii) a second independently selected MOD (e.g., a variant MOD with reduced affinity for its cognate Co-MOD); iii) a HLA β2 polypeptide; and iv) an Ig Fc polypeptide. As one non-limiting example, a m-TMAPP of the present disclosure can comprise: a) a first polypeptide comprising, in order from N-terminus to C-terminus: i) an epitope; ii) a HLA DRB1 β1 polypeptide; iii) a HLA DRA α1 polypeptide; and iv) a HLA DRA α2 polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); iii) a HLA DRB1 β2 polypeptide; and iv) an IgG Fc polypeptide. The m-TMAPP can include a variant IgG Fc polypeptide. For example, a m-TMAPP of the present disclosure can comprise: a) a first polypeptide comprising, in order from N-terminus to C-terminus: i) an epitope covalently bound to ii) a HLA DRB1 β1 polypeptide; iii) a HLA DRA α1 polypeptide; and iv) a HLA DRA α2 polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); iii) a HLA DRB1 β2 polypeptide; and iv) an IgG1 Fc polypeptide comprising L234A and L235A substitutions. The m-TMAPP can include one or more linkers. For example, a m-TMAPP of the present disclosure can comprise: a) a first polypeptide comprising, in order from N-terminus to C-terminus: i) an epitope covalently bound (directly or indirectly via a linker) to ii) a peptide linker; iii) a HLA DRB1 β1 polypeptide; iv) a peptide linker; v) a HLA DRA α1 polypeptide; and vi) a HLA DRA α2 polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); iii) a peptide linker; iv) a HLA DRB1 β2 polypeptide; v) a peptide linker; and vi) an Ig Fc polypeptide (e.g., an IgG1 Fc polypeptide comprising L234A and L235A substitutions). For example, a m-TMAPP of the present disclosure can comprise: a) a first polypeptide comprising, in order from N-terminus to C-terminus: i) an epitope covalently bound (directly or indirectly via a linker) to ii) the peptide linker (GGGGS)₃; iii) a HLA DRB1 β1 polypeptide; iv) the peptide linker GGGGS; v) a HLA DRA α1 polypeptide; and vi) a HLA DRA α2 polypeptide; and b) a second polypeptide comprising, in order from N-terminus to C-terminus: i) a first MOD (e.g., a variant IL-2 polypeptide comprising H16A and F42A substitutions); ii) a second independently selected MOD (e.g., a variant IL-2 polypeptide comprising H16A and F42A substitutions); iii) the peptide linker (GGGGS)₄; iv) a HLA DRB1 β2 polypeptide; v) the peptide linker (GGGGS)₆; and vi) an Ig Fc polypeptide (e.g., an IgG1 Fc polypeptide comprising L234A and L235A substitutions). For example, a m-TMAPP of the present disclosure can comprise: a) a first polypeptide comprising, in order from N-terminus to C-terminus: i) an epitope covalently bound (directly or indirectly via a linker) to ii) the peptide linker (GGGGS)₃; iii) a HLA DRB1 β1 polypeptide; iv) the peptide linker GGGGS; v) a HLA DRA α1 polypeptide; and vi) an HLA DRA α2 polypeptide; and b) a second polypeptide comprising, in order from N-terminus to C-terminus: i) a first variant IL-2 polypeptide comprising H16A and F42A substitutions; ii) a second variant IL-2 polypeptide comprising H16A and F42A substitutions (e.g., where the first and the second variant IL-2 polypeptides comprise the same amino acid sequence); iii) the peptide linker (GGGGS)₄; iv) a HLA DRB1 β2 polypeptide; v) the peptide linker (GGGGS)₆; and vi) an IgG1 Fc polypeptide comprising L234A and L235A substitutions. In some cases, the HLA DRB1 β1 polypeptide comprises the following amino acid sequence: DTRPRFLWQHKFECHFFNGTERVRLLERCIYNQEESVRFDSDVGEYRAVTELGRPDAEYWNSQ KDLLEQRRAAVDTYCRHNYGVGESFTVQR (SEQ ID NO:177). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGALANIA VDKANLEIMTKRSNY (amino acids 1-79 of SEQ ID NO:178). In some cases, the HLA DRA α2 polypeptide comprises the following amino acid sequence: EVTVLTNSPVELREPNVLICFIDKFTPPVVNVTWLRNGKPVTTGVSETVFLPREDHLFRKFHYLP FLPSTEDVYDCRVEHWGLDEPLLKHWEFDA (amino acids 4-94 of SEQ ID NO:176). In some cases, the HLA DRB1 β2 polypeptide comprises the following amino acid sequence: VEPKVTVYPSKTQPLQHHNLLVCSVSGFYPGSIEVRWFRNGQEEKAGVVSTGLIQNGDWTFQT LVMLETVPRSGEVYTCQVEHPSVTSPLTVEWRARSESAQSKM (SEQ ID NO:183). In some cases, the first and the second independently selected MODs are variant IL-2 polypeptides, both comprising the amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:27 with H16A and F42A), where the H16A and the F42A substitutions are underlined. In some cases, the Fc polypeptide is an IgG1 Fc polypeptide comprising L234A and L235A substitutions, and comprises the amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPGK (SEQ ID NO:187, comprising L234A and L235A substitutions relative to SEQ ID NO:181).

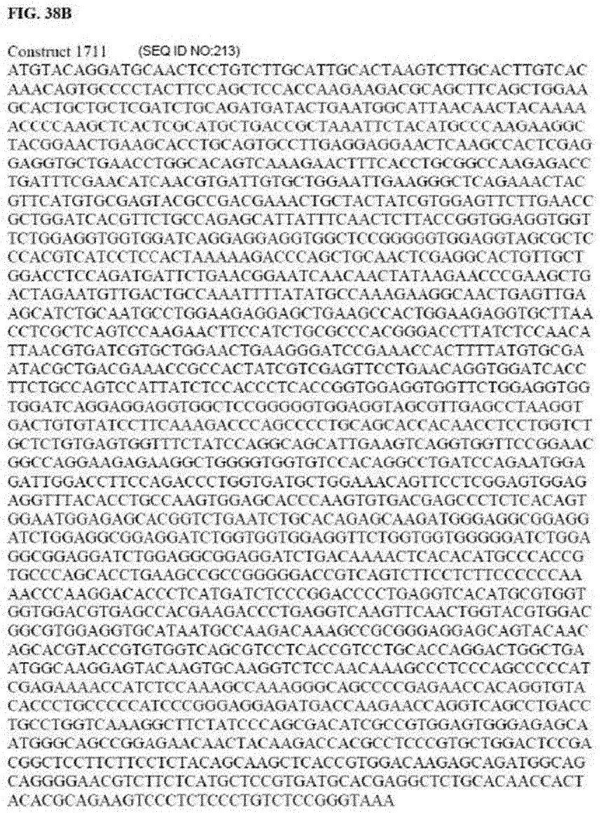

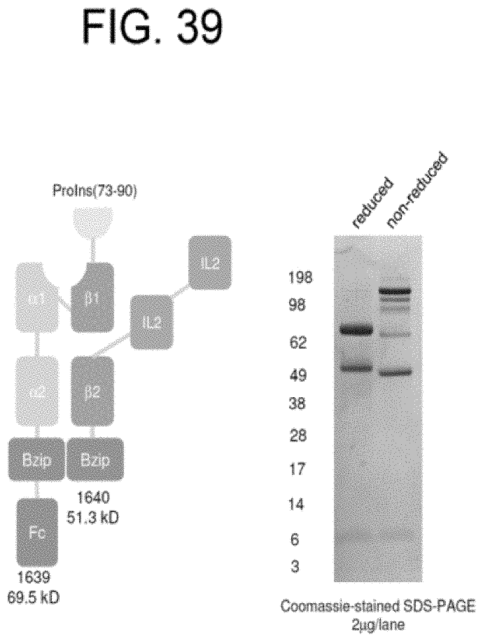

The amino acid sequences of the first polypeptide may be organized in a fashion similar to amino acids 21-328 of protein/polypeptide construct 1705 depicted in FIG. 37A (note that in TMAPP that has not been conjugated with an epitope there is a chemical conjugation site in the epitope's place, and that a mature TMAPP is without the leader sequence). The amino acid sequences of the second polypeptide may be organized in a fashion similar to amino acids 21-688 of protein/polypeptide construct 1711 depicted in FIG. 38A (without the leader sequence).

MOD-Containing sc-TMAPPs

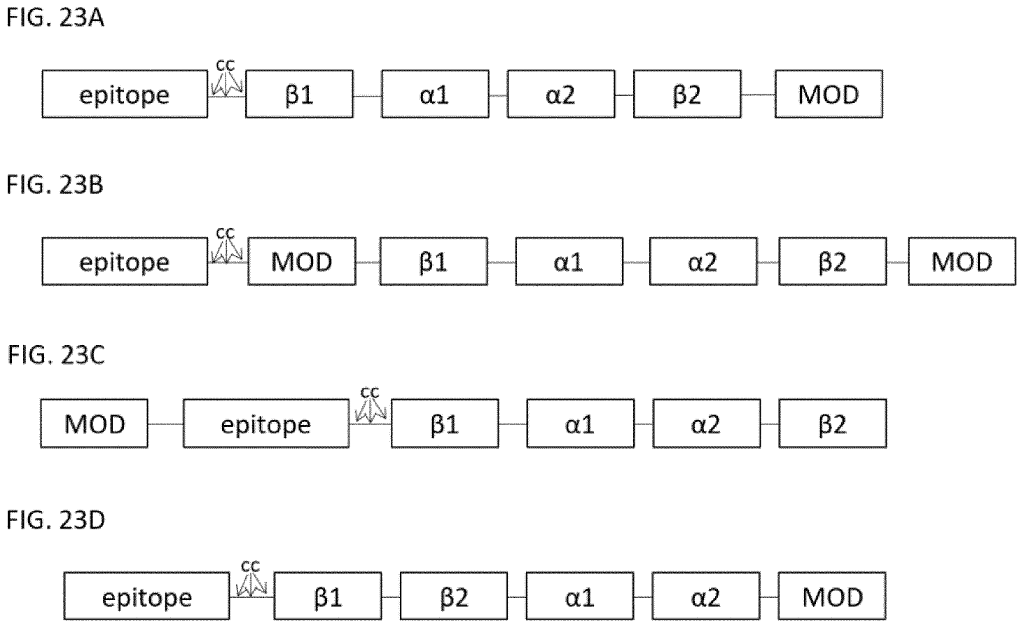

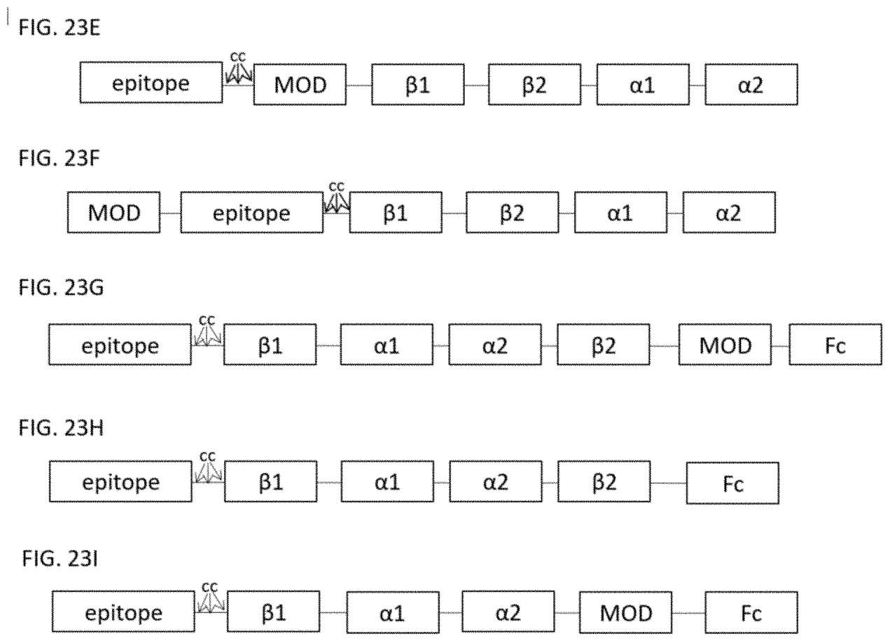

As noted above, in some cases, a TMAPP-epitope conjugate comprises a single polypeptide chain and is denoted as a sc-TMAPP. The sc-TMAPP polypeptides set forth in this section comprise one or more MODs. Non-limiting examples are depicted schematically in FIGS. 23A-23F. Any of the sc-TMAPP-epitope conjugates described in this section, or the following section directed to Exemplary sc-TMAPPs Comprising One Or More MODs, can include one or more linkers between any two adjacent polypeptides, including, but not limited to, between: an epitope (such as a peptide antigen) and a MOD, between a MOD and a MHC Class II polypeptide (e.g., MHC Class II α1, α2, β1, or β2 polypeptide), between two MHC Class II polypeptides, between a MOD and an Ig Fc polypeptide, and between a first MOD and a second independently selected MOD.

Throughout this section on sc-TMAPPs comprising one or more MODs and the section directed to exemplary sc-TMAPPs comprising one or more MODs that follows, unless stated otherwise when a sc-TMAPP has not been conjugated to an epitope (e.g., a peptide antigen that is capable of being recognized and bound by a TCR), it comprises one or more chemical conjugation sites (e.g., in the optional linker and/or the MHC Class II β1 polypeptide sequence); and when converted to its sc-TMAPP-epitope conjugate, it comprises an epitope covalently attached (directly or indirectly through a linker) to at least one of those one or more chemical conjugation sites (e.g., at or near the N-terminus of the optional linker or the β1 polypeptide).

In some cases, a MOD-containing sc-TMAPP having a chemical conjugation site, or its epitope conjugate, comprises a single polypeptide chain comprising (e.g., from N- to C-terminus): i) an optional linker; ii) a MHC Class II α1 polypeptide; iii) a MHC Class II α2 polypeptide; iv) a MHC Class II β1 polypeptide; and v) one or more MODs. In some cases, a sc-TMAPP having a chemical conjugation site, or its epitope conjugate, comprises a single polypeptide chain comprising (e.g., from N- to C-terminus): i) an optional linker; ii) a MHC Class II α1 polypeptide; iii) a MHC Class II α2 polypeptide; iv) a MHC Class II β1 polypeptide; and v) one or more immunomodulatory polypeptides. In some cases, a sc-TMAPP having a chemical conjugation site, or its epitope conjugate, comprises a single polypeptide chain comprising (e.g., from N- to C-terminus): i) an optional linker; ii) a MHC Class II α1 polypeptide; iii) a MHC Class II α2 polypeptide; iv) a MHC Class II β1 polypeptide; v) a MHC Class II β2 polypeptide; and vi) one or more MODs; wherein when the sc-TMAPP has not been conjugated to an epitope the optional linker and/or the MHC Class II β1 polypeptide comprise one or more chemical conjugation sites. In some cases, a sc-TMAPP having a chemical conjugation site, or its epitope conjugate, comprises a single polypeptide chain comprising (e.g., from N- to C-terminus): i) an optional linker; ii) a MHC Class II α1 polypeptide; iii) a MHC Class II α2 polypeptide; iv) a MHC Class II β1 polypeptide; v) a MHC Class II β2 polypeptide; vi) one or more MODs; and vii) an Ig or a non-Ig scaffold polypeptide. In some cases, a sc-TMAPP having a chemical conjugation site, or its epitope conjugate, comprises a single polypeptide chain comprising (e.g., from N- to C-terminus): i) an optional linker; ii) a MHC Class II α1 polypeptide; iii) a MHC Class II α2 polypeptide; iv) a MHC Class II β1 polypeptide; v) a MHC Class II β2 polypeptide; vi) one or more MODs; and vii) a dimerizing polypeptide. In some cases, the sc-TMAPP comprises a linker (an L1) between a MHC polypeptide and an Ig Fc polypeptide; exemplary suitable linkers include (GGGGS)n, where n=1, 2, 3, 4, 5, 6, 7, 8, 9, or 10. In some cases, the sc-TMAPP comprises a linker (an L2) between a MOD and a MHC polypeptide; exemplary suitable linkers include (GGGGS)n, where n=1, 2, 3, 4, 5, 6, 7, or 8. In some cases, where the sc-TMAPP comprises two MODs, the two MODs are separated by a linker (an L3); exemplary suitable linkers include (GGGGS)n, where n=1, 2, 3, 4, 5, 6, 7, or 8.

In some cases, a MOD-containing sc-TMAPP having a chemical conjugation site, or its epitope conjugate, comprises a single polypeptide chain comprising (e.g., from N- to C-terminus): i) an optional linker; ii) a MHC Class II β1 polypeptide; iii) a MHC Class II α1 polypeptide; iv) a MHC Class II α2 polypeptide; v) a MHC Class II β2 polypeptide; and vi) one or more MODs. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; and v) one or more MODs. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; vi) one or more MODs; and vii) an Ig Fc polypeptide. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; vi) a first MOD; vii) a second independently selected MOD; and viii) an Ig Fc polypeptide. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a first MOD; vi) a second independently selected MOD; and vii) an Ig Fc polypeptide. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; vi) one or more MODs; and vii) a dimerizing polypeptide. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; vi) one or more MODs; vii) a dimerizing polypeptide; and viii) a second dimerizing polypeptide. In some cases, the sc-TMAPP comprises a linker (an L1) between a MHC polypeptide and an Ig Fc polypeptide; exemplary suitable linkers include (GGGGS)n, where n=1, 2, 3, 4, 5, 6, 7, or 8. In some cases, the sc-TMAPP comprises a linker (an L2) between a MOD and a MHC polypeptide, where exemplary suitable linkers include (GGGGS)n, where n=1, 2, 3, 4, 5, 6, 7, or 8. In some cases, where the sc-TMAPP comprises two MODs, the two MODs are separated by a linker (an L3), where exemplary suitable linkers include (GGGGS)n, where n=1, 2, 3, 4, 5, 6, 7, or 8.

In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; and vi) a MOD. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; and vi) an Ig Fc polypeptide. In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; and vi) 2 MODs (which may be the same or selected independently). In some cases, a sc-TMAPP having a chemical conjugation site, or its epitope conjugate, comprises, in order from N-terminus to C-terminus: i) an optional linker; ii) a MHC Class II β1 polypeptide; iii) a MHC Class II α1 polypeptide; iv) a MHC Class II α2 polypeptide; v) a MHC Class II β2 polypeptide; vi) 2 MODs (which may be the same or selected independently); and v) an Ig Fc polypeptide.

In some cases, a MOD-containing sc-TMAPP having a chemical conjugation site, or its epitope conjugate, comprises a single polypeptide chain comprising (e.g., from N- to C-terminus): i) an optional linker; ii) a MOD; iii) a MHC Class II β1 polypeptide; iv) a MHC Class II α1 polypeptide; v) a MHC Class II α2 polypeptide; vi) a MHC Class II β2 polypeptide; and v) a second independently selected MOD. In some cases, a sc-TMAPP having a chemical conjugation site, or its epitope conjugate, comprises, in order from N-terminus to C-terminus: i) an optional linker; ii) a MOD; iii) a MHC Class II β1 polypeptide; iv) a MHC Class II α1 polypeptide; v) a MHC Class II α2 polypeptide; vi) a MHC Class II β2 polypeptide; vii) a second independently selected MOD; and viii) an immunoglobulin or non-immunoglobulin scaffold polypeptide. In some cases, a sc-TMAPP having a chemical conjugation site, or its epitope conjugate, comprises, in order from N-terminus to C-terminus: i) an optional linker; ii) a MOD; iii) a MHC Class II β1 polypeptide; iv) a MHC Class II α1 polypeptide; v) a MHC Class II α2 polypeptide; vi) a MHC Class II β2 polypeptide; vii) a second independently selected MOD; and viii) an Ig Fc polypeptide.

In some cases, a polypeptide comprising, from N-terminus to C-terminus, i) a MOD (first MOD) and ii) an epitope (a MOD-epitope peptide) is conjugated with a MOD-containing sc-TMAPP having one or more chemical conjugation sites, with one at or near (e.g., within 30, 20, 10 or 5 aa) its N-terminus, such as in an N-terminal linker. In one embodiment, the sc-TMAPP for conjugation to the MOD-epitope peptide comprises a chemical conjugation at or near its N-terminus (e.g., as part of an N-terminal linker), and comprises, in order from N-terminus to C-terminus: i) an optional linker that when present is bound to ii) a MHC Class II β1 polypeptide; iii) a MHC Class II α1 polypeptide; iv) a MHC Class II α2 polypeptide; and v) a MHC Class II β2 polypeptide. Following conjugation, the sc-TMAPP-epitope conjugate comprises, in order from N-terminus to C-terminus: i) a MOD; ii) an epitope (e.g., a peptide antigen that is recognized (e.g., is capable of being recognized and bound) by a TCR); iii) a MHC Class II β1 polypeptide; iv) a MHC Class II α1 polypeptide; v) a MHC Class II α2 polypeptide; and vi) a MHC Class II β2 polypeptide.

Other MOD-containing sc-TMAPPs comprising a chemical conjugation site that can be conjugated to a MOD-epitope peptide (which includes the first MOD as part of the MOD-epitope peptide) include:
A) a sc-TMAPP comprising, from N-terminus to C-terminus, i) a MOD and ii) an epitope that includes:
   1) in order from N-terminus to C-terminus: i) an optional linker that when present is bound to ii) a MHC Class II β1 polypeptide; iii) a MHC Class II α1 polypeptide; iv) a MHC Class II α2 polypeptide; v) a MHC Class II β2 polypeptide; and vi) an immunoglobulin or non-immunoglobulin scaffold polypeptide;
B) in order from N-terminus to C-terminus: i) an optional linker that when present is bound to ii) a MHC Class II β1 polypeptide; iii) a MHC Class II α1 polypeptide; iv) a MHC Class II α2 polypeptide; v) a MHC Class II β2 polypeptide; and vi) an Ig Fc polypeptide;
C) in order from N-terminus to C-terminus: i) an optional linker that when present is bound to ii) a MHC Class II β1 polypeptide; iii) a MHC Class II α1 polypeptide; iv) a MHC Class II α2 polypeptide; v) a MHC Class II β2 polypeptide; and vi) a MOD;
D) in order from N-terminus to C-terminus: i) an optional linker that when present is bound to ii) a MHC Class II β1 polypeptide; iii) a MHC Class II α1 polypeptide; iv) a MHC Class II α2 polypeptide; v) a MHC Class II β2 polypeptide; vi) a MOD; and vii) an immunoglobulin or non-immunoglobulin scaffold polypeptide;
E) in order from N-terminus to C-terminus: i) an optional linker that when present is bound to ii) a MHC Class II β1 polypeptide; iii) a MHC Class II α1 polypeptide; iv) a MHC Class II α2 polypeptide; v) a MHC Class II β2 polypeptide; vi) a MOD; and vii) an Ig Fc polypeptide;
F) in order from N-terminus to C-terminus: i) an optional linker; ii) a MHC Class II β1 polypeptide; iii) a MHC Class II β2 polypeptide; iv) a MHC Class II α1 polypeptide; v) a MHC Class II α2 polypeptide; and vi) a MOD;
G) in order from N-terminus to C-terminus: i) an optional linker; ii) a MHC Class II β1 polypeptide; iii) a MHC Class II β2 polypeptide; iv) a MHC Class II α1 polypeptide; v) a MHC Class II α2 polypeptide; vi) a MOD; and vii) an immunoglobulin or non-immunoglobulin scaffold polypeptide;
H) in order from N-terminus to C-terminus: i) an optional linker; ii) a MHC Class II β1 polypeptide; iii) a MHC Class II β2 polypeptide; iv) a MHC Class II α1 polypeptide; v) a MHC Class II α2 polypeptide; vi) a MOD; and vii) an Ig Fc polypeptide;
I) in order from N-terminus to C-terminus: i) an optional linker that when present is bound to ii) a MHC Class II β1 polypeptide; iii) a MHC Class II β2 polypeptide; iv) a MHC Class II α1 polypeptide; and v) a MHC Class II α2 polypeptide.;
J) in order from N-terminus to C-terminus: i) an optional linker that when present is bound to ii) a MHC Class II β1 polypeptide; iii) a MHC Class II β2 polypeptide; iv) a MHC Class II α1 polypeptide; v) a MHC Class II α2 polypeptide; and vi) an immunoglobulin or non-immunoglobulin scaffold polypeptide;
K) in order from N-terminus to C-terminus: i) an optional linker that when present is bound to ii) a MHC Class II β1 polypeptide; iii) a MHC Class II β2 polypeptide; iv) a MHC Class II α1 polypeptide; v) a MHC Class II α2 polypeptide; and vi) an Ig Fc polypeptide;
L) in order from N-terminus to C-terminus: i) an optional linker that when present is bound to ii) a MHC Class II β1 polypeptide; iii) a MHC Class II β2 polypeptide; iv) a MHC Class II α1 polypeptide; v) a MHC Class II α2 polypeptide; and vi) a second independently selected MOD;
M) in order from N-terminus to C-terminus: i) an optional linker that when present is bound to ii) a MHC Class II β1 polypeptide; iii) a MHC Class II β2 polypeptide; iv) a MHC Class II α1 polypeptide; v) a MHC Class II α2 polypeptide; vi) a second independently selected MOD; and vii) an immunoglobulin or non-immunoglobulin scaffold polypeptide;
N) in order from N-terminus to C-terminus: i) an optional linker that when present is bound to ii) a MHC Class II β1 polypeptide; iii) a MHC Class II β2 polypeptide; iv) a MHC Class II α1 polypeptide; v) a MHC Class II α2 polypeptide; vi) a second independently selected MOD; and vii) an Ig Fc polypeptide; and
O) in order from N-terminus to C-terminus: i) an optional linker that when present is bound to ii) a MHC Class II β1 polypeptide; iii) a MHC Class II α2 polypeptide; iv) an Ig Fc polypeptide; and v) a MHC Class II α2 polypeptide; and vi) a second independently selected MOD.

A MOD-containing sc-TMAPP (e.g., any of the above-mentioned MOD-less m-TMAPPs) having at least one chemical conjugation site (e.g., at the N-terminus, or within the optional linker) may be reacted with an epitope to produce a MOD-containing sc-TMAPP-epitope conjugate having the epitope covalently bound at one or more chemical conjugation sites (e.g., one chemical conjugation site that permits the epitope to be bound and recognized by a TCR). After conjugation, the MOD-containing sc-TMAPP-epitope conjugates may contain additional chemical conjugation sites (e.g., for conjugation of a payload). Accordingly, the specification also provides for and includes such MOD-containing sc-TMAPP epitope conjugates.

Exemplary sc-TMAPPs Comprising One or More MODs

The following are non-limiting examples of sc-TMAPPs comprising one or more independently selected MODs of the present disclosure. It should be noted that any TMAPP to be administered to an individual in need thereof will generally not include a leader sequence or a histidine tag.

1) In some cases, a sc-TMAPP-epitope conjugate comprises, in order from N-terminus to C-terminus: i) an epitope covalently bound (directly or indirectly via a linker) to ii) a HLA β1 polypeptide; iii) a HLA α1 polypeptide; iv) a HLA α2 polypeptide; v) a HLA β2 polypeptide; vi) a MOD (e.g., a variant MOD with reduced affinity for its Co-MOD); and vii) an Ig Fc polypeptide. As one non-limiting example, a sc-TMAPP-epitope conjugate can comprise, in order from N-terminus to C-terminus: i) an epitope covalently bound (directly or indirectly via a linker) to ii) a HLA DRB 1 β1 polypeptide; iii) a HLA DRA α1 polypeptide; iv) a HLA DRA α2 polypeptide; v) a HLA DRB β2 polypeptide; vi) a MOD (e.g., a variant IL-2 polypeptide comprising H16A and F42A substitutions); and vii) an IgG1 Fc polypeptide. In some cases, the epitope is a hemagglutinin epitope (e.g., PKYVKQNTLKLAT; SEQ ID NO:85). In some cases, the HLA DRB 1 β1 polypeptide comprises the following amino acid sequence: DTRPRFLWQHKFECHFFNGTERVRLLERCIYNQEESVRFDSDVGEYRAVTELGRPDAEYWNSQ KDLLEQRRAAVDTYCRHNYGVGESFTVQRRVEP (SEQ ID NO:188). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGALANIA VDKANLEIMTKRSNYTPITN (SEQ ID NO:178). In some cases, the HLA DRB β2 polypeptide comprises the following amino acid sequence: KVTVYPSKTQPLQHHNLLVCSVSGFYPGSIEVRWFRNGQEEKAGVVSTGLIQNGDWTFQTLVM LETVPRSGEVYTCQVEHPSVTSPLTVEWRARS (amino acids 4-98 of SEQ ID NO:183). In some cases, the variant IL-2 polypeptide comprises the following amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:27 with H16A and F42A substitutions), where the H16A and F42A substitutions are underlined. In some cases, the IgG1 Fc polypeptide comprises the following amino acid sequence:

(SEQ ID NO: 181)
DKTHTCPPCPAPELLGGPSVFLFPPKPKDTLMISRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

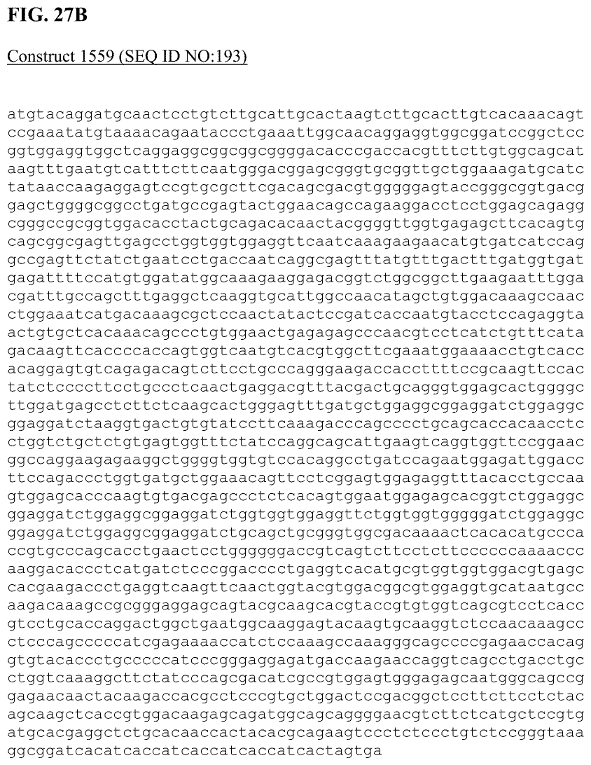

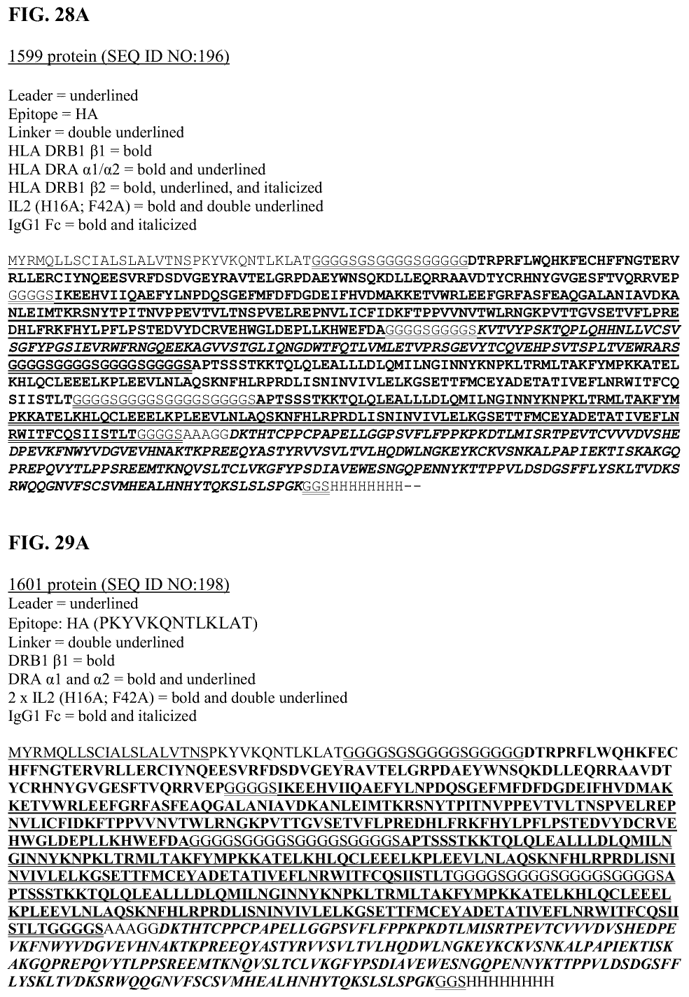

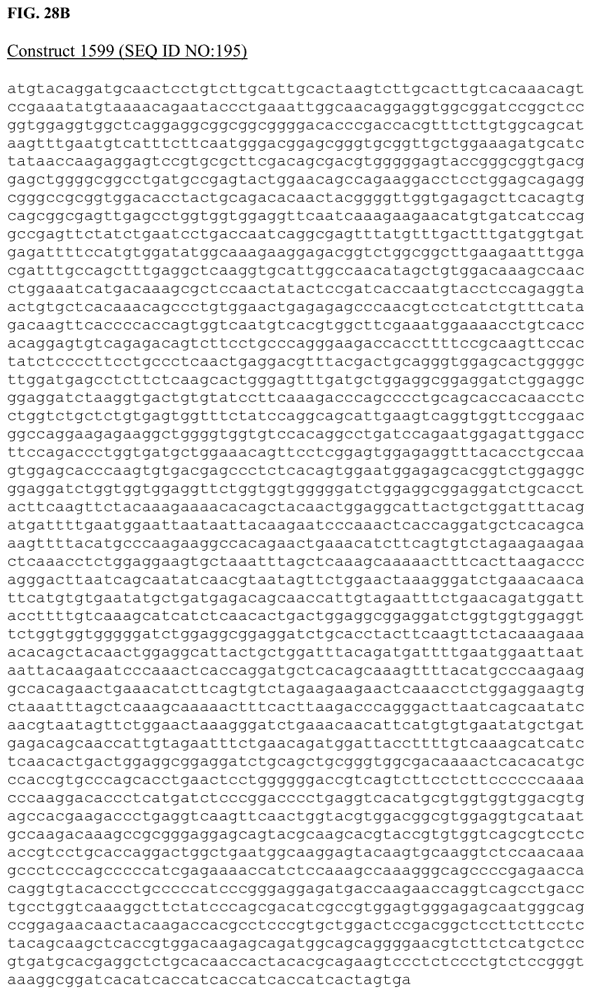

In one such case, the sc-TMAPP may be organized in a fashion similar to amino acids 21 to 981 of protein/polypeptide construct 1599 depicted in FIG. 28A (when the sc-TMAPP has not been conjugated with an epitope there is a chemical conjugation site in the epitope's place, without the leader sequence, and without the C-terminal linker and histidine tag). In some cases, the sc-TMAPP-epitope conjugate does not include a hemagglutinin epitope (e.g., PKYVKQNTLKLAT; SEQ ID NO:85); instead, the epitope is substituted with a different epitope. FIG. 27A is the MOD-less counterpart to the protein in FIG. 28A prepared by expression of the nucleic acid sequence in FIG. 27B.

2) In some cases, a sc-TMAPP-epitope conjugate comprises, in order from N-terminus to C-terminus: i) an epitope covalently bound (directly or indirectly via a linker) to ii) a HLA β1 polypeptide; iii) a HLA α1 polypeptide; iv) a HLA α2 polypeptide; v) a first MOD (e.g., a variant MOD with reduced affinity for its Co-MOD); vi) a second independently selected MOD (e.g., a variant MOD with reduced affinity for its Co-MOD); and vii) an Ig Fc polypeptide. As one non-limiting example, a sc-TMAPP-epitope conjugate can comprise, in order from N-terminus to C-terminus: i) an epitope covalently bound (directly or indirectly via a linker) to ii) a HLA DRB1 β1 polypeptide; iii) a HLA DRA α1 polypeptide; iv) n HLA DRA α2 polypeptide; v) a first MOD (e.g., a variant IL-2 polypeptide comprising H16A and F42A substitutions); vi) a second independently selected MOD (e.g., a variant IL-2 polypeptide comprising H16A and F42A substitutions); and vii) an IgG1 Fc polypeptide. In some cases, the epitope is a hemagglutinin epitope (e.g., PKYVKQNTLKLAT; SEQ ID NO:85). In some cases, the HLA DRB1 β1 polypeptide comprises the following amino acid sequence: DTRPRFLWQHKFECHFFNGTERVRLLERCIYNQEESVRFDSDVGEYRAVTELGRPDAEYWNSQ KDLLEQRRAAVDTYCRHNYGVGESFTVQRRVEP (SEQ ID NO:188). In some cases, the HLA DRA α1 polypeptide comprises the following amino acid sequence: IKEEHVIIQAEFYLNPDQSGEFMFDFDGDEIFHVDMAKKETVWRLEEFGRFASFEAQGALANIA VDKANLEIMTKRSNYTPITN (SEQ ID NO:178). In some cases, the HLA DRA α2 polypeptide comprises the following amino acid sequence: VPPEVTVLTNSPVELREPNVLICFIDKFTPPVVNVTWLRNGKPVTTGVSETVFLPREDHLFRKFH YLPFLPSTEDVYDCRVEHWGLDEPLLKHWEFDA (SEQ ID NO:179). In some cases, the variant IL-2 polypeptide comprises the following amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:27 with H16A and F42A), where the H16A and F42A substitutions are underlined. In some cases, the IgG1 Fc polypeptide comprises the following amino acid sequence:

(SEQ ID NO: 181)
DKTHTCPPCPAPELLGGPSVFLFPPKPKDTLMISRTPEVTCVVVDVSHE
DPEVKFNWYVDGVEVHNAKTKPREEQYASTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

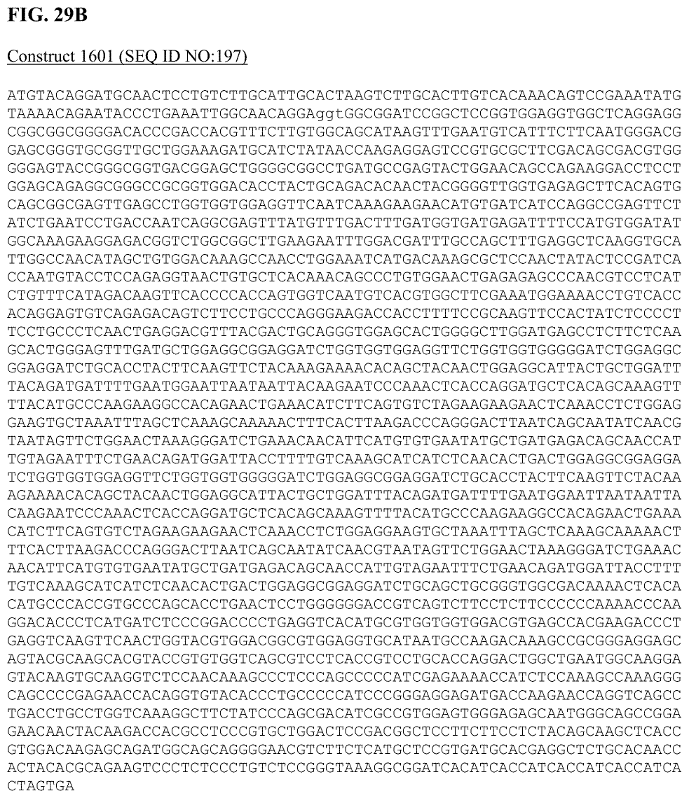

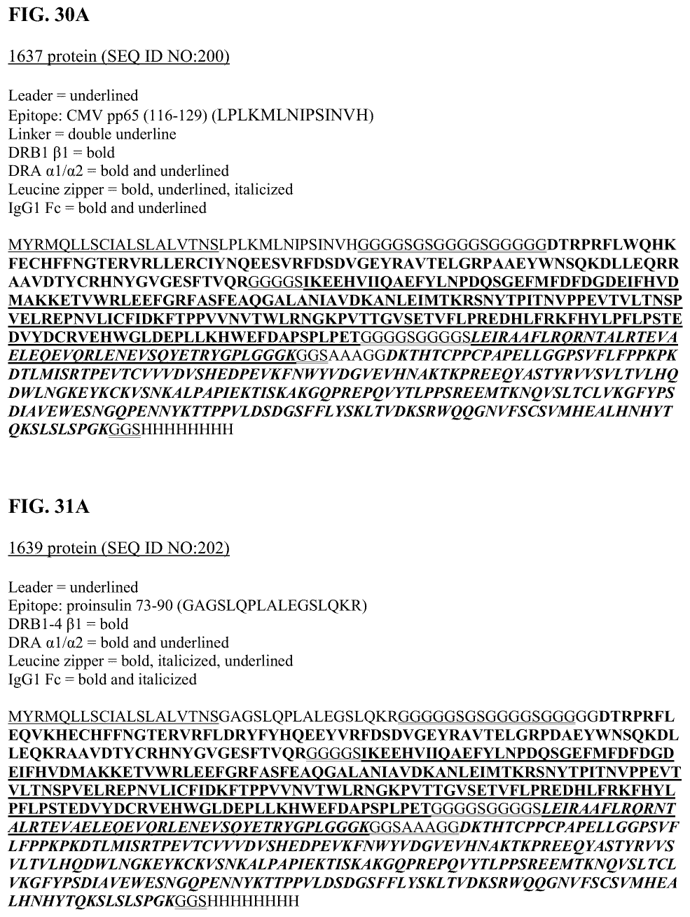

In one such case, the sc-TMAPP may be organized in a fashion similar to amino acids 21 to 876 of protein/polypeptide construct 1601 depicted in FIG. 29A (when the sc-TMAPP has not been conjugated with an epitope there is a chemical conjugation site in the epitope's place, without the leader sequence, and without the C-terminal linker and histidine tag). In some cases, the sc-TMAPP-epitope conjugate does not include a hemagglutinin epitope (e.g., PKYVKQNTLKLAT; SEQ ID NO:85); instead, the epitope is substituted with a different epitope.

Immunomodulatory Polypeptides as TMAPP Domains—MODs

MODs that are suitable for inclusion in a TMAPP having a chemical conjugation site, or its epitope conjugate, as described herein include, but are not limited to, IL-2, CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, lymphotoxin beta receptor, 3/TR6, ILT3, ILT4, and HVEM.

In some cases, the MOD is selected from a 4-1BBL polypeptide, a B7-1 polypeptide; a B7-2 polypeptide, an ICOS-L polypeptide, an OX-40L polypeptide, a CD80 polypeptide, a CD86 polypeptide, a PD-L1 polypeptide, a FasL polypeptide, and a PD-L2 polypeptide. The MOD can comprise only the extracellular portion of a full-length MOD. Thus, for example, the MOD can in some cases exclude one or more of a signal peptide, a transmembrane domain, and an intracellular domain normally found in a naturally-occurring MOD.

In some cases, a MOD suitable for inclusion in a TMAPP having a chemical conjugation site, or its epitope conjugate, comprises all or a portion of (e.g., an extracellular portion of) the amino acid sequence of a naturally-occurring MOD. In other instances, a MOD suitable for inclusion in a TMAPP having a chemical conjugation site, or its epitope conjugate, is a variant MOD that comprises at least one amino acid substitution compared to the amino acid sequence of a naturally-occurring MOD. In some instances, a variant MOD exhibits a binding affinity for a Co-MOD that is lower than the affinity of a corresponding naturally-occurring MOD (e.g., a MOD not comprising the amino acid substitution(s) present in the variant) for the Co-MOD.

Variant MODs with Reduced Affinity

Suitable MODs that exhibit reduced affinity for a Co-MOD can have from 1 amino acid (aa) to 20 aa differences from a wild-type immunomodulatory domain. For example, in some cases, a variant MOD present in a TMAPP having a chemical conjugation site, or its epitope conjugate, may differ in amino acid sequence by, for example, 1 aa, 2 aa, 3 aa, 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa (e.g., from 1 aa to 5 aa, from 5 aa to 10 aa, or from 10 aa to 20 aa) from a corresponding wild-type MOD. As an example, in some cases, a variant MOD present in a TMAPP having a chemical conjugation site, or its epitope conjugate, has and/or includes: 1, 2, 3, 4, 5, 6, 7, 8, 9, 10, 11, 12, 13, 14, 15, 16, 17, 18, 19, or 20 aa (e.g., from 1 to 5, from 2 to 5, from 3 to 5, from 5 to 10, or from 10 to 20) aa substitutions, compared to a corresponding reference (e.g., wild-type) MOD. In some cases, a variant MOD present in a TMAPP having a chemical conjugation site, or its epitope conjugate, includes a single amino acid substitution compared to a corresponding reference (e.g., wild-type MOD). In some cases, a variant MOD present in a TMAPP has and/or includes, relative to a corresponding wild-type reference (e.g., a wild-type MOD): 1 to 2 aa substitutions; 1 to 3 aa substitutions; 1 to 4 aa substitutions; 1 to 5 aa substitutions; 1 to 6 aa substitutions; 1 to 7 aa substitutions; 1 to 8 aa substitutions; 1 to 9 aa substitutions; 1 to 10 aa substitutions; 1 to 11 aa substitutions; 1 to 12 aa substitutions; 1 to 13 aa substitutions; 1 to 14 aa substitutions; 1 to 15 aa substitutions; 1 to 16 aa substitutions; 1 to 17 aa substitutions; 1 to 18 aa substitutions; 1 to 19 aa substitutions, or 1 to 20 aa substitutions.

As discussed above, a variant MOD suitable for inclusion in a TMAPP having a chemical conjugation site, or its epitope conjugate, exhibits reduced affinity for a Co-MOD, compared to the affinity of a corresponding wild-type MOD for the Co-MOD. Exemplary pairs of MOD and Co-MOD include, but are not limited to entries (a) to (r) listed in the following table:

### Exemplary Pairs of MODs and Co-MODs

a) 4-1BBL (MOD) and 4-1BB (Co-MOD);
b) PD-L1 (MOD) and PD1 (Co-MOD);
c) IL-2 (MOD) and IL-2 receptor (Co-MOD);
d) CD80 (MOD) and CD28 (Co-MOD);
e) CD86 (MOD) and CD28 (Co-MOD);
f) OX40L (CD252) (MOD) and OX40 (CD 134) (Co-MOD);
g) Fas ligand (MOD) and Fas (Co-MOD);
h) ICOS-L (MOD) and ICOS (Co-MOD);
i) ICAM (MOD) and LFA-1 (Co-MOD);
j) CD30L (MOD) and CD30 (Co-MOD);
k) CD40 (MOD) and CD40L (Co-MOD);
l) CD83 (MOD) and CD83L (Co-MOD);
m) HVEM (CD270) (MOD) and CD 160 (Co-MOD);
n) JAGI (CD339) (MOD) and Notch (Co-MOD);
o) JAGI (CD339) (MOD) and CD46 (Co-MOD);
p) CD70 (MOD) and CD27 (Co-MOD);
q) CD80 (MOD) and CTLA4 (Co-MOD); and
r) CD86 (MOD) and CTLA4 (Co-MOD)

In some cases, a variant MOD present in a TMAPP having a chemical conjugation site, or its epitope conjugate, has a binding affinity for a Co-MOD that is from 1 nM to 100 μM. For example, in some cases, a variant MOD present in a TMAPP having a chemical conjugation site, or its epitope conjugate, has a binding affinity for a Co-MOD that is from about 1 nM to about 5 nM, from about 5 nM to about 10 nM, from about 10 nM to about 50 nM, from about 50 nM to about 100 nM, from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

Binding affinity between a MOD and its Co-MOD can be determined by bio-layer interferometry (BLI) using purified MOD and purified Co-MOD. Binding affinity between a TMAPP comprising a MOD (e.g., sc-TMAPP-epitope conjugate or m-TMAPP-epitope conjugate) and a Co-MOD can be determined by BLI using purified sc- or m-TMAPP-epitope conjugates and the Co-MOD. BLI methods are well known to those skilled in the art. See, e.g., Lad et al. (2015) J. Biomol. Screen. 20(4):498-507; and Shah and Duncan (2014) J. Vis. Exp. 18:e51383. The specific and relative binding affinities described in this disclosure between a MOD and its Co-MOD, or between a TMAPP comprising a MOD and its cognate Co-MOD, can be determined using the following procedures.

To determine binding affinity between a MOD-containing sc-TMAPP or m-TMAPP and its cognate Co-MOD, a BLI assay can be carried out using an Octet RED 96 (Pal FortéBio) instrument, or a similar instrument, as follows. A TMAPP comprising a MOD (e.g., a sc- or m-TMAPP-epitope conjugate of the present disclosure comprising a variant MOD; or a control sc- or m-TMAPP-epitope conjugate comprising a wild-type MOD) is immobilized onto an insoluble support (a “biosensor”). The immobilized TMAPP comprising a MOD is the “target” Immobilization can be effected by immobilizing a capture antibody onto the insoluble support, where the capture antibody immobilizes the TMAPP comprising a MOD. For example, immobilization can be effected by immobilizing anti-Fc (e.g., anti-human IgG Fc) antibodies onto the insoluble support, where the immobilized anti-Fc antibodies bind to and immobilize a TMAPP comprising a MOD and an IgFc polypeptide. A Co-MOD is applied, at several different concentrations, to the immobilized TMAPP, and the support's response recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the co-immunomodulatory polypeptide to the immobilized polypeptide is conducted at 30° C. As a positive control for binding affinity, an anti-MHC Class I monoclonal antibody can be used. For example, an anti-HLD-DR3 monoclonal antibody such as the 16-23 antibody (Sigma; also referred to as “16.23”; see, e.g., Pious et al. (1985) J. Exp. Med. 162:1193; Mellins et al. (1991) J. Exp. Med. 174:1607; ECACC hybridoma collection 16-23, ECACC 99043001) can be used as a positive control for binding affinity. As another example, a pan-HLA Class II antibody, such as the HKB1 antibody (Immunotools; Holte et al. (1989) Eur. J. Immunol. 19:1221) can be used as a positive control for binding affinity. A standard curve can be generated using serial dilutions of the anti-MHC Class II monoclonal antibody. The co-immunomodulatory polypeptide, or the anti-MHC Class II mAb, is the “analyte.” BLI analyzes the interference pattern of white light reflected from two surfaces: i) the immobilized polypeptide (“target”); and ii) an internal reference layer. A change in the number of molecules (“analyte”; e.g., co-immunomodulatory polypeptide; anti-HLA antibody) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/kₐ) gives rise to the affinity constant K_{D}.

As noted above, determining binding affinity between a MOD (e.g., IL-2 or an IL-2 variant) and its Co-MOD (e.g., IL-2R) also can be determined by BLI. The assay is similar to that described above for the TMAPP comprising a MOD. A BLI assay can be carried out using an Octet RED 96 (Pal FortéBio) instrument, or a similar instrument, as follows. A component MOD of a TMAPP that comprises a MOD (e.g., a variant IL-2 polypeptide of the present disclosure) and a control wild-type MOD (e.g., wild-type IL-2) are each immobilized onto an insoluble support (a “biosensor”). The MOD is the “target” Immobilization can be effected by immobilizing a capture antibody onto the insoluble support, where the capture antibody immobilizes the MOD. For example, if the target is fused to an immuno-affinity tag (e.g., FLAG, human IgG Fc), immobilization can be effected by immobilizing with the appropriate antibody to the immuno-affinity tag (e.g., anti-human IgG Fc) onto the insoluble support, where the immobilized antibodies bind to and immobilize the MOD (where the MOD comprises an IgFc polypeptide). A Co-MOD (or polypeptide) is applied, at several different concentrations, to the immobilized MOD, and the biosensor's response recorded. Alternatively, a Co-MOD (or polypeptide) is immobilized to the biosensor (e.g., for the IL-2 receptor heterotrimer, as a monomeric subunit, heterodimeric subcomplex, or the complete heterotrimer); the MOD is applied, at several different concentrations, to the immobilized coMOD(s), and the biosensor's response is recorded. Assays are conducted in a liquid medium comprising 25 mM HEPES pH 6.8, 5% poly(ethylene glycol) 6000, 50 mM KCl, 0.1% bovine serum albumin, and 0.02% Tween 20 nonionic detergent. Binding of the Co-MOD to the immobilized MOD is conducted at 30° C. BLI analyzes the interference pattern of white light reflected from two surfaces: i) the immobilized polypeptide (“target”); and ii) an internal reference layer. A change in the number of molecules (“analyte”; e.g., Co-MOD) bound to the biosensor tip causes a shift in the interference pattern; this shift in interference pattern can be measured in real time. The two kinetic terms that describe the affinity of the target/analyte interaction are the association constant (kₐ) and dissociation constant (k_{d}). The ratio of these two terms (k_{d}/kₐ) gives rise to the affinity constant K_{D}. Determining the binding affinity of both a wild-type MOD (e.g., IL-2) for its receptor (e.g., IL-2R) and a variant MOD (e.g., an IL-2 variant as disclosed herein) for its Co-MOD (e.g., its receptor) (e.g., IL-2R) thus allows one to determine the relative binding affinity of the variant MOD, as compared to the wild-type MOD, for their Co-MOD. That is, one can determine whether the binding affinity of a variant MOD for its receptor (its cognate Co-MOD) is reduced as compared to the binding affinity of the wild-type MOD for the same Co-MOD, and, if so, the amount (e.g., percentage) of reduction from the binding affinity of the wild-type Co-MOD.

The BLI assay is carried out in a multi-well plate. To run the assay, the plate layout is defined, the assay steps are defined, and biosensors are assigned in Octet Data Acquisition software. The biosensor assembly is hydrated. The hydrated biosensor assembly and the assay plate are equilibrated for 10 minutes on the Octet instrument. Once the data are acquired, the acquired data are loaded into the Octet Data Analysis software. The data are processed in the Processing window by specifying a method for reference subtraction, y-axis alignment, inter-step correction, and Savitzky-Golay filtering. Data are analyzed in the Analysis window by specifying steps to analyze (Association and Dissociation), selecting curve fit model (1:1), fitting method (global), and window of interest (in seconds). The quality of fit is evaluated. K_{D} values for each data trace (analyte concentration) can be averaged if within a 3-fold range. K_{D} error values should be within one order of magnitude of the affinity constant values; R² values should be above 0.95. See, e.g., Abdiche et al. (2008) J. Anal. Biochem. 377:209.

In some cases, the ratio of: i) the binding affinity of a control TMAPP comprising a wild-type MOD to a Co-MOD to ii) the binding affinity of a TMAPP comprising a variant of the wild-type MOD to the Co-MOD, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1. In some cases, the ratio of: i) the binding affinity of a control TMAPP comprising a wild-type MOD to a Co-MOD to ii) the binding affinity of a TMAPP comprising a variant of the wild-type MOD to the Co-MOD, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1.

In some embodiments, an epitope (e.g., a peptide antigen) that will become part of a TMAPP-epitope conjugate binds to a T-cell receptor (TCR) on a T-cell with an affinity of at least 100 μM (e.g., at least 10 μM, at least 1 μM, at least 100 nM, at least 10 nM, or at least 1 nM). In some embodiments, the epitope binds to a TCR on a T-cell with an affinity of from about 10⁻⁴ M to about 5×10⁻⁴ M, from about 5×10⁻⁴ M to about 10⁻⁵ M, from about 10⁻⁵ M to about 5×10⁻⁵ M, from about 5×10⁻⁵ M to about 10⁻⁶ M, from about 10⁻⁶M to about 5×10⁻⁶M, from about 5×10⁻⁶ M to about 10⁻⁷M, from about 10⁻⁷ M to about 5×10⁻⁷ M, from about 5×10⁻⁷ M to about 10⁻⁸M, or from about 10⁻⁸M to about 10⁻⁹ M. Expressed another way, in some embodiments, the epitope, which after conjugation will be present in a TMAPP-epitope conjugate, binds to a TCR on a T-cell with an affinity of from about 1 nM to about 5 nM, from about 5 nM to about 10 nM, from about 10 nM to about 50 nM, from about 50 nM to about 100 nM, from about 0.1 μM to about 0.5 μM, from about 0.5 μM to about 1 μM, from about 1 μM to about 5 μM, from about 5 μM to about 10 μM, from about 10 μM to about 25 μM, from about 25 μM to about 50 μM, from about 50 μM to about 75 μM, or from about 75 μM to about 100 μM.

In some cases, a variant MOD, which may be present in a TMAPP comprising a MOD, has a binding affinity for a Co-MOD that is from about 1 nM to about 100 nM, or from about 100 nM to about 100 μM (e.g., by BLI assay). For example, in some embodiments, a variant MOD present in a TMAPP has a binding affinity for a Co-MOD that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM. In some embodiments, a variant MOD present in a TMAPP has a binding affinity for a Co-MOD that is from about 1 nM to about 5 nM, from about 5 nM to about 10 nM, from about 10 nM to about 50 nM, or from about 50 nM to about 100 nM.

PD-L1 Variants

As one non-limiting example, in some cases, a variant MOD present in a TMAPP having a chemical conjugation site, or its epitope conjugate, is a variant PD-L1 polypeptide. Wild-type PD-L1 binds to PD1.

A wild-type human PD-L1 polypeptide can comprise the following amino acid sequence:

(SEQ ID NO: 13)
MRIFAVFIFM TYWHLLNAFT VTVPKDLYVV EYGSNMTIEC
KFPVEKQLDL AALIVYWEME DKNIIQFVHG EEDLKVQHSS
YRQRARLLKD QLSLGNAALQ ITDVKLQDAG VYRCMISYGG
ADYKRITVKV NAPYNKINQR ILVVDPVTSE HELTCQAEGY
PKAEVIWTSS DHQVLSGKTT TTNSKREEKL FNVTSTLRIN
TTTNEIFYCT FRRLDPEENH TAELVIPGNI LNVSIKICLT
LSPST.

A wild-type human PD-L1 ectodomain can comprise the following amino acid sequence: FT

(SEQ ID NO: 14)
VTVPKDLYVV EYGSNMTIEC KFPVEKQLDL AALIVYWEME
DKNIIQFVHG EEDLKVQHSS YRQRARLLKD QLSLGNAALQ
ITDVKLQDAG VYRCMISYGG ADYKRITVKV NAPYNKINQR
ILVVDPVTSE HELTCQAEGY PKAEVIWTSS DHQVLSGKTT
TTNSKREEKL FNVTSTLRIN TTTNEIFYCT FRRLDPEENH
TAELVIPGNI LNVSIKI.

A wild-type PD-1 polypeptide (NCBI Accession No. NP 005009.2, aas 2-288) can comprise the following amino acid sequence:

(SEQ ID NO: 15)
PGWFLDSPDR PWNPPTFSPA LLVVTEGDNA TFTCSFSNTS
ESFVLNWYRM SPSNQTDKLA AFPEDRSQPG QDCRFRVTQL
PNGRDFHMSV VRARRNDSGT YLCGAISLAP KAQIKESLRA
ELRVTERRAE VPTAHPSPSP RPAGQFQTLV VGVVGGLLGS
LVLLVWVLAV ICSRAARGTI GARRTGQPLK EDPSAVPVFS
VDYGELDFQW REKTPEPPVP CVPEQTEYAT IVFPSGMGTS
SPARRGSADG PRSAQPLRPE DGHCSWPL.

In some cases, a variant PD-L1 polypeptide exhibits reduced binding affinity to PD-1 (e.g., a PD-1 polypeptide comprising the amino acid sequence set forth in SEQ ID NO:15), compared to the binding affinity of a PD-L1 polypeptide comprising the amino acid sequence set forth in SEQ ID NO:13 or SEQ ID NO:14. For example, in some cases, a variant PD-L1 polypeptide of the present disclosure binds PD-1 (e.g., a PD-1 polypeptide comprising the amino acid sequence set forth in SEQ ID NO:15) with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a PD-L1 polypeptide comprising the amino acid sequence set forth in SEQ ID NO:13 or SEQ ID NO:14.

In some cases, a variant PD-L1 polypeptide has a binding affinity to PD-1 that is from 1 nM to 1 mM. In some cases, a variant PD-L1 polypeptide of the present disclosure has a binding affinity to PD-1 that is from about 100 nM to about 100 μM. As another example, in some cases, a variant PD-L1 polypeptide has a binding affinity for PD1 (e.g., a PD1 polypeptide comprising the amino acid sequence set forth in SEQ ID NO:15) that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant PD-L1 polypeptide has a single amino acid substitution compared to the PD-L1 amino acid sequence set forth in SEQ ID NO:13 or SEQ ID NO:14. In some cases, a variant PD-L1 polypeptide has from 2 to 10 amino acid substitutions compared to the PD-L1 amino acid sequence set forth in SEQ ID NO:13 or SEQ ID NO:14. In some cases, a variant PD-L1 polypeptide has 2, 3, 4, 5, 6, 7, 8, 9, or 10 amino acid substitutions compared to the PD-L1 amino acid sequence set forth in SEQ ID NO:13 or SEQ ID NO:14.

A suitable PD-L1 variant includes a polypeptide that comprises an amino acid sequence having at least 90%, at least 95%, at least 98%, at least 99%, or 100% amino acid sequence identity to the following amino acid sequence:

FT VTVPKXLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:14), where X is any amino acid other than Asp. In some cases, X is Ala. In some cases, X is Arg.

A suitable PD-L1 variant includes a polypeptide that comprises an amino acid sequence having at least 90%, at least 95%, at least 98%, at least 99%, or 100% amino acid sequence identity to the following amino acid sequence:

FT VTVPKDLYVV EYGSNMTIEC KFPVEKQLDL AALXVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:14), where X is any amino acid other than Ile. In some cases, X is Asp.

A suitable PD-L1 variant includes a polypeptide that comprises an amino acid sequence having at least 90%, at least 95%, at least 98%, at least 99%, or 100% amino acid sequence identity to the following amino acid sequence:

FT VTVPKDLYVV EYGSNMTIEC KFPVEKQLDL AALIVYWEME DKNIIQFVHG EXDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:14), where X is any amino acid other than Glu. In some cases, X is Arg.

CD80 Variants

In some cases, a variant MOD present in a TMAPP of the present disclosure is a variant CD80 polypeptide. Wild-type CD80 binds to CD28.

A wild-type amino acid sequence of the ectodomain of human CD80 can be as follows:

(SEQ ID NO: 16)
VIHVTK EVKEVATLSC GHNVSVEELA QTRIYWQKEK KMVLTMMSGD
MNIWPEYKNR TIFDITNNLS IVILALRPSD EGTYECVVLK YEKDAFKREH LAEVTLSVKA
DFPTPSISDF EIPTSNIRRI ICSTSGGFPE PHLSWLENGE ELNAINTTVS QDPETELYAV
SSKLDFNMTT NHSFMCLIKY GHLRVNQTFN WNTTKQEHFP DN.

A wild-type CD28 amino acid sequence can be as follows

(SEQ ID NO: 17)
MLRLLLALNL FPSIQVTGNK ILVKQSPMLV AYDNAVNLSC
KYSYNLFSRE FRASLHKGLD SAVEVCVVYG NYSQQLQVYS
KTGFNCDGKL GNESVTFYLQ NLYVNQTDIY FCKIEVMYPP
PYLDNEKSNG TIIHVKGKHL CPSPLFPGPS KPFWVLVVVG
GVLACYSLLV TVAFIIFWVR SKRSRLLHSD YMNMTPRRPG
PTRKHYQPYA PPRDFAAYRS.

A wild-type CD28 amino acid sequence can be as follows:

(SEQ ID NO: 18)
MLRLLLALNL FPSIQVTGNK ILVKQSPMLV AYDNAVNLSW
KHLCPSPLFP GPSKPFWVLV VVGGVLACYS LLVTVAFIIF
WVRSKRSRLL HSDYMNMTPR RPGPTRKHYQ PYAPPRDFAA YRS

A wild-type CD28 amino acid sequence can be as follows:

(SEQ ID NO: 19)
MLRLLLALNL FPSIQVTGKH LCPSPLFPGP SKPFWVLVVV
GGVLACYSLL VTVAFIIFWV RSKRSRLLHS DYMNMTPRRP
GPTRKHYQPY APPRDFAAYR S.

In some cases, a variant CD80 polypeptide exhibits reduced binding affinity to CD28, compared to the binding affinity of a CD80 polypeptide comprising the amino acid sequence set forth in SEQ ID NO:16 for CD28. For example, in some cases, a variant CD80 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD80 polypeptide comprising the amino acid sequence set forth in SEQ ID NO:16 for CD28 (e.g., a CD28 polypeptide comprising the amino acid sequence set forth in one of SEQ ID NOs:17, 18, pr 19).

In some cases, a variant CD80 polypeptide has a binding affinity to CD28 that is from about 100 nM to about 100 μM. As another example, in some cases, a variant CD80 polypeptide of the present disclosure has a binding affinity for CD28 (e.g., a CD28 polypeptide comprising the amino acid sequence set forth in SEQ ID NO:17, SEQ ID NO:18, or SEQ ID NO:19) that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD80 polypeptide has a single amino acid substitution compared to the CD80 amino acid sequence set forth in SEQ ID NO:16. In some cases, a variant CD80 polypeptide has from 2 to 10 amino acid substitutions compared to the CD80 amino acid sequence set forth in SEQ ID NO:4. In some cases, a variant CD80 polypeptide has 2, 3, 4, 5, 6, 7, 8, 9, or 10 amino acid substitutions compared to the CD80 amino acid sequence set forth in SEQ ID NO:16.

Suitable CD80 variants include a polypeptide that comprises an amino acid sequence having at least 90%, at least 95%, at least 98%, at least 99%, or 100%, amino acid sequence identity to any one of the following amino acid sequences:

VIHVTK EVKEVATLSC GHXVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Asn. In some cases, X is Ala;

VIHVTK EVKEVATLSC GHNVSVEELA QTRIYWQKEK KMVLTMMSGD MNIWPEYKNR TIFDITXNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Asn. In some cases, X is Ala;

VIHVTK EVKEVATLSC GHNVSVEELA QTRIYWQKEK KMVLTMMSGD MNIWPEYKNR TIFDITNNLS XVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Ile. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLX YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Lys. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS XDPETELYAV SSKLDFNMTT NHSFMCLIKY GHLRVNQTFN WNTTKQEHFP DN (SEQ ID NO:16), where X is any amino acid other than Gln. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QXPETELYAV SSKLDFNMTT NHSFMCLIKY GHLRVNQTFN WNTTKQEHFP DN (SEQ ID NO:16), where X is any amino acid other than Asp. In some cases, X is Ala;

VIHVTK EVKEVATLSC GHNVSVEEXA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Leu. In some cases, X is Ala;

VIHVTK EVKEVATLSC GHNVSVEELA QTRIXWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Tyr. In some cases, X is Ala;

VIHVTK EVKEVATLSC GHNVSVEELA QTRIYWXKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Gln. In some cases, X is Ala;

VIHVTK EVKEVATLSC GHNVSVEELA QTRIYWQKEK KXVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Met. In some cases, X is Ala;

VIHVTK EVKEVATLSC GHNVSVEELA QTRIYWQKEK KMXLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Val. In some cases, X is Ala;

VIHVTK EVKEVATLSC GHNVSVEELA QTRIYWQKEK KMVLTMMSGD MNXWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Ile. In some cases, X is Ala;

VIHVTK EVKEVATLSC GHNVSVEELA QTRIYWQKEK KMVLTMMSGD MNIWPEXKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Tyr. In some cases, X is Ala;

VIHVTK EVKEVATLSC GHNVSVEELA QTRIYWQKEK KMVLTMMSGD MNIWPEYKNR TIFXITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Asp. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DXPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Phe. In some cases, X is Ala;

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVX QDPETELYAV SSKLDFNMTT NHSFMCLIKY GHLRVNQTFN WNTTKQEHFP DN (SEQ ID NO:16), where X is any amino acid other than Ser. In some cases, X is Ala; and

VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTXSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:16), where X is any amino acid other than Pro. In some cases, X is Ala.

CD86 Variants

In some cases, a variant MOD present in a TMAPP having a chemical conjugation site, or its epitope conjugate, is a variant CD86 polypeptide. Wild-type CD86 binds to CD28.

The amino acid sequence of the full ectodomain of a wild-type human CD86 can be as follows:

(SEQ ID NO: 20)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVLANFSQPEIVPISNITENVYINLTCSSIHGYPEPKKMS
VLLRTKNSTIEYDGIMQKSQDNVTELYDVSISLSVSFPDVTSNMTIFCI
LETDKTRLLSSPFSIELEDPQPPPDHIP.

The amino acid sequence of the IgV domain of a wild-type human CD86 can be as follows:

(SEQ ID NO: 21)
APLKIQAYFNETADLPCQFANSQNQSLSELVVFWQDQENLVLNEVYLGK
EKFDSVHSKYMNRTSFDSDSWTLRLHNLQIKDKGLYQCIIHHKKPTGMI
RIHQMNSELSVL.

In some cases, a variant CD86 polypeptide exhibits reduced binding affinity to CD28, compared to the binding affinity of a CD86 polypeptide comprising the amino acid sequence set forth in SEQ ID NO:20 or SEQ ID NO:21 for CD28. For example, in some cases, a variant CD86 polypeptide binds CD28 with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a CD86 polypeptide comprising the amino acid sequence set forth in SEQ ID NO:20 or SEQ ID NO:21 for CD28 (e.g., a CD28 polypeptide comprising the amino acid sequence set forth in one of SEQ ID NOs:17, 18, or 19).

In some cases, a variant CD86 polypeptide has a binding affinity to CD28 that is from about 100 nM to about 100 μM. As another example, in some cases, a variant CD86 polypeptide of the present disclosure has a binding affinity for CD28 (e.g., a CD28 polypeptide comprising the amino acid sequence set forth in one of SEQ ID NOs:17, 18, or 19) that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant CD86 polypeptide has a single amino acid substitution compared to the CD86 amino acid sequence set forth in SEQ ID NO:20. In some cases, a variant CD86 polypeptide has from 2 to 10 amino acid substitutions compared to the CD86 amino acid sequence set forth in SEQ ID NO:20. In some cases, a variant CD86 polypeptide has 2, 3, 4, 5, 6, 7, 8, 9, or 10 amino acid substitutions compared to the CD86 amino acid sequence set forth in SEQ ID NO:20.

In some cases, a variant CD86 polypeptide has a single amino acid substitution compared to the CD86 amino acid sequence set forth in SEQ ID NO:21. In some cases, a variant CD86 polypeptide has from 2 to 10 (2, 3, 4, 5, 6, 7, 8, 9, or 10) amino acid substitutions compared to the CD86 amino acid sequence set forth in SEQ ID NO:21. In some cases, a variant CD86 polypeptide has 2, 3, 4, 5, 6, 7, 8, 9, or 10 amino acid substitutions compared to the CD86 amino acid sequence set forth in SEQ ID NO:21.

Suitable CD86 variants include a polypeptide that comprises an amino acid sequence having at least 90%, at least 95%, at least 98%, at least 99%, or 100%, amino acid sequence identity to any one of the following amino acid sequences:

APLKIQAYFNETADLPCQFANSQNQSLSELVVFWQDQENLVLNEVYLGKEKFDSVHS KYMXRTSFDSDSWTLRLHNLQIKDKGLYQCIIHHKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than Asn. In some cases, X is Ala;

APLKIQAYFNETADLPCQFANSQNQSLSELVVFWQDQENLVLNEVYLGKEKFDSVHS KYMNRTSFXSDSWTLRLHNLQIKDKGLYQCIIHHKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than Asp. In some cases, X is Ala;

APLKIQAYFNETADLPCQFANSQNQSLSELVVFWQDQENLVLNEVYLGKEKFDSVHS KYMNRTSFDSDSXTLRLHNLQIKDKGLYQCIIHHKKPTGMIRIHQMNSELSVLANFSQPEIVPISN ITENVYINLTCSSIHGYPEPKKMSVLLRTKNSTIEYDGIMQKSQDNVTELYDVSISLSVSFPDVTS NMTIFCILETDKTRLLSSPFSIELEDPQPPPDHIP (SEQ ID NO:20), where X is any amino acid other than Trp. In some cases, X is Ala;

APLKIQAYFNETADLPCQFANSQNQSLSELVVFWQDQENLVLNEVYLGKEKFDSVHS KYMNRTSFDSDSWTLRLHNLQIKDKGLYQCIIHXKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than His. In some cases, X is Ala;

APLKIQAYFNETADLPCQFANSQNQSLSELVVFWQDQENLVLNEVYLGKEKFDSVHS KYMXRTSFDSDSWTLRLHNLQIKDKGLYQCIIHHKKPTGMIRIHQMNSELSVL (SEQ ID NO:20), where X is any amino acid other than Asn. In some cases, X is Ala;

APLKIQAYFNETADLPCQFANSQNQSLSELVVFWQDQENLVLNEVYLGKEKFDSVHS KYMNRTSFXSDSWTLRLHNLQIKDKGLYQCIIHHKKPTGMIRIHQMNSELSVL (SEQ ID NO:20), where X is any amino acid other than Asp. In some cases, X is Ala;

APLKIQAYFNETADLPCQFANSQNQSLSELVVFWQDQENLVLNEVYLGKEKFDSVHS KYMNRTSFDSDSXTLRLHNLQIKDKGLYQCIIHHKKPTGMIRIHQMNSELSVL (SEQ ID NO:20), where X is any amino acid other than Trp. In some cases, X is Ala;

APLKIQAYFNETADLPCQFANSQNQSLSELVVFWQDQENLVLNEVYLGKEKFDSVHS KYMNRTSFDSDSWTLRLHNLQIKDKGLYQCIIHXKKPTGMIRIHQMNSELSVL (SEQ ID NO:20), where X is any amino acid other than His. In some cases, X is Ala;

APLKIQAYFNETADLPCQFANSQNQSLSELVVFWQDQENLXLNEVYLGKEKFDSVHS KYMNRTSFDSDSWTLRLHNLQIKDKGLYQCIIHHKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than Val. In some cases, X is Ala;

APLKIQAYFNETADLPCQFANSQNQSLSELVVFWQDQENLXLNEVYLGKEKFDSVHS KYMNRTSFDSDSWTLRLHNLQIKDKGLYQCIIHHKKPTGMIRIHQMNSELSVL (SEQ ID NO:20), where X is any amino acid other than Val. In some cases, X is Ala;

APLKIQAYFNETADLPCQFANSQNQSLSELVVFWXDQENLVLNEVYLGKEKFDSVHS KYMNRTSFDSDSWTLRLHNLQIKDKGLYQCIIHHKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than Gln. In some cases, X is Ala;

APLKIQAYFNETADLPCQFANSQNQSLSELVVFWXDQENLVLNEVYLGKEKFDSVHS KYMNRTSFDSDSWTLRLHNLQIKDKGLYQCIIHHKKPTGMIRIHQMNSELSVL (SEQ ID NO:20), where X is any amino acid other than Gln. In some cases, X is Ala;

APLKIQAYFNETADLPCQFANSQNQSLSELVVXWQDQENLVLNEVYLGKEKFDSVHS KYMNRTSFDSDSWTLRLHNLQIKDKGLYQCIIHHKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than Phe. In some cases, X is Ala;

APLKIQAYFNETADLPCQFANSQNQSLSELVVXWQDQENLVLNEVYLGKEKFDSVHS KYMNRTSFDSDSWTLRLHNLQIKDKGLYQCIIHHKKPTGMIRIHQMNSELSVL (SEQ ID NO:20), where X is any amino acid other than Phe. In some cases, X is Ala;

APLKIQAYFNETADLPCQFANSQNQSLSELVVFWQDQENLVLNEVYLGKEKFDSVHS KYMNRTSFDSDSWTXRLHNLQIKDKGLYQCIIHHKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than Leu. In some cases, X is Ala;

APLKIQAYFNETADLPCQFANSQNQSLSELVVFWQDQENLVLNEVYLGKEKFDSVHS KYMNRTSFDSDSWTXRLHNLQIKDKGLYQCIIHHKKPTGMIRIHQMNSELSVL (SEQ ID NO:20), where X is any amino acid other than Leu. In some cases, X is Ala;

APLKIQAYFNETADLPCQFANSQNQSLSELVVFWQDQENLVLNEVYLGKEKFDSVHS KXMNRTSFDSDSWTLRLHNLQIKDKGLYQCIIHHKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where X is any amino acid other than Tyr. In some cases, X is Ala;

APLKIQAYFNETADLPCQFANSQNQSLSELVVFWQDQENLVLNEVYLGKEKFDSVHS KXMNRTSFDSDSWTLRLHNLQIKDKGLYQCIIHHKKPTGMIRIHQMNSELSVL (SEQ ID NO:20), where X is any amino acid other than Tyr. In some cases, X is Ala;

APLKIQAYFNETADLPCQFANSQNQSLSELVVFWQDQENLVLNEVYLGKEKFDSVHS KXMXRTSFDSDSWTLRLHNLQIKDKGLYQCIIHXKKPTGMIRIHQMNSELSVLANFSQPEIVPIS NITENVYINLTCSSIHGYPEPKKMSVLLRTKNSTIEYDGIMQKSQDNVTELYDVSISLSVSFPDVT SNMTIFCILETDKTRLLSSPFSIELEDPQPPPDHIP (SEQ ID NO:20), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHS KYMXRTSFDSDSWTLRLHNLQIKDKGLYQCIIHXKKPTGMIRIHQMNSELSVL (SEQ ID NO:20), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHS KYMNRTSFX₁SDSWTLRLHNLQIKDKGLYQCIIHX₂KKPTGMIRIHQMNSELSVLANFSQPEIVPI SNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:20), where X₁ is any amino acid other than Asp, and X₂ is any amino acid other than His. In some cases, X₁ is Ala and X₂ is Ala;

APLKIQAYFNETADLPCQFANSQNQSLSELVVFWQDQENLVLNEVYLGKEKFDSVHS KYMNRTSFX₁SDSWTLRLHNLQIKDKGLYQCIIHX₂KKPTGMIRIHQMNSELSVL (SEQ ID NO:20), where the first X is any amino acid other than Asn and the second X is any amino acid other than His. In some cases, the first and the second X are both Ala;

APLKIQAYFNETADLPCQFANSQNQSLSELVVFWQDQENLVLNEVYLGKEKFDSVHS KYMX₁RTSFX₂SDSWTLRLHNLQIKDKGLYQCIIHX₃KKPTGMIRIHQMNSELSVLANFSQPEIVPI SNITENVYINLTCSSIHGYPEPKKMSVLLRTKNSTIEYDGIMQKSQDNVTELYDVSISLSVSFPDV TSNMTIFCILETDKTRLLSSPFSIELEDPQPPPDHIP (SEQ ID NO:20), where X₁ is any amino acid other than Asn, X₂ is any amino acid other than Asp, and X₃ is any amino acid other than His. In some cases, X₁ is Ala, X₂ is Ala, and X₃ is Ala; and

APLKIQAYFNETADLPCQFANSQNQSLSELVVFWQDQENLVLNEVYLGKEKFDSVHS KYMX₁RTSFX₂SDSWTLRLHNLQIKDKGLYQCIIHX₃KKPTGMIRIHQMNSELSVL (SEQ ID NO:20), where X₁ is any amino acid other than Asn, X₂ is any amino acid other than Asp, and X₃ is any amino acid other than His. In some cases, X₁ is Ala, X₂ is Ala, and X₃ is Ala.

4-1BBL Variants

In some cases, a variant MOD present in a TMAPP having a chemical conjugation site, or its epitope conjugate, is a variant 4-1BBL polypeptide. Wild-type 4-1BBL binds to 4-1BB (CD137).

A wild-type 4-1BBL amino acid sequence can be as follows:

(SEQ ID NO: 22)
MEYASDASLD PEAPWPPAPR ARACRVLPWA LVAGLLLLLL
LAAACAVFLA CPWAVSGARA SPGSAASPRL REGPELSPDD
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE.

In some cases, a variant 4-1BBL polypeptide is a variant of the tumor necrosis factor (TNF) homology domain (THD) of human 4-1BBL.

A wild-type amino acid sequence of the THD of human 4-1BBL can be, e.g., one of SEQ ID NOS:23-25, as follows:

(SEQ ID NO: 23)
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
(SEQ ID NO: 24)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE;
or
(SEQ ID NO: 25)
D PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPA.

A wild-type 4-1BB amino acid sequence can be as follows:

(SEQ ID NO: 26)
MGNSCYNIVA TLLLVLNFER TRSLQDPCSN CPAGTFCDNN
RNQICSPCPP NSFSSAGGQR TCDICRQCKG VFRTRKECSS
TSNAECDCTP GFHCLGAGCS MCEQDCKQGQ ELTKKGCKDC
CFGTFNDQKR GICRPWTNCS LDGKSVLVNG TKERDVVCGP
SPADLSPGAS SVTPPAPARE PGHSPQIISF FLALTSTALL
FLLFFLTLRF SVVKRGRKKL LYIFKQPFMR PVQTTQEEDG
CSCRFPEEEE GGCEL.

In some cases, a variant 4-1BBL polypeptide exhibits reduced binding affinity to 4-1BB, compared to the binding affinity of a 4-1BBL polypeptide comprising the amino acid sequence set forth in one of SEQ ID NOs:22-25. For example, in some cases, a variant 4-1BBL polypeptide of the present disclosure binds 4-1BB with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of a 4-1BBL polypeptide comprising the amino acid sequence set forth in one of SEQ ID NOs:22-25 for a 4-1BB polypeptide (e.g., a 4-1BB polypeptide comprising the amino acid sequence set forth in SEQ ID NO:26), when assayed under the same conditions.

In some cases, a variant 4-1BBL polypeptide has a binding affinity to 4-1BB that is from about 100 nM to about 100 μM. As another example, in some cases, a variant 4-1BBL polypeptide has a binding affinity for 4-1BB (e.g., a 4-1BB polypeptide comprising the amino acid sequence set forth in SEQ ID NO:26) that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant 4-1BBL polypeptide has a single amino acid substitution compared to the 4-1BBL amino acid sequence set forth in one of SEQ ID NOs:22-25. In some cases, a variant 4-1BBL polypeptide has from 2 to 10 (2, 3, 4, 5, 6, 7, 8, 9, or 10) amino acid substitutions compared to the 4-1BBL amino acid sequence set forth in one of SEQ ID NOs:22-25. In some cases, a variant 4-1BBL polypeptide has 2, 3, 4, 5, 6, 7, 8, 9, or 10 amino acid substitutions compared to the 4-1BBL amino acid sequence set forth in one of SEQ ID NOs:22-25.

Suitable 4-1BBL variants include a polypeptide that comprises an amino acid sequence having at least 90%, at least 95%, at least 98%, at least 99%, or 100%, amino acid sequence identity to any one of the following amino acid sequences:

PAGLLDLRQG MFAQLVAQNV LLIDGPLSWY SDPGLAGVSL TGGLSYXEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Lys. In some cases, X is Ala;

PAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWXLTQ GATVLGLFRV TPEIPAGLPS PRSE (SEQ ID NO:23), where X is any amino acid other than Gln. In some cases, X is Ala;

PAGLLDLRQG XFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Met. In some cases, X is Ala;

PAGLLDLRQG MXAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Phe. In some cases, X is Ala;

PAGLLDLRQG MFAXLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gln. In some cases, X is Ala;

PAGLLDLRQG MFAQXVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLXAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Val. In some cases, X is Ala;

PAGLLDLRQG MFAQLVAXNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gln. In some cases, X is Ala;

PAGLLDLRQG MFAQLVAQXV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Asn. In some cases, X is Ala;

PAGLLDLRQG MFAQLVAQNX LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Val. In some cases, X is Ala;

PAGLLDLRQG MFAQLVAQNV XLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LXIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLXDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Ile. In some cases, X is Ala;

PAGLLDLRQG MFAQLVAQNV LLIXGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Asp. In some cases, X is Ala;

PAGLLDLRQG MFAQLVAQNV LLIDXPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGXLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Pro. In some cases, X is Ala;

PAGLLDLRQG MFAQLVAQNV LLIGGPXSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLXWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSXY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Trp. In some cases, X is Ala;

PAGLLDLRQG MFAQLVAQNV LLIGGPLSWX SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Tyr. In some cases, X is Ala;

PAGLLDLRQG MFAQLVAQNV LLIGGPLSWY XDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSWY SXPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23/), where X is any amino acid other than Asp. In some cases, X is Ala;

PAGLLDLRQG MFAQLVAQNV LLIGGPLSWY SDXGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Pro. In some cases, X is Ala;

PAGLLDLRQG MFAQLVAQNV LLIGGPLSWY SDPXLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGXAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAXVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGLAGXSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Val. In some cases, X is Ala;

PAGLLDLRQG MFAQLVAQNV LLIGGPLSWY SDPGLAGVXL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSWY SDPGLAGVSX TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL XGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Thr. In some cases, X is Ala;

PAGLLDLRQG MFAQLVAQNV LLIGGPLSWY SDPGLAGVSL TXGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGLAGVSL TGXLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGLAGVSL TGGXSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL TGGLXYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSWY SDPGLAGVSL TGGLSXKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Tyr. In some cases, X is Ala;

PAGLLDLRQG MFAQLVAQNV LLIGGPLSWY SDPGLAGVSL TGGLSYKXDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Glu. In some cases, X is Ala;

PAGLLDLRQG MFAQLVAQNV LLIGGPLSWY SDPGLAGVSL TGGLSYKEXT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Asp. In some cases, X is Ala;

PAGLLDLRQG MFAQLVAQNV LLIGGPLSWY SDPGLAGVSL TGGLSYKEDX KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Thr. In some cases, X is Ala;

PAGLLDLRQG MFAQLVAQNV LLIGGPLSWY SDPGLAGVSL TGGLSYKEDT XELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Lys. In some cases, X is Ala;

PAGLLDLRQG MFAQLVAQNV LLIGGPLSWY SDPGLAGVSL TGGLSYKEDT KXLVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Glu. In some cases, X is Ala;

PAGLLDLRQG MFAQLVAQNV LLIGGPLSWY SDPGLAGVSL TGGLSYKEDT KELVVAKAGV YYVXFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFXQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFFXLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQXELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLXLR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLEXR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELX RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR XVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RXVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVXAGEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVVAXEGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGXGSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEXSGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGXGS VSLALHLQPL RSAAGAAALA LTVDLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVXLPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Asp. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDXPPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLXPASS EARNSAFGFQ GRLLHLSAGQ RLGVHLHTEA RARHAWQLTQ GATVLGLFRV TPEIPAGLPS PRSE (SEQ ID NO:23), where X is any amino acid other than Pro. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPAXS EARNSAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASX EARNSAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS XARNSAFGFQ GRLLHLSAGQ RLGVHLHTEA RARHAWQLTQ GATVLGLFRV TPEIPAGLPS PRSE (SEQ ID NO:23), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EAXNSAFGFQ GRLLHLSAGQ RLGVHLHTEA RARHAWQLTQ GATVLGLFRV TPEIPAGLPS PRSE (SEQ ID NO:23), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARXSAFGFQ GRLLHLSAGQ RLGVHLHTEA RARHAWQLTQ GATVLGLFRV TPEIPAGLPS PRSE (SEQ ID NO:23), where X is any amino acid other than Asn. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNXAFGFQ GRLLHLSAGQ RLGVHLHTEA RARHAWQLTQ GATVLGLFRV TPEIPAGLPS PRSE (SEQ ID NO:23), where X is any amino acid other than Ser. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAXGFQ GRLLHLSAGQ RLGVHLHTEA RARHAWQLTQ GATVLGLFRV TPEIPAGLPS PRSE (SEQ ID NO:23), where X is any amino acid other than Phe. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGX RLGVHLHTEA RARHAWQLTQ GATVLGLFRV TPEIPAGLPS PRSE (SEQ ID NO:23), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ XLGVHLHTEA RARHAWQLTQ GATVLGLFRV TPEIPAGLPS PRSE (SEQ ID NO:23), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RXGVHLHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLXVHLHTEA RARHAWQLTQ GATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGXHLHTEA RARHAWQLTQ GATVLGLFRV TPEIPAGLPS PRSE (SEQ ID NO:23), where X is any amino acid other than Val. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVXLHTEA RARHAWQLTQ GATVLGLFRV TPEIPAGLPS PRSE (SEQ ID NO:23), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHXHTEA RARHAWQLTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLXTEA RARHAWQLTQ GATVLGLFRV TPEIPAGLPS PRSE (SEQ ID NO:23), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHXEA RARHAWQLTQ GATVLGLFRV TPEIPAGLPS PRSE (SEQ ID NO:23), where X is any amino acid other than Thr. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTXA RARHAWQLTQ GATVLGLFRV TPEIPAGLPS PRSE (SEQ ID NO:23), where X is any amino acid other than Glu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA XARHAWQLTQ GATVLGLFRV TPEIPAGLPS PRSE (SEQ ID NO:23), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RAXHAWQLTQ GATVLGLFRV TPEIPAGLPS PRSE (SEQ ID NO:23), where X is any amino acid other than Arg. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARXAWQLTQ GATVLGLFRV TPEIPAGLPS PRSE (SEQ ID NO:23), where X is any amino acid other than His. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAXQLTQ GATVLGLFRV TPEIPAGLPS PRSE (SEQ ID NO:23), where X is any amino acid other than Trp. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQXTQ GATVLGLFRV TPEIPAGLPS PRSE (SEQ ID NO:23), where X is any amino acid other than Leu. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLXQ GATVLGLFRV TPEIPAGLPS PRSE (SEQ ID NO:23), where X is any amino acid other than Thr. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTX GATVLGLFRV TPEIPAGLPS PRSE (SEQ ID NO:23), where X is any amino acid other than Gln. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ XATVLGLFRV TPEIPAGLPS PRSE (SEQ ID NO:23), where X is any amino acid other than Gly. In some cases, X is Ala;

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GAXVLGLFRV TPEIPAGLPS PRSE (SEQ ID NO:23), where X is any amino acid other than Thr. In some cases, X is Ala; and

PAGLLDLRQG MFAQLVAQNV LLIGGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATXLGLFRV TPEIPAGLPS PRSE (SEQ ID NO:23), where X is any amino acid other than Val. In some cases, X is Ala.

IL-2 Variants

In some cases, a variant MOD present in a TMAPP having a chemical conjugation site, or its epitope conjugate, is a variant IL-2 polypeptide. Wild-type IL-2 binds to an IL-2 receptor (IL-2R).

A wild-type IL-2 amino acid sequence can be as follows:

(SEQ ID NO: 27)
APTSSSTKKT QLQLEHLLLD LQMILNGINN YKNPKLTRML
TFKFYMPKKA TELKHLQCLEEELKPLEEVL NLAQSKNFHL
RPRDLISNIN VIVLELKGSE TTFMCEYADE
TATIVEFLNRWITFCQSIIS TLT.

Wild-type IL2 binds to an IL2 receptor (IL2R) on the surface of a cell. An IL2 receptor is in some cases a heterotrimeric polypeptide comprising an alpha chain (IL-2Rα; also referred to as CD25), a beta chain (IL-2Rβ; also referred to as CD122) and a gamma chain (IL-2Rγ; also referred to as CD132). Amino acid sequences of human IL-2Rα, IL2Rβ, and IL-2Rγ can be as follows.

Human IL-2Rα:
(SEQ ID NO: 28)
ELCDDDPPE IPHATFKAMA YKEGTMLNCE CKRGFRRIKS
GSLYMLCTGN SSHSSWDNQC QCTSSATRNT TKQVTPQPEE
QKERKTTEMQ SPMQPVDQAS LPGHCREPPP WENEATERIY
HFVVGQMVYY QCVQGYRALH RGPAESVCKM THGKTRWTQP
QLICTGEMET SQFPGEEKPQ ASPEGRPESE TSCLVTTTDF
QIQTEMAATM ETSIFTTEYQ VAVAGCVFLL ISVLLLSGLT
WQRRQRKSRR TI.
Human IL-2Rβ:
(SEQ ID NO: 29)
VNG TSQFTCFYNS RANISCVWSQ DGALQDTSCQ VHAWPDRRRW
NQTCELLPVS QASWACNLIL GAPDSQKLTT VDIVTLRVLC
REGVRWRVMA IQDFKPFENL RLMAPISLQV VHVETHRCNI
SWEISQASHY FERHLEFEAR TLSPGHTWEE APLLTLKQKQ
EWICLETLTP DTQYEFQVRV KPLQGEFTTW SPWSQPLAFR
TKPAALGKDT IPWLGHLLVG LSGAFGFIIL VYLLINCRNT
GPWLKKVLKC NTPDPSKFFS QLSSEHGGDV QKWLSSPFPS
SSFSPGGLAP EISPLEVLER DKVTQLLLQQ DKVPEPASLS
SNHSLTSCFT NQGYFFFHLP DALEIEACQV YFTYDPYSEE
DPDEGVAGAP TGSSPQPLQP LSGEDDAYCT FPSRDDLLLF
SPSLLGGPSP PSTAPGGSGA GEERMPPSLQ ERVPRDWDPQ
PLGPPTPGVP DLVDFQPPPE LVLREAGEEV PDAGPREGVS
FPWSRPPGQG EFRALNARLP LNTDAYLSLQ ELQGQDPTHL V.
Human IL-2Rγ:
(SEQ ID NO: 30)
LNTTILTP NGNEDTTADF FLTTMPTDSL SVSTLPLPEV
QCFVFNVEYM NCTWNSSSEP QPTNLTLHYW YKNSDNDKVQ
KCSHYLFSEE ITSGCQLQKK EIHLYQTFVV QLQDPREPRR
QATQMLKLQN LVIPWAPENL TLHKLSESQL ELNWNNRFLN
HCLEHLVQYR TDWDHSWTEQ SVDYRHKFSL PSVDGQKRYT
FRVRSRFNPL CGSAQHWSEW SHPIHWGSNT SKENPFLFAL
EAVVISVGSM GLIISLLCVY FWLERTMPRI PTLKNLEDLV
TEYHGNFSAW SGVSKGLAES LQPDYSERLC LVSEIPPKGG
ALGEGPGASP CNQHSPYWAP PCYTLKPET.

In some cases, where a sc- or m-TMAPP of the present disclosure comprises a variant IL-2 polypeptide, a Co-MOD is an IL-2R comprising polypeptides comprising the amino acid sequences of SEQ ID NOs:28 29, and 30.

In some cases, a variant IL-2 polypeptide exhibits reduced binding affinity to IL-2R, compared to the binding affinity of an IL-2 polypeptide comprising the amino acid sequence set forth in SEQ ID NO:27. For example, in some cases, a variant IL-2 polypeptide binds IL-2R with a binding affinity that is at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the amino acid sequence set forth in SEQ ID NO:27 for an IL-2R (e.g., an IL-2R comprising polypeptides comprising the amino acid sequence set forth in SEQ ID NOs:28-30), when assayed under the same conditions.

In some cases, a variant IL-2 polypeptide has a binding affinity to IL-2R that is from about 100 nM to about 100 μM. As another example, in some cases, a variant IL-2 polypeptide has a binding affinity for IL-2R (e.g., an IL-2R comprising polypeptides comprising the amino acid sequence set forth in SEQ ID NOs:28-30) that is from about 100 nM to about 150 nM, from about 150 nM to about 200 nM, from about 200 nM to about 250 nM, from about 250 nM to about 300 nM, from about 300 nM to about 350 nM, from about 350 nM to about 400 nM, from about 400 nM to about 500 nM, from about 500 nM to about 600 nM, from about 600 nM to about 700 nM, from about 700 nM to about 800 nM, from about 800 nM to about 900 nM, from about 900 nM to about 1 μM, from about 1 μM to about 5 μM, from about 5 μM to about 10 μM, from about 10 μM to about 15 μM, from about 15 μM to about 20 μM, from about 20 μM to about 25 μM, from about 25 μM to about 50 μM, from about 50 μM to about 75 μM, or from about 75 μM to about 100 μM.

In some cases, a variant IL-2 polypeptide has a single amino acid substitution compared to the IL-2 amino acid sequence set forth in SEQ ID NO:27. In some cases, a variant IL-2 polypeptide has from 2 to 10 amino acid substitutions compared to the IL-2 amino acid sequence set forth in SEQ ID NO:27. In some cases, a variant IL-2 polypeptide has 2, 3, 4, 5, 6, 7, 8, 9, or 10 amino acid substitutions compared to the IL-2 amino acid sequence set forth in SEQ ID NO:27.

Suitable IL-2 variants include a polypeptide that comprises an amino acid sequence having at least 90%, at least 95%, at least 98%, at least 99%, or 100% amino acid sequence identity to any one of the following amino acid sequences:

APTSSSTKKT QLQLEHLLLD LQMILNGINN YKNPKLTRML TXKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X is any amino acid other than Phe. In some cases, X is Ala;

APTSSSTKKT QLQLEHLLLX LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X is any amino acid other than Asp. In some cases, X is Ala;

APTSSSTKKT QLQLXHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X is any amino acid other than Glu. In some cases, X is Ala;

APTSSSTKKT QLQLEXLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X is any amino acid other than His. In some cases, X is Ala. In some cases, X is Arg. In some cases, X is Asn. In some cases, X is Asp. In some cases, X is Cys. In some cases, X is Glu. In some cases, X is Gln. In some cases, X is Gly. In some cases, X is Ile. I n some cases, X is Lys. In some cases, X is Leu. In some cases, X is Met. In some cases, X is Phe. In some cases, X is Pro. In some cases, X is Ser. In some cases, X is Thr. In some cases, X is Tyr. In some cases, X is Trp. In some cases, X is Val;

APTSSSTKKT QLQLEHLLLD LQMILNGINN YKNPKLTRML TFKFXMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X is any amino acid other than Tyr. In some cases, X is Ala;

APTSSSTKKT QLQLEHLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCXSIIS TLT (SEQ ID NO:27), where X is any amino acid other than Gln. In some cases, X is Ala;

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X₁ is any amino acid other than His, and where X₂ is any amino acid other than Phe. In some cases, X₁ is Ala. In some cases, X₂ is Ala. In some cases, X₁ is Ala; and X₂ is Ala;

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X₁ is any amino acid other than Asp; and where X₂ is any amino acid other than Phe. In some cases, X₁ is Ala. In some cases, X₂ is Ala. In some cases, X₁ is Ala; and X₂ is Ala;

APTSSSTKKT QLQLX₁HLLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X₁ is any amino acid other than Glu; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEX₁LLLX₂ LQMILNGINN YKNPKLTRML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X₁ is any amino acid other than His; where X₂ is any amino acid other than Asp; and where X₃ is any amino acid other than Phe. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLLX₁ LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:27), where X₁ is any amino acid other than Asp; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X₁ is any amino acid other than Asp; where X₂ is any amino acid other than Phe; and where X₃ is any amino acid other than Tyr. In some cases, X₁ is Ala. In some cases, X₂ is Ala. In some cases, X₃ is Ala. In some cases, X₁ is Ala; X₂ is Ala; and X₃ is Ala;

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:27), where X₁ is any amino acid other than His; where X₂ is any amino acid other than Asp; where X₃ is any amino acid other than Phe; and where X₄ is any amino acid other than Tyr. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLEHLLLX₁ LQMILNGINN YKNPKLTRML TX₂KFX₃MPKKA TELKHLQCLE EELKPLEEVL NLAQSKNFHL RPRDLISNIN VIVLELKGSE TTFMCEYADE TATIVEFLNR WITFCX₄SIIS TLT (SEQ ID NO:27), where X₁ is any amino acid other than Asp; where X₂ is any amino acid other than Phe; where X₃ is any amino acid other than Tyr; and where X₄ is any amino acid other than Gln. In some cases, X₁ is Ala. In some cases, X₂ is Ala. In some cases, X₃ is Ala. In some cases, X₄ is Ala. In some cases, X₁ is Ala; X₂ is Ala; X₃ is Ala; and X₄ is Ala;

APTSSSTKKT QLQLEX₁LLLX₂ LQMILNGINN YKNPKLTRML TX₃KFX₄MPKKA TELKHLQCLE EELKPLEEVL NLAQSKNFHL RPRDLISNIN VIVLELKGSE TTFMCEYADE TATIVEFLNR WITFCX₅SIIS TLT (SEQ ID NO:27), where X₁ is any amino acid other than His; where X₂ is any amino acid other than Asp; where X₃ is any amino acid other than Phe; where X₄ is any amino acid other than Tyr; and where X₅ is any amino acid other than Gln. In some cases, X₁ is Ala. In some cases, X₂ is Ala. In some cases, X₃ is Ala. In some cases, X₄ is Ala. In some cases, X₅ is Ala. In some cases, X₁ is Ala; X₂ is Ala; X₃ is Ala; X₄ is Ala; X₅ is Ala; and

APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCX₃SIIS TLT (SEQ ID NO:27), where X₁ is any amino acid other than His; where X₂ is any amino acid other than Phe; and where X₃ is any amino acid other than Gln. In some cases, X₁ is Ala. In some cases, X₂ is Ala. In some cases, X₃ is Ala. In some cases, X₁ is Ala; X₂ is Ala; and X₃ is Ala.

In any of the wild-type or variant IL-2 sequences provided herein the cysteine at position 125 may be substituted with an alanine (a C125A substitution). In addition to any stability provided by the substitution, it may be employed where, for example, an epitope containing peptide or payload is to be conjugated to a cysteine residue elsewhere in any TMAPP, thereby avoiding competition from the C125 of the IL-2 MOD sequence.

Dimerizer Pairs

As noted above, in some cases, a TMAPP may comprise a dimerizer pair (or dimerization pair) of polypeptides. For example, in any TMAPP that is a multimeric polypeptide comprising at least a first and a second polypeptide, the first polypeptide may comprise a first member of a dimerization pair, and the second polypeptide may comprise a second member of the dimerization pair.

Dimerization peptides are known in the art; and any known dimerization peptide is suitable for use. Dimerization peptides include polypeptides of the collectin family (e.g., ACRP30 or ACRP30-like proteins) which contain collagen domains consisting of collagen repeats Gly-Xaa-Xaa. Other dimerization peptides include coiled-coil domains and leucine-zipper domains. A collagen domain can comprise (Gly-Xaa-Xaa)ₙ, where Xaa is any amino acid, and where n is an integer from 10 to 40. In some cases, a collagen domain comprises (Gly-Xaa-Pro)ₙ, where Xaa is any amino acid and n is an integer from 10 to 40. Dimerization peptides are well known in the art; see, e.g., U.S. Patent Publication No. 2003/0138440.

In some cases, a dimerization pair includes two leucine-zipper polypeptides that bind to one another. Non-limiting examples of leucine-zipper polypeptides include, e.g., a peptide of any one of the following amino acid sequences:

(SEQ ID NO: 86)
RMKQIEDKIEEILSKIYHIENEIARIKKLIGER;
(SEQ ID NO: 87)
LSSIEKKQEEQTSWLIWISNELTLIRNELAQS;
(SEQ ID NO: 88)
LSSIEKKLEEITSQLIQISNELTLIRNELAQ;
(SEQ ID NO: 89)
LSSIEKKLEEITSQLIQIRNELTLIRNELAQ;
(SEQ ID NO: 90)
LSSIEKKLEEITSQLQQIRNELTLIRNELAQ;
(SEQ ID NO: 91)
LSSLEKKLEELTSQLIQLRNELTLLRNELAQ;
and
(SEQ ID NO: 92)
ISSLEKKIEELTSQIQQLRNEITLLRNEIAQ.

In some cases, a leucine-zipper polypeptide comprises the following amino acid sequence:

(SEQ ID NO: 93)
LEIEAAFLERENTALETRVAELRQRVQRLRNRVSQYRTRYGPLGGGK.

Additional leucine-zipper polypeptides are known in the art, any of which is suitable for use in any TMAPP of the present disclosure.

A collagen oligomerization peptide can comprise the following amino acid sequence:

(SEQ ID NO: 94)
VTAFSNMDDMLQKAHLVIEGTFIYLRDSTEFFIRVRDGWKKLQLGELIPIP
ADSPPPPALSSNP.

Coiled-coil dimerization peptides are known in the art. For example, a coiled-coil dimerization peptide can be a peptide of any one of the following amino acid sequences:

(SEQ ID NO: 95)
LKSVENRLAVV ENQLKTVIEELKTVKDLLSN;
(SEQ ID NO: 96)
LARIEEKLKTIKAQLSEIASTLNMIREQLAQ;
(SEQ ID NO: 97)
VSRLEEKVKTLKSQVTELASTVSLLREQVAQ;
(SEQ ID NO: 98)
IQSEKKIEDISSLIGQIQSEITLIRNEIAQ;
and
(SEQ ID NO: 99)
LMSLEKKLEELTQTLMQLQNELSMLKNELAQ.

In some cases, a dimerization peptide comprises at least one cysteine residue. Examples include, e.g.:

(SEQ ID NO: 100)
VDLEGSTSNGRQCAGIRL;
(SEQ ID NO: 101)
EDDVTTTEELAPALVPPPKGTCAGWMA;
and
(SEQ ID NO: 102)
GHDQETTTQGPGVLLPLPKGACTGQMA.

Additional Polypeptides

A polypeptide chain of any TMAPP of the present disclosure can include one or more polypeptides in addition to those described above. Suitable additional polypeptides include epitope tags and affinity domains. The one or more additional polypeptides can be included at, for example, i) the N-terminus of a polypeptide chain of any TMAPP of the present disclosure, ii) the C-terminus of a polypeptide chain of any TMAPP of the present disclosure, or iii) internally within a polypeptide chain of any TMAPP of the present disclosure.

Epitope Tag

Suitable epitope tags include, but are not limited to, hemagglutinin (HA; e.g., YPYDVPDYA (SEQ ID NO:31); FLAG (e.g., DYKDDDDK (SEQ ID NO:32); c-myc (e.g., EQKLISEEDL; SEQ ID NO:33), and the like.

Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel SEPHAROSE®. Exemplary affinity domains include His5 (HHHHH) (SEQ ID NO:34), HisX6 (HHHHHH) (SEQ ID NO:35), C-myc (EQKLISEEDL) (SEQ ID NO:33), Flag (DYKDDDDK) (SEQ ID NO:32), StrepTag (WSHPQFEK) (SEQ ID NO:36), hemagglutinin, e.g., HA Tag (YPYDVPDYA) (SEQ ID NO:31), glutathione-S-transferase (GST), thioredoxin, cellulose binding domain, RYIRS (SEQ ID NO:37), Phe-His-His-Thr (SEQ ID NO:38), chitin binding domain, S-peptide, T7 peptide, SH2 domain, C-end RNA tag, WEAAAREACCRECCARA (SEQ ID NO:39), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, 5100 proteins, parvalbumin, calbindin D9K, calbindin D28K, and calretinin, inteins, biotin, streptavidin, MyoD, Id, leucine zipper sequences, and maltose binding protein.

Chemical Conjugation Sites and Chemical Conjugation

The chemical conjugation sites in the TMAPPs described herein may be selected from any suitable site known in the art that can be modified upon treatment with a reagent and/or catalyst, such as an enzyme, that permits the formation of a covalent linkage to the TMAPPs.

Chemical conjugation sites may be added to any portion of a sc-TMMP or m-TMAPP including, but not limited to, the MHC Class II α1, α2, β1 or β2 polypeptide, or if present, a Fc or other non-Ig scaffold peptide, or a peptide linker attached directly or indirectly to any of the foregoing.

In an embodiment, where chemical conjugation is used to prepare a sc- or m-TMAPP-epitope conjugate, at least one chemical conjugation site may be within or at the N-terminus of a MHC Class II β1 polypeptide, or within or at the N-terminus of a linker (an optional linker) attached to the N-terminus of the MHC Class II β1 polypeptide. In addition, chemical conjugation sites can be located anywhere in a sc- or m-TMAPP molecule, such as attached to (e.g., at the N- or C-terminus) or within, the sequence of a MHC Class II α1, α2, or β2 polypeptide of the present disclosure, a Fc or other non-Ig scaffold peptide of the present disclosure, or a linker attached directly or indirectly to any of the foregoing. Chemical conjugation sites can be used to prepare conjugates other than epitope conjugates, including drug and/or diagnostic (e.g., detectable label) conjugates.

In an embodiment, a sc- or m-TMAPP may have only one chemical conjugation site.

In an embodiment, sc- or m-TMAPPs comprise at least one chemical conjugation site within or at the amino terminus of the sequence of a naturally occurring human HLA Class II β1 domain or a sequence having at least 85%, 90%, 95%, 98%, or 99% amino acid sequence identity with it before the addition of any chemical conjugation site. In an embodiment, sc- or m-TMAPPs comprise at least one chemical conjugation site within or at the amino terminus of a HLA Class II β1 domain sequence selected from the sequences set forth in FIG. 7A-J, 8A-8C, 9, 10, 12, 14, 16, 19A-19B or 20A-20B, or a sequence having at least 85%, at least 90%, at least 95%, at least 98%, at least 99% or 100% amino acid sequence identity to a sequence provided in those figures before the addition of the chemical conjugation site. In an embodiment, sc- or m-TMAPPs comprise at least one chemical conjugation site within or at the amino terminus of a polypeptide having at least 50, 60, 70, or 80 contiguous amino acids of a HLA Class II β1 domain sequence selected from the sequences set forth in any one of FIG. 7A-J, 8A-8C, 9, 10, 12, 14, 16, 19A-19B or 20A-20B. In an embodiment, sc- or m-TMAPPs comprise at least one chemical conjugation site within or at the amino terminus of a polypeptide comprising a sequence with at least 85%, 90%, 95%, 98%, 99% or 100% amino acid sequence identity to a sequence having at least 50, 60, 70, or 80 contiguous amino acids of a sequence set forth in any one of FIG. 7A-J, 8A-8C, 9, 10, 12, 14, 16, 19A-19B or 20A-20B. As an alternative to the chemical conjugation site being located within or at the amino terminus of the MHC Class II β1 domain or a sequence recited above (e.g., FIG. 7A-J, 8A-8C, 9, 10, 12, 14, 16, 19A-19B or 20A-20B or sequences with at least 85% amino acid identity thereto), the chemical conjugation site may be in, or at, the N-terminus of a linker attached to the MHC Class II β1 domain (the linker itself may be attached to the N-terminus of the MHC Class II β1 polypeptide).

In an embodiment one or more chemical conjugation site(s) may be selected independently from the group consisting of: a) a peptide sequence that acts as an enzyme modification sequence (e.g., sulfatase, sortase, and/or transglutaminase sequences); b) non-natural amino acids and/or selenocysteines; c) engineered amino acid chemical conjugation sites; d) carbohydrate or oligosaccharide moieties; and e) IgG nucleotide binding sites.

Sulfatase Motifs

In those embodiments where enzymatic modification is chosen as the means of providing one or more chemical conjugation sites, a sulfatase motif may be incorporated into the TMAPPs at any of the locations described above. Sulfatase motifs are usually 5 or 6 amino acids in length, and are described, for example, in U.S. Pat. No. 9,540,438 and U.S. Pat. Pub. No. 2017/0166639 A1, which are incorporated by reference for their disclosure and use of sulfatase motifs. Insertion of the motif results in the formation of a protein or polypeptide that is sometimes referred to as “aldehyde tagged” or having an “aldehyde tag.” The motif may be acted on by formylglycine generating enzyme(s) (“FGE” or “FGEs”) that convert a cysteine or serine in the motif to a formylglycine residue (“fGly” although sometimes denoted “FGly”), which is an aldehyde containing amino acid residue that may be utilized for selective (e.g., site specific) chemical conjugation reactions. Accordingly, as used herein, “aldehyde tag” or “aldehyde tagged” polypeptides refer to an amino acid sequence comprising an unconverted sulfatase motif, as well as to an amino acid sequence comprising a sulfatase motif in which the cysteine or the serine residue of the motif has been converted to fGly by action of an FGE. In addition, where a sulfatase motif is provided in the context of an amino acid sequence, it is understood as providing disclosure of both the amino acid sequence (e.g., polypeptide) containing the unconverted motif as well as its fGly-containing counterpart produced by FGE conversion. Once incorporated into a polypeptide, a fGly residue may be reacted with molecules comprising a variety of reactive groups, including but not limited to thiosemicarbazide, aminooxy, hydrazide, and hydrazino groups, to form a conjugate (e.g., a sc- or m-TMAPP-epitope conjugate) having a covalent bond between the polypeptide (via its fGly residue) and the molecule.

In embodiments, the sulfatase motif is at least 5 or 6 amino acid residues, but can be, for example, from 5 to 16 (e.g., 6-16, 5-14, 6-14, 5-12, 6-12, 5-10, 6-10, 5-8, or 6-8) amino acids in length. The sulfatase motif may be limited to a length less than 16, 14, 12, 10, or 8 amino acid residues.

In an embodiment, the sulfatase motif contains the sequence shown in Formula (I):

X1Z1X2Z2X3Z3 (I) (SEQ ID NO:45), where
Z1 is cysteine or serine;
Z2 is either a proline or alanine residue (which can also be represented by “P/A”);
Z3 is a basic amino acid (arginine, lysine, or histidine, usually lysine), or an aliphatic amino acid (alanine, glycine, leucine, valine, isoleucine, or proline, usually A, G, L, V, or I);
X1 is present or absent and, when present, can be any amino acid, though usually an aliphatic amino acid, a sulfur-containing amino acid, or a polar uncharged amino acid (e.g., other than an aromatic amino acid or a charged amino acid), usually L, M, V, S or T, more usually L, M, S or V, with the proviso that, when the sulfatase motif is at the N-terminus of the target polypeptide, X1 is present; and
X2 and X3 independently can be any amino acid, though usually an aliphatic amino acid, a polar, uncharged amino acid, or a sulfur containing amino acid (e.g., other than an aromatic amino acid or a charged amino acid), usually S, T, A, V, G or C, more usually S, T, A, V or G.

Accordingly, in one embodiment, FGly containing polypeptides may be prepared using a sulfatase motif having Formula I, where:
Z1 is cysteine or serine;
Z2 is a proline or alanine residue;
Z3 is an aliphatic amino acid or a basic amino acid;
X1 is present or absent and, when present, is any amino acid, with the proviso that, when the sulfatase motif is at an N-terminus of the polypeptide, X1 is present; and
X2 and X3 are each independently any amino acid, wherein the sequence is within or adjacent to a solvent accessible loop region of the Ig constant region, and wherein the sequence is not at the C-terminus of the Ig heavy chain.

Where the aldehyde tag is present at a location other than the N-terminus of a target polypeptide, X1 of the sulfatase motif may be provided by an amino acid of the sequence in which the target polypeptide is incorporated. Accordingly, in some embodiments, where the motif is present at a location other than the N-terminus of a target polypeptide, the sulfatase motif may be of the formula:

(C/S)X2(P/A)X3Z3, Formula (II) (SEQ ID NO:46),

where: X1 is absent, and X2, X3 and Z3 are as defined above.

Where peptides containing a sulfatase motif are being prepared for conversion into fGly-containing peptides by a eukaryotic FGE, for example by expression and conversion of the peptide in a eukaryotic cell or cell free system using a eukaryotic FGE, sulfatase motifs amenable to conversion by a eukaryotic FGE may advantageously be employed. In general, sulfatase motifs amenable to conversion by a eukaryotic FGE contain a cysteine and proline at Z1 and Z2 respectively in Formula (I) above (e.g., X1CX2PX3Z3, SEQ ID NO:47); and in CX2PX3Z3, SEQ ID NO:48 (encompassed by Formula (II) above). Peptides bearing those motifs can be modified by “SUMF1-type” FGEs.

In an embodiment where the FGE is a eukaryotic FGE, the sulfatase motif may comprise an amino acid sequence selected from the group consisting of:
X1CX2PX3R or CX2PX3R (SEQ ID NOs:47 and 48, where Z3 is R, and X1 is present or absent);
X1CX2PX3K or CX2PX3K (SEQ ID NOs:47 and 48, where Z3 is K, and X1 is present or absent);
X1CX2PX3H or CX2PX3H (SEQ ID NOs:47 and 48, where Z3 is H, and X1 is present or absent);
X1CX2PX3L or CX2PX3L (SEQ ID NOs:47 and 48, where Z3 is L, and X1 is present or absent); where X1, X2 and X3 are as defined above.

In an embodiment, the sulfatase motif comprises the sequence: X1C(X2)P(X3)Z3 (see SEQ ID NO:47), where:
X1 is present or absent and, when present, is any amino acid, provided that, when the sulfatase motif is at an N-terminus of a polypeptide, X1 is present; and
X2 and X3 are independently selected serine, threonine, alanine or glycine residues.

Sulfatase motifs of Formula (I) and Formula II amenable to conversion by a prokaryotic FGE often contain a cysteine or serine at Z1 and a proline at Z2 may be modified either by the “SUMP I-type” FGE or the “AtsB-type” FGE, respectively. Other sulfatase motifs of Formula (I) or (II) susceptible to conversion by a prokaryotic FGE contain a cysteine or serine at Z1, and a proline or alanine at Z2 (each of which are selected independently), with the remaining amino acids of the sequence as descried for Formulas (I) and (II); and are susceptible to modification by, for example, a FGE from Clostridium perfringens (a cysteine type enzyme), Klebisella pneumoniae (a Serine-type enzyme) or a FGE of Mycobacterium tuberculosis.

Sulfatase motifs may be incorporated into any desired location in a sc-TMMP or m-TMAPP and used not only to incorporate an epitope, but also in the formation of conjugates with drugs and diagnostic molecules as discussed below. Epitopes and other molecules may be conjugated directly to the TMAPP, or attached indirectly through a linker which reacts with the aldehyde group.

In an embodiment, a sulfatase motif may be added to, at, or near the N-terminus of a TMAPP's MHC Class II polypeptide as set forth in FIG. 7A-J, 8A-8C, 9, 10, 12, 14, 16, 19A-19B or 20A-20B, or to a polypeptide linker attached to the N-terminus of those sequences as discussed above. In an embodiment a sulfatase motif is incorporated into a sequence having at least 85% (e.g., at least 90%, 95%, 98% or 99%, or even 100%) amino acid sequence identity to a sequence shown in any one of FIG. 7A-J, 8A-8C, 9, 10, 12, 14, 16, 19A-19B or 20A-20B, before the addition of the sulfatase motif sequence.

In another embodiment, the one or more copies of the sulfatase motif of Formula (I) or Formula (II) may be incorporated into an IgFc region. In one such embodiment they may be utilized as sites for the conjugation of, for example, epitopes and/or other molecules such as drugs, either directly or indirectly through a peptide or chemical linker.

As indicated above, a sulfatase motif of an aldehyde tag is at least 5 or 6 amino acid residues, but can be, for example, from 5 to 16 amino acids in length. The motif can contain additional residues at one or both of the N- and C-termini, such that the aldehyde tag includes both a sulfatase motif and an “auxiliary motif.” In an embodiment, the sulfatase motif includes a C-terminal auxiliary motif (e.g., following the Z3 position of the motif), and may include 1, 2, 3, 4, 5, 6, or all 7 contiguous residues of an amino acid sequence selected from the group consisting of AALLTGR (SEQ ID NO:49), SQLLTGR (SEQ ID NO:50), AAFMTGR (SEQ ID NO:51), AAFLTGR (SEQ ID NO:52), and GSLFTGR (SEQ ID NO:53); numerous other auxiliary motifs have been described in, for example, the references cited herein. The auxiliary motif amino acid residues are not required for FGE mediated conversion of the sulfatase motif, and thus may be specifically excluded from the aldehyde tags described herein.

U.S. Pat. No. 9,540,438 discusses the incorporation of sulfatase motifs into the various immunoglobulin sequences, including Fc region polypeptides, and is herein incorporated by reference for its teachings on sulfatase motifs and modification of Fc polypeptides and other polypeptides. That patent is also incorporated by reference for its guidance on FGE enzymes, and their use in forming FGly residues as well as the chemistry related to the coupling of molecules, such as epitopes and other molecules (e.g., drugs and diagnostic agents), to FGly residues.

The incorporation of a sulfatase motif may be accomplished by incorporating a nucleic acid sequence encoding the motif at the desired location in a nucleic acid encoding all or part of the TMAPP described herein. As discussed below, the nucleic acid sequence may be placed under the control of a transcriptional regulatory sequence(s) (a promoter), and provided with regulatory elements that direct its expression. The expressed protein may be treated with one or more FGEs after expression and partial or complete purification. Alternatively, expression of the nucleic acid in cells that express a FGE recognizing the sulfatase motif results in the conversion of the cysteine or serine of the motif to fGly, which is sometimes called oxoalanine. Where two or more different sulfatase motifs are present (e.g., a first and second sulfatase motif), it is also possible to conduct the conversion of each motif during cellular expression, or each motif after cellular expression and partial or complete purification. Using two or more FGE enzymes with different motif selectivity and motifs preferentially converted by each of the FGEs, it is also possible to sequentially convert at least one sulfatase motif during cellular expression and at least one sulfatase motif after partial or complete purification, or to separately convert sulfatase motifs to fGly residues after expression. As discussed below, the ability to separately convert different sulfatase motifs and chemically couple them to epitopes and/or payloads in a sequential fashion permits the use of sulfatase coupling to incorporate different epitopes or payloads at the locations of different motifs.

Host cells for production of unconverted or (where the host cell expresses a suitable FGE) converted fGly-containing polypeptides include those of prokaryotic and eukaryotic organisms. Non-limiting examples include Escherichia coli strains, Bacillus spp. (e.g., B. subtilis, and the like), yeast or fungi (e.g., S. cerevisiae, Pichia spp., and the like). Examples of other host cells, including those derived from a higher organism, such as insects and vertebrates, particularly mammals, include, but are not limited to, CHO cells, HEK cells, and the like (e.g., American Type Culture Collection (ATCC) No. CCL-2), CHO cells (e.g., ATCC Nos. CRL9618 and CRL9096), CHO DG44 cells, CHO-Kl cells (ATCC CCL-61), human embryonic kidney (HEK) 293 cells (e.g., ATCC No. CRL-1573), Vero cells, NIH 3T3 cells (e.g., ATCC No. CRL-1658), Hnh-7 cells, BHK cells (e.g., ATCC No. CCL1O), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

A variety of FGEs may be employed for the conversion (oxidation) of cysteine or serine in a sulfatase motif to FGly. As used herein, the term formylglycine generating enzyme, or FGE, refers to FGly-generating enzymes that catalyze the conversion of a cysteine or serine of a sulfatase motif to FGly. As discussed in U.S. Pat. No. 9,540,438, the literature often uses the term formylglycine-generating enzymes for those enzymes that convert a cysteine of the motif to FGly, whereas enzymes that convert a serine in a sulfatase motif to FGly are referred to as Ats-B-like.

FGEs may be divided into two categories, aerobic and anaerobic. The aerobic enzymes, which include the eukaryotic enzyme (e.g., the human enzyme), convert a cysteine residue to fGly, where the cysteine is generally in the context of a sulfatase motif of the formula X1CX2PX3Z3 (SEQ ID NO:47). Eukaryotic FGEs are of the “SUMF1-type” and are encoded in humans by the SUMF1 gene. The anaerobic enzymes are of the AtsB type most often from prokaryotic sources (e.g., Clostridium perfringens, Klebsiella pneumoniae, or Mycobacterium tuberculosis) and appear to be able to convert a cysteine or a serine in their sulfatase motif to fGly using a mechanism that is different from the aerobic form.

The ability to catalyze serine or cysteine conversion to FGly depends on the enzyme and the sulfatase motifs. Because of the differences in the ability of FGEs to convert serine and cysteine, it is possible that different sulfatase motifs may be used as different chemical conjugation sites. For example, it may be possible to incorporate into a sc-TMAPP or m-TMAPP a sequence encoding both a cysteine containing site amenable to conversion by the eukaryotic aerobic SUMF1-type FGE and a serine containing site amenable to conversion by an AtsB-type FGE. In a eukaryotic cell expressing a SumF1-type FGE, the cysteine motif will bear a fGly residue that may be subject to a first chemical conjugation with an epitope or payload. Following the first chemical conjugation, the sc-TMAPP or m-TMAPP conjugate would be treated with an AtsB-type serine-type enzyme in a cell free system, and the FGly produced from the serine containing motif can then be subjected to chemical conjugation with a molecule that is the same as or different from the molecule used in the first chemical conjugation.

In view of the foregoing, this disclosure provides for sc- or m-TMAPPs comprising one or more fGly residues incorporated into the sequence of the first or second polypeptide chain as discussed above. The fGly residues may, for example, be in the context of the sequence X1(fGly)X2Z2X3Z3, where: fGly is the formylglycine residue; and Z2, Z3, X1, X2 and X3 are as defined in Formula (I) above.

After chemical conjugation, the sulfatase motif containing TMAPPs comprise one or more FGly′ residues incorporated into their sequence in the context of, for example, the sequence X1FGly′X2Z2X3Z3, where the FGly′ residue is formylglycine that has undergone a chemical reaction and now has a covalently attached moiety (e.g., epitope or therapeutic).

A number of chemistries and commercially available reagents can be utilized to conjugate a molecule (e.g., an epitope or other molecule such as a drug) to a FGly residue, including, but not limited to, the use of thiosemicarbazide, aminooxy, hydrazide, hydrazino, or derivatives of the molecules to be coupled at a FGly-containing chemical conjugation site. For example, epitopes (e.g., epitope peptides) and/or other molecules (e.g., drugs and/or diagnostic agents) bearing thiosemicarbazide, aminooxy, hydrazide, hydrazino or hydrazinyl functional groups (e.g., attached directly to an amino acid of a peptide or via a linker such as a PEG) can be reacted with a FGly-containing sc-TMAPP or m-TMAPP to form a covalently linked epitope. Similarly, payloads such as drugs and therapeutics can be incorporated using, for example, biotin hydrazide as a linking agent.

In an embodiment, a peptide is modified to incorporate a nucleophile-containing moiety (e.g., an aminooxy or hydrazide moiety) that reacts with the FGly-containing amino acid residues incorporated into the polypeptide(s) of a sc- or m-TMAPP. The reaction results in the formation of a conjugate in which a peptide of a sc-TMAPP or m-TMAPP and the epitope (or another molecule) are covalently linked (e.g., by hydrazone or oxime linkage). (See, e.g., U.S. Pat. Nos. 9,238,878 and 7,351,797; Interchem, Aminooxy & Aldehyde PEO/PEG reagents for Biorthogonal Conjugation and Labeling featuring Oxime Formation (undated), available at http://www.interchim.fr/ft/J/JV2290.pdf, accessed Sep. 2, 2017).

In an embodiment, an epitope (e.g., peptide epitope) and/or another molecule (e.g., a drug or diagnostic agent), such as a drug bearing a thiosemicarbazide, aminooxy, hydrazide, or hydrazino group, is reacted with a FGly-containing polypeptide of a sc- or m-TMAPP. The reaction results in the formation of a covalent bond between the TMAPP and the epitope and/or the other molecule (e.g., a drug or diagnostic agent). As discussed in U.S. Pat. No. 9,540,438 and U.S. Pat. Pub. No. 2017/0166639 A1, the resulting conjugates may contain a structure (modified amino acid residue) of the form:

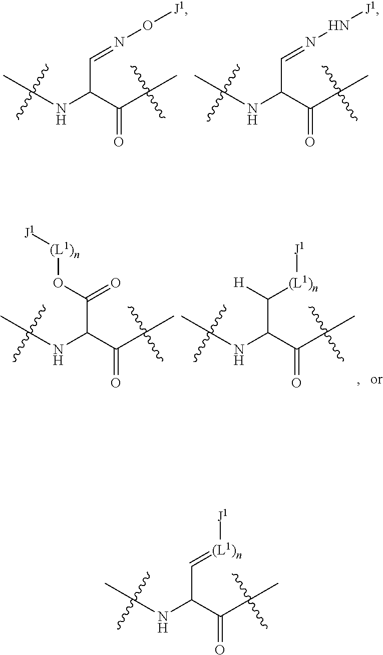

where:
J¹ is a covalently bound moiety;
each L¹ is a divalent moiety independently selected from alkylene, substituted alkylene, alkenylene, substituted alkenylene, alkynylene, substituted alkynylene, arylene, substituted arylene, cycloalkylene, substituted cycloalkylene, heteroarylene, substituted heteroarylene, heterocyclene, substituted heterocyclene, acyl, amido, acyloxy, urethanylene, thioester, sulfonyl, sulfonamide, sulfonyl ester, —O—, —S—, —NH—, and substituted amine; and
n is a number selected from zero to 40 (e.g., 1-5, 5-10, 10-20, 20-30, or 30-40).

In an embodiment, epitopes and/or other molecules (e.g., drug or diagnostic agents) may be modified to include a covalently bound hydrazinyl group, including those bearing cyclic substituents (e.g., indoles), that permits their covalent attachment to a sc-TMAPP or m-TMAPP bearing FGly amino acid residues. In one embodiment the hydrazinal compounds are compounds of Formula (III):

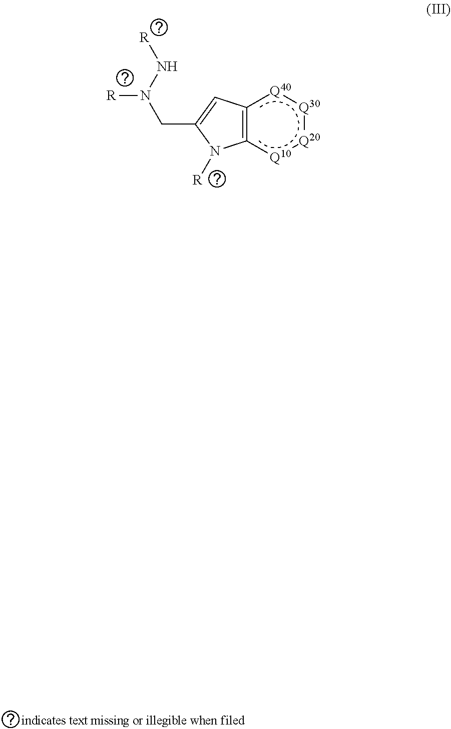

wherein
R′″ may be a payload or epitope of interest that is to be conjugated to the FGly containing polypeptide;
R′ and R″ may each independently be any desired substituent including, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl; and
Q¹⁰, Q²⁰, Q³⁰ and Q⁴⁰ may be CR¹¹, NR¹², N, O or S; and
   wherein one of Q¹⁰, Q²⁰, Q³⁰ and Q⁴⁰ is optional, and R¹¹ and R¹² may be any desired substituent. See, U.S. Pat. Pub. No. 2015/0352225.

In other embodiments the hydrazinyl group modified epitopes and payloads (e.g., drugs and/or diagnostic agents) have a structure given by Formula (IV), (V), (Va), (VI), or (VIa). See U.S. Pat. No. 9,310,374, which is incorporated by reference for its teachings on the preparation and use of hydrazinyl compounds in the formation of biological conjugates including conjugates involving peptides and polypeptides.

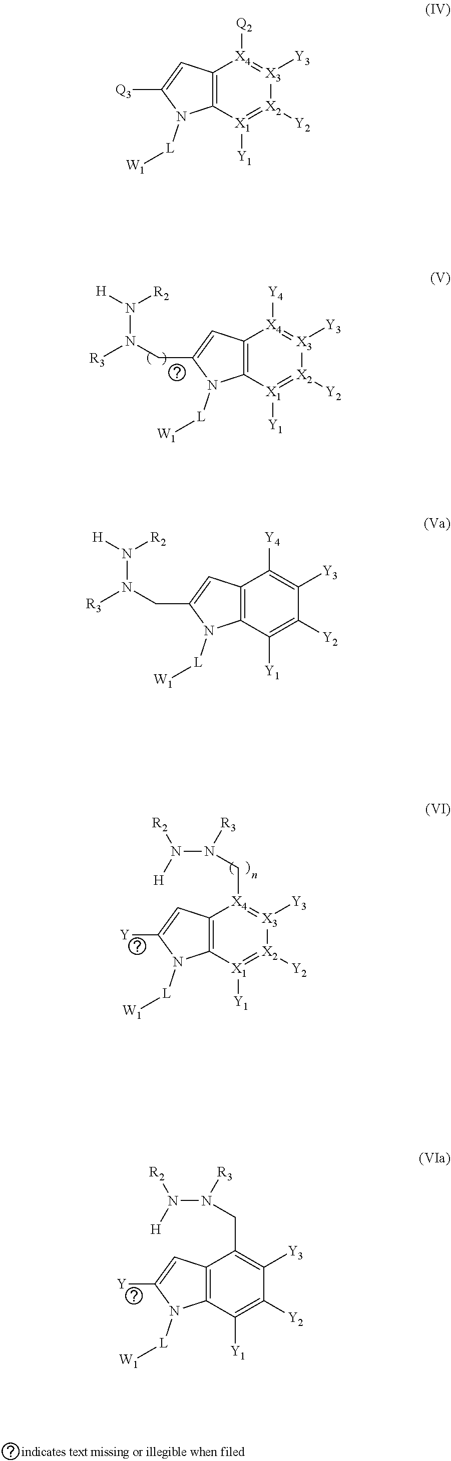

wherein, for the purpose of Formulas (IV), (V), (Va), (VI), or (VIa) recited in this section:
one of Q₂ and Q₃ is —(CH₂) nNR₃NHR₂ and the other is Y₄;
n is 0 or 1;
R₂ and R₃ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
X₁, X₂, X₃ and X₄ are each independently selected from C, N, O and S;
Y₁, Y₂, Y₃ and Y₄ are each independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L is an optional linker; and
W₁ is selected from an epitope (e.g., epitope polypeptide), a drug, a diagnostic agent or other payload.

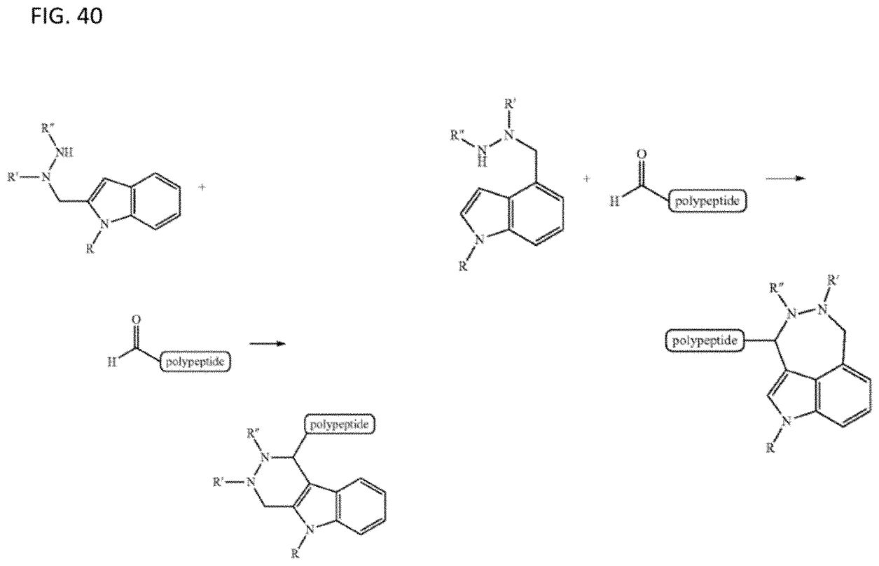

Reactions of hydrazinyl indoles, which fall within those structures, with aldehyde functionalized peptides are shown schematically in FIG. 40.

In an embodiment, Q₂ is —(CH₂)ₙNR₃NHR₂ and Q₃ is Y₄. In an embodiment, Q₃ is —(CH₂)ₙNR₃NHR₂ and Q₂ is Y₄. In an embodiment, n is 1. In an embodiment, R₂ and R₃ are each independently selected from alkyl and substituted alkyl. In an embodiment, R₂ and R₃ are each methyl. In an embodiment, X₁, X₂, X₃ and X₄ are each C. In an embodiment, Y₁, Y₂, Y₃ and Y₄ are each H.

In an embodiment, L is present and includes a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In an embodiment, L is present and includes a polymer. In an embodiment, the polymer is a polyethylene glycol.

For the purposes of Formulas (IV), (V), (Va), (VI), or VIa):
1. “Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂₄ isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).
2. The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), or —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.
3. “Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.
4. R¹⁰ is H or alkyl (e.g., H, —CH₃, —CH₂CH₃ or —CH₂CH₂CH₃).
5. “Substituted alkylene” refers to an alkylene group having from 1 to 3 hydrogens replaced with substituents as described for carbons in the definition of “substituted” below.
6. The term “alkane” refers to alkyl groups and alkylene groups, as defined herein.
7. The terms “alkylaminoalkyl,” “alkylaminoalkenyl” and “alkylaminoalkynyl” refer to the groups R′NHR″— where R′ is an alkyl group as defined herein and R″ is an alkylene, alkenylene or alkynylene group as defined herein.
8. The term “alkaryl” or “aralkyl” refers to the groups -alkylene-aryl and -substituted alkylene-aryl where alkylene, substituted alkylene and aryl are defined herein.
9. “Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.
10. The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.
11. The term “alkoxyamino” refers to the group —NH-alkoxy, wherein alkoxy is defined herein.
12. The term “haloalkoxy” refers to the group alkyl-O— wherein one or more hydrogen atoms on the alkyl group have been substituted with a halo group and include, by way of examples, groups such as trifluoromethoxy, and the like.
13. The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.
14. The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.
15. The term “alkylthioalkoxy” refers to the groups -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.
16. “Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers and mixtures of these isomers.
17. The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.
18. “Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂CECH).
19. The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.
20. “Alkynyloxy” refers to the group —O-alkynyl, wherein alkynyl is as defined herein. Alkynyloxy includes, by way of example, ethynyloxy, propynyloxy, and the like.
21. “Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—.
22. “Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
23. “Aminocarbonyl” or the term “aminoacyl” refers to the group —C(O)NR²¹R²², wherein R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
24. “Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.
25. The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.
26. The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.
27. “Aminosulfonyl” refers to the group —SO₂NR²¹R²², wherein R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.
28. “Sulfonylamino” refers to the group —NR²¹SO₂R²², wherein R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
29. “Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl), which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted to form “substituted aryl” groups with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.
30. “Aryloxy” refers to the group —O-aryl, wherein aryl is as defined herein, including, by way of example, phenoxy, naphthoxy, and the like, including optionally substituted aryl groups as also defined herein.
31. “Amino” refers to the group —NH₂.
32. The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.
33. The term “azido” refers to the group —N₃.
34. “Carboxyl,” “carboxy” or “carboxylate” refers to —CO₂H or salts thereof.
35. “Carboxyl ester” or “carboxy ester” or the terms “carboxyalkyl” or ““carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
36. “(Carboxyl ester)oxy” or “carbonate” refers to the groups —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
37. “Cyano” or “nitrile” refers to the group —CN.
38. “Cycloalkyl” refers to cyclic alkyl groups of from 3 to 10 carbon atoms having single or multiple cyclic rings including fused, bridged, and spiraling systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.
39. The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.
40. “Cycloalkenyl” refers to non-aromatic cyclic alkyl groups of from 3 to 10 carbon atoms having single or multiple rings and having at least one double bond and preferably from 1 to 2 double bonds.
41. The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.
42. “Cycloalkynyl” refers to non-aromatic cycloalkyl groups of from 5 to 10 carbon atoms having single or multiple rings and having at least one triple bond.
43. “Cycloalkoxy” refers to —O-cycloalkyl.
44. “Cycloalkenyloxy” refers to —O-cycloalkenyl.
45. “Halo” or “halogen” refers to fluoro, chloro, bromo, and iodo.
46. “Hydroxy” or “hydroxyl” refers to the group —OH.
47. “Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as pyridinyl, imidazolyl or furyl) or multiple condensed rings in a ring system (for example as in groups such as indolizinyl, quinolinyl, benzofuran, benzimidazolyl or benzothienyl), wherein at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N—>O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted to form “substituted heteroaryl” groups with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.
48. The term “heteroaralkyl” refers to the group -alkylene-heteroaryl where alkylene and heteroaryl are defined herein. This term includes, by way of example, pyridylmethyl, pyridylethyl, indolylmethyl, and the like.
49. “Heteroaryloxy” refers to —O-heteroaryl.
50. “Heterocycle,” “heterocyclic,” “heterocycloalkyl,” and “heterocyclyl” refer to a saturated or unsaturated group having a single ring or multiple condensed rings, including fused, bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.
51. Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.
52. Unless otherwise constrained by the definition for the heterocyclic substituent, such heterocyclic groups can be optionally substituted with from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.
53. “Heterocyclyloxy” refers to the group —O-heterocyclyl.
54. The term “heterocyclylthio” refers to the group heterocyclic-S—.
55. The term “heterocyclene” refers to the diradical group formed from a heterocycle, as defined herein.
56. The term “hydroxyamino” refers to the group —NHOH.
57. “Nitro” refers to the group —NO₂.
58. “Oxo” refers to the atom (═O).
59. “Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.
60. “Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cycloalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.
61. The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.
62. “Thiol” refers to the group —SH.
63. “Thioxo” or the term “thioketo” refers to the atom (═S).
64. “Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.
65. The term “substituted thioalkoxy” refers to the group —S-substituted alkyl.
66. The term “thioaryloxy” refers to the group aryl-S— wherein the aryl group is as defined herein including optionally substituted aryl groups also defined herein.
67. The term “thioheteroaryloxy” refers to the group heteroaryl-S— wherein the heteroaryl group is as defined herein including optionally substituted aryl groups as also defined herein.
68. The term “thioheterocyclooxy” refers to the group heterocyclyl-S— wherein the heterocyclyl group is as defined herein including optionally substituted heterocyclyl groups as also defined herein.
69. In addition to the disclosure herein, the term “substituted,” when used to modify a specified group or radical, can also mean that one or more hydrogen atoms of the specified group or radical are each, independently of one another, replaced with the same or different substituent groups as defined below.
70. In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰,—SO₂O⁻M⁺, —OSO₂R⁷⁰,—OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰ C(NR⁷⁰ NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or, alternatively, two R⁸⁰s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“_{0.5}” means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.
71. In addition to the disclosure herein, substituent groups for hydrogens on unsaturated carbon atoms in “substituted” alkene, alkyne, aryl and heteroaryl groups are, unless otherwise specified, —R⁶⁰, halo,—O⁻M⁺, —SR⁷⁰,—S⁻M⁺, —NR⁸⁰R⁸⁰, trihalomethyl, —CF₃, —CN, —OCN, —SCN, —NO, —NO₂, —N₃, —SO₂R⁷⁰,—SO₃⁻M⁺, —SO₃R⁷⁰, —OSO₂R⁷⁰,—OSO₃⁻M⁺, —OSO₃R⁷⁰, —PO₃⁻2(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂M⁺, —NR⁷⁰CO₂R⁷⁰, —NR⁷⁰ C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that, in the case of substituted alkene or alkyne, the substituents are not —O⁻M⁺, —SR⁷⁰, or —S⁻M⁺.
72. In addition to the groups disclosed with respect to the individual terms herein, substituent groups for hydrogens on nitrogen atoms in “substituted” heteroalkyl and cycloheteroalkyl groups are, unless otherwise specified, —R⁶⁰,—O⁻M⁺, —SR⁷⁰,—S⁻M⁺, —NR⁸⁰R⁸⁰, trihalomethyl, —CF₃, —CN, —NO, —NO₂, —S(O)₂R⁷⁰, —S(O)₂O⁻M⁺, —OS(O)₂R⁷⁰, —OS(O)₂O⁻M⁺, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰, where R⁶⁰, R⁷⁰, R⁸⁰ and M⁺ are as previously defined.

In an embodiment, an epitope (e.g., peptide epitope) and/or payload to be conjugated with a fGly containing polypeptide has the form of Formula (III), (IV), (V), (Va), (VI), or (VIa). In some embodiments an epitope is covalently bound in a compound of Formula (III), (IV), (V), (Va), (VI), or (VIa). In one such embodiment the epitope is a peptide comprising the amino acid sequence of an epitope (e.g., a viral or cancer epitope). In an embodiment the peptide epitope has a length from about 4 amino acids (aa) to about 20 aa (e.g., 4 aa, 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, or 20 aa) in length.

The disclosure provides for methods of preparing a sc- or m-TMAPP-epitope conjugate and other TMAPP conjugates (e.g., with drugs or diagnostics) comprising:
a) incorporating a sequence encoding a sulfatase motif including a serine or cysteine (e.g., a sulfatase motif of Formula (I) or (II) such as X1CX2PX3Z3, SEQ ID NO:47; CX1PX2Z3, SEQ ID NO:48, discussed above) into a nucleic acid encoding all or part of a sc- or m-TMAPP;
b) expressing the sulfatase motif-containing polypeptide in a cell that
   i) expresses a FGE and converts the serine or cysteine of the sulfatase motif to a FGly, and partially or completely purifying the FGly-containing polypeptide(s), or
   ii) does not express a FGE that converts a serine or cysteine of the sulfatase motif to a FGly, contacting the purified or partially purified polypeptide(s) with a FGE that converts the serine or cysteine of the sulfatase motif to a FGly; and
c) contacting the FGly-containing polypeptides with an epitope and/or payload that has been functionalized with a group that forms a covalent bond between the aldehyde of the FGly and the epitope and/or payload,
thereby forming a sc- or m-TMAPP-epitope conjugate and/or a sc- or m-TMAPP-molecule (e.g., drug or diagnostic agent) conjugate.

In such a method the epitope and/or payload may be functionalized by any suitable function group that reacts selectively with an aldehyde group. Such groups may, for example, be selected from the group consisting of thiosemicarbazide, aminooxy, hydrazide, and hydrazino. In embodiments, epitope and or payload is part of a compound of the hydrazinyl of Formula (III), (IV), (V), (Va), (VI), or (VIa). In one such embodiment the sulfatase motif is incorporated into a sc-TMAPP or m-TMAPP MHC Class II β1 polypeptide or a linker attached thereto (e.g., within 10, 20, 30, 40, 50, 60, 70, 80, 90, or 100 aa of the N-terminus). In an embodiment a sulfatase motif is incorporated into a sequence having at least 85% (e.g., at least 90%, 95%, 98% or 99%, or even 100%) amino acid sequence identity to a sequence shown in any of FIG. 7A-J, 8A-8C, 9, 10, 12, 14, 16, 19A-19B or 20A-20B before the addition of the sulfatase motif sequence.

In another embodiment, a method of preparing a sc-TMAPP or m-TMAPP conjugate comprises incorporating a sulfatase motif (e.g., SEQ ID NO:45 (Formula (I)) or SEQ ID NO:46 (Formula (II)) into an IgFc region. In an embodiment a sulfatase motif is incorporated into a sequence having at least 85% (e.g., at least 90%, 95%, 98% or 99%, or even 100%) amino acid sequence identity to a sequence shown in FIGS. 21A-21G, before the addition of the sulfatase motif sequence.

Sortase A Enzyme Sites

Epitopes and other molecules (e.g., drugs and/or diagnostic agents) may be attached at the N- and/or C-termini of the polypeptide(s) of a sc-TMAPP or m-TMAPP by incorporating sites for Sortase A conjugation at those locations.

Sortase A recognizes a C-terminal pentapeptide sequence LP(X5)TG/A (SEQ ID NO:54, with X5 being any single amino acid, and G/A being a glycine or alanine), and creates an amide bond between the threonine within the sequence and glycine or alanine in the N-terminus of the conjugation partner. Advantageously, the recognition sequences can be incorporated into either conjugation partner, permitting either the amino or carboxyl terminus of the sc-TMAPP or m-TMAPP polypeptide to serve as a chemical conjugation site. Further, the LP(X5)TG/A sequence does not require any non-natural amino acids, allowing expression to be carried out under a wide variety of conditions in diverse cell types. A potential disadvantage of Sortase A enzymatic ligation is that it employs bacterial transglutaminases (mTGs) that can also catalyze the coupling of glutamine side chains to alkyl primary amines, such as lysine. Bacterial mTGs appear unable to modify glutamine residues in native IgG1 but may result in secondary modifications of the polypeptide sequences when employed.

For attachment of epitopes or other molecules (e.g., drugs and/or diagnostic agents) to the carboxy terminus of a sc- or m-TMAPP, a LP(X5)TG/A is engineered into the carboxy terminal portion of the desired peptide(s). An exposed stretch of glycines or alanines (e.g., (G)₃₋₅ (SEQ ID NOs:55 and 56) when using Sortase A from Staphylococcus aureus or (A)₃₋₅ (SEQ ID NOs:57 and 58) when using Sortase A from Streptococcus pyogenes) is engineered into the N-terminus of a peptide that comprises an epitope (or a linker attached thereto), a peptide payload (or a linker attached thereto), or a peptide covalently attached to a non-peptide epitope or payload (e.g., a drug or diagnostic agent).

For attachment of epitopes or other molecules (e.g., drugs and diagnostic agents) to the amino terminus of a sc- or m-TMAPP, an exposed stretch of glycines (e.g., (G)_{2, 3, 4, or 5}) or alanines (e.g., (A)_{2, 3, 4, or 5}) is engineered to appear at the N-terminus of the desired polypeptide(s), and a LP(X5)TG/A is engineered into the carboxy terminal portion of a peptide that comprises an epitope (or a linker attached thereto), a peptide payload (or a linker attached thereto), or a peptide covalently attached to a non-peptide epitope or payload.

Combining Sortase A with the amino and carboxy engineered peptides results in a cleavage between the Thr and Gly/Ala residues in the LP(X5)TG/A sequence, forming a thioester intermediate with the carboxy labeled peptide. Nucleophilic attack by the N-terminally modified polypeptide results in the formation of a covalently coupled complex of the form: carboxy-modified polypeptide-LP(X5)T*G/A-amino-modified polypeptide, where the “*” represents the bond formed between the threonine of the LP(X5)TG/A motif and the glycine or alanine of the N-terminal modified peptide. In view of the foregoing, this disclosure contemplates compositions containing and the use of sc- or m-TMAPPs having:
at least one LPXTG/A amino acid sequence at the carboxy terminus of a TMAPP or an epitope polypeptide disclosed herein;
at least one oligoglycine (e.g., (G)_{2, 3, 4 or 5}) at the amino terminus of a TMAPP or an epitope polypeptide disclosed herein;
at least one oligo alanine (e.g., (A)_{2, 3, 4 or 5}) at the amino terminus of a TMAPP or an epitope polypeptide disclosed herein;
at least one LP(X5)TA (e.g., LPETA, SEQ ID NO:54 where X5 is E and the last position is A) amino acid sequence of a TMAPP or an epitope polypeptide disclosed herein; and/or
at least one LP(X5)TG (e.g., LPETG, SEQ ID NO:54 where X5 is E and the last position is G) amino acid sequence of a TMAPP or an epitope polypeptide disclosed herein.

In place of LP(X5)TG/A, a LPETGG (SEQ ID NO:59) peptide may be used for S. aureus Sortase A coupling, or a LPETAA (SEQ ID NO:60) peptide may be used for S. pyogenes Sortase A coupling. The conjugation reaction is still between the threonine and the amino terminal oligoglycine or oligoalanine peptide to yield a carboxy-modified polypeptide-LP(X5)T*G/A-amino-modified polypeptide, where the “*” represents the bond formed between the threonine and the glycine or alanine of the N-terminal modified peptide.

In one embodiment, a MHC Class II β1 polypeptide contains an oligoglycine (e.g., (G)_{2, 3, 4 or 5}) or an oligoalanine (e.g., (A)_{2, 3, 4 or 5}) at the N-terminus of the polypeptide, or at the N-terminus of a polypeptide linker attached to it. The oligoglycine or oligoalanine may be used as a Sortase A chemical conjugation site to form a sc- or m-TMAPP-epitope conjugate by conjugating it with an epitope comprising a polypeptide bearing a LP(X5)TG/A in its carboxy terminal region.

Where a polypeptide bearing an oligoglycine at its N-terminus is prepared by expression in a cell based system, and the initial methionine is not removed or not completely removed, a thrombin cleavage site (Leu-Val-Pro-Arg-Gly, SEQ ID NO:61) may be inserted to precede the glycine. As thrombin cleaves between the Arg and Gly residues, it ensures that upon cleavage the glycines are exposed on the protein molecule to be labeled, provided there are no other thrombin sites in the polypeptide.

Transglutaminase Enzyme Sites

Transglutaminases (mTGs) catalyze the formation of a covalent bond between the amide group on the side chain of a glutamine residue and a primary amine donor (e.g., a primary alkyl amine, such as is found on the side chain of a lysine residue in a polypeptide). Transglutaminases may be employed to conjugate epitopes and other molecules (e.g., drugs and/or diagnostic agents) to a peptide of a sc- or m-TMAPP, either directly or indirectly via a linker comprising a free primary amine. As such, glutamine residue present in the polypeptide(s) of a sc-TMMP or m-TMAPP may be considered as chemical conjugation sites when they can be accessed by enzymes such as Streptoverticillium mobaraense transglutaminase. That enzyme (EC 2.3.2.13) is a stable, calcium-independent enzyme catalyzing the γ-acyl transfer of glutamine to the ε-amino group of lysine. Glutamine residues appearing in a sequence are, however, not always accessible for enzymatic modification. The limited accessibility can be advantageous as it limits the number of locations where modification may occur. For example, bacterial mTGs are generally unable to modify glutamine residues in native IgG1s; however, Schibli and co-workers (Jeger, S., et al. Angew Chem (Int Engl). 2010; 49:99957 and Dennler P, et al. Bioconjug Chem. 2014; 25(3):569-78) found that deglycosylating IgG1s at N297 rendered glutamine residue N295 accessible and permitted enzymatic ligation to create an antibody drug conjugate. Further, by producing a N297 to Q297 IgG1 mutant, they introduce two sites for enzymatic labeling by transglutaminase.

Accordingly, where a polypeptide of a sc-TMMP or m-TMAPP does not contain a glutamine that may be employed as a chemical conjugation site (e.g., it is not accessible to a transglutaminase or not placed in the desired location), a glutamine residue, a sequence comprising an accessible glutamine that can act as a substrate of a transglutaminase (sometimes referred to as a “glutamine tag” or a “Q-tag”) may be incorporated into the polypeptide. The added glutamine or Q-tag may act as a chemical conjugation site for covalently attaching an epitope to form a sc- or m-TMAPP-epitope conjugate. Alternatively, the added glutamine or Q-tag may be used to form a sc-TMMP or m-TMAPP conjugate with other molecules (e.g., drugs and/or diagnostic agents). US Pat. Pub. No. 2017/0043033 A1 describes the incorporation of glutamine residues and Q-tags and the use of transglutaminase for modifying polypeptides, and is incorporated herein for those teachings.

Incorporation of glutamine residues and Q-tags may be accomplished chemically where the peptide is synthesized, or by modifying a nucleic acid that encodes the polypeptide and expressing the modified nucleic acid in a cell or cell free system.

In an embodiment, where a chemical conjugation site is a glutamine or Q-tag, the glutamine or Q-tag may be at any of the locations indicated for locating a chemical conjugation site in a sc-TMMP or m-TMAPP described above.

In an embodiment, the added glutamine residue or Q-tag is attached to (e.g., at the N- or C-terminus), or within, the sequence of the MHC Class II β1 polypeptide of a sc-TMMP or m-TMAPP or, if present, a linker attached to it. Additionally, chemical conjugation sites may be present (attached to or within) any location on the polypeptide(s) of a sc- or m-TMAPP. In an embodiment an added glutamine or Q-tag is incorporated within 20, 15, or 10 amino acids of the N-terminus of the MHC Class II β1 polypeptide of a sc- or m-TMAPP, including the sequences set forth in FIG. 7A-J, 8A-8C, 9, 10, 12, 14, 16, 19A-19B or 20A-20B as discussed above, including sequence variation. In an embodiment, the added glutamine residue or Q-tag is attached to (e.g., at the N- or C-terminus), or within, the sequence of a sc-TMMP or m-TMAPP of the present disclosure, such as a MHC Class II α1, α2, β1 or β2 polypeptide or, if present, a Fc or other non-Ig scaffold peptide, or linker attached directly or indirectly to any of the foregoing. In an embodiment, the glutamine or Q-tag is present within a polypeptide linker or in a Fc polypeptide.

In embodiments, the glutamine-containing tag comprises an amino acid sequence selected from the group consisting of LQG, LLQGG (SEQ ID NO:62), LLQG (SEQ ID NO:63), LSLSQG (SEQ ID NO:64), and LLQLQG (SEQ ID NO:65) (numerous others are available).

Other molecules (e.g., drugs and/or diagnostic agents) and epitopes that contain, or have been modified to contain, a primary amine group may be used as the amine donor in a transglutaminase catalyzed reaction forming a covalent bond between a glutamine residue (e.g., a glutamine residue in a Q-tag) and the epitope or payload.

Where an epitope or payload does not comprise a suitable primary amine to permit it to act as the amine donor, the epitope or payload may be chemically modified to incorporate an amine group (e.g., modified to incorporate a primary amine by linkage to a lysine, aminocaproic acid, cadaverine etc.). Where an epitope or payload comprises a peptide and requires a primary amine to act as the amine donor, a lysine, or other amine containing compounds that a primary amine with a transglutaminase can act on, may be incorporated into the peptide. Other amine containing compounds that may provide a primary amine group and that may be incorporated into, or at the end of, an alpha amino acid chain include, but are not limited to, homolysine, 2,7-diaminoheptanoic acid, aminoheptanoic acid. Alternatively, the epitope or payload may be attached to a peptide or non-peptide linker that comprises a suitable amine group. Examples of suitable non-peptide linkers include an alkyl linker and a PEG (polyethylene glycol) linker.

Transglutaminase can be obtained from a variety of sources, and include enzymes from: mammalian liver (e.g., guinea pig liver); fungi (e.g., Oomycetes, Actinomycetes, Saccharomyces, Candida, Cryptococcus, Monascus, or Rhizopus transglutaminases); myxomycetes (e.g., Physarum polycephalum transglutaminase); and/or bacteria (e.g., Streptoverticillium mobarensis, Streptoverticillium griseocarneum, Streptoverticillium ladakanum, Streptomyces mobarensis, Streptomyces viridis, Streptomyces ladakanum, Streptomyces caniferus, Streptomyces platensis, Streptomyces hygroscopius, Streptomyces netropsis, Streptomyces fradiae, Streptomyces roseovertivillatus, Streptomyces cinnamaoneous, Streptomyces griseocarneum, Streptomyces lavendulae, Streptomyces lividans, Streptomyces lydicus, S. mobarensis, Streptomyces sioyansis, Actinomadura sp., Bacillus circulars, Bacillus subtilis, Coryneba cterium ammoniagenes, Corynebacterium glutamicum, Clostridium, Enterobacter sp., Micrococcus). In some embodiments, the transglutaminase is a calcium independent transglutaminase which does not require calcium to induce enzyme conformational changes and allow enzyme activity.

As discussed above, a glutamine or Q-tag may be incorporated into any desired location in a polypeptide of a sc- or m-TMAPP. In an embodiment, a glutamine or Q-tag may be added at or near the N-terminus of a MHC Class II β1 polypeptide or to a polypeptide linker attached to the N-terminus of a MHC Class II polypeptide of a sc-TMMP or m-TMAPP described herein.

Selenocysteine and Non-Natural Amino Acids as Chemical Conjugation Sites

One strategy for providing site-specific chemical conjugation sites in the sc- and m-TMAPPs employs the insertion of amino acids with reactivity distinct from the other amino acids present in the polypeptide. Such amino acids include, but are not limited to, the non-natural amino acids acetylphenylalanine (p-acetyl-L-phenylalanine, pAcPhe), parazido phenylalanine, and propynyl-tyrosine, and the naturally occurring amino acid, selenocysteine (Sec).

Thanos et al in US Pat. Publication No. 20140051836 A1 discuss some other non-natural amino acids including O-methyl-L-tyrosine, L-3-(2-naphthyl)alanine, a 3-methyl-phenylalanine, an O-4-allyl-L-tyrosine, a 4-propyl-L-tyrosine, a tri-O-acetyl-GlcNAcβ-serine, L-Dopa, a fluorinated phenylalanine, an isopropyl-L-phenylalanine, a p-acyl-L-phenylalanine, a p-benzoyl-L-phenylalanine, L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine. Other non-natural amino acids include reactive groups including amino, carboxy, acetyl, hydrazino, hydrazido, semicarbazido, sulfanyl, azido and alkynyl. See, e.g., US Pat. Publication No. 20140046030 A1

In addition to directly synthesizing polypeptides in the laboratory, two methods utilizing stop codons have been developed to incorporate non-natural amino acids into proteins and polypeptides utilizing transcription-translation systems. The first incorporates selenocysteine (Sec) by pairing the opal stop codon, UGA, with a Sec insertion sequence. The second incorporates non-natural amino acids into a polypeptide generally through the use of amber, ochrecodon, or opal stop codons. The use of other types of codons such as a unique codon, a rare codon, an unnatural codon, a five-base codon, and a four-base codon, and the use of nonsense and frameshift suppression have also been reported. See, e.g., US Pat. Publication No. 20140046030 A1 and Rodriguez et al., PNAS 103(23)8650-8655(2006). By way of example, the non-natural amino acid acetylphenylalanine may be incorporated at an amber codon using a tRNA/aminoacyl tRNA synthetase pair in an in vivo or cell free transcription-translation system.

Incorporation of both selenocysteine and non-natural amino acids requires engineering the necessary stop codon(s) into nucleic acid coding sequence of the sc- and m-TMAPPs at the desired location(s), after which the coding sequence is used to express the polypeptide(s) in an in vivo or cell free transcription-translation system.

In vivo systems generally rely on engineered cell-lines to incorporate non-natural amino acids that act as bio-orthogonal chemical conjugation sites into polypeptides and proteins. See, e.g., International Published Application No. 2002/085923 entitled “In vivo incorporation of unnatural amino acids.” In vivo non-natural amino acid incorporation relies on a tRNA and an aminoacyl tRNA synthetase (aaRS) pair that is orthogonal to all the endogenous tRNAs and synthetases in the host cell. The non-natural amino acid of choice is supplemented to the media during fermentation, making cell-permeability and stability important considerations.

Various cell-free synthesis systems provided with the charged tRNA may also be utilized to incorporate non-natural amino acids. Such systems include those described in US Published Pat. Application No. 20160115487A1; Gubens et al., RNA. 2010 August; 16(8): 1660-1672; Kim, D M. and Swartz, J. R. Biotechnol. Bioeng. 66:180-8 (1999); Kim, D M. and Swartz, J. R. Biotechnol. Prog. 16:385-90 (2000); Kim, D M. and Swartz, J. R. Biotechnol. Bioeng. 74:309-16 (2001); Swartz et al, Methods Mol. Biol. 267:169-82 (2004); Kim, D M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

Once selenocysteines and non-natural amino acids are incorporated into a sc- or m-TMAPP(s) as chemical conjugation sites, epitopes and/or other molecules (e.g., drugs and diagnostic agents) bearing groups reactive with the selenocysteine or non-natural amino acid are brought into contact with the selenocysteines and non-natural amino of the TMAPP under suitable conditions to form a covalent bond. By way of example, the keto group of the pAcPhe is reactive towards alkoxy-amines and, via oxime coupling, can be conjugated directly to alkoxyamine containing epitopes and/or other molecules (e.g., drugs and diagnostic agents), or indirectly to epitopes and other molecules (e.g., drugs and diagnostic agents) via an alkoxyamine containing linker. Selenocysteine reacts with, for example, primary alkyl iodides (e.g., iodoacetamide which can be used as a linker), maleimides, and methylsulfone phenyloxadiazole groups. Accordingly, epitopes and/or other molecules (e.g., drugs and/or diagnostic agents) bearing those groups or bound to linkers bearing those groups can be covalently bound to polypeptide chains bearing selenocysteines.

As discussed above for other chemical conjugation sites, selenocysteines and/or non-natural amino acids may be incorporated into any desired location in the sc- and m-TMAPPs. In an embodiment, selenocysteines and/or non-natural amino acids may be added at or near the terminus of any element in the sc- and m-TMAPPs, such as the MHC Class II α1, α2, β1 or β2 polypeptide or, if present, a Fc or other non-Ig scaffold peptide, or linker attached directly or indirectly to any of the foregoing. In embodiments selenocysteines and/or non-natural amino acids may be incorporated into or at the amino terminus of a MHC Class II β1 polypeptide or a linker attached at the N-terminus of that polypeptide for the conjugation of epitope polypeptides and/or other molecules.

In addition to linkers associated with the sc- and m-TMAPPs, which when added by protein expression will be polypeptide linker, linkers may be attached to the epitopes or other molecules (e.g., drugs and/or diagnostic agents). Linkers attached to the epitopes of other molecules may include, in addition to amino acid sequences, chemical linkers including, but not limited to, polyethylene oxide, polyethylene glycol and the like.

In an embodiment, sc- and m-TMAPPs contain at least one selenocysteine and/or non-natural amino acid to be used as a chemical conjugation site engineered into a sc- or m-TMAPP. In an embodiment, the sc- and m-TMAPPs contain at least one selenocysteine and/or non-natural amino acid to be used as a chemical conjugation site engineered into a MHC Class II β1 polypeptide sequence in any one of FIG. 7A-J, 8A-8C, 9, 10, 12, 14, 16, 19A-19B or 20A-20B as described above (including sequences with variations thereof). In another embodiment, selenocysteines and/or non-natural amino acids may be incorporated into any IgFc region present as chemical conjugation sites. In one such embodiment, sites in the FC region may be utilized as sites for the conjugation of epitopes and/or other molecules (e.g., drugs and/or diagnostic agents), which may be conjugated to the sites either directly or indirectly through a peptide or chemical linker.

Engineered Amino Acid Chemical Conjugation Sites

Any of the variety of functionalities (e.g., —SH, —NH₃, —OH, —COOH, and the like) present in the side chains of naturally occurring amino acids, or at the termini of polypeptides can be used as chemical conjugation sites. This includes the side chains of lysine and cysteine which are readily modifiable by reagents including N-hydroxysuccinimide and maleimide functionalities respectively. The main disadvantages of utilizing such amino acid residues is the potential variability and heterogeneity of the products. For example, an IgG has over 80 lysines, with over 20 at solvent-accessible sites. See e.g., McComb and Owen AAPS J. 117(2): 339-351. Cysteines tend to be less widely distributed; they tend to be engaged in disulfide bonds and may be inaccessible and not located where it is desirable to place a chemical conjugation site. Accordingly, it is possible to engineer sc- and m-TMAPPs to incorporate non-naturally occurring amino acids within the desired locations for selective modification of the sc- or m-TMAPPs. Engineering may take the form of direct chemical synthesis of the polypeptides, or the epitopes or other molecules to be conjugated. Chemical synthesis may employ the coupling of appropriately blocked amino acids. Alternatively, engineering may take the form of modifying the sequence of a nucleic acid encoding the polypeptide and expressing it in a cell or cell free system. Accordingly, the specification includes and provides for the preparation of a sc- or m-TMAPP polypeptide by transcription/translation and joining to the C- or N-terminus the translated polypeptide an engineered polypeptide bearing a non-natural or natural (including selenocysteine) amino acid to be used as a chemical conjugation site (e.g., for epitopes or peptides). The engineered peptide may be joined by any suitable method, including the use of a sortase as described for epitope peptides above, and may include a linker peptide sequence. In an embodiment the engineered peptide may comprise a sequence of 2, 3, 4, or 5 alanines or glycines that may serve for sortase conjugation and/or as part of a linker sequence.

In one embodiment, the sc- and m-TMAPPs contain at least one naturally occurring amino acid to be used as a chemical conjugation site engineered into a MHC Class II β1 polypeptide sequence in any one of FIG. 7A-J, 8A-8C, 9, 10, 12, 14, 16, 19A-19B or 20A-20B as described above (including sequences with variations thereof).

Any method known in the art may be used to couple payloads or epitopes to amino acids engineered to incorporate amino acids in sc- and m-TMAPPs. By way of example, maleimides may be utilized to couple to sulfhydryls, N-hydroxysuccinimids may be utilized to couple to amine groups, acid anhydrides or chlorides may be used to couple to alcohols or amines, and dehydrating agents may be used to couple alcohols or amines to carboxylic acid groups. Accordingly, by using such chemistry, an epitope or other molecule may be coupled directly or indirectly through a linker (e.g., a homo- or hetero-bifunctional crosslinker) to a location on a sc- or m-TMAPP. By way of example, an epitope peptide (or a peptide-containing payload), including a maleimide amino acid can be conjugated to a sulfhydryl of a chemical conjugation site (e.g., a cysteine residue) naturally occurring or engineered into a TMAPP. Using a Diels-Alder/retro-Diels-Alder protecting scheme, it is possible to directly incorporate maleimide amino acid into a peptide (e.g., an epitope peptide) using solid phase peptide synthesis techniques. See, e.g., Koehler, Kenneth Christopher, “Development and Implementation of Clickable Amino Acids” (2012). Chemical & Biological Engineering Graduate Theses & Dissertations. 31. https://scholar.colorado.edu/chbe_gradetds/31. Accordingly, in one embodiment an epitope peptide comprises a maleimide amino acid that is coupled to a cysteine present in the binding pocket of a sc- or m-TMAPP.

A pair of sulfhydryl groups may be employed simultaneously to create a chemical conjugate to sc- and m-TMAPPs. In such an embodiment a TMAPP that has a disulfide bond, or has two cysteines (or selenocysteines) engineered into locations proximate to each other, may be utilized as a chemical conjugation site through the use of bis-thiol linkers. Bis-thiol linkers, described by Godwin and co-workers, avoid the instability associated with reducing disulfide bonds by forming a bridging group in its place and at the same time permitting the incorporation of another molecule, which can be an epitope or payload. See, e.g., the article by Badescu G, et al., Bioconjug Chem. 2014; 25(6):1124-36 entitled “Bridging disulfides for stable and defined antibody drug conjugates,” describing the use of bis-sulfone reagents, which incorporate a hydrophilic linker (e.g., PEG (polyethyleneglycol) linker) for attachment of epitopes and other molecules (e.g., drugs and/or diagnostic agents).

Where a sc-TMAPP or a m-TMAPP comprises a disulfide bond, the bis-thiol linker may be used to incorporate an epitope or payload by reducing the bond, generally with stoichiometric or near stoichiometric amounts of dithiol reducing agents (e.g., dithiothreitol) and allowing the linker to react with both cysteine residues. Where multiple disulfide bonds are present, the use of stoichiometric or near stoichiometric amounts of reducing agents may allow for selective modification at one site. See, e.g., Brocchini, et al., Adv. Drug. Delivery Rev. (2008) 60: 3-12. Where polypeptides of a sc-TMAPP or m-TMAPP do not comprise a pair of cysteines and/or selenocysteines (e.g., a cysteine and selenocysteine pair), they may be engineered into the polypeptide (by introducing one or both of the cysteines or selenocysteines) to provide a pair of residues that can interact with a bis-thiol linker. The cysteines and/or selenocysteines should be located such that a bis-thiol linker can bridge them (e.g., at a location where two cysteines could form a disulfide bond). Any combination of cysteines and selenocysteines may be employed (i.e. two cysteines, two selenocysteines, or a selenocysteine and a cysteine). The cysteines and/or selenocysteines may both be on a single polypeptide. Alternatively, the cysteines and/or selenocysteines for reaction with a bis-thiol linker may be present on different polypeptides of a m-TMAPP.

In an embodiment a pair of cysteines and/or selenocysteines are incorporated into a MHC Class II β1 polypeptide sequence having at least 85% (e.g., at least 90%, 95%, 98% or 99%, or even 100%) amino acid sequence identity to a MHC Class II β1 polypeptide sequence in any one of FIG. 7A-J, 8A-8C, 9, 10, 12, 14, 16, 19A-19B or 20A-20B as described above. In one such embodiment the pair of cysteines and/or selenocysteines may be utilized as a bis-thiol linker coupling site for the conjugation of, for example, epitopes and/or other molecules (e.g., drugs and/or diagnostic agents) either directly or indirectly through a peptide or chemical linker (bis-thiol linkers may incorporate linkers such as PEG which improves their solubility in water). In one embodiment, the pair of cysteines and/or selenocysteines is located within 10, 20, 30, 40 or 50 amino acids of the amino terminus of a sc- or m-TMAPP.

In another embodiment, a pair of cysteines and/or selenocysteines are incorporated into the IgFC or a non-immunoglobulin scaffold polypeptide of a sc- or m-TMAPP. In one such embodiment, the pair of cysteines and/or selenocysteines may be utilized as a bis-thiol linker coupling site for the conjugation of, for example, epitopes and/or other molecules (e.g., drugs and/or diagnostic agents) either directly or indirectly through a peptide or chemical linker.

Other Chemical Conjugation Sites

Carbohydrate Chemical Conjugation Sites

Many proteins prepared by cellular expression contain added carbohydrates (e.g., oligosaccharides of the type added to antibodies expressed in mammalian cells). Accordingly, where sc- or m-TMAPPs are prepared by cellular expression of their polypeptides, carbohydrates may be present and available as site selective chemical conjugation sites in glycol-conjugation reactions. McCombs and Owen, AAPS Journal, (2015) 17(2): 339-351, and references cited therein describe the use of carbohydrate residues for glycol-conjugation of molecules to antibodies.

The addition and modification of carbohydrate residues may also be conducted ex vivo, through the use of chemicals that alter the carbohydrates (e.g., periodate, which introduces aldehyde groups), or by the action of enzymes (e.g., fucosyltransferases) that can incorporate chemically reactive carbohydrates or carbohydrate analogs for use as chemical conjugation sites.

In an embodiment, the incorporation of an IgFc scaffold with known glycosylation sites may be used to introduce site specific chemical conjugation sites into a sc- or m-TMAPP.

This disclosure includes and provides for sc- or m-TMAPPs having carbohydrates as chemical conjugation (glycol-conjugation) sites. The disclosure also includes and provides for the use of such molecules in forming conjugates with epitopes and with other molecules such as drugs and diagnostic agents, and the use of those molecule in methods of treatment and diagnosis.

Nucleotide Binding Sites

Nucleotide binding sites offer site-specific functionalization through the use of a UV-reactive moiety that can covalently link to the binding site. Bilgicer et al., Bioconjug Chem. 2014; 25(7):1198-202, reported the use of an indole-3-butyric acid (IBA) moiety can be covalently linked to an IgG at a nucleotide binding site. By incorporation of the sequences required to form a nucleotide binding site, chemical conjugates of any TMAPP with suitably modified epitopes and/or other molecules (e.g., drugs or diagnostic agents) bearing a reactive nucleotide may be employed to prepare TMAPP-epitope conjugates.

This disclosure includes and provides for sc- or m-TMAPPs having nucleotide binding sites as chemical conjugation sites. The disclosure also includes and provides for the use of such molecules in forming conjugates with epitopes and with other molecules such as drugs and diagnostic agents, and the use of those molecule in methods of treatment and diagnosis.

Non-Epitope Conjugates

A broad variety of molecules in addition to epitopes may be conjugated to any TMAPP comprising a chemical conjugation site (in addition to any chemical conjugation site for use in forming an epitope conjugate). Furthermore, where TMAPPs multimerize to form higher order species, it may be possible to incorporate monomers conjugated with more than one type of molecule in a multimer. Accordingly, in addition to the epitopes, it is possible to introduce one or more types of non-epitope molecules selected from the group consisting of: therapeutic agents, chemotherapeutic agents, diagnostic agents, labels and the like. It will be apparent that some molecules may fall into more than one category (e.g., a radio label may be useful as a diagnostic and as a therapeutic for selectively irradiating a specific tissue or cell type).

As noted above, various polypeptides of any TMAPP (e.g., a scaffold or Fc polypeptide) can be modified at chemical conjugation sites to incorporate molecules in addition to epitope peptides. In addition to the specific chemistries discussed above for modification of chemical conjugation sites, crosslinking reagents may be employed to attach epitope and non-epitope “other molecules” to sites in any TMAPP. Such crosslinking agents (bifunctional agents) include, but are not limited to, succinimidyl 4-(N-maleimidomethyl)-cyclohexane-1-carboxylate (SMCC), sulfo-SMCC, maleimidobenzoyl-N-hydroxysuccinimide ester (MBS), sulfo-MBS and succinimidyl-iodoacetate.

Some bifunctional linkers for introducing molecules into any TMAPP having a chemical conjugation site, or its epitope conjugate, include cleavable linkers and non-cleavable linkers. In some cases, the linker is a proteolytically cleavable linker. Some suitable proteolytically cleavable linkers comprise an amino acid sequence selected from the group consisting of: a) LEVLFQGP (SEQ ID NO:40); b) ENLYTQS (SEQ ID NO:41); c) DDDDK (SEQ ID NO:42); d) LVPR (SEQ ID NO:43); and e) GSGATNFSLLKQAGDVEENPGP (SEQ ID NO:44). Suitable linkers, particularly for payloads (sometimes called “payload linkers”) include, e.g., peptides (e.g., from 2 to 10 amino acids in length; e.g., 2, 3, 4, 5, 6, 7, 8, 9, or 10 amino acids in length), alkyl chains, poly(ethylene glycol), disulfide groups, thioether groups, acid labile groups, photolabile groups, peptidase labile groups, and esterase labile groups. Non-limiting examples of suitable linkers are: N-succinimidyl-RN-maleimidopropionamido)-tetraethyleneglycoflester (NHS-PEG4-maleimide); N-succinimidyl 4-(2-pyridyldithio)butanoate (SPDB); disuccinimidyl suberate (DSS); disuccinimidyl glutarate (DGS); dimethyl adipimidate (DMA); N-succinimidyl 4-(2-pyridyldithio)2-sulfobutanoate (sulfo-SPDB); N-succinimidyl 4-(2-pyridyldithio) pentanoate (SPP); N-succinimidyl-4-(N-maleimidomethyl)-cyclohexane-1-carboxy-(6-amidocaproate) (LC-SMCC); K-maleimidoundecanoic acid N-succinimidyl ester (KMUA); γ-maleimide butyric acid N-succinimidyl ester (GMBS); ε-maleimidocaproic acid N-hydroxysuccinimide ester (EMCS); m-maleimide benzoyl-N-hydroxysuccinimide ester (MBS); N-(α-maleimidoacetoxy)-succinimide ester (AMAS); succinimidyl-6-(β-maleimidopropionamide)hexanoate (SMPH); N-succinimidyl 4-(p-maleimidophenyl)butyrate (SMPB); N-(p-maleimidophenyl)isocyanate (PMPI); N-succinimidyl 4(2-pyridylthio)pentanoate (SPP); N-succinimidyl(4-iodo-acetyl)aminobenzoate (SIAB); 6-maleimidocaproyl (MC); maleimidopropanoyl (MP); p-aminobenzyloxycarbonyl (PAB); N-succinimidyl 4-(maleimidomethyl)cyclohexanecarboxylate (SMCC); succinimidyl 3-(2-pyridyldithio)propionate (SPDP); PEG4-SPDP (PEGylated, long-chain SPDP crosslinker); BS(PEG)₅ (PEGylated bis(sulfosuccinimidyl)suberate); BS(PEG)₉ (PEGylated bis(sulfosuccinimidyl)suberate); maleimide-PEG₆-succinimidyl ester; maleimide-PEG₈-succinimidyl ester; maleimide-PEG₁₂-succinimidyl ester; PEG₄-SPDP (PEGylated, long-chain SPDP crosslinker); PEG₁₂-SPDP (PEGylated, long-chain SPDP crosslinker); N-succinimidyl-4-(N-maleimidomethyl)-cyclohexane-1-carboxy-(6-amidocaproate), a “long chain” analog of SMCC (LC-SMCC); 3-maleimidopropanoic acid N-succinimidyl ester (BMPS); N-succinimidyl iodoacetate (SIA); N-succinimidyl bromoacetate (SBA); and N-succinimidyl 3-(bromoacetamido)propionate (SBAP).

Control of the stoichiometry of the reaction may result in some selective modification where engineered sites with chemistry orthogonal to other groups in the molecule are not utilized. Reagents that display far more selectivity, such as the bis-thio linkers discussed above tend to permit more precise control of the location and stoichiometry than reagents that react with single lysine, or cysteine residues.

In embodiments where a any TMAPP of the present disclosure comprises an Fc polypeptide, the Fc polypeptide can comprise one or more covalently attached molecules of payload attached directly or indirectly through a linker. By way of example, where a sc- or a m-TMAPP (comprising a MOD or MOD-less) comprises a Fc polypeptide, the polypeptide chain comprising the Fc polypeptide can be of the formula (A)-(L)-(C), where (A) is the polypeptide chain comprising the Fc polypeptide; where (L), if present, is a linker; and where (C) is a payload (e.g., a cytotoxic agent). (L), if present, links (A) to (C). In some cases, the polypeptide chain comprising the Fc polypeptide can comprise more than one molecule of payload (e.g., cytotoxic agent), for example 2, 3, 4, 5, or more than 5, molecules of payload).

In an embodiment, the non-epitope molecules (e.g., a payload) conjugated to any TMAPP are selected from the group consisting of: biologically active agents or drugs, diagnostic agents or labels, nucleotide or nucleoside analogs, nucleic acids or synthetic nucleic acids (e.g., antisense nucleic acids, small interfering RNA, double stranded (ds)DNA, single stranded (ss)DNA, ssRNA, dsRNA), toxins, liposomes (e.g., incorporating a chemotherapeutic such as 5-fluorodeoxyuridine), nanoparticles (e.g., gold or other metal bearing nucleic acids or other molecules, lipids, particle bearing nucleic acids or other molecules), and combinations thereof.

In an embodiment, the non-epitope molecules conjugated to any TMAPP are selected from the group consisting of: biologically active agents or drugs selected independently from the group consisting of: therapeutic agents (e.g., drug or prodrug), chemotherapeutic agents, cytotoxic agents, antibiotics, antivirals, cell cycle synchronizing agents, ligands for cell surface receptor(s), immunomodulatory agents (e.g., immunosuppressants such as cyclosporine), pro-apoptotic agents, anti-angiogenic agents, cytokines, chemokines, growth factors, proteins or polypeptides, antibodies or an antigen binding fragment thereof, enzymes, proenzymes, hormones and combinations thereof.

In an embodiment the non-epitope molecules conjugated to any TMAPP are selected from the group consisting of: biologically active agents or drugs selected independently from the group consisting of: therapeutic diagnostic agents or labels, selected independently from the group consisting of photodetectable labels (e.g., dyes, fluorescent labels, phosphorescent labels, luminescent labels), contrast agents (e.g., iodine or barium containing materials), radiolabels, imaging agents, paramagnetic labels/imaging agents (gadolinium containing magnetic resonance imaging labels), ultrasound labels and combinations thereof.

Drug Conjugates—Therapeutic Agents and Chemotherapeutic Agents

A polypeptide chain of any TMAPP of the present disclosure may comprise a small molecule drug or any other therapeutic or chemotherapeutic agent conjugated (covalently bound) to the polypeptide chain as a payload. For example, where any TMAPP of the present disclosure comprises a Fc polypeptide, the Fc polypeptide can comprise a covalently linked small molecule drug. In some cases, the small molecule drug is a cancer chemotherapeutic agent, e.g., a cytotoxic agent. A polypeptide chain of any TMAPP can comprise a cytotoxic agent linked (e.g., covalently attached) to the polypeptide chain. For example, where any TMAPP comprises a Fc polypeptide, the Fc polypeptide can comprise a covalently linked cytotoxic agent. Cytotoxic agents include prodrugs. Direct linkage can involve linkage directly to an amino acid side chain. Indirect linkage can be linkage via a linker.

Suitable therapeutic agents include, e.g., rapamycin, retinoids, such as all-trans retinoic acid (ATRA); vitamin D3; a vitamin D3 analog; and the like. As noted above, in some cases, a drug is a cytotoxic agent. Cytotoxic agents are known in the art. A suitable cytotoxic agent can be any compound that results in the death of a cell, or induces cell death, or in some manner decreases cell viability, and includes, for example, maytansinoids and maytansinoid analogs, benzodiazepines, taxoids, CC-1065 and CC-1065 analogs, duocarmycins and duocarmycin analogs, enediynes, such as calicheamicins, dolastatin and dolastatin analogs including auristatins, tomaymycin derivatives, leptomycin derivatives, methotrexate, cisplatin, carboplatin, daunorubicin, doxorubicin, vincristine, vinblastine, melphalan, mitomycin C, chlorambucil and morpholino doxorubicin.

For example, in some cases, the cytotoxic agent is a compound that inhibits microtubule formation in eukaryotic cells. Such agents include, e.g., maytansinoid, benzodiazepine, taxoid, CC-1065, duocarmycin, a duocarmycin analog, calicheamicin, dolastatin, a dolastatin analog, auristatin, tomaymycin, and leptomycin, or a pro-drug of any one of the foregoing. Maytansinoid compounds include, e.g., N(2′)-deacetyl-N(2′)-(3-mercapto-1-oxopropyl)-maytansine (DM1); N(2′)-deacetyl-N(2′)-(4-mercapto-1-oxopentyl)-maytansine (DM3); and N(2)-deacetyl-N2-(4-mercapto-4-methyl-1-oxopentyl)-maytansine (DM4). Benzodiazepines include, e.g., indolinobenzodiazepines and oxazolidinobenzodiazepines.

Cytotoxic agents include taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; maytansine or an analog or derivative thereof; an auristatin or a functional peptide analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite; 6 mercaptopurine; 6 thioguanine; cytarabine; fludarabin; 5 fluorouracil; decarbazine; hydroxyurea; asparaginase; gemcitabine; cladribine; an alkylating agent; a platinum derivative; duocarmycin A; duocarmycin SA; rachelmycin (CC-1065) or an analog or derivative thereof; an antibiotic; pyrrolo[2,1-c][1,4]-benzodiazepines (PDB); diphtheria toxin; ricin toxin; cholera toxin; a Shiga-like toxin; LT toxin; C3 toxin; Shiga toxin; pertussis toxin; tetanus toxin; soybean Bowman-Birk protease inhibitor; Pseudomonas exotoxin; alorin; saporin; modeccin; gelanin; abrin A chain; modeccin A chain; alpha-sarcin; Aleurites fordii proteins; dianthin proteins; Phytolacca americana proteins; Momordica charantia inhibitor; curcin; crotin; Sapaonaria officinalis inhibitor; gelonin; mitogellin; restrictocin; phenomycin; enomycin toxins; ribonuclease (RNase); DNase I; Staphylococcal enterotoxin A; pokeweed antiviral protein; diphtherin toxin; and Pseudomonas endotoxin.

Diagnostic Agents and Labels

Any TMAPP can be conjugated to one or more independently selected molecules of a photodetectable label (e.g., dyes, fluorescent labels, phosphorescent labels, luminescent labels), contrast agents (e.g., iodine or barium containing materials), radiolabels, imaging agents, spin labels, Forster Resonance Energy Transfer (FRET)-type labels, paramagnetic labels/imaging agents (e.g., gadolinium containing magnetic resonance imaging labels), ultrasound labels and combinations thereof.

In some embodiments, the conjugate moiety comprises a label that is or includes radioisotope. Examples of a radioisotope or other labels include, but are not limited to, ³H, ¹¹C, ¹⁴C, ¹⁵N, ³⁵S, ¹⁸F, ³²P, ³³P, ⁶⁴Cu ⁶⁸Ga, ⁹⁰Y, ⁹⁹Tc, ¹²³I, ¹²⁴I, ¹²⁵I, ¹¹³I, ¹¹¹In, ¹³¹In, ¹⁵³Sm, ¹⁸⁶Re, ¹⁸⁸Re, ²¹¹At, ²¹²Bi, and ¹⁵³Pb.

Nucleic Acids

The present disclosure provides nucleic acids comprising a nucleotide sequence encoding any unconjugated TMAPP comprising one or more chemical conjugation sites (e.g., MOD-containing or MOD-less sc-TMAPPs or m-TMAPPs, comprising one or more chemical conjugation sites).

Nucleic Acids Encoding Sc-TMAPPs of the Present Disclosure

As described above, in some cases, a sc-TMAPP of the present disclosure comprises a single polypeptide chain and may have chemical conjugation sites that may be utilized, for example, to incorporate a payload, epitope, or MOD and epitope. The present disclosure provides a nucleic acid comprising a nucleotide sequence encoding unconjugated sc-TMAPP comprising one or more chemical conjugation sites (including unconjugated sc-TMAPPs comprising a MOD or that are MOD-less).

Nucleic Acid(s) Encoding m-TMAPPs of the Present Disclosure

As described above, in some cases, a m-TMAPP of the present disclosure comprises at least 2 separate polypeptide chains, one or more of which may have chemical conjugation sites that may be utilized, for example, to incorporate a payload, epitope, or MOD and epitope. The present disclosure provides nucleic acids comprising nucleotide sequences encoding unconjugated m-TMAPP comprising one or more chemical conjugation sites (including unconjugated m-TMAPP comprising a MOD or that are MOD-less). In some cases, the individual polypeptide chains of a m-TMAPP are encoded in separate nucleic acids. In some cases, all polypeptide chains of a m-TMAPP are encoded in a single nucleic acid. In some cases, a first nucleic acid comprises a nucleotide sequence encoding a first polypeptide of a m-TMAPP; and a second nucleic acid comprises a nucleotide sequence encoding a second polypeptide of a m-TMAPP. In some cases, a single nucleic acid comprises a nucleotide sequence encoding a first polypeptide of m-TMAPP and a second polypeptide of a m-TMAPP. Regardless of the number of nucleic acids encoding the multimeric polypeptide, at least one, if not two or more, comprises a sequence encoding at least one chemical conjugation site.

Separate Nucleic Acids Encoding Individual Polypeptide Chains of a m-TMAPP

The present disclosure provides nucleic acids comprising nucleotide sequences encoding any TMAPP comprising one or more chemical conjugation sites of the present disclosure. As noted above, in some cases, the individual polypeptide chains of a m-TMAPP (which may comprise a MOD or be MOD-less) are encoded in separate nucleic acids. In some cases, nucleotide sequences encoding the separate polypeptide chains of an unconjugated m-TMAPP are operably linked to transcriptional control elements, e.g., promoters, such as promoters that are functional in a eukaryotic cell, where the promoter can be a constitutive promoter or an inducible promoter.

For example, the present disclosure provides a first nucleic acid and a second nucleic acid, where the first nucleic acid comprises a nucleotide sequence encoding the first polypeptide of a m-TMAPP, and where the second nucleic acid comprises a nucleotide sequence encoding the second polypeptide of the m-TMAPP; wherein at least one of the sequences encoding the first polypeptide and the second polypeptide comprises a sequence encoding a chemical conjugation site. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

As one non-limiting example, the present disclosure provides a first nucleic acid and a second nucleic acid, where the first nucleic acid comprises a nucleotide sequence encoding a first polypeptide of a m-TMAPP, where the first polypeptide comprises, in order from N-terminus to C-terminus: a) an optional linker; b) a first MHC Class II polypeptide; and c) a MOD (e.g., a reduced-affinity variant, as described above); and where the second nucleic acid comprises a nucleotide sequence encoding the second polypeptide of the m-TMAPP, where the second polypeptide comprises, in order from N-terminus to C-terminus: a) a second MHC Class II polypeptide; and b) an Ig Fc polypeptide; wherein at least one of the sequences encoding the first polypeptide and the second polypeptide comprises a sequence encoding a chemical conjugation site. Suitable linkers, MHC polypeptides, immunomodulatory polypeptides, and Ig Fc polypeptides, are described above. In some cases, the nucleotide sequences encoding the first and second polypeptides are operably linked to transcriptional control elements. In some cases, the transcriptional control element is a promoter that is functional in a eukaryotic cell. In some cases, the nucleic acids are present in separate expression vectors.

Nucleic Acid Encoding Two or More Polypeptides Present in a m-TMAPP

The present disclosure provides a nucleic acid comprising nucleotide sequences encoding at least the first polypeptide and the second polypeptide of a m-TMAPP (which may comprise a MOD or be MOD-less); wherein at least one of the sequences encoding the first polypeptide and the second polypeptide comprises a sequence encoding a chemical conjugation site. In some cases, where a m-TMAPP includes a first, second, and third polypeptide, the nucleic acid includes a nucleotide sequence encoding the first, second, and optionally third polypeptides; wherein at least one of the sequences encoding the first polypeptide, second polypeptide, and third polypeptide comprises a sequence encoding a chemical conjugation site. In some cases, the nucleotide sequences encoding the first polypeptide and the second polypeptide include a proteolytically cleavable linker interposed between the nucleotide sequence encoding the first polypeptide and the nucleotide sequence encoding the second polypeptide. In some cases, the nucleotide sequences encoding the first polypeptide and the second polypeptide include an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide include a ribosome skipping signal (or cis-acting hydrolase element, CHYSEL) interposed between the nucleotide sequence encoding the first polypeptide and the nucleotide sequence encoding the second polypeptide. In an embodiment where a proteolytically cleavable linker is provided between nucleotide sequences encoding the first polypeptide and the second polypeptide, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a MOD-containing or MOD-less m-TMAPP; and a second nucleic acid (e.g., a recombinant expression vector, an mRNA, a viral RNA, etc.) comprises a nucleotide sequence encoding a second polypeptide chain of a m-TMAPP. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

Recombinant Expression Vectors

The present disclosure provides recombinant expression vectors comprising nucleic acids of the present disclosure. In some cases, the recombinant expression vector is a non-viral vector. In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors include, but are not limited to, viral vectors (e.g., viral vectors based on vaccinia virus; poliovirus; adenovirus) (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544).

In some cases, a nucleotide sequence encoding an unconjugated TMAPP (which may comprise a MOD or be MOD-less) is operably linked to a control element, e.g., a transcriptional control element, such as a promoter. The transcriptional control element may be functional in either a eukaryotic cell, e.g., a mammalian cell; or a prokaryotic cell (e.g., a bacterial or archaeal cell). In some cases, a nucleotide sequence encoding a DNA-targeting RNA and/or a site-directed modifying polypeptide is operably linked to multiple control elements that allow expression of the nucleotide sequence encoding a DNA-targeting RNA and/or a site-directed modifying polypeptide in both prokaryotic and eukaryotic cells.

Non-limiting examples of suitable eukaryotic promoters (promoters functional in a eukaryotic cell) include those from cytomegalovirus (CMV) immediate early genes, herpes simplex virus (HSV), thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

Genetically Modified Host Cells

The present disclosure provides a genetically modified host cell, where the host cell is genetically modified with a nucleic acid(s) of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT/cells, mouse L cells (ATCC No. CCLI.3), human embryonic kidney (HEK) cells (ATCC No. CRL1573), HLHepG2 cells, and the like.

Genetically modified host cells can be used to produce unconjugated TMAPP of the present disclosure. For example, a genetically modified host cell can be used to produce unconjugated sc-TMAPPs or m-TMAPPs of the present disclosure. In an embodiment, production of unconjugated sc- and m-TMAPPs is accomplished by introducing one or more expression vector(s) comprising nucleotide sequences encoding the polypeptides of the unconjugated sc- or m-TMAPPs into a host cell, generating a genetically modified host cell, which genetically modified host cell produces the unconjugated sc- or m-TMAPPs.

Compositions

The present disclosure provides compositions, including pharmaceutical compositions, comprising any TMAPP of the present disclosure, such as a sc- or m-TMAPP-epitope conjugate. The present disclosure provides compositions, including pharmaceutical compositions, comprising a nucleic acid or a recombinant expression vector of the present disclosure.

Compositions Comprising a TMAPP

A composition of the present disclosure can comprise, in addition to any TMAPP of the present disclosure (e.g., a sc- or m-TMAPP-epitope conjugate that has a MOD or is MOD-less) one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy,” 19^{th} Ed. (1995), or latest edition, Mack Publishing Co; A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise: i) any TMAPP of the present disclosure (e.g., a sc- or m-TMAPP-epitope conjugate that has a MOD or is MOD-less); and ii) a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium, saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

The formulations and compositions of the present disclosure may also include surfactants. The use of surfactants in drug products, formulations and emulsions is well known in the art. Surfactants and their uses are further described in U.S. Pat. No. 6,287,860.

For example, compositions may include aqueous solutions, powders, granules, tablets, pills, suppositories, capsules, suspensions, sprays, and the like. The composition may be formulated according to the various routes of administration described below.

Where any TMAPP (e.g., a MOD-containing or MOD-less sc- or m-TMAPP-epitope conjugate) of the present disclosure is administered as an injectable (e.g., subcutaneously, intraperitoneally, intramuscularly, intralymphatically, and/or intravenously) directly into a tissue, a formulation can be provided as a ready-to-use dosage form, or as a non-aqueous form (e.g., a reconstitutable storage-stable powder) or aqueous form, such as liquid composed of pharmaceutically acceptable carriers and excipients. The protein-containing formulations may also be provided so as to enhance serum half-life of the subject protein following administration. For example, the protein may be provided in a liposome formulation, or prepared as a colloid or by using other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

In some cases, a composition of the present disclosure comprises: a) any TMAPP (e.g., a MOD-containing or MOD-less sc- or m-TMAPP-epitope conjugate); and b) saline (e.g., 0.9% NaCl). In some cases, the composition is sterile. In some cases, the composition is suitable for administration to a human subject, e.g., where the composition is sterile and is free of detectable pyrogens and/or other toxins. Thus, the present disclosure provides a composition comprising: a) any TMAPP (e.g., a MOD-containing or MOD-less sc- or m-TMAPP-epitope conjugate); and b) saline (e.g., 0.9% NaCl), where the composition is sterile and is free of detectable pyrogens and/or other toxins.

Other examples of formulations suitable for parenteral administration include isotonic sterile injection solutions, anti-oxidants, bacteriostats, and solutes that render the formulation isotonic with the blood of the intended recipient, suspending agents, solubilizers, thickening agents, stabilizers, and preservatives. For example, a subject pharmaceutical composition can be presented in a container, e.g., a sterile container, such as a syringe. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The concentration of any TMAPP (e.g., a MOD-containing or MOD-less sc- or m-TMAPP-epitope conjugate) in a formulation can vary widely (e.g., from less than about 0.1%, usually at or at least about 2% to as much as 20% to 50% or more by weight) and will usually be selected primarily based on fluid volumes, viscosities, and patient-based factors in accordance with the particular mode of administration selected and the patient's needs.

The present disclosure provides a container comprising a composition of the present disclosure, e.g., a liquid composition. The container can be, e.g., a syringe, an ampoule, and the like. In some cases, the container is sterile. In some cases, both the container and the composition are sterile.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers, surfactants and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

Compostions Comprising a Nucleic Acid or a Recombinant Expression Vector

The present disclosure provides compositions, e.g., pharmaceutical compositions, comprising a nucleic acid or a recombinant expression vector of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A composition of the present disclosure can include: a) one or more nucleic acids or one or more recombinant expression vectors comprising nucleotide sequences encoding an unconjugated sc- or m-TMAPP (that can be MOD-containing or MOD-less) of the present disclosure; and b) one or more of: a buffer, a surfactant, an antioxidant, a hydrophilic polymer, a dextrin, a chelating agent, a suspending agent, a solubilizer, a thickening agent, a stabilizer, a bacteriostatic agent, a wetting agent, and a preservative. Suitable buffers include, but are not limited to, N,N-bis(2-hydroxyethyl)-2-aminoethanesulfonic acid (BES), bis(2-hydroxyethyl)amino-tris(hydroxymethyl)methane (BIS-Tris), N-(2-hydroxyethyl)piperazine-N′3-propanesulfonic acid (EPPS or HEPPS), glycylglycine, N-2-hydroxyehtylpiperazine-N′-2-ethanesulfonic acid (HEPES), 3-(N-morpholino)propane sulfonic acid (MOPS), piperazine-N,N′-bis(2-ethane-sulfonic acid) (PIPES), sodium bicarbonate, 3-(N-tris(hydroxymethyl)-methyl-amino)-2-hydroxy-propanesulfonic acid) TAPSO, (N-tris(hydroxymethyl)methyl-2-aminoethanesulfonic acid (TES), N-tris(hydroxymethyl)methyl-glycine (Tricine), tris(hydroxymethyl)-aminomethane (Tris). Suitable salts include, e.g., NaCl, MgCl₂, KCl, MgSO₄, etc.

A pharmaceutical formulation of the present disclosure can include a nucleic acid or recombinant expression vector of the present disclosure in an amount of from about 0.001% to about 90% (w/w). In the description of formulations, below, “subject nucleic acid or recombinant expression vector” will be understood to include a nucleic acid or recombinant expression vector of the present disclosure. For example, in some embodiments, a subject formulation comprises a nucleic acid or recombinant expression vector of the present disclosure.

A subject nucleic acid or recombinant expression vector can be admixed, encapsulated, conjugated or otherwise associated with other compounds or mixtures of compounds; such compounds can include, e.g., liposomes or receptor-targeted molecules. A subject nucleic acid or recombinant expression vector can be combined in a formulation with one or more components that assist in uptake, distribution and/or absorption.

A subject nucleic acid or recombinant expression vector composition can be formulated into any of many possible dosage forms such as, but not limited to, tablets, capsules, gel capsules, liquid syrups, soft gels, suppositories, and enemas. A subject nucleic acid or recombinant expression vector composition can also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

A formulation comprising a subject nucleic acid or recombinant expression vector can be a liposomal formulation. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes that can interact with negatively charged DNA molecules to form a stable complex. Liposomes that are pH sensitive or negatively charged are believed to entrap DNA rather than complex with it. Both cationic and noncationic liposomes can be used to deliver a subject nucleic acid or recombinant expression vector.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein by reference in its entirety.

The formulations and compositions of the present disclosure may also include surfactants. The use of surfactants in drug products, formulations and emulsions is well known in the art. Surfactants and their uses are further described in U.S. Pat. No. 6,287,860.

In one embodiment, various penetration enhancers are included, to effect the efficient delivery of nucleic acids. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein by reference in its entirety.

Compositions and formulations for oral administration include powders or granules, microparticulates, nanoparticulates, suspensions or solutions in water or non-aqueous media, capsules, gel capsules, sachets, tablets, or minitablets. Thickeners, flavoring agents, diluents, emulsifiers, dispersing aids or binders may be desirable. Suitable oral formulations include those in which a subject antisense nucleic acid is administered in conjunction with one or more penetration enhancers, surfactants, and chelators. Suitable surfactants include, but are not limited to, fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. An exemplary suitable combination is the sodium salt of lauric acid, capric acid, and UDCA. Further penetration enhancers include, but are not limited to, polyoxyethylene-9-lauryl ether, and polyoxyethylene-20-cetyl ether. Suitable penetration enhancers also include propylene glycol, dimethylsulfoxide, triethanoiamine, N,N-dimethylacetamide, N,N-dimethylformamide, 2-pyrrolidone and derivatives thereof, tetrahydrofurfuryl alcohol, and AZONE™.

Methods

The present disclosure provides for the use of any TMAPP of the present disclosure for various research and diagnostic purposes. For example, any TMAPP (e.g., a MOD-containing or MOD-less sc- or m-TMAPP-epitope conjugate) of the present disclosure can be used to label, directly or indirectly, an antigen-specific T-cell.

Any TMAPP (e.g., a MOD-containing or MOD-less sc- or m-TMAPP-epitope conjugate) of the present disclosure is useful for modulating an activity of a T-cell. Thus, the present disclosure provides methods of modulating an activity of a T-cell, the methods generally involving contacting a target T-cell with a TMAPP-epitope conjugate of the present disclosure.

Methods of Detecting an Antigen-Specific T-Cell

The present disclosure provides methods of detecting an antigen-specific T-cell. The methods comprise contacting a T-cell with a sc- or m-TMAPP-epitope conjugate (which has a MODs or is MOD-less); and detecting binding of the epitope conjugate to the T-cell.

The present disclosure provides a method of detecting an antigen-specific T-cell, the method comprising contacting a sc- or m-TMAPP-epitope conjugate (which has a MODs or is MOD-less); and detecting specific binding; wherein binding of the TMAPP-epitope conjugate to the T-cell indicates that the T-cell is specific for the epitope present in the TMAPP-epitope conjugate.

Any TMAPP (e.g., a sc- or m-TMAPP-epitope conjugate that has a MOD or is MOD-less) may comprise a detectable label, that can be used in detecting binding of a TMAPP. Suitable detectable labels include, but are not limited to, a radioisotope, a fluorescent polypeptide, an enzyme that generates a fluorescent product, and an enzyme that generates a colored product. Where a sc- or m-TMAPP-epitope conjugate (which has a MODs or is MOD-less) comprises a detectable label, binding of those TMAPP to, for example, a T-cell is detected by detecting the detectable label.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of a sc- or m-TMAPP-epitope conjugate (which has a MODs or is MOD-less) to the T-cell is detected using a detectably labeled antibody specific for the epitope conjugate. An antibody specific for the epitope conjugate can comprise a detectable label such as a radioisotope, a fluorescent polypeptide, an enzyme that generates a fluorescent product, or an enzyme that generates a colored product.

In some cases, the T-cell being detected is present in a sample comprising a plurality of T-cells. For example, a T-cell being detected can be present in a sample comprising from 10 to 10⁹ T-cells, e.g., from 10 to 10², from 10² to 10⁴, from 10⁴ to 10⁶, from 10⁶ to 10⁷, from 10⁷ to 10⁸, from 10⁸ to 10⁹, or more than 10⁹, T-cells.

Methods of Modulating T-Cell Activity

The present disclosure provides a method of selectively modulating the activity of an epitope-specific T-cell, the method comprising contacting the T-cell with a TMAPP-epitope conjugate (e.g., a sc- or m-TMAPP-epitope conjugate, either of which has a MOD or is MOD-less), where contacting the T-cell with the epitope conjugate selectively modulates the activity of the epitope-specific T-cell. In some cases, the contacting occurs in vitro. In some cases, the contacting occurs in vivo. In some cases, the contacting occurs ex vivo.

In some cases, the T-cell being contacted with a TMAPP-epitope conjugate (e.g., a sc- or m-TMAPP-epitope conjugate, either of which has a MOD or is MOD-less) is a regulatory T-cell (Treg). Suitable Tregs include CD4⁺, FOXP3⁺, and CD25⁺. Tregs can suppress autoreactive T-cells. In some cases, a method of the present disclosure activates Tregs, thereby reducing autoreactive T-cell activity.

The present disclosure provides a method of increasing proliferation of Tregs, the method comprising contacting Tregs with a TMAPP-epitope conjugate (e.g., a sc- or m-TMAPP-epitope conjugate, either of which has a MOD or is MOD-less), where the contacting increases proliferation of Tregs. The present disclosure provides a method of increasing the number of Tregs in an individual, the method comprising administering to the individual a TMAPP-epitope conjugate (e.g., a sc- or m-TMAPP-epitope conjugate, either of which has a MOD or is MOD-less), where the administering results in an increase in the number of Tregs in the individual. For example, the number of Tregs can be increased by at least 5%, at least 10%, at least 15%, at least 20%, at least 25%, at least 30%, at least 40%, at least 50%, at least 60%, at least 70%, at least 80%, at least 90%, at least 2-fold, at least 2.5-fold, at least 5-fold, at least 10-fold, or more than 10-fold.

In some cases, the cell being contacted is a helper T-cell, where contacting the helper T-cell with a TMAPP-epitope conjugate (e.g., a sc- or m-TMAPP-epitope conjugate, either of which has a MOD or is MOD-less) results in activation of the helper T-cell. In some cases, activation of the helper T-cell results in an increase in the activity and/or number of CD8⁺ cytotoxic T-cells, e.g., CD8⁺ cytotoxic T-cells that target and kill a cancer cell.

Treatment Methods

The present disclosure provides a treatment method for selectively modulating the activity of an epitope-specific T-cell in an individual, the method comprising administering to the individual an amount of any TMAPP of the present disclosure, such as a sc- or m-TMAPP-epitope conjugate (e.g., comprising a MOD or MOD-less) of the present disclosure, effective to selectively modulate the activity of an epitope-specific T-cell in an individual, and to treat the individual. In some cases, a treatment method of the present disclosure comprises administering to an individual in need thereof one or more recombinant expression vectors comprising nucleotide sequences encoding a MOD-containing sc- or m-TMAPP (e.g., comprising a MOD) of the present disclosure. In some cases, a treatment method of the present disclosure comprises administering to an individual in need thereof one or more mRNA molecules comprising nucleotide sequences encoding a MOD-containing sc- or m-TMAPP of the present disclosure. In some cases, a treatment method of the present disclosure comprises administering to an individual in need thereof any TMAPP of the present disclosure (e.g., a sc- or m-TMAPP-epitope conjugate that has a MOD or is MOD-less). Conditions that can be treated include cancer and autoimmune disorders.

The present disclosure provides a method of selectively modulating the activity of an epitope-specific T-cell in an individual, the method comprising administering to the individual an effective amount of a TMAPP-epitope conjugate described herein, such as a sc- or m-TMAPP-epitope conjugate (which has a MODs or is MOD-less), where the epitope conjugate selectively modulates the activity of the epitope-specific T-cell in the individual. Selectively modulating the activity of an epitope-specific T-cell can treat a disease or disorder in the individual. Thus, the present disclosure provides a treatment method comprising administering to an individual in need thereof an effective amount of a TMAPP-epitope conjugate (e.g., sc- or m-TMAPP-epitope conjugates that have a MOD or are MOD-less). In some cases, the disease or disorder is an autoimmune disease or disorder. In some cases, the disease or disorder is cancer.

In some cases, the MOD is an activating polypeptide, and the TMAPP-epitope conjugate activates the epitope-specific T-cell. In some cases, the epitope is a cancer-associated epitope, and the TMAPP (e.g., a TMAPP-epitope conjugate comprising a MOD that is an activating polypeptide) increases the activity of a T-cell specific for the cancer-associated epitope including, for example, epitope-specific T-cell proliferation and/or release of any one or more cytokine, chemokine, lymphokine and the like.

The present disclosure provides a method of treating cancer in an individual, the method comprising administering to the individual an effective amount of any TMAPP or any TMAPP-epitope conjugate (e.g., sc- or m-TMAPP-epitope conjugates that have a MOD or are MOD-less) of the present disclosure; where a TMAPP-epitope conjugate employed for such a treatment comprises a T-cell epitope that is a cancer epitope, and comprises a stimulatory MOD. In some cases, an “effective amount” of any TMAPP or any TMAPP-epitope conjugate of the present disclosure is an amount that, when administered in one or more doses to an individual in need thereof, reduces the number of cancer cells in the individual. For example, in some cases, an “effective amount” of any TMAPP or any TMAPP-epitope conjugate of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before the administration, or in the absence of administration of any TMAPP or any TMAPP-epitope conjugate. In some cases, an “effective amount” of any TMAPP or any TMAPP-epitope conjugate of the present disclosure is an amount that, when administered in one or more doses to an individual in need thereof, reduces the number of cancer cells in the individual to undetectable levels. In some cases, an “effective amount” of any TMAPP or any TMAPP-epitope conjugate of the present disclosure is an amount that, when administered in one or more doses to an individual in need thereof, reduces the tumor mass in the individual. For example, in some cases, an “effective amount” of any TMAPP or any TMAPP-epitope conjugate of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before the administration, or in the absence of administration of any TMAPP or any TMAPP-epitope conjugate. In some cases, an “effective amount” of any TMAPP or any TMAPP-epitope conjugate of the present disclosure is an amount that, when administered in one or more doses to an individual in need thereof (an individual having a tumor), reduces the tumor volume in the individual. For example, in some cases, an “effective amount” of any TMAPP or any TMAPP-epitope conjugate of the present disclosure is an amount that, when administered in one or more doses to an individual in need thereof (an individual having a tumor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before the administration, or in the absence of administration of any TMAPP or any TMAPP-epitope conjugate. In some cases, an “effective amount” of any TMAPP or any TMAPP-epitope conjugate of the present disclosure is an amount that, when administered in one or more doses to an individual in need thereof, increases survival time of the individual. For example, in some cases, an “effective amount” of any TMAPP or any TMAPP-epitope conjugate of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of the administration.

In some cases, the MOD is an inhibitory polypeptide, and any MOD-containing TMAPP epitope conjugate inhibits activity of the epitope-specific T-cell. In some cases, the epitope is a self-epitope, and the MOD-containing TMAPP-epitope conjugate selectively inhibits the activity of a T-cell specific for the self-epitope.

The present disclosure provides a method of treating an autoimmune disorder in an individual, the method comprising administering to the individual an effective amount of a TMAPP-epitope conjugate (e.g., a sc- or m-TMAPP-epitope conjugate that has a MOD or is MOD-less) of the present disclosure, where the TMAPP-epitope conjugate comprises a T-cell epitope that is a self epitope, and where the TMAPP-epitope conjugate comprises an inhibitory MOD. In some cases, an “effective amount” of such a TMAPP-self epitope conjugate with an inhibitory MOD is an amount that, when administered in one or more doses to an individual in need thereof, reduces the number of self-reactive T-cells by at least 10%, at least 15%, at least 20%, at least 25%, at least 30%, at least 40%, at least 50%, at least 60%, at least 70%, at least 80%, at least 90%, or at least 95%, compared to the number of self-reactive T-cells in the individual before the administration, or in the absence of administration of the TMAPP-self epitope conjugate with an inhibitory MOD. In some cases, an “effective amount” of a TMAPP-self epitope conjugate with an inhibitory MOD is an amount that, when administered in one or more doses to an individual in need thereof, reduces production of Th2 cytokines in the individual. In some cases, an “effective amount” of a TMAPP-self epitope conjugate with an inhibitory MOD is an amount that, when administered in one or more doses to an individual in need thereof, ameliorates one or more symptoms associated with an autoimmune disease in the individual.

As noted above, in some cases, in carrying out a subject treatment method with any TMAPP of the present disclosure (e.g., a sc- or m-TMAPP-epitope conjugate that has a MOD or is MOD-less), the TMAPP is administered to an individual in need thereof, as the polypeptide per se. In other instances, in carrying out a subject treatment method, one or more nucleic acids comprising nucleotide sequences encoding an unconjugated TMAPP (e.g., an sc- or m-TMAPP that has a MOD or is MOD-less) of the present disclosure is/are administered to an individual in need thereof. Thus, in other instances, one or more nucleic acids of the present disclosure, e.g., one or more recombinant expression vectors of the present disclosure, is/are administered to an individual in need thereof.

Formulations

Suitable formulations are described above, where suitable formulations include a pharmaceutically acceptable excipient. In some cases, a suitable formulation comprises: a) any TMAPP of the present disclosure (e.g., a sc- or m-TMAPP-epitope conjugate that has a MOD or is MOD-less); and b) a pharmaceutically acceptable excipient. Suitable pharmaceutically acceptable excipients are described above. In some cases, a suitable formulation comprises: a) a nucleic acid comprising a nucleotide sequence encoding an unconjugated sc- or m-TMAPP (that has a MOD or is MOD-less); and b) a pharmaceutically acceptable excipient; in some instances, the nucleic acid is an mRNA. In some cases, a suitable formulation comprises: a) a first nucleic acid comprising a nucleotide sequence encoding the first polypeptide of an unconjugated sc- or m-TMAPP (that has a MOD or is MOD-less); b) a second nucleic acid comprising a nucleotide sequence encoding the second polypeptide of a TMAPP of the present disclosure; and c) a pharmaceutically acceptable excipient. In some cases, a suitable formulation comprises: a) a recombinant expression vector comprising a nucleotide sequence encoding an unconjugated sc- or m-TMAPP (that has a MOD or is MOD-less); and b) a pharmaceutically acceptable excipient. In some cases, a suitable formulation comprises: a) a first recombinant expression vector comprising a nucleotide sequence encoding the first polypeptide of a TMAPP of the present disclosure; b) a second recombinant expression vector comprising a nucleotide sequence encoding the second polypeptide of a TMAPP of the present disclosure; and c) a pharmaceutically acceptable excipient.

Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide to be administered, sex of the patient, time, and route of administration, general health, and other drugs being administered concurrently. Any TMAPP of the present disclosure (e.g., a sc- or m-TMAPP-epitope conjugate that has a MOD or is MOD-less) may be administered in amounts between 1 ng/kg body weight and 20 mg/kg body weight per dose, e.g., between 0.1 mg/kg body weight to 10 mg/kg body weight (e.g., between 0.5 mg/kg body weight to 5 mg/kg body weight); however, doses below or above this exemplary range are envisioned, especially considering the aforementioned factors. If the regimen is a continuous infusion, it can also be in the range of 1 μg to 10 mg per kilogram of body weight per minute. A TMAPP-epitope conjugate of the present disclosure can be administered in an amount of from about 1 mg/kg body weight to about 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of any TMAPP of the present disclosure (e.g., a sc- or m-TMAPP-epitope conjugate that has a MOD or is MOD-less) is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMAPP required for maintenance (e.g., a sc- or m-TMAPP-epitope conjugate that has a MOD or is MOD-less) is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific TMAPP, the severity of the symptoms and the susceptibility of the subject to side effects. Preferred dosages for a given compound are readily determinable by those of skill in the art by a variety of means.

In some cases, multiple doses of any TMAPP of the present disclosure, nucleic acid of the present disclosure, or recombinant expression vector of the present disclosure are administered. The frequency of administration of any TMAPP of the present disclosure, nucleic acid of the present disclosure, or recombinant expression vector of the present disclosure can vary depending on any of a variety of factors, e.g., severity of the symptoms, etc. For example, in some embodiments, any TMAPP of the present disclosure, nucleic acid of the present disclosure, or recombinant expression vector of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of any TMAPP of the present disclosure, a nucleic acid of the present disclosure, or a recombinant expression vector of the present disclosure, e.g., the period of time over which any TMAPP of the present disclosure, a nucleic acid of the present disclosure, or a recombinant expression vector of the present disclosure is administered, can vary, depending on any of a variety of factors, e.g., patient response, etc. For example, any TMAPP of the present disclosure, nucleic acid of the present disclosure, or recombinant expression vector of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Routes of Administration

An active agent including any TMAPP of the present disclosure (e.g., a sc- or m-TMAPP-epitope conjugate that has a MOD or is MOD-less), nucleic acid of the present disclosure, or recombinant expression vector of the present disclosure, is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intralymphatic, intracranial, subcutaneous, intradermal, topical application, intravenous, intraarterial, rectal, nasal, oral, and other enteral and parenteral routes of administration. Routes of administration may be combined, if desired, or adjusted depending upon the active agent and the desired effect. Any TMAPP (e.g, a sc- or m-TMAPP-epitope conjugate that has a MOD or is MOD-less) of the present disclosure, or a nucleic acid or recombinant expression vector of the present disclosure, can be administered in a single dose or in multiple doses.

Any TMAPP (e.g, a sc- or m-TMAPP-epitope conjugate that has a MOD or is MOD-less) of the present disclosure, nucleic acid of the present disclosure, or recombinant expression vector of the present disclosure, may be administered intravenously, intramuscularly, locally, intratumorally, peritumorally, intracranially, subcutaneously, and/or intralymphatically.

Any TMAPP of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, intralymphatic, peritumoral, and intravenous routes, i.e., any route of administration other than through the alimentary canal. Parenteral administration can be conducted to effect systemic or local delivery of any TMAPP of the present disclosure. Where systemic delivery is desired, administration typically involves invasive or systemically absorbed topical or mucosal administration of pharmaceutical preparations.

Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment.

Subjects suitable for treatment with a method of the present disclosure include individuals who have an autoimmune disease, including individuals who have been diagnosed as having an autoimmune disease, and individuals who have been treated for an autoimmune disease but who failed to respond to the treatment. Autoimmune diseases that can be treated with a method of the present disclosure include, but are not limited to, celiac disease, multiple sclerosis, rheumatoid arthritis, type I autoimmune diabetes (IDDM), Crohn's disease, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, and Sjogren's syndrome.

### Certain Embodiments

1. A MOD-less m-TMAPP comprising:
a) a first polypeptide comprising:
   i) an optional linker (which may be present or absent); and
   ii) a first major MHC Class II polypeptide (e.g., any one or more of an α1, α2, β1, and/or β2 polypeptide); and
b) a second polypeptide comprising:
   i) a second MHC Class II polypeptide (e.g., any one or more of an α1, α2, β1, and/or β2 polypeptide); and
   ii) an optional linker (which may be present or absent);
wherein the first polypeptide and/or the second polypeptide comprises one or more chemical conjugation sites located
   A) at or near (e.g., within 5, 10, 15, or 20 amino acids) the N-terminus or C-terminus of the first polypeptide,
   B) at or near (e.g., within 5, 10, 15, or 20 amino acids) the N-terminus or C-terminus of the second polypeptide,
   C) within the first or second polypeptide (including any linker therein), and/or
   D) wherein when at least one of the first polypeptides or the second polypeptides further comprises a N- or C-terminal linker (e.g., a polypeptide linker), at a location within, at the N-terminus of the linker, and/or at the C-terminus of the linker; and
wherein the first polypeptide and/or the second polypeptide may further comprise an immunoglobulin (Ig) Fc polypeptide or a non-Ig scaffold (e.g., at the C-terminus of the peptide); wherein the first polypeptide and second polypeptide taken together comprise a MHC Class II α1 polypeptide, a MHC Class II α2 polypeptide, a MHC Class II β1 polypeptide, and a MHC Class II β2 polypeptide.
   2. The MOD-less m-TMAPP of embodiment 1, wherein:
a) the first polypeptide comprises, in order from N-terminus to C-terminus:
   i) the optional linker;
   ii) the MHC Class II α1 polypeptide; and
   iii) the MHC Class II α2 polypeptide; and
b) the second polypeptide comprises, in order from N-terminus to C-terminus:
   i) the MHC Class II β1 polypeptide; and
   ii) the MHC Class II β2 polypeptide.
      3. The MOD-less m-TMAPP of embodiment 1, wherein:
a) the first polypeptide comprises, in order from N-terminus to C-terminus:
   i) the optional linker;
   ii) the MHC Class II β1 polypeptide; and
   iii) the MHC Class II β2 polypeptide; and
b) the second polypeptide comprises, in order from N-terminus to C-terminus:
   i) the MHC Class II α1 polypeptide; and
   ii) the MHC Class II α2 polypeptide.
      4. The MOD-less m-TMAPP of embodiment 1, wherein:
a) the first polypeptide comprises, in order from N-terminus to C-terminus:
   i) the optional linker;
   ii) the MHC Class II β1 polypeptide;
   iii) the MHC Class II α1 polypeptide; and
   iv) the MHC Class II α2 polypeptide; and
b) the second polypeptide comprises the MHC Class II β2 polypeptide.
   5. The MOD-less m-TMAPP of any one of embodiments 1-4, wherein the first polypeptide comprises an Ig Fc polypeptide at the C-terminus.
   6. The MOD-less m-TMAPP of any one of embodiments 1-5, comprising a non-immunoglobin scaffold or an immunoglobulin Fc polypeptide at the C-terminus of the first or second polypeptide.
   7. The MOD-less m-TMAPP of any one of embodiments 1-6, wherein the at least one chemical conjugation site is:
a) at the N-terminus of the first or second polypeptide;
b) within 5, 10, 15, or 20 amino acids of the N-terminus of the first or second polypeptide;
c) at the N-terminus of, or within a linker polypeptide attached to the N-terminus of the first or second polypeptide;
d) at the C-terminus of the first or second polypeptide;
e) within 5, 10, 15, or 20 amino acids of the C-terminus of the first or second polypeptide; or
f) at the C-terminus of, or within, a linker polypeptide attached to the C-terminus of the first or second polypeptide.
   8. The MOD-less m-TMAPP of any one of embodiments 1-7, further comprising an epitope (e.g., a cancer, viral, autoantigen epitope peptide) conjugated to one of the one or more chemical conjugation sites either directly or via a linker to form a MOD-less m-TMAPP-epitope conjugate.
   9. The MOD-less m-TMAPP of embodiment 8, wherein the epitope is conjugated to the linker (e.g., an optional linker) and the linker is between the epitope and the Class II MHC polypeptide (e.g., at the β1 polypeptide N-terminus).
   10. A MOD-less sc-TMAPP polypeptide comprising:
i) a MHC Class II α1 polypeptide;
ii) a Class II MHC α2 polypeptide;
iii) a Class II MHC β1 polypeptide;
iv) an optional Class II MHC β2 polypeptide;
v) an optional linker (which may be present or absent);
vi) optionally an immunoglobulin (Ig) Fc polypeptide or a non-immunoglobulin scaffold; and
vii) one or more chemical conjugation sites;
wherein at least one of the one or more chemical conjugation sites is located
   A) at the N-terminus or C-terminus of polypeptide,
   B) within the polypeptide including any linker therein, and/or
   C) wherein when the polypeptide further comprises a N- or C-terminal linker (e.g., a polypeptide linker), within the linker, at the N-terminus of the linker, and/or at the C-terminus of the linker.
      11. The MOD-less sc-TMAPP of embodiment 10, wherein the polypeptide comprises, in order from N-terminus to C-terminus:
i) the optional linker;
ii) the Class II MHC β1 polypeptide;
iii) the Class II MHC α1 polypeptide;
iv) the Class II MHC α2 polypeptide; and
v) the Class II MHC β2 polypeptide.
   12. The MOD-less sc-TMAPP of embodiment 10, wherein the polypeptide comprises, in order from N-terminus to C-terminus:
i) the optional linker;
ii) the Class II MHC β1 polypeptide;
iii) the Class II MHC β2 polypeptide;
iv) the Class II MHC α1 polypeptide; and
v) the Class II MHC α2 polypeptide.
   13. The MOD-less sc-TMAPP of any one of embodiments 10-12, comprising a non-immunoglobin scaffold or an immunoglobulin Fc polypeptide at the C-terminus of the sc-TMAPP polypeptide.
   14. The MOD-less sc-TMAPP of any one of embodiments 12-13 wherein the at least one chemical conjugation site is:
a) at the N-terminus of the MOD-less sc-TMAPP;
b) within 5, 10, 15, or 20 amino acids of the N-terminus of the MOD-less sc-TMAPP;
c) at the N-terminus of, or within a linker polypeptide attached to the N-terminus of the MOD-less sc-TMAPP;
d) at the C-terminus of the MOD-less sc-TMAPP;
e) within 5, 10, 15, or 20 amino acids of the C-terminus of the MOD-less sc-TMAPP; or
f) at the C-terminus of, or within, a linker polypeptide attached to the C-terminus of MOD-less sc-TMAPP.
   15. The MOD-less sc-TMAPP of any one of embodiments 10-14, further comprising an epitope (e.g., a cancer, viral, autoantigen epitope peptide) conjugated to one of the one or more chemical conjugation sites either directly or via a linker to form a MOD-less sc-TMAPP-epitope conjugate.
   16. The MOD-less sc-TMAPP of embodiment 15, wherein the epitope is conjugated to the linker and the linker is between the epitope and the Class II MHC β1 polypeptide (e.g., at the β1 polypeptide N-terminus).
   17. A MOD-containing m-TMAPP comprising:
a) a first polypeptide comprising:
   i) an optional linker (which may be present or absent); and
   ii) a first major MHC Class II polypeptide (e.g., any one or more of an α1, α2, β1, and/or β2 polypeptide); and
b) a second polypeptide comprising:
   i) a second MHC Class II polypeptide (e.g., any one or more of an α1, α2, β1, and/or β2 polypeptide); and
   ii) an optional linker (which may be present or absent);
wherein the first polypeptide and/or the second polypeptide comprise one or more chemical conjugation sites located
   A) at or near (e.g., within 5, 10, 15, or 20 amino acids) the N-terminus or C-terminus of the first polypeptide,
   B) at or near (e.g., within 5, 10, 15, or 20 amino acids) the N-terminus or C-terminus of the second polypeptide,
   C) within the first or second polypeptide (including any linkers therein), and/or
   D) wherein when at least one of the first polypeptides or the second polypeptides further comprises a N- or C-terminal linker (e.g., a polypeptide linker), at a location within, at the N-terminus of the linker, and/or at the C-terminus of the linker;
wherein the first polypeptide and/or the second polypeptide comprise one or more independently selected wild-type or variant MOD polypeptides;
wherein the first polypeptide and/or the second polypeptide may further comprise an immunoglobulin (Ig) Fc polypeptide or a non-Ig scaffold (e.g., at the C-terminus of the peptide); and
wherein the first polypeptide and second polypeptide taken together comprise a MHC Class II α1 polypeptide, a MHC Class II α2 polypeptide, a MHC Class II polypeptide, and a MHC Class II β2 polypeptide.
(See for example the embodiments in FIG. 22A to FIG. 22L)
   18. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II β2 polypeptide; and
   iv) one or more independently selected wild-type or variant MODs; and
b) the second polypeptide comprises, in order from N-terminus to C-terminus:
   i) the MHC Class II α1 polypeptide;
   ii) the MHC Class II α2 polypeptide.
      19. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II β2 polypeptide; and
   iv) one or more independently selected wild-type or variant MODs; and
b) the second polypeptide comprises, in order from N-terminus to C-terminus:
   i) the MHC Class II α1 polypeptide;
   ii) the MHC Class II α2 polypeptide; and
   iii) an Ig Fc polypeptide.
      20. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II β2 polypeptide;
   iv) one or more independently selected wild-type or variant MODs; and
   v) a first dimerization polypeptide; and
b) the second polypeptide comprises, in order from N-terminus to C-terminus:
   i) the MHC Class II α1 polypeptide;
   ii) the MHC Class II α2 polypeptide; and
   iii) a second dimerization polypeptide.
      21. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II β2 polypeptide; and
b) the second polypeptide comprises, in order from N-terminus to C-terminus:
   i) one or more independently selected wild-type or variant MODs;
   ii) the MHC Class II α1 polypeptide; and
   iii) the MHC Class II α2 polypeptide.
      22. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II β2 polypeptide; and
b) the second polypeptide comprises, in order from N-terminus to C-terminus:
   i) one or more independently selected wild-type or variant MODs;
   ii) the MHC Class II α1 polypeptide;
   iii) the MHC Class II α2 polypeptide; and
   iv) an Ig Fc polypeptide.
      23. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II β2 polypeptide; and
   iv) a first dimerization polypeptide; and
b) the second polypeptide comprises, in order from N-terminus to C-terminus:
   i) one or more independently selected wild-type or variant MODs;
   ii) the MHC Class II α1 polypeptide;
   iii) the MHC Class II α2 polypeptide; and
   iv) a second dimerization polypeptide.
      24. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II α1 polypeptide;
   iv) the MHC Class II α2 polypeptide; and
b) the second polypeptide comprises, in order from N-terminus to C-terminus:
   i) one or more independently selected wild-type or variant MODs; and
   ii) the MHC Class II β2 polypeptide.
      25. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II α1 polypeptide;
   iv) the MHC Class II α2 polypeptide; and
b) the second polypeptide comprises, in order from N-terminus to C-terminus:
   i) one or more independently selected wild-type or variant MODs;
   ii) the MHC Class II β2 polypeptide; and
   iii) an Ig Fc polypeptide.
      26. The MOD-containing m-TMAPP of embodiment 17, wherein:
a) the first polypeptide comprises, in order from N-terminus to C-terminus:
   i) the optional linker;
   ii) the MHC Class II β1 polypeptide;
   iii) the MHC Class II α1 polypeptide;
   iv) the MHC Class II α2 polypeptide; and
   v) a first dimerization polypeptide; and
b) the second polypeptide comprises, in order from N-terminus to C-terminus:
   i) one or more independently selected wild-type or variant MODs;
   ii) the MHC Class II β2 polypeptide; and
   iii) a second dimerization polypeptide.
      27. The MOD-containing m-TMAPP of any one of embodiments 17-26, comprising a non-immunoglobin scaffold or an immunoglobulin Fc polypeptide at the C-terminus of the first or second polypeptide.
      28. The MOD-containing m-TMAPP of any one of embodiments 17-27, wherein the at least one chemical conjugation site is:
a) at the N-terminus of the first or second polypeptide;
b) within 5, 10, 15, or 20 amino acids of the N-terminus of the first or second polypeptide;
c) at the N-terminus of, or within a linker polypeptide attached to the N-terminus of the first or second polypeptide;
d) at the C-terminus of the first or second polypeptide;
e) within 5, 10, 15, or 20 amino acids of the C-terminus of the first or second polypeptide; or
f) at the C-terminus of, or within, a linker polypeptide attached to the C-terminus of the first or second polypeptide.
   29. The MOD-containing m-TMAPP of any one of embodiments 17-28, further comprising an epitope (e.g., a cancer, viral, autoantigen epitope peptide) conjugated to one of the one or more chemical conjugation sites either directly or via a linker to form a MOD-less m-TMAPP-epitope conjugate.
   30. The MOD-less m-TMAPP of embodiment 29, wherein the epitope is conjugated to the linker (e.g., an optional linker) and the linker is between the epitope and the Class II MHC polypeptide (e.g., at the β1 polypeptide N-terminus).
   31. A MOD-containing sc-TMAPP polypeptide comprising:
i) an optional linker (which may be present or absent)
ii) a major histocompatibility complex (MHC) Class II α1 polypeptide;
iii) a MHC Class II α2 polypeptide;
iv) a MHC Class II β1 polypeptide;
v) a MHC Class II β2 polypeptide;
vi) one or more independently selected wild-type or variant MODs;
vii) optionally an immunoglobulin (Ig) Fc polypeptide or a non-immunoglobulin scaffold; and
viii) one or more chemical conjugation sites;
wherein at least one of the one or more chemical conjugation sites is located
   A) at the N-terminus of the MOD-containing sc-TMAPP,
   B) within the MOD-containing sc-TMAPP, or
   C) within the optional linker, at the N-terminus of the linker, or at the C-terminus of the linker when the optional linker is present.
   (See for example the examples in FIG. 23A to FIG. 23H)
      32. The MOD-containing sc-TMAPP of embodiment 31, comprising, in order from N-terminus to C-terminus:
i) the optional linker;
ii) the MHC Class II β1 polypeptide;
iii) the MHC Class II α1 polypeptide;
iv) the MHC Class II α2 polypeptide;
v) the MHC Class II β2 polypeptide; and
vi) the one or more independently selected wild-type or variant MOD.
   33. The MOD-containing sc-TMAPP of embodiment 31, comprising, in order from N-terminus to C-terminus:
i) the optional linker;
ii) a first MOD;
iii) the MHC Class II β1 polypeptide;
iv) the MHC Class II α1 polypeptide;
v) the MHC Class II α2 polypeptide;
vi) the MHC Class II β2 polypeptide; and
vii) a second MOD, wherein the first and the second MODs comprise the same amino acid sequence.
   34. The MOD-containing sc-TMAPP of embodiment 31, comprising, in order from N-terminus to C-terminus:
i) the one or more independently selected wild-type or variant MOD;
ii) the optional linker;
iii) the MHC Class II β1 polypeptide;
iv) the MHC Class II α1 polypeptide;
v) the MHC Class II α2 polypeptide; and
vi) the MHC Class II β2 polypeptide.
   35. The MOD-containing sc-TMAPP of embodiment 31, comprising, in order from N-terminus to C-terminus:
i) the optional linker;
ii) the MHC Class II β1 polypeptide;
iii) the MHC Class II β2 polypeptide;
iv) the MHC Class II α1 polypeptide;
v) the MHC Class II α2 polypeptide; and
vi) the one or more independently selected wild-type or variant MOD.
   36. The MOD-containing sc-TMAPP of embodiment 31, comprising, in order from N-terminus to C-terminus:
i) the optional linker;
ii) the MOD;
iii) the MHC Class II β1 polypeptide;
iv) the MHC Class II β2 polypeptide;
v) the MHC Class II α1 polypeptide; and
vi) the MHC Class II α2 polypeptide.
   37. The MOD-containing sc-TMAPP of embodiment 31, comprising, in order from N-terminus to C-terminus:
i) the one or more independently selected wild-type or variant MOD;
ii) the optional linker;
iii) the MHC Class 11β1 polypeptide;
iv) the MHC Class II β2 polypeptide;
v) the MHC Class II α1 polypeptide; and
vi) the MHC Class II α2 polypeptide.
   38. The MOD-less sc-TMAPP of any one of embodiments 31-37, comprising a non-immunoglobin scaffold or an immunoglobulin Fc polypeptide at the C-terminus of the sc-TMAPP polypeptide.
   39. The MOD-containing sc-TMAPP of any one of embodiments 31-38, wherein the at least one chemical conjugation site is:
a) at the N-terminus of the MOD-containing sc-TMAPP;
b) within 5, 10, 15, or 20 amino acids of the N-terminus of the MOD-containing sc-TMAPP;
c) a the N-terminus of, or within a the optional linker polypeptide attached to the N-terminus of the MOD-containing sc-TMAPP;
d) at the C-terminus of the MOD-containing sc-TMAPP;
e) within 5, 10, 15, or 20 amino acids of the C-terminus of the MOD-containing sc-TMAPP;
f) at the C-terminus of, or within, the optional linker polypeptide attached to the C-terminus of MOD-containing sc-TMAPP;
or
g) within the linker.
   40. The MOD-containing sc-TMAPP of any one of embodiments 31-39, further comprising an epitope (e.g., a cancer, viral, autoantigen epitope peptide) conjugated to one of the one or more chemical conjugation sites either directly or via a linker to form a MOD-containing sc-TMAPP-epitope conjugate.
   41. The MOD-containing sc-TMAPP of embodiment 40, wherein the epitope is conjugated to the linker and the linker is between the epitope and the Class II MHC β1 polypeptide.
   42. The MOD-containing m-TMAPPs of any one of embodiments 17-30 or the MOD-containing sc-TMAPPs of any one of embodiments 31-41, comprising one or more (e.g., 1, 2, or 3) independently selected wild-type and/or variant MODs.
   43. The MOD-containing m-TMAPPs or the MOD-containing sc-TMAPPs of embodiment 42, wherein at least one of the one or more independently selected MODs is a wild-type MOD.
   44. The MOD-containing m-TMAPPs or the MOD-containing sc-TMAPPs of embodiment 43, wherein at least one of the one or more MODs is selected from the group consisting of TGFβ, JAG1, IL-2, CD7, CD80, CD86, PD-L1, PD-L2, 4-1BBL, OX40L, FasL, ICOS-L, ICAM, CD30L, CD40, CD70, CD83, HLA-G, MICA, MICB, HVEM, lymphotoxin beta receptor, 3/TR6, ILT3, ILT4, HVEM, and a fragment of any of the foregoing comprising at least 30, 40, 50, 60 or 70 contiguous amino acids thereof.
   45. The MOD-containing m-TMAPP or the MOD-containing sc-TMAPP of embodiment 43, wherein at least one of the one or more MODs comprises an amino acid sequence of a naturally-occurring MOD selected from:
a 4-1BBL polypeptide of SEQ ID NOs:22, 23, 24, or 25;
a CD80 polypeptide of SEQ ID NO:16;
an IL-2 polypeptide of SEQ ID NO:29;
a CD86 polypeptide of SEQ ID NO:20;
a PD-L1 polypeptide of SEQ ID NO:14 or SEQ ID NO:15; or
or a fragment of any of the foregoing comprising at least 30, 40, 50, 60 or 70 contiguous amino acids of the recited sequences.
   46. The MOD-containing m-TMAPPs or the MOD-containing sc-TMAPPs of embodiment 42, wherein at least one (e.g., 1, 2, or 3) of the one or more independently selected MODs is a variant MOD.
   47. The MOD-containing m-TMAPPs or the MOD-containing sc-TMAPPs of embodiment 46, wherein at least one (e.g., 1, 2, or 3) of the one or more independently selected MODs is a:
variant MOD comprises an amino acid sequence having from 1 to 10 amino acid substitutions, deletions or insertions compared to the amino acid sequence of a naturally-occurring (wild-type) MOD;
variant MOD comprises an amino acid sequence having from 1 to 10 amino acid substitutions, deletions or insertions relative to a polypeptide comprising at least 30, 40, 50, 60 or 70 contiguous amino acids of a naturally-occurring (wild-type) MOD; or
variant MOD comprises an amino acid sequence having at least 90%, 95%, 98%, 99% or 100% amino acid sequence identity to a polypeptide comprising at least 30, 40, 50, 60 or 70 contiguous amino acids of wild-type MOD; and
   wherein the variant MOD has reduced affinity for a Co-MOD, compared to the affinity of the naturally-occurring MOD for the Co-MOD.
   48. The MOD-containing m-TMAPPs or the MOD-containing sc-TMAPPs of embodiment 46 or 47, wherein:
the ratio of: i) the binding affinity of a control MOD-containing m-TMAPP or sc-TMAPP comprising a wild-type MOD to a Co-MOD to ii) the binding affinity of a MOD-containing M-TMAPP or sc-TMAPP comprising a variant of the wild-type MOD to the Co-MOD, when measured by BLI (as described above), is at least 1.5:1, at least 2:1, at least 5:1, at least 10:1, at least 15:1, at least 20:1, at least 25:1, at least 50:1, at least 100:1, at least 500:1, at least 10²:1, at least 5×10²:1, at least 10³:1, at least 5×10³:1, at least 10⁴:1, at least 10⁵:1, or at least 10⁶:1;
or
the ratio of: i) the binding affinity of a control MOD-containing m-TMAPP or sc-TMAPP comprising a wild-type MOD to a Co-MOD to ii) the binding affinity of a MOD-containing m-TMAPP or sc-TMAPP comprising a variant of the wild-type MOD to the Co-MOD, when measured by BLI, is in a range of from 1.5:1 to 10⁶:1, e.g., from 1.5:1 to 10:1, from 10:1 to 50:1, from 50:1 to 10²:1, from 10²:1 to 10³:1, from 10³:1 to 10⁴:1, from 10⁴:1 to 10⁵:1, or from 10⁵:1 to 10⁶:1).
   49. The MOD-containing m-TMAPPs or the MOD-containing sc-TMAPPs of any one of embodiments 46-48, wherein at least one (e.g., 1, 2, or 3) of the one or more independently selected MODs is a variant of a MOD selected from the group consisting of TGFβ, JAG1, IL-2, CD7, CD80, CD86, PD-L1, PD-L2, 4-1BBL, OX40L, FasL, ICOS-L, ICAM, CD30L, CD40, CD70, CD83, HLA-G, MICA, MICB, HVEM, lymphotoxin beta receptor, 3/TR6, ILT3, ILT4, HVEM, and a fragment of any of the foregoing comprising at least 30, 40, 50, 60 or 70 contiguous amino acids thereof.
   50. The MOD-containing m-TMAPP or the MOD-containing sc-TMAPP of embodiment 49, wherein at least one (e.g., 1, 2, or 3) of the one or more independently selected MODs is a variant of a MOD polypeptide sequence selected from:
a 4-1BBL polypeptide of SEQ ID NOs:22, 23, 24 or 25;
a CD80 polypeptide of SEQ ID NO:16;
an IL-2 polypeptide of SEQ ID NO:29;
a CD86 polypeptide of SEQ ID NO:20;
a PD-L1 polypeptide of SEQ ID NO:14 or SEQ ID NO:15; and/or
or a fragment of any of the foregoing comprising at least 30, 40, 50, 60 or 70 contiguous amino acids of the polypeptide.
   51. MOD-containing m-TMAPPs or the MOD-containing sc-TMAPPs of any one of embodiments 46-48, wherein at least one (e.g., 1, 2, or 3) of the one or more independently selected MODs is a variant of a 4-1BBL polypeptide (e.g., SEQ ID NO:22, 23, 24, 25, or 26).
   52. MOD-containing m-TMAPPs or the MOD-containing sc-TMAPPs of any one of embodiments 46-48, wherein at least one (e.g., 1, 2, or 3) of the one or more independently selected MODs is a variant of a IL-2 polypeptide (e.g., SEQ ID NO:29).
   53. MOD-containing m-TMAPPs or the MOD-containing sc-TMAPPs of any one of embodiments 46-48, wherein at least one (e.g., 1, 2, or 3) of the one or more independently selected MODs is a variant of a CD80 polypeptide (e.g., SEQ ID NO:16) or a CD86 polypeptide (e.g., SEQ ID NO:20).
   54. MOD-containing m-TMAPPs or the MOD-containing sc-TMAPPs of any one of embodiments 46-48, wherein at least one (e.g., 1, 2, or 3) of the one or more independently selected MODs is a variant of a PD-L1 polypeptide (e.g., SEQ ID NO:14 or SEQ ID NO:15).
   55. The MOD-containing m-TMAPPs or the MOD-containing sc-TMAPPs of any one of embodiments 42-54, comprising two independently selected wildtype and/or variant MODs.
   56. The MOD-containing m-TMAPPs or the MOD-containing sc-TMAPPs of embodiment 55, wherein the two MODs comprise the same amino acid sequence.
   57. The TMAPPs of any of embodiments 1-56, wherein the MHC Class II α1 polypeptide comprises an amino acid sequence having at least 90%, 95%, 98%, 99% or 100% amino acid sequence identity to either: a MHC Class II α1 polypeptide depicted in any one of FIGS. 6, 11, 13, 15, 17, and 18; or to a polypeptide having at least 30, 40, 50, 60 or 70 contiguous amino acids of any one of the MHC Class II α1 polypeptide depicted in any one of FIGS. 6, 11, 13, 15, 17, and 18.
   58. The TMAPPs of any of embodiments 1-57, wherein the MHC Class II α2 polypeptide comprises an amino acid sequence having at least 90%, 95%, 98%, 99% or 100% amino acid sequence identity to either: a MHC Class II α2 polypeptide depicted in any one of FIGS. 6, 11, 13, 15, 17, and 18; or to a polypeptide having at least 30, 40, 50, 60 or 70 contiguous amino acids of any one of the MHC Class II α2 polypeptide depicted in any one of FIGS. 6, 11, 13, 15, 17, and 18.
   59. The TMAPPs of any of embodiments 1-58, wherein the MHC Class II β1 polypeptide comprises an amino acid sequence having at least 90%, 95%, 98%, 99% or 100% amino acid sequence identity to either: a MHC Class II β1 polypeptide depicted in any one of FIGS. 7A-7J, FIGS. 8A-8B, FIG. 9, FIG. 10, FIG. 12, FIG. 14, FIG. 16, FIGS. 19A-19B, and FIGS. 20A-20B; or to a polypeptide having at least 30, 40, 50, 60 or 70 contiguous amino acids of any one of the MHC Class II β1 polypeptide depicted in any one of FIGS. 7A-7J, FIGS. 8A-8B, FIG. 9, FIG. 10, FIG. 12, FIG. 14, FIG. 16, FIGS. 19A-19B, and FIGS. 20A-20B.
   60. The TMAPPs of any of embodiments 1-59, wherein the MHC Class II β2 polypeptide comprises an amino acid sequence having at least 90%, 95%, 98%, 99% or 100% amino acid sequence identity to either: a MHC Class II β2 polypeptide depicted in any one of FIGS. 7A-7J, FIGS. 8A-8B, FIG. 9, FIG. 10, FIG. 12, FIG. 14, FIG. 16, FIGS. 19A-19B, and FIGS. 20A-20B; or to a polypeptide having at least 30, 40, 50, 60 or 70 contiguous amino acids of any one of the MHC Class II β2 polypeptide depicted in any one of FIGS. 7A-7J, FIGS. 8A-8B, FIG. 9, FIG. 10, FIG. 12, FIG. 14, FIG. 16, FIGS. 19A-19B, and FIGS. 20A-20B.
   61. The TMAPPs of any of embodiments 6, 13, 27, or 38, wherein the immunoglobulin Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide.
   62. The TMAPPs of any of embodiments 6, 13, 27, or 38, wherein the non-immunoglobin scaffold is an albumin, an XTEN (extended recombinant) polypeptide, transferrin, a Fc receptor polypeptide, or elastin-like polypeptide.
   63. The TMAPPs of any of embodiments 1-62, further comprising a dimerization polypeptide (dimerizer polypeptide) as part of any sc-TMAPP polypeptide, a dimerization polypeptide (dimerizer polypeptide) as part of the first and/or second polypeptide of any m-TMAPP; wherein the dimerization polypeptide is selected from the group consisting of collectin family which contain collagen domains consisting of collagen repeats (e.g., ACRP30 or ACRP30-like proteins, coiled-coil domains and leucine-zipper domains.
   64. The TMAPPs of any of embodiments 1-64, comprising at least one linker between:
a) any two adjacent MHC polypeptides;
b) an MHC polypeptide and a non-immunoglobin scaffold or an immunoglobulin Fc polypeptide;
c) in a sc- or m-TMAPP epitope conjugate between the epitope and a MHC, a non-immunoglobin scaffold, or an immunoglobulin Fc polypeptide;
d) when a MOD is present, between the MOD and a MHC, a non-immunoglobin scaffold, or an immunoglobulin Fc polypeptide, and/or
e) when a first MOD and a second MOD are present (e.g., adjacent), between the first and second MODs.
   65. The TMAPP of any of embodiments 1-65, wherein the linker is a peptide and has a length of from 3 aa to 50 aa (e.g., from 3 to 10, from 10 to 20, from 20 to 30, from 30 to 40, or from 40 to 50).
   66. The TMAPP of embodiment 65, wherein the linker comprises a glycine polymer, glycine-serine polymer, glycine-alanine polymer, alanine-serine polymer,
   67. The TMAPP of embodiment 65, wherein the linker comprises a peptide of the sequence (GS)n, (GSGGS)n, (GGGS)n, (GGSG)n, (GGSGG)n, (GSGSG)n, (GSGGG)n, (GGGSG)n (GSSSG)n, (GSSSS)n, and/or (AAAGG)n where n is 1, 2, 3, 4, 5, 6, 7, 8, 9, or 10.
   68. The TMAPP of embodiment 65, wherein the linker peptide further comprises a cysteine.
   69. The TMAPP of embodiment 68, wherein the linker peptide comprises the amino acid sequence GCGASGGGGSGGGGS, GCGGSGGGGSGGGGSGGGGS, or GCGGSGGGGSGGGGS.
   70. The TMAPP of embodiments 1-69, wherein at least one of the one or more chemical conjugation sites is selected from the group consisting of:
a) peptide sequence that acts as an enzyme modification sequence;
b) non-natural amino acids and/or selenocysteines;
c) engineered amino acid chemical conjugation sites;
d) carbohydrate or oligosaccharide covalently bound to either the MOD-containing m-TMAPP, or to the MOD-containing sc-TMAPP; and
e) IgG nucleotide binding sites.
   71. The TMAPP of embodiment 70, wherein at least one of the chemical conjugation sites is an enzyme modification sequence selected from the group consisting of: a sulfatase motif; a Sortase A enzyme site; and a transglutaminase site.
   72. The TMAPP of embodiment 70, wherein at least one of the chemical conjugation sites is a sulfatase motif; a Sortase A enzyme site; and a transglutaminase site.
   73. The TMAPP of embodiment 70, wherein at least one of the chemical conjugation sites is a non-natural amino acid or a selenocysteines.
   74. The TMAPP of embodiment 70, wherein at least one of the chemical conjugation sites is an engineered amino acid chemical conjugation site.
   75. The TMAPP of embodiment 70, wherein at least one of the chemical conjugation sites is a carbohydrate or oligosaccharide which is covalently bound to a carbohydrate or oligosaccharide
   76. The TMAPP of embodiment 70, wherein at least one of the chemical conjugation sites is an IgG nucleotide binding sites.
   77. The TMAPP-epitope conjugates of any of embodiments 8, 9, 15, 16, 29, 30, 40 or 41, wherein the chemical conjugation site to which the epitope is conjugated (covalently bound) is not located at the N-terminus or C-terminus of an amino acid sequence at least 10, at least 20, at least 30, at least 40, at least 50, at least 75, or at least 100 amino acid long having 100% amino acid identity to a portion of any one of:
the MHC Class II α1 or the MHC Class II α2 polypeptides of FIGS. 6, 11, 13, 15, 17 and 18; or
the MHC Class II β1 or the MHC Class II β2 polypeptide comprises FIGS. 7A-7J, FIGS. 8A-8B, FIG. 9, FIG. 10, FIG. 12, FIG. 14, FIG. 16, FIGS. 19A-19B, and FIGS. 20A-20B.
   78. The TMAPP-epitope conjugates of any of embodiments 8, 9, 15, 16, 29, 30, 40 or 41, wherein the chemical conjugation site to which the epitope is conjugated (covalently bound) is not a lysine, cysteine, serine, threonine, arginine, aspartic acid, glutamic acid, asparagine, or glutamine located in a 10, 20, 30, 40, 50, 60, or 70 amino acid long sequence having 100% amino acid identity to a portion of any one of:
the MHC Class II α1 or the MHC Class II α2 polypeptides of FIGS. 6, 11, 13, 15, 17 and 18; or
the MHC Class II β1 or the MHC Class II β2 polypeptide comprises FIGS. 7A-7J, FIGS. 8A-8B, FIG. 9, FIG. 10, FIG. 12, FIG. 14, FIG. 16, FIGS. 19A-19B, and FIGS. 20A-20B.
   79. The TMAPP of embodiments 1-78, further comprising a payload other than an epitope (e.g., an epitope polypeptide) chemically conjugated (covalently bound) to the polypeptide of a sc-TMAPP or the first or second polypeptide of a m-TMAPP (e.g., a MOD-less sc- or m-TMAPP-epitope conjugate, or a MOD-containing sc- or m-TMAPP-epitope conjugate).
   80. The TMAPP of embodiment 79, wherein the payload conjugated to the polypeptide is a therapeutic agent, chemotherapeutic agent, cytotoxic agent, diagnostic agent or label.
   81. The TMAPP of any of embodiments 79-81, comprising an immunoglobulin Fc polypeptide, wherein the payload is conjugated to the Ig Fc polypeptide.
   82. The TMAPP of embodiments 1-82, wherein the epitope is a cancer epitope or a virus epitope. (see e.g., the section of the disclosure titled “Epitope-presenting peptides”)
   83. The TMAPP of embodiments 1-82, wherein the epitope is an auto-epitope. (see e.g., the section of the disclosure titled “Epitope-presenting peptides”)
   84. A composition comprising:
a) the TMAPP of any one of embodiments 1-83; and
b) a buffer or a pharmaceutically acceptable excipient.
   85. A composition comprising:
a) the MOD-containing m-TMAPP or the MOD-containing sc-TMAPP of any one of embodiments 17-83; and
b) saline.
   86. A composition comprising:
a) the MOD-containing m-TMAPP-epitope conjugate, or the MOD-containing sc-TMAPP-epitope conjugate of any of embodiments 29, 30 or 40-83; and
b) a buffer or a pharmaceutically acceptable excipient.
   87. A composition comprising:
a) the MOD-less m-TMAPP-epitope conjugate, or a MOD-less sc-TMAPP-epitope conjugate of any of embodiments 8, 9, 15, 16 or 42-83; and
b) a buffer or a pharmaceutically acceptable excipient
   88. The composition of any one of embodiments 84-87, wherein the buffer or a pharmaceutically acceptable excipient comprises saline (e.g., about 0.9% NaCl).
   89. The composition of any one of embodiments 84-88, wherein the composition is sterile.
   90. A method of selectively modulating the activity of an epitope-specific T-cell, the method comprising:
contacting the T-cell with the MOD-containing sc-TMAPP and/or the MOD-containing m-TMAPP (e.g., a MOD-containing sc-TMAPP having a chemical conjugation site or its epitope conjugate and/or a MOD-containing m-TMAPP having a chemical conjugation site or its epitope conjugate) of any one of embodiments 17-83, or a composition of embodiments 85-86;
wherein said contacting selectively modulates the activity of the epitope-specific T-cell.
   91. The method of embodiment 90, wherein said contacting is in vitro.
   92. The method of embodiment 90, wherein said contacting is in vivo.
   93. The method of any one of embodiments 90-92, wherein the T-cell is a regulatory T-cell (Treg).
   94. The method of embodiment 93, wherein said contacting activates the Treg and reduces activity of an autoreactive T-cell.
   95. The method of any one of embodiments 90-94, wherein the T-cell is a CD4⁺ T helper cell, and wherein said contacting activates the CD4⁺ T-cell.
   96. The method of embodiment 95, wherein said activated CD4⁺ T-cell activates a CD8⁺ T-cell.
   97. The method of embodiment 96, wherein the CD8⁺ T-cell is specific for a cancer epitope presented by the TMAPP.
   98. The method of any one of embodiments 92-98, comprising administering the TMAPP to an individual in need thereof.
   99. The method of embodiment 98, wherein said administering is systemic.
   100. The method of embodiment 98, wherein said administering is local.
   101. The method of embodiment 98, wherein said administering is peritumoral.
   102. The method of embodiment 98, wherein said administering is via intravenous administration.
   103. The method of any one of embodiments 98-102, wherein the individual is a human
   104. The method of any one of embodiments 98-103, wherein the individual has an autoimmune disease.
   105. The method of embodiment of any one of embodiments 98-103, wherein the individual has a cancer (e.g., the epitope is present or absent).
   106. The method of embodiment of any one of embodiments 98-103, wherein the individual has a viral infection (e.g., the epitope is present or absent, and may be a viral epitope).
   107. A treatment method, the method comprising administering to an individual in need thereof an effective amount of a MOD-containing m-TMAPP-epitope conjugate, or a MOD-containing sc-TMAPP-epitope conjugate of any one of embodiments 29-30 or 40-83, wherein said administering treats the individual.
   108 The method of embodiment 107, wherein the individual has cancer, and wherein said administering treats the cancer.
   109. The method of embodiment 107, wherein the individual has an autoimmune disorder, and wherein said administering treats the autoimmune disorder.
   110. The method of any one of embodiments 107-109, wherein said administering is via intravenous administration.
   111. The method of any one of embodiments 107-109, wherein said administering is via local administration.
   112. The method of any one of embodiments 107-109, wherein said administering is via systemic administration.
   113. One or more nucleic acids comprising nucleotide sequences encoding an unconjugated sc-TMAPP polypeptide, or at least one of the first or second polypeptides of an unconjugated m-TMAPP of any one of embodiments 1-83.
   114. One or more recombinant expression vectors comprising the one or more nucleic acids of embodiment 113
   115. A host cell genetically modified with the one or more nucleic acids of embodiment 105 or the one or more recombinant expression vectors of embodiment 114.
   116. The host cell of embodiment 115, wherein the host cell is a eukaryotic cell.
   117. One or more nucleic acids comprising nucleotide sequences encoding an unconjugated sc-TMAPP polypeptide, or at least one of the first or second polypeptides of an unconjugated m-TMAPP of any one of embodiments 1-83.
   118 One or more recombinant expression vectors comprising the one or more nucleic acids of embodiment 117.
   119. A host cell genetically modified with the one or more nucleic acids of embodiment 117 or the one or more recombinant expression vectors of embodiment 118.
   120. The host cell of embodiment 119, wherein the host cell is a eukaryotic cell.
   121. A method of detecting an antigen-specific T-cell, the method comprising contacting a T-cell with the TMAPP-epitope conjugate of any one of embodiments 8, 9, 15, 15 or 16, wherein binding of the TMAPP to the T-cell indicates that the T-cell is specific for the epitope present in the TMAPP.
   122. The method of embodiment 121, wherein the TMAPP comprises a detectable label (e.g., a detectable label covalently attached at one or more chemical conjugation sites or an epitope tag).
   123. The method of embodiment 122, wherein the detectable label is a radioisotope, a fluorescent polypeptide, or an enzyme that generates a fluorescent product, an enzyme that generates a colored product.
   124. The method of embodiment 121 or 122, wherein binding of the TMAPP to the T-cell is detected using a detectably labeled antibody specific for the TMAPP.
   125. The method of any one of embodiments 121-124, wherein the T-cell is present in a sample comprising a plurality of T-cells.
   126. A method of preparing a MOD-containing or MOD-less sc-TMAPP-epitope conjugate, or a MOD-containing or MOD-less m-TMAPP-epitope conjugate comprising:
a) incorporating a nucleotide sequence encoding chemical conjugation site into a nucleic acid encoding the polypeptide of an unconjugated sc-TMAPP polypeptide (which may or may not comprise a MOD), or the first and/or second polypeptide of an m-TMAPP (which may or may not comprise a MOD);
b) introducing the nucleic acid into a cell to express the sc-TMAPP polypeptide, or the first and/or second polypeptide of an m-TMAPP and obtain an unconjugated sc-TMAPP or an unconjugated m-TMAPP, and optionally purify the unconjugated sc-TMAPP or the unconjugated m-TMAPP (partially or completely);
c) where the chemical conjugation site requires enzymatic activation or chemical conversion, activating or converting the chemical conjugation site (e.g., with an enzyme); and
d) contacting the unconjugated sc-TMAPP or the unconjugated m-TMAPP with an epitope (or an epitope with an attached linker) capable capable of undergoing a reaction with the chemical conjugation site under reaction conditions suitable to cause formation of a covalent bond (e.g., in the presence of an enzyme or catalyst) between the chemical conjugation site and the epitope (or the linker attached to the epitope) to produce the MOD-containing or MOD-less sc-TMAPP-epitope conjugate, or the MOD-containing or MOD-less m-TMAPP-epitope conjugate.
   127. The method of embodiment 126, wherein the chemical conjugation site is a sulfatase motif (e.g., a sulfatase motif of Formula (I) or (II) such as X1CX2PX3Z3; CX1PX2Z3).
   128. The method of embodiment 127, wherein the cell:
i) expresses a FGE and converts the serine or cysteine of the sulfatase motif to a FGly, or
ii) does not express a FGE that converts a serine or cysteine of the sulfatase motif to a FGly, and the method further includes contacting the unconjugated sc- or m-TMAPP with a FGE that converts the serine or cysteine of the sulfatase motif to a FGly; and
iii) contacting the FGly-containing polypeptides with an epitope and/or payload that has been functionalized with a group that forms a covalent bond between the aldehyde of the FGly and the epitope and/or payload,
thereby forming a sc- or m-TMAPP-epitope conjugate and/or a sc- or m-TMAPP-molecule (e.g., drug or diagnostic agent) conjugate.

In any of the above-recited embodiments, a linker (e.g., a peptide linker) may be placed between any two recited components of any TMAPP. In the accompanying figures that lines connecting elements of any TMAPP depicted therein represent optional linkers (see e.g., FIG. 22 and FIG. 23).

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-[xxx] are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. An immunomodulatory polypeptide sequence (MOD)-containing multimeric T-Cell modulatory antigen-presenting polypeptide (m-TMAPP) comprising:
a) a first polypeptide comprising:
i) an optional linker; and
ii) a first major histocompatibility complex (MHC) Class II polypeptide; and
b) a second polypeptide comprising:
i) a second MHC Class II polypeptide and
ii) an optional linker;
wherein the first polypeptide and/or the second polypeptide comprise a chemical conjugation site located
A) at the N-terminus or C-terminus of the first polypeptide,
B) at the N-terminus or C-terminus of the second polypeptide,
C) within the first or second polypeptide (including any linkers therein), and/or
D) wherein, when at least one of the first polypeptide or the second polypeptide further comprises an N- or C-terminal linker, within the linker, at the N-terminus of the linker, or at the C-terminus of the linker;
wherein the first polypeptide and/or second polypeptide of the m-TMAPP comprises one or more independently selected wild-type or variant MOD polypeptides,
wherein the first polypeptide and/or second polypeptide optionally comprise an immunoglobulin (Ig) Fc polypeptide or a non-immunoglobulin scaffold polypeptide; and
wherein the first polypeptide and second polypeptide taken together comprise a MHC Class II α1 polypeptide, a MHC Class II α2 polypeptide, a MHC Class II β1 polypeptide, and a MHC Class II β2 polypeptide.

2. A MOD-containing single-chain T-Cell modulatory antigen-presenting polypeptide (sc-TMAPP) comprising:
i) an optional linker capable of being bound by a T-cell receptor (TCR);
ii) a MHC Class II α1 polypeptide;
iii) a MHC Class II α2 polypeptide;
iv) a MHC Class 11β1 polypeptide;
v) a MHC Class II β2 polypeptide;
vi) one or more independently selected wild-type or variant MOD polypeptides;
vii) optionally an immunoglobulin (Ig) Fc polypeptide or a non-Ig scaffold; and
viii) a chemical conjugation site;
wherein the chemical conjugation site is
A) at the N-terminus of the MOD-containing sc-TMAPP,
B) within the MOD-containing sc-TMAPP, or
C) within the optional linker, at the N-terminus of the linker, or at the C-terminus of the linker when the optional linker is present.

3. The MOD-containing sc-TMAPP of claim 2, wherein the chemical conjugation site is selected from the group consisting of:
a) peptide sequence that acts as an enzyme modification sequence;
b) non-natural amino acids and/or selenocysteines;
c) engineered amino acid chemical conjugation sites;
d) carbohydrate or oligosaccharide covalently bound to the MOD-containing sc-TMAPP; and
e) IgG nucleotide binding sites.

4. The MOD-containing sc-TMAPP of claim 3, wherein the MHC Class II α1 polypeptide comprises an amino acid sequence having at least 95% amino acid sequence identity to either: the MHC Class II α1 polypeptide depicted in any one of SEQ ID NOs: 104, 122, 124, 126, 128 and 129; or to a polypeptide having at least 70 contiguous amino acids of any one of the MHC Class II α1 polypeptides depicted in any one of SEQ ID NOs: 104, 122, 124, 126, 128 and 129.

5. The MOD-containing sc-TMAPP of claim 4, wherein the MHC Class II α2 polypeptide comprises an amino acid sequence having at least 95% amino acid sequence identity to either: the MHC Class II α2 polypeptide depicted in any one of SEQ ID NOs: 104, 122, 124, 126, 128 and 129; or to a polypeptide having at least 70 contiguous amino acids of any one of the MHC Class II α2 polypeptides depicted in any one of SEQ ID NOs: 104, 122, 124, 126, 128 and 129.

6. The MOD-containing sc-TMAPP of claim 5, wherein the MHC Class II β1 polypeptide comprises an amino acid sequence having at least 95% amino acid sequence identity to either: the MHC Class II β1 polypeptide depicted in any one of SEQ ID NOs:107-118, 120-121, 123, 125, 127, and 130-133; or to a polypeptide having at least 70 contiguous amino acids of any one of the MHC Class II β1 polypeptide depicted in any one of SEQ ID NOs:107-118, 120-121, 123, 125, 127, and 130-133.

7. The MOD-containing sc-TMAPP of claim 6, wherein the MHC Class II β2 polypeptide comprises an amino acid sequence having at least 95% amino acid sequence identity to either: the MHC Class II β2 polypeptide depicted in any one of SEQ ID NOs:107-118, 120-121, 123, 125, 127, and 130-133; or to a polypeptide having at least 70 contiguous amino acids of any one of the MHC Class II β2 polypeptide depicted in any one of SEQ ID NOs:107-118, 120-121, 123, 125, 127, and 130-133.

8. The MOD-containing sc-TMAPP of claim 7, comprising one or more independently selected wild-type or variant MODs.

9. The MOD-containing sc-TMAPP of claim 8, wherein the wild type MODs are selected from the group consisting of: TGFβ, JAG1, IL-2, CD7, CD80, CD86, PD-L1, PD-L2, 4-1BBL, OX40L, FasL, ICOS-L, ICAM, CD30L, CD40, CD70, CD83, HLA-G, MICA, MICB, lymphotoxin beta receptor, 3/TR6, ILT3, ILT4, and HVEM, and at least one of the one or more MODs is a variant MOD thereof comprising:
an amino acid sequence having from 1 to 10 amino acid substitutions, deletions or insertions relative to a polypeptide comprising at least 70 contiguous amino acids of a wild-type MOD; or
an amino acid sequence having at least 95% amino acid sequence identity to a polypeptide comprising at least 60 contiguous amino acids of the wild-type MOD; and
wherein the variant MOD has reduced affinity for a Co-MOD, compared to the affinity of the naturally-occurring MOD for the Co-MOD where
the ratio of: i) the binding affinity of a control MOD-containing sc-TMAPP comprising a wild-type MOD to a Co-MOD to ii) the binding affinity of a MOD-containing sc-TMAPP comprising a variant of the wild-type MOD to the Co-MOD, when measured by bio-layer interferometry (BLI), is at least 2:1;
or
the ratio of: i) the binding affinity of a control MOD-containing sc-TMAPP comprising a wild-type MOD to a Co-MOD to ii) the binding affinity of a MOD-containing sc-TMAPP comprising a variant of the wild-type MOD to the Co-MOD, when measured by BLI, is in a range of from 1.5:1 to 10⁶:1.

10. The MOD-containing sc-TMAPP of claim 7, wherein the variant MOD is a variant of:
a 4-1BBL polypeptide of SEQ ID NOs:22, 23, 24, or 25;
a CD80 polypeptide of SEQ ID NO:16;
an IL-2 polypeptide of SEQ ID NO:29;
a CD86 polypeptide of SEQ ID NO:20; or
a PD-L1 polypeptide of SEQ ID NO:14 or SEQ ID NO:15.

11. The MOD-containing sc-TMAPP of claim 9, wherein the chemical conjugation site is:
a sulfatase motif; a Sortase A enzyme site; or a transglutaminase site;
a non-natural amino acid or a selenocysteine; or
an engineered amino acid chemical conjugation site.

12. The MOD-containing sc-TMAPP of claim 9, further comprising an epitope covalently bound directly, or indirectly through the optional linker, to the chemical conjugation site to form a MOD-containing sc-TMAPP-epitope conjugate.

13. The MOD-containing sc-TMAPP of claim 3, further comprising an epitope covalently bound directly, or indirectly through the optional linker, to the chemical conjugation site to form a sc-TMAPP-epitope conjugate.

14. The MOD-containing sc-TMAPP-epitope conjugate of claim 13, wherein the epitope is a cancer epitope, a virus epitope, or an auto-epitope.

15. A composition comprising:
a) the MOD-containing sc-TMAPP-epitope conjugate of claim 14; and
b) a buffer or a pharmaceutically acceptable excipient.

16. A method comprising: administering to an individual in need thereof an effective amount of the MOD-containing sc-TMAPP-epitope conjugate of claim 14, wherein said administering treats the individual.

17. The method of claim 16, wherein the individual has:
cancer, and wherein said administering treats the cancer;
a viral infection, and said administering treats the viral infection; or
an autoimmune disorder, and said administering treats the autoimmune disorder.

18. A MOD-less TMAPP, selected from
(I) a MOD-less m-TMAPP comprising:
a) a first polypeptide comprising:
i) a first MHC Class II polypeptide; and
b) a second polypeptide comprising:
i) a second MHC Class II polypeptide; and
ii) optionally an immunoglobulin (Ig) Fc polypeptide or a non-Ig scaffold;
wherein at least one of the first polypeptide or the second polypeptide comprises one or more chemical conjugation sites
A) at the N-terminus of the first polypeptide,
B) at the N-terminus of the second polypeptide, or
C) within the first or second polypeptide;
or
wherein at least one of the first polypeptide or the second polypeptide further comprises a linker comprising one or more chemical conjugation sites within the linker, at the N-terminus of the linker, or at the C-terminus of the linker; or
(II) a MOD-less sc-TMAPP comprising:
i) a Class II MHC α1 polypeptide;
ii) a Class II MHC α2 polypeptide;
iii) a Class II MHC β1 polypeptide;
iv) a Class II MHC β2 polypeptide;
v) an optional linker capable of being bound by a T-cell receptor (TCR);
vi) optionally an immunoglobulin (Ig) Fc polypeptide or a non-Ig scaffold; and
(vii) one or more chemical conjugations sites.

19. (canceled)

20. The MOD-containing m-TMAPP of claim 1, further comprising an epitope covalently bound directly, or indirectly through the optional linker, to the chemical conjugation site to form a MOD-containing m-TMAPP-epitope conjugate;
wherein the chemical conjugation site is selected from the group consisting of:
a) peptide sequence that acts as an enzyme modification sequence;
b) non-natural amino acids and/or selenocysteines;
c) engineered amino acid chemical conjugation sites;
d) carbohydrate or oligosaccharide covalently bound to the MOD-containing m-TMAPP; and
e) IgG nucleotide binding sites.

21. A method comprising: administering to an individual in need thereof an effective amount of the MOD-containing m-TMAPP-epitope conjugate of claim 20, wherein said administering treats the individual.

22. A nucleic acid encoding at least one of the first or second polypeptides of an unconjugated m-TMAPP polypeptide of claim 1.

23. A nucleic acid comprising nucleotide sequences encoding an unconjugated sc-TMAPP polypeptide of claim 2.
